SEQUENCE LISTING (1 sequence)

SEQ ID NO: 1 - protein, 26 aa, Artificial Sequence
  Synthetic Polypeptide
FAMAHAKEEQSQITSQVTGQIGWRNH

(19) United States
(12) Patent Application Publication
(10) Pub. No.: US 2017/0239249 A1
(43) Pub. Date: Aug. 24, 2017

(54) QUINAZOLINE-BASED KINASE INHIBITORS

(71) Applicant: Bristol-Myers Squibb Company, Princeton, NJ (US)

(72) Inventors: Richard A. HARTZ, Middletown, CT (US); Vijay T. AHUJA, Princeton, NJ (US); John E. MACOR, Washington Crossing, PA (US); Joanne J. BRONSON, Durham, CT (US); Bireshwar DASGUPTA, East Hamptom, CT (US); Carolyn Diane DZIERBA, Middletown, CT (US); Susheel Jethanand NARA, Mumbai (IN); Maheswaran Sivasamban KARATHOLUVHU, Bangalore (IN)

(21) Appl. No.: 15/514,728

(22) PCT Filed: Sep. 25, 2015
(86) PCT No.: PCT/US2015/052335

§ 371 Date: Mar. 27, 2017

Related U.S. Application Data
(60) Provisional application No. 62/057,640, filed on Sep. 30, 2014.

(51) Int. Cl. A61K 31/517

(52) U.S. Cl. CPC A61K 31/517

(57) ABSTRACT

The present disclosure is generally directed to compounds which can inhibit AAK1 (adaptor associated kinase 1), compositions comprising such compounds, and methods for inhibiting AAK1.

### CROSS-REFERENCE TO RELATED APPLICATIONS

This application claims priority to U.S. Provisional Patent Application 62/057,640 filed Sep. 30, 2014, hereby incorporated by reference in its entirety.

The present disclosure is generally directed to compounds which can inhibit adaptor associated kinase 1 (AAK1), compositions comprising such compounds, and methods for inhibiting AAK1.

Adaptor associated kinase 1 (AAK1) is a member of the Ark1/Prk1 family of serine/threonine kinases. AAK1 mRNA exists in two splice forms termed short and long. The long form predominates and is highly expressed in brain and heart (Henderson and Conner, Mol. Biol. Cell. 2007, 18, 2698-2706). AAK1 is enriched in synaptosomal preparations and is co-localized with endocytic structures in cultured cells. AAK1 modulates clatherin coated endocytosis, a process that is important in synaptic vesicle recycling and receptor-mediated endocytosis. AAK1 associates with the AP2 complex, a hetero-tetramer which links receptor cargo to the clatherin coat. The binding of clatherin to AAK1 stimulates AAK1 kinase activity (Conner et. al., Traffic 2003, 4, 885-890; Jackson et. al., J. Cell. Biol. 2003, 163, 231-236). AAK1 phosphorylates the mu-2 subunit of AP-2, which promotes the binding of mu-2 to tyrosine containing sorting motifs on cargo receptors (Ricotta et. al., J. Cell Bio. 2002, 156, 791-795; Conner and Schmid, J. Cell Bio. 2002, 156, 921-929). Mu2 phosphorylation is not required for receptor uptake, but phosphorylation enhances the efficiency of internalization (Motely et. al., Mol. Biol. Cell. 2006, 17, 5298-5308).

AAK1 has been identified as an inhibitor of Neuregulin-1/ErbB4 signaling in PC12 cells. Loss of AAK1 expression through RNA interference mediated gene silencing or treatment with the kinase inhibitor K252a (which inhibits AAK1 kinase activity) results in the potentiation of Neuregulin-1 induced neurite outgrowth. These treatments result in increased expression of ErbB4 and accumulation of ErbB4 in or near the plasma membrane (Kuai et. al., Chemistry and Biology 2011, 18, 891-906). NRG1 and ErbB4 are putative schizophrenia susceptibility genes (Buonanno, Brain Res. Bull. 2010, 83, 122-131). SNPs in both genes have been associated with multiple schizophrenia endophenotypes (Greenwood et. al., Am. J. Psychiatry 2011, 168, 930-946). Neuregulin 1 and ErbB4 KO mouse models have shown schizophrenia relevant morphological changes and behavioral phenotypes (Jaaro-Peled et. al., Schizophrenia Bulletin 2010, 36, 301-313; Wen et. al., Proc. Natl. Acad. Sci. USA. 2010, 107, 1211-1216). In addition, a single nucleotide polymorphism in an intron of the AAK1 gene has been associated with the age of onset of Parkinson's disease (Latourelle et. al., BMC Med. Genet. 2009, 10, 98). These results suggest that inhibition of AAK1 activity may have utility in the treatment of schizophrenia, cognitive deficits in schizophrenia, Parkinson's disease, neuropathic pain, bipolar disorder, and Alzheimer's disease.

In a first aspect the present disclosure provides a method for treating or managing a disease or a disorder mediated by AAK1 activity, the method comprising administering to a patient in need thereof a therapeutically effective amount of a compound of formula (I)

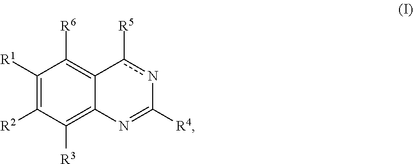

or a pharmaceutically acceptable salt thereof, wherein:

R¹ is selected from imidazopyridazine, isoquinolinyl, oxazolyl, pyridinyl, pyrimidinyl, pyrazolyl, pyrrolopyridinyl, and quinolinyl, wherein each ring is optionally substituted with C₁-C₃acylamino, C₁-C₃alkyl, amino, C₁-C₃alkoxy, C₁-C₃alkylamino, C₃-C₆cycloalkyl, C₃-C₆cycloalkylamino, C₁-C₃dialkylamino, —NHCO₂(C₁-C₃)alkyl, and phenylcarbonylamino optionally substituted with a halo or haloalkyl group;

R² is selected from hydrogen, C₁-C₃alkoxy, and C₁-C₃alkyl;

R³ is selected from hydrogen, C₁-C₃alkoxy, C₁-C₃alkyl, cyano, and halo;

R⁴ is selected from C₃-C₆alkyl optionally substituted with one or two groups independently selected from amino, haloalkyloxy, haloalkyl, hydroxy and oxo; and C₃-C₆cycloalkylC₁-C₃alkyl optionally substituted with amino;

when [symbol] N is a single bond, R⁵ is ═S or ═O.

when [symbol] N is a double bond, R⁵ is selected from hydrogen, C₁-C₆alkoxy, C₁-C₃alkoxyC₁-C₃alkyl, C₁-C₃alkoxyC₁-C₃alkylamino, C₁-C₃alkoxycarbonylC₁-C₃alkylamino, C₁-C₆alkyl, C₁-C₆alkylamino, C₁-C₆alkylsulfanyl, amido, aminoC₁-C₃alkylamino, cyano, C₃-C₆cycloalkyl, C₁-C₆dialkylamido, C₁-C₆dialkylamino, C₁-C₆dialkylaminoC₁-C₃alkylamino, halo, hydroxyC₁-C₃alkyl, hydroxyC₁-C₃alkylamino, pyrrolidinylC₁-C₃alkylamino, pyrazinylC₁-C₃alkylamino optionally substituted with methyl, and a ring selected from

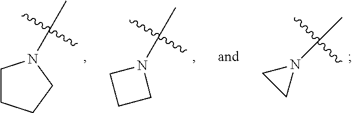

and
R⁶ is hydrogen or C₁-C₃alkoxy.

In a first embodiment of the first aspect R¹ is selected from oxazolyl, pyridinyl, and pyrazolyl, wherein each ring is optionally substituted with C₁-C₃acylamino, C₁-C₃alkoxy, and C₁-C₃alkylamino. In a second embodiment R² and R³ are selected from hydrogen and C₁-C₃alkoxy. In a third embodiment R⁴ is selected from C₃-C₆alkyl optionally substituted with one or two groups independently selected from amino, and haloalkyl; and C₃-C₆cycloalkylC₁-C₃alkyl optionally substituted with amino.

In a fourth embodiment of the first aspect the disease or disorder is selected from Alzheimer's disease, bipolar disorder, pain, Parkinson's disease, and schizophrenia. In a fifth embodiment the pain is neuropathic pain. In a sixth embodiment the neuropathic pain is fibromyalgia or peripheral neuropathy.

In a second aspect the present disclosure provides a method of inhibiting adaptor associated kinase 1 (AAK1) activity, comprising contacting AAK1 with a compound of formula (I)

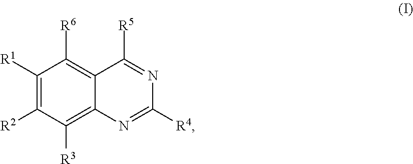

or a pharmaceutically acceptable salt thereof, wherein:

R¹ is selected from imidazopyridazine, isoquinolinyl, oxazolyl, pyridinyl, pyrimidinyl, pyrazolyl, pyrrolopyridinyl, and quinolinyl, wherein each ring is optionally substituted with C₁-C₃acylamino, C₁-C₃alkyl, amino, C₁-C₃alkoxy, C₁-C₃alkylamino, C₃-C₆cycloalkyl, C₃-C₆cycloalkylamino, C₁-C₃dialkylamino, —NHCO₂(C₁-C₃)alkyl, and phenylcarbonylamino optionally substituted with a halo or haloalkyl group;

R² is selected from hydrogen, C₁-C₃alkoxy, and C₁-C₃alkyl;

R³ is selected from hydrogen, C₁-C₃alkoxy, C₁-C₃alkyl, cyano, and halo;

R⁴ is selected from C₃-C₆alkyl optionally substituted with one or two groups independently selected from amino, haloalkyloxy, haloalkyl, hydroxy and oxo; and C₃-C₆cycloalkylC₁-C₃alkyl optionally substituted with amino;

when

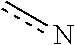

is a single bond, R⁵ is ═S or ═O.

when

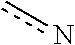

is a double bond, R⁵ is selected from hydrogen, C₁-C₆alkoxy, C₁-C₃alkoxyC₁-C₃alkyl, C₁-C₃alkoxyC₁-C₃alkylamino, C₁-C₃alkoxycarbonylC₁-C₃alkylamino, C₁-C₆alkyl, C₁-C₆alkylamino, C₁-C₆alkylsulfanyl, amido, aminoC₁-C₃alkylamino, cyano, C₃-C₆cycloalkyl, C₁-C₆dialkylamido, C₁-C₆dialkylamino, C₁-C₆dialkylaminoC₁-C₃alkylamino, halo, hydroxyC₁-C₃alkyl, hydroxyC₁-C₃alkylamino, pyrrolidinylC₁-C₃alkylamino, pyrazinylC₁-C₃alkylamino optionally substituted with methyl, and a ring selected from

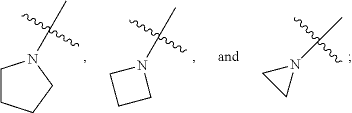

and
R⁶ is hydrogen or C₁-C₃alkoxy.

Other aspects of the present disclosure may include suitable combinations of embodiments disclosed herein.

Yet other aspects and embodiments may be found in the description provided herein.

This disclosure is based, in part, on the discovery that AAK1 knockout mice exhibit a high resistance to pain. That discovery prompted research that ultimately led to the discovery of AAK1 inhibitors, compositions comprising them, and methods of their use.

The description of the present disclosure herein should be construed in congruity with the laws and principals of chemical bonding. In some instances it may be necessary to remove a hydrogen atom in order to accommodate a substituent at any given location.

It should be understood that the compounds encompassed by the present disclosure are those that are suitably stable for use as pharmaceutical agent.

As used in the present specification, the following terms have the meanings indicated:

All patents, patent applications, and literature references cited in the specification are herein incorporated by reference in their entirety. In the case of inconsistencies, the present disclosure, including definitions, will prevail.

As used herein, the singular forms “a”, “an”, and “the” include plural reference unless the context clearly dictates otherwise.

In some instances, the number of carbon atoms in any particular group is denoted before the recitation of the group. For example, the term “C₁₋₆ alkyl” denotes an alkyl group containing one to six carbon atoms. Where these designations exist they supercede all other definitions contained herein.

The term “acylamino,” as used herein, refers to —NHC(O)R wherein R is an alkyl group.

The term “alkoxy,” as used herein, refers to an alkyl group attached to the parent molecular moiety through an oxygen atom.

The term “alkoxyalkyl,” as used herein, refers to an alkoxy group attached to the parent molecular moiety through an alkyl group.

The term “alkoxyalkylamino,” as used herein, refers to —NHR, wherein R is an alkoxyalkyl group.

The term “alkoxycarbonyl,” as used herein, refers to an alkoxy group attached to the parent molecular moiety through a carbonyl group.

The term “alkoxycarbonylalkyl,” as used herein, refers to an alkoxycarbonyl group attached to the parent molecular moiety through an alkyl group.

The term “alkoxycarbonylalkylamino,” as used herein, refers to —NHR, wherein R is an alkoxycarbonylalkyl group.

The term “alkyl,” as used herein, refers to a group derived from a straight or branched chain saturated hydrocarbon.

The term “alkylamino,” as used herein, refers to —NHR wherein R is an alkyl group.

The term “alkylsulfanyl,” as used herein, refers to an alkyl group attached to the parent molecular moiety through a sulfur atom.

The term “amido,” as used herein, refers to —C(O)NH₂.

The term “amino,” as used herein, refers to —NH₂.

The term “aminoalkyl,” as used herein, refers to an amino group attached to the parent molecular moiety through an alkyl group.

The term “aminoalkylamino,” as used herein, refers to —NHR, wherein R is an aminoalkyl group.

The term “carbonyl,” as used herein, refers to —C(O)—.

The term “cyano,” as used herein, refers to —CN.

The term “cycloalkyl,” as used herein, refers to a saturated monocyclic hydrocarbon ring system having zero heteroatoms. Representative examples of cycloalkyl groups include, but are not limited to, cyclopropyl, cyclopentyl, and cyclohexyl.

The term “cycloalkylalkyl,” as used herein, refers to a cycloalkyl group attached to the parent molecular moiety through an alkyl group.

The term “cycloalkylamino,” as used herein, refers to —NHR wherein R is a cycloalkyl group.

The term “dialkylamido,” as used herein, refers to —C(O)NR₂, wherein each R is an alkyl group. The two alkyl groups are the same or different.

The term “dialkylamino,” as used herein, refers to NR₂, wherein each R is an alkyl group. The two alkyl groups are the same or different.

The term “dialkylaminoalkyl,” as used herein, refers to a dialkylamino group attached to the parent molecular moiety through an alkyl group.

The term “dialkylaminoalkylamino,” as used herein, refers to —NHR wherein R is a dialkylaminoalkyl group.

The term “halo,” as used herein, refers to Br, Cl, F, and/or I.

The term “haloalkoxy,” as used herein, refers to a haloalkyl group attached to the parent molecular moiety through an oxygen atom.

The term “haloalkoxyalkyl,” as used herein, refers to a haloalkoxy group attached to the parent molecular moiety through an alkyl group.

The term “haloalkyl,” as used herein, refers to an alkyl group substituted by one, two, three, or four halogen atoms.

The term “hydroxy,” as used herein, refers to —OH.

The term “hydroxyalkyl,” as used herein, refers to a hydroxy group attached to the parent molecular moiety through an alkyl group.

The term “hydroxyalkylamino,” as used herein, refers to —NHR, wherein R is a hydroxyalkyl group.

The term “oxo,” as used herein, refers to ═O.

The term “phenylcarbonylamino,” as used herein, refers to —NHC(O)-Ph, wherein Ph is a phenyl group.

The term “pyrazinylalkyl,” as used herein, refers to a pyrazinyl group attached to the parent molecular moiety through an alkyl group.

The term “pyrazinylalkylamino,” as used herein, refers to —NHR, wherein R is a pyrazinylalkyl group.

The term “pyrrolidinylalkyl,” as used herein, refers to a pyrrolidinyl group attached to the parent molecular moiety through an alkyl group.

The term “pyrrolidinylalkylamino,” as used herein, refers to —NHR, wherein R is a pyrrolidinylalkyl group.

Asymmetric centers may exist in the compounds of the present disclosure. It should be understood that the disclosure encompasses all stereochemical isomeric forms, or mixtures thereof, which possess the ability to inhibit AAK1. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, or direct separation of enantiomers on chiral chromatographic columns.

Starting compounds of particular stereochemistry are either commercially available or can be made and resolved by techniques known in the art.

Certain compounds of the present disclosure may also exist in different stable conformational forms which may be separable. Torsional asymmetry due to restricted rotation about an asymmetric single bond, for example because of steric hindrance or ring strain, may permit separation of different conformers. The present disclosure includes each conformational isomer of these compounds and mixtures thereof.

The term “compounds of the present disclosure”, and equivalent expressions, are meant to embrace compounds of formula (I), and pharmaceutically acceptable enantiomers, diastereomers, and salts thereof. Similarly, references to intermediates are meant to embrace their salts where the context so permits.

The present disclosure is intended to include all isotopes of atoms occurring in the present compounds. Isotopes include those atoms having the same atomic number but different mass numbers. By way of general example and without limitation, isotopes of hydrogen include deuterium and tritium. Isotopes of carbon include ¹³C and ¹⁴C. Isotopically-labeled compounds of the disclosure can generally be prepared by conventional techniques known to those skilled in the art or by processes analogous to those described herein, using an appropriate isotopically-labeled reagent in place of the non-labeled reagent otherwise employed. Such compounds may have a variety of potential uses, for example as standards and reagents in determining biological activity. In the case of stable isotopes, such compounds may have the potential to favorably modify biological, pharmacological, or pharmacokinetic properties.

The compounds of the present disclosure can exist as pharmaceutically acceptable salts. The term “pharmaceutically acceptable salt,” as used herein, represents salts or zwitterionic forms of the compounds of the present disclosure which are water or oil-soluble or dispersible, which are, within the scope of sound medical judgment, suitable for use in contact with the tissues of patients without excessive toxicity, irritation, allergic response, or other problem or complication commensurate with a reasonable benefit/risk ratio, and are effective for their intended use. The salts can be prepared during the final isolation and purification of the compounds or separately by reacting a suitable nitrogen atom with a suitable acid.

Representative acid addition salts include acetate, adipate, alginate, citrate, aspartate, benzoate, benzenesulfonate, bisulfate, butyrate, camphorate, camphorsulfonate; digluconate, dihydrobromide, diydrochloride, dihydroiodide, glycerophosphate, hemisulfate, heptanoate, hexanoate, formate, fumarate, hydrochloride, hydrobromide, hydroiodide, 2-hydroxyethanesulfonate, lactate, maleate, mesitylenesulfonate, methanesulfonate, naphthylenesulfonate, nicotinate, 2-naphthalenesulfonate, oxalate, palmoate, pectinate, persulfate, 3-phenylproprionate, picrate, pivalate, propionate, succinate, tartrate, trichloroacetate, trifluoroacetate, phosphate, glutamate, bicarbonate, para-toluenesulfonate, and undecanoate. Examples of acids which can be employed to form pharmaceutically acceptable addition salts include inorganic acids such as hydrochloric, hydrobromic, sulfuric, and phosphoric, and organic acids such as oxalic, maleic, succinic, and citric.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of pharmaceutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, and N,N′-dibenzylethylenediamine. Other representative organic amines useful for the formation of base addition salts include ethylenediamine, ethanolamine, diethanolamine, piperidine, and piperazine.

One embodiment of this disclosure encompasses methods of inhibiting adaptor associated kinase 1 (AAK1), both in vitro and in vivo, which comprise contacting AAK1 with a compound of formula I or a pharmaceutically acceptable salt thereof.

When it is possible that, for use in therapy, therapeutically effective amounts of a compound of formula (I), as well as pharmaceutically acceptable salts thereof, may be administered as the raw chemical, it is possible to present the active ingredient as a pharmaceutical composition. Accordingly, the disclosure further provides pharmaceutical compositions, which include therapeutically effective amounts of compounds of formula (I) or pharmaceutically acceptable salts thereof, and one or more pharmaceutically acceptable carriers, diluents, or excipients.

Unless otherwise indicated, a “therapeutically effective amount” of a compound is an amount sufficient to provide a therapeutic benefit in the treatment or management of a disease or condition, or to delay or minimize one or more symptoms associated with the disease or condition. The term “therapeutically effective amount” can encompass an amount that improves overall therapy, reduces or avoids symptoms or causes of a disease or condition, or enhances the therapeutic efficacy of another therapeutic agent.

The term “therapeutically effective amount,” as used herein, refers to an amount of a compound or compounds sufficient to provide a therapeutic benefit in the treatment or management of a disease or condition, or to delay or minimize one or more symptoms associated with the disease or condition. A “therapeutically effective amount” of a compound means an amount of therapeutic agent, alone or in combination with other therapies, that provides a therapeutic benefit in the treatment or management of the disease or condition. The term “therapeutically effective amount” can encompass an amount that improves overall therapy, reduces or avoids symptoms or causes of a disease or condition, or enhances the therapeutic efficacy of another therapeutic agent. When applied to an individual active ingredient, administered alone, the term refers to that ingredient alone. When applied to a combination, the term refers to combined amounts of the active ingredients that result in the therapeutic effect, whether administered in combination, serially, or simultaneously. The compounds of formula (I) and pharmaceutically acceptable salts thereof, are as described above. The carrier(s), diluent(s), or excipient(s) must be acceptable in the sense of being compatible with the other ingredients of the formulation and not deleterious to the recipient thereof. In accordance with another aspect of the present disclosure there is also provided a process for the preparation of a pharmaceutical formulation including admixing a compound of formula (I), or a pharmaceutically acceptable salt thereof, with one or more pharmaceutically acceptable carriers, diluents, or excipients. The term “pharmaceutically acceptable,” as used herein, refers to those compounds, materials, compositions, and/or dosage forms which are, within the scope of sound medical judgment, suitable for use in contact with the tissues of patients without excessive toxicity, irritation, allergic response, or other problem or complication commensurate with a reasonable benefit/risk ratio, and are effective for their intended use.

Pharmaceutical formulations may be presented in unit dose forms containing a predetermined amount of active ingredient per unit dose. Dosage levels of between about 0.01 and about 250 milligram per kilogram (“mg/kg”) body weight per day, preferably between about 0.05 and about 100 mg/kg body weight per day of the compounds of the present disclosure are typical in a monotherapy for the prevention and treatment of disease. Typically, the pharmaceutical compositions of this disclosure will be administered from about 1 to about 5 times per day or alternatively, as a continuous infusion. Such administration can be used as a chronic or acute therapy. The amount of active ingredient that may be combined with the carrier materials to produce a single dosage form will vary depending on the condition being treated, the severity of the condition, the time of administration, the route of administration, the rate of excretion of the compound employed, the duration of treatment, and the age, gender, weight, and condition of the patient. Preferred unit dosage formulations are those containing a daily dose or sub-dose, as herein above recited, or an appropriate fraction thereof, of an active ingredient. Treatment may be initiated with small dosages substantially less than the optimum dose of the compound. Thereafter, the dosage is increased by small increments until the optimum effect under the circumstances is reached. In general, the compound is most desirably administered at a concentration level that will generally afford effective results without causing any harmful or deleterious side effects.

When the compositions of this disclosure comprise a combination of a compound of the present disclosure and one or more additional therapeutic or prophylactic agent, both the compound and the additional agent are usually present at dosage levels of between about 10 to 150%, and more preferably between about 10 and 80% of the dosage normally administered in a monotherapy regimen.

Compounds of the disclosure may be administered in combination with one or more additional therapeutic or prophylactic agents. For example, when used for the treatment of pain, possible additional agents include immunosuppressive agents, anti-inflammatory agents, and/or other agents used in the treatment of pain.

Immunosuppressants suitable for use in the methods and compositions of this disclosure include those known in the art. Examples include aminopterin, azathioprine, cyclosporin A, D-penicillamine, gold salts, hydroxychloroquine, leflunomide, methotrexate, minocycline, rapamycin, sulfasalazine, tacrolimus (FK506), and pharmaceutically acceptable salts thereof. A particular immunosuppressant is methotrexate.

Additional examples of immunosuppressants include anti-TNF antibodies, such as adalimumab, certolizumab pegol, etanercept, and infliximab. Others include interleukin-1 blockers, such as anakinra. Others include anti-B cell (CD20) antibodies, such as rituximab. Others include T cell activation blockers, such as abatacept.

Other immunosuppressants include inosine monophosphate dehydrogenase inhibitors, such as mycophenolate mofetil (CellCept®) and mycophenolic acid (Myfortic®).

Anti-inflammatory drugs suitable for use in the methods and compositions of this disclosure include those known in the art. Examples include glucocorticoids and NSAIDs. Examples of glucocorticoids include aldosterone, beclometasone, betamethasone, cortisone, deoxycorticosterone, dexamethasone, fludrocortisones, hydrocortisone, methylprednisolone, prednisolone, prednisone, triamcinolone, and pharmaceutically acceptable salts thereof.

Examples of NSAID include salicylates (e.g., aspirin, amoxiprin, benorilate, choline magnesium salicylate, diflunisal, faislamine, methyl salicylate, magnesium salicylate, salicyl salicylate, and pharmaceutically acceptable salts thereof), arylalkanoic acids (e.g., diclofenac, aceclofenac, acemetacin, bromfenac, etodolac, indometacin, nabumetone, sulindac, tolmetin, and pharmaceutically acceptable salts thereof), arylpropionic acids (e.g., ibuprofen, carprofen, fenbufen, fenoprofen, flurbiprofen, ketoprofen, ketorolac, loxoprofen, naproxen, oxaprozin, tiaprofenic acid, suprofen, and pharmaceutically acceptable salts thereof), arylanthranilic acids (e.g., meclofenamic acid, mefenamic acid, and pharmaceutically acceptable salts thereof), pyrazolidine derivatives (e.g., azapropazone, metamizole, oxyphenbutazone, phenylbutazone, sulfinprazone, and pharmaceutically acceptable salts thereof), oxicams (e.g., lornoxicam, meloxicam, piroxicam, tenoxicam, and pharmaceutically acceptable salts thereof), COX-2 inhibitors (e.g., celecoxib, etoricoxib, lumiracoxib, parecoxib, rofecoxib, valdecoxib, and pharmaceutically acceptable salts thereof), and sulphonanilides (e.g., nimesulide and pharmaceutically acceptable salts thereof).

Other agents used in the treatment of pain (including but not limited to neuropathic and inflammatory pain) include, but are not limited to, agents such as pregabalin, lidocaine, duloxetine, gabapentin, carbamazepine, capsaicin, and other serotonin/norepinephrine/dopamine reuptake inhibitors, and opiates (such as oxycontin, morphine, and codeine).

In the treatment of pain caused by a known disease or condition, such as diabetes, infection (e.g., herpes zoster or HIV infection), or cancer, compounds of the disclosure may be administered in combination with one or more additional therapeutic or prophylactic agents directed at the underlying disease or condition.

For example, when used to treat diabetic neuropathy, compounds of the disclosure may be administered in combination with one or more anti-diabetic agents, anti-hyperglycemic agents, hypolipidemic/lipid lowering agents, anti-obesity agents, anti-hypertensive agents and appetite suppressants. Examples of anti-diabetic agents include biguanides (e.g., metformin, phenformin), glucosidase inhibitors (e.g., acarbose, miglitol), insulins (including insulin secretagogues and insulin sensitizers), meglitinides (e.g., repaglinide), sulfonylureas (e.g., glimepiride, glyburide, gliclazide, chlorpropamide, and glipizide), biguanide/glyburide combinations (e.g., Glucovance), thiazolidinediones (e.g., troglitazone, rosiglitazone, and pioglitazone), PPAR-alpha agonists, PPAR-gamma agonists, PPAR alpha/gamma dual agonists, glycogen phosphorylase inhibitors, inhibitors of fatty acid binding protein (aP2), glucagon-like peptide-1 (GLP-1) or other agonists of the GLP-1 receptor, dipeptidyl peptidase IV (DPP4) inhibitors, and sodium-glucose co-transporter 2 (SGLT2) inhibitors (e.g., dapagliflozin, canagliflozin, and LX-42H).

Pharmaceutical formulations may be adapted for administration by any appropriate route, for example by the oral (including buccal or sublingual), rectal, nasal, topical (including buccal, sublingual, or transdermal), vaginal, or parenteral (including subcutaneous, intracutaneous, intramuscular, intra-articular, intrasynovial, intrasternal, intrathecal, intralesional, intravenous, or intradermal injections or infusions) route. Such formulations may be prepared by any method known in the art of pharmacy, for example by bringing into association the active ingredient with the carrier(s) or excipient(s). Oral administration or administration by injection are preferred.

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil emulsions.

For instance, for oral administration in the form of a tablet or capsule, the active drug component can be combined with an oral, non-toxic pharmaceutically acceptable inert carrier such as ethanol, glycerol, water, and the like. Powders are prepared by comminuting the compound to a suitable fine size and mixing with a similarly comminuted pharmaceutical carrier such as an edible carbohydrate, as, for example, starch or mannitol. Flavoring, preservative, dispersing, and coloring agent can also be present.

Capsules are made by preparing a powder mixture, as described above, and filling formed gelatin sheaths. Glidants and lubricants such as colloidal silica, talc, magnesium stearate, calcium stearate, or solid polyethylene glycol can be added to the powder mixture before the filling operation. A disintegrating or solubilizing agent such as agar-agar, calcium carbonate, or sodium carbonate can also be added to improve the availability of the medicament when the capsule is ingested.

Moreover, when desired or necessary, suitable binders, lubricants, disintegrating agents, and coloring agents can also be incorporated into the mixture.

Suitable binders include starch, gelatin, natural sugars such as glucose or beta-lactose, corn sweeteners, natural and synthetic gums such as acacia, tragacanth or sodium alginate, carboxymethylcellulose, polyethylene glycol, and the like.

Lubricants used in these dosage forms include sodium oleate, sodium chloride, and the like. Disintegrators include, without limitation, starch, methyl cellulose, agar, betonite, xanthan gum, and the like. Tablets are formulated, for example, by preparing a powder mixture, granulating or slugging, adding a lubricant and disintegrant, and pressing into tablets. A powder mixture is prepared by mixing the compound, suitable comminuted, with a diluent or base as described above, and optionally, with a binder such as carboxymethylcellulose, an aliginate, gelating, or polyvinyl pyrrolidone, a solution retardant such as paraffin, a resorption accelerator such as a quaternary salt and/or and absorption agent such as betonite, kaolin, or dicalcium phosphate. The powder mixture can be granulated by wetting with a binder such as syrup, starch paste, acadia mucilage, or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc, or mineral oil. The lubricated mixture is then compressed into tablets. The compounds of the present disclosure can also be combined with a free flowing inert carrier and compressed into tablets directly without going through the granulating or slugging steps. A clear or opaque protective coating consisting of a sealing coat of shellac, a coating of sugar or polymeric material, and a polish coating of wax can be provided. Dyestuffs can be added to these coatings to distinguish different unit dosages.

Oral fluids such as solution, syrups, and elixirs can be prepared in dosage unit form so that a given quantity contains a predetermined amount of the compound.

Syrups can be prepared by dissolving the compound in a suitably flavored aqueous solution, while elixirs are prepared through the use of a non-toxic vehicle.

Solubilizers and emulsifiers such as ethoxylated isostearyl alcohols and polyoxyethylene sorbitol ethers, preservatives, flavor additive such as peppermint oil or natural sweeteners, or saccharin or other artificial sweeteners, and the like can also be added.

Where appropriate, dosage unit formulations for oral administration can be microencapsulated. The formulation can also be prepared to prolong or sustain the release as for example by coating or embedding particulate material in polymers, wax, or the like.

The compounds of formula (I), and pharmaceutically acceptable salts thereof, can also be administered in the form of liposome delivery systems, such as small unilamellar vesicles, large unilamellar vesicles, and multilamellar vesicles.

Liposomes can be formed from a variety of phopholipids, such as cholesterol, stearylamine, or phophatidylcholines.

The compounds of formula (I) and pharmaceutically acceptable salts thereof may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropylmethacrylamidephenol, polyhydroxyethylaspartamidephenol, or polyethyleneoxidepolylysine substituted with palitoyl residues. Furthermore, the compounds may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates, and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research 1986, 3(6), 318.

Pharmaceutical formulations adapted for topical administration may be formulated as ointments, creams, suspensions, lotions, powders, solutions, pastes, gels, sprays, aerosols, or oils.

Pharmaceutical formulations adapted for rectal administration may be presented as suppositories or as enemas.

Pharmaceutical formulations adapted for nasal administration wherein the carrier is a solid include a course powder having a particle size for example in the range 20 to 500 microns which is administered in the manner in which snuff is taken, i.e., by rapid inhalation through the nasal passage from a container of the powder held close up to the nose. Suitable formulations wherein the carrier is a liquid, for administration as a nasal spray or nasal drops, include aqueous or oil solutions of the active ingredient.

Pharmaceutical formulations adapted for administration by inhalation include fine particle dusts or mists, which may be generated by means of various types of metered, dose pressurized aerosols, nebulizers, or insufflators.

Pharmaceutical formulations adapted for vaginal administration may be presented as pessaries, tampons, creams, gels, pastes, foams, or spray formulations.

Pharmaceutical formulations adapted for parenteral administration include aqueous and non-aqueous sterile injection solutions which may contain anti-oxidants, buffers, bacteriostats, and soutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets.

It should be understood that in addition to the ingredients particularly mentioned above, the formulations may include other agents conventional in the art having regard to the type of formulation in question, for example those suitable for oral administration may include flavoring agents.

The term “patient” includes both human and other mammals. Unless otherwise indicated, the terms “manage,” “managing”, and “management” encompass preventing the recurrence of the specified disease or disorder in a patient who has already suffered from the disease or disorder, and/or lengthening the time that a patient who has suffered from the disease or disorder remains in remission. The terms encompass modulating the threshold, development and/or duration of the disease or disorder, or changing the way that a patient responds to the disease or disorder.

The term “treating” refers to: (i) preventing a disease, disorder or condition from occurring in a patient that may be predisposed to the disease, disorder, and/or condition but has not yet been diagnosed as having it; (ii) inhibiting the disease, disorder, or condition, i.e., arresting its development; and (iii) relieving the disease, disorder, or condition, i.e., causing regression of the disease, disorder, and/or condition.

This disclosure is intended to encompass compounds having Formula (I) when prepared by synthetic processes or by metabolic processes including those occurring in the human or animal body (in vivo) or processes occurring in vitro.

The abbreviations used in the present application, including particularly in the illustrative schemes and examples which follow, are well-known to those skilled in the art. Some of the abbreviations used are as follows: BOC or Boc for tert-butoxycarbonyl; RT or rt or r.t. for room temperature or retention time (context will dictate); t_{R} for retention time; TosMIC (tosylmethyl isocyanide), HATU for O-(7-azabenzotriazol-1-yl)-N,N,N′,N′-tetramethyluronium hexafluorophosphate; BOP for benzotriazol-1-yloxytris(dimethylamino)phosphonium hexafluorophosphate; EDC or EDCI for 1-(3-dimethylaminopropyl)-3-ethylcarbodiimide hydrochloride; TBTU for O-benzotriazol-1-yl-N,N,N′,N′-tetramethyluronium tetrafluoroborate; SPhos for 2-dicyclohexylphosphino-2′,6′-dimethoxybiphenyl; XPhos for 2-dicyclohexylphosphino-2′,4′,6′-triisopropylbiphenyl; i-Pr or iPr for isopropyl; THF for tetrahydrofuran; EtOH for ethanol; Ac for acetyl; DMAP for N,N-dimethylaminopyridine; TEA or Et₃N for triethylamine; DIEA or i-Pr₂NEt for N,N-diisopropylethylamine; TPAP for tetrapropylammonium perruthenate; Me for methyl; TFA for trifluoroacetic acid; Ph for phenyl; DMF for N,N-dimethylformamide; DMSO for dimethylsulfoxide; NMP for N-methylpyrrolidine; MeCN for acetonitrile; DIAD for diisopropyl azodicarboxylate; HOBt for 1-hydroxybenzotriazole; dppf for 1,1′-bis(diphenylphosphanyl) ferrocene; Et for ethyl; h or hr or hrs for hours; min or mins for minutes; EtOAc for ethyl acetate; DCM for dichloromethane; MeOH for methanol; DME for dimethoxyethane; AcOH for acetic acid; and MeOD for CD₃OD.

### EXAMPLES

The present disclosure will now be described in connection with certain embodiments which are not intended to limit its scope. On the contrary, the present disclosure covers all alternatives, modifications, and equivalents as can be included within the scope of the claims. Thus, the following examples, which include specific embodiments, will illustrate one practice of the present disclosure, it being understood that the examples are for the purposes of illustration of certain embodiments and are presented to provide what is believed to be the most useful and readily understood description of its procedures and conceptual aspects.

The compounds of the present disclosure may be prepared using the reactions and techniques described in this section as well as other synthetic methods known to those of ordinary skill in the art. The reactions are performed in solvents appropriate to the reagents and materials employed and suitable for the transformation being affected. Also, in the description of the synthetic methods described below, it is to be understood that all proposed reaction conditions, including choice of solvents, reaction temperature, duration of the experiment and workup procedures, are chosen to be the conditions standard for that reaction, which should be readily recognized by one skilled in the art. It is understood by one skilled in the art of organic synthesis that the functionality present on various portions of the molecule must be compatible with the reagents and reactions proposed. Such restrictions to the substituents which are compatible with the reaction conditions will be readily apparent to one skilled in the art and alternate methods must then be used.

An intermediate (7) for the synthesis of compounds of formula (I), wherein R¹=oxazol-5-yl, can be prepared by the methods shown in Scheme I. Treatment of 2, prepared as described by Rao et al. (Tetrahedron Lett., 1992, 33, 4799-4802), with TosMIC in the presence of a base such as potassium carbonate in a solvent such as methanol affords compound 3. The bromide of 3 is replaced with a vinyl group by via a palladium catalyzed coupling reaction using reaction conditions familiar to those skilled in the art, such as a Suzuki reaction, Stille reaction, or Negishi reaction.

Reaction conditions include reaction of 3 with 2,4,6-trivinyl-1,3,5,2,4,6-trioxatriborinane pyridine complex in the presence of a base such as sodium carbonate, potassium carbonate, or cesium carbonate and a catalyst such as Pd(PPh₃)₄, Pd₂(dba)₃, or PdCl₂(PPh₃)₂ in a solvent such as toluene, dichloroethane, THF, DMF, methanol, ethanol, water or a combination thereof at temperatures ranging from 20° C. to 150° C. to form compound 4. The coupling reaction can be carried out by heating the reaction mixture using standard laboratory methods or by heating the reaction mixture in a microwave. Reduction of the nitro group in 4 is accomplished using standard conditions such as, but not limited to, H₂ and Pd/C, zinc with ammonium chloride, or tin chloride in an appropriate solvent such as methanol or ethanol at temperatures ranging from 0° C. to 100° C. to give compound 5.

Compound 5 can be coupled with a carboxylic acid (R⁴CO₂H) using standard peptide coupling reagents such as HATU, BOP, EDC, or TBTU in the presence of a base such as N,N-diisopropylethylamine and a solvent such as THF at temperatures ranging from 20° C. to 80° C. to form compounds of formula 6. Compound 5 can also be coupled with a carboxylic acid (R⁴CO₂H) under conditions wherein the acid is converted to an acid chloride in situ. Reaction of the acid with N-(chloromethylene)-N-methylmethanaminium, chloride salt, prepared as described by Jarrahpour et al. (Tetrahedron 2009, 65, 2927-2934), in the presence of triethylamine or reaction of the acid with phosphorus oxychloride in the presence of pyridine and compound 5 leads to the formation of compounds of formula 6. Alternatively, compound 5 can be coupled directly with an acid chloride (R⁴C(O)Cl) to form compounds of formula 6. Oxidation of the olefin in 6 using osmium tetroxide and sodium periodate in the presence of 2,6-lutidine or using ozone furnishes compounds of formula 7.

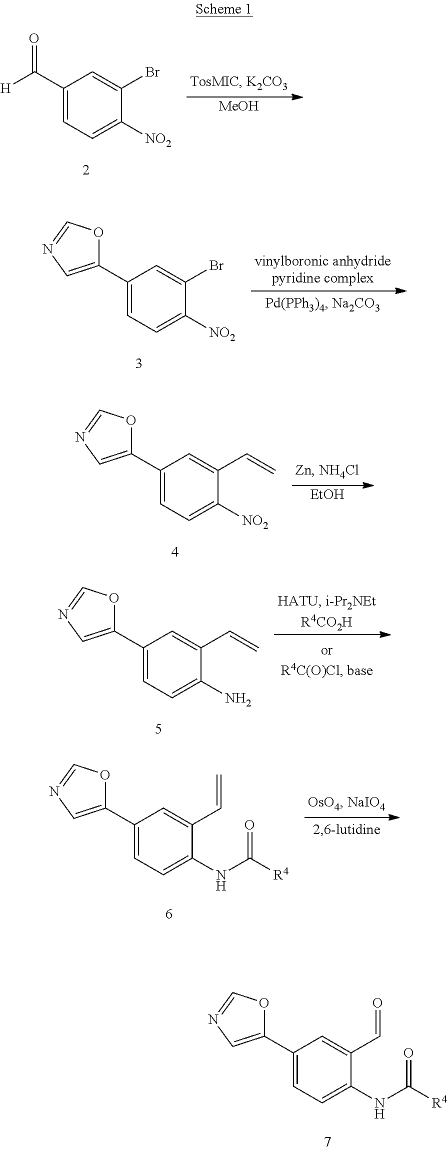

Compounds of formula (I), wherein R¹=oxazol-5-yl and R⁵═H, can be prepared from compounds of formula 7 by the methods shown in Scheme 2. Heating compounds of formula 7 in the presence of ammonium acetate and acetic acid at temperatures ranging from 50 to 120° C. furnishes compounds of formula 8. In many cases, the R⁴ group of compound 8 contains a protected amine. Preferred protecting groups are tert-butyl carbamate (Boc) and benzyl carbamate (Cbz). If R⁴ contains an amine group that is protected, the protecting group is removed by treating the substrate with the appropriate reagents as described in Protective Groups in Organic Synthesis (Greene, Wuts; 3^{rd} ed., 1999, John Wiley & Sons, Inc.) to provide compounds of formula (I) wherein R¹=oxazol-5-yl and R⁵═H. Preferred conditions for removing the Boc protecting group from 8 are TFA in a solvent such as dichloromethane. Conditions for removal of the Cbz protecting group from 8 include stirring 8 under a hydrogen atmosphere in the presence of palladium on carbon, treatment of 8 with BBr₃ in dichloromethane, or treatment of 8 with methanesulfonic acid in the presence of anisole in dichloromethane.

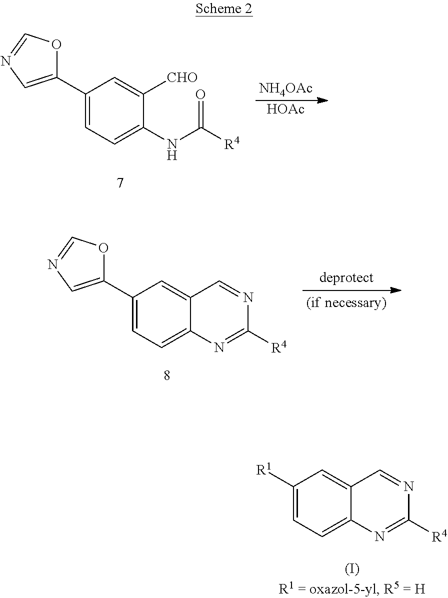

Compounds of formula (I), wherein R¹=pyridyl or pyrazole and R⁵═H, can be prepared from compounds of formula 9 by the methods shown in Scheme 3. Coupling of compounds of formula 9 with a pyridyl boronic acid or a pyrazole boronic acid in the presence of a palladium catalyst such as Pd(PPh₃)₄ or Pd(OAc)₂ and a base such as sodium carbonate, cesium carbonate, potassium carbonate, or potassium phosphate in the presence or absence of a ligand such as SPhos or XPhos and in a solvent such as DME, DMF, toluene, THF, dioxane, methanol, ethanol, n-butanol, or water or a combination thereof at temperatures ranging from 20° C. to 150° C. furnishes compounds of formula 10. The coupling reaction is carried out by heating the reaction mixture using standard laboratory methods or by heating the reaction mixture in a microwave. Heating compounds of formula 10 in the presence of ammonium acetate and acetic acid at temperatures ranging from 50 to 120° C. furnishes compounds of formula 11. Conversion of compounds of formula 11 to compounds of formula (I) can be carried out as described for the conversion of compounds of formula 8 to compounds of formula (I) in Scheme 2.

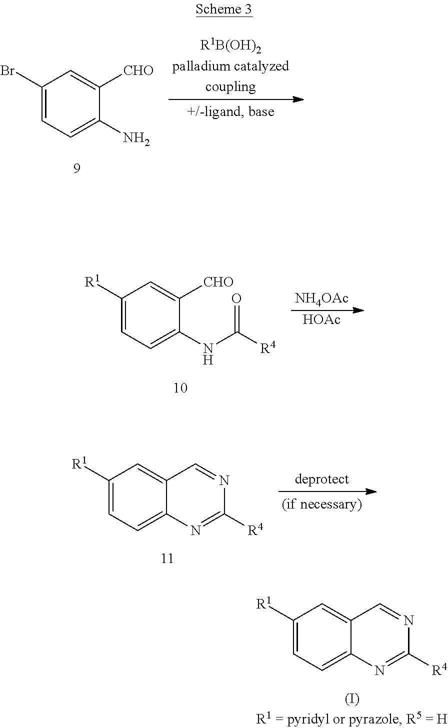

Compounds of formula (I), wherein R¹=oxazol-5-yl and R⁵=alkyl, can be prepared from compounds of formula 7 by the methods shown in Scheme 4. Treatment of 7 with a Grignard reagent in a solvent such as THF or diethyl ether furnishes compounds of formula 12. Oxidation of the resultant alcohol to the ketone using conditions such as a Swern oxidation, TPAP, manganese dioxide, Dess-Martin periodinane or chromium trioxide furnishes compounds of formula 13. Conversion of compounds of formula 13 to compounds of formula (I) can be carried out as described for the conversion of compounds of formula 7 to compounds of formula (I) in Scheme 2.

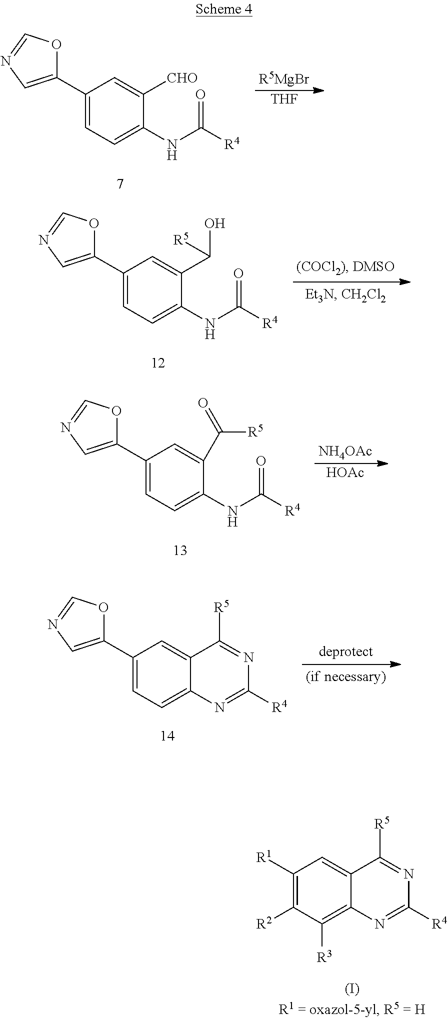

Alternatively, compounds wherein R¹=pyridyl or pyrazole and R⁵=alkyl can be prepared as shown in Scheme 5. Treatment of 15 with a Grignard reagent in a solvent such as THF or diethyl ether furnishes compounds of formula 16. When R⁵=Et, compounds of formula 16 can also be prepared by the method described by Kapa et al. (Kapa et al. Org. Proc. Res. Dev. 2003, 7, 723-732). Compounds of formula 16 can be coupled with a carboxylic acid (R⁴CO₂H) under conditions wherein the acid is converted to an acid chloride in situ. Reaction of the acid with N-(chloromethylene)-N-methylmethanaminium, chloride salt, prepared as described by Jarrahpour et al. (Tetrahedron 2009, 65, 2927-2934), in the presence of triethylamine or reaction of the acid with phosphorus oxychloride in the presence of pyridine and compounds of formula 16 leads to the formation of compounds of formula 17. Alternatively, compounds of formula 16 can be coupled directly with an acid chloride (R⁴C(O)Cl) to form compounds of formula 17. Heating compound 17 in the presence of ammonium acetate and acetic acid at temperatures ranging from 50 to 120° C. furnishes compounds of formula 18. Coupling of compounds of formula 18 with a pyridyl boronic acid or a pyrazole boronic acid in the presence of a palladium catalyst such as Pd(PPh₃)₄ or Pd(OAc)₂ and a base such as sodium carbonate, cesium carbonate, potassium carbonate, or potassium phosphate in the presence or absence of a ligand such as SPhos or XPhos and in a solvent such as DME, DMF, toluene, THF, dioxane, methanol, ethanol, n-butanol, or water or a combination thereof at temperatures ranging from 20° C. to 150° C. furnishes compounds of formula 19. The coupling reaction is carried out by heating the reaction mixture using standard laboratory methods or by heating the reaction mixture in a microwave. Conversion of compounds of formula 19 to compounds of formula (I) can be carried out as described for the conversion of compounds of formula 8 to compounds of formula (I) in Scheme 2.

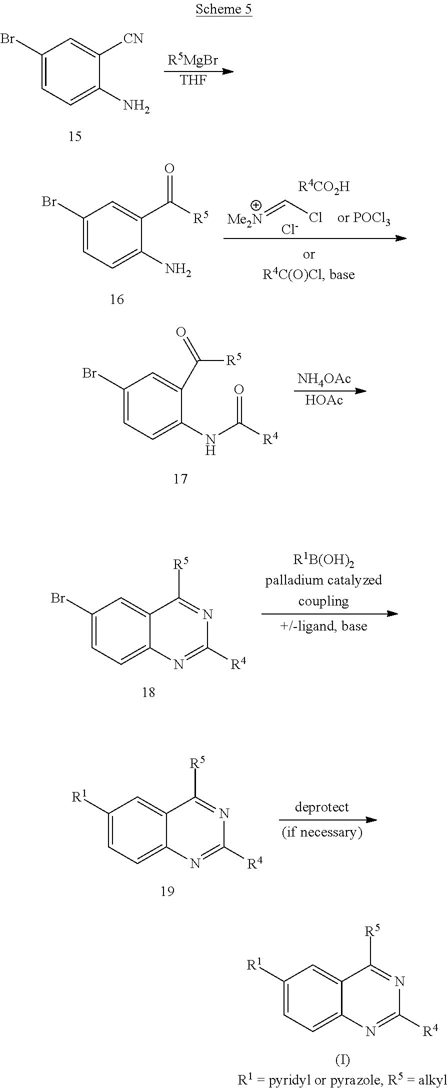

Compounds of formula (I), wherein R³=methoxy and R⁵═H or alkyl, can be prepared by the route shown in Scheme 6. Reduction of the nitro group in 20 is accomplished using standard conditions such as, but not limited to, H₂ and Pd/C, zinc with ammonium chloride, or tin chloride in an appropriate solvent such as methanol or ethanol at temperatures ranging from 0° C. to 100° C. to give compounds of formula 21. Treatment of 21 with a Grignard reagent in a solvent such as THF or diethyl ether furnishes compounds of formula 22. Oxidation of the resultant alcohol to the ketone using conditions such as a Swern oxidation, TPAP, manganese dioxide, Dess-Martin reagent or chromium trioxide, preferably manganese dioxide, furnishes compounds of formula 23. Bromination of 23 with NBS in solvents such as DMF furnishes compounds of formula 24. Compounds of formula 24 can be coupled with a carboxylic acid (R⁴CO₂H) under conditions wherein the acid is converted to an acid chloride in situ. Reaction of the acid with N-(chloromethylene)-N-methylmethanaminium, chloride salt, prepared as described by Jarrahpour et al. (Tetrahedron 2009, 65, 2927-2934), in the presence of triethylamine or reaction of the acid with phosphorus oxychloride in the presence of pyridine and compounds of formula 24 leads to the formation of compounds of formula 25. Alternatively, compounds of formula 24 can be coupled directly with an acid chloride (R⁴C(O)Cl) to form compounds of formula 25. Heating compounds of formula 25 in the presence of ammonium acetate and acetic acid at temperatures ranging from 50 to 120° C. furnishes compounds of formula 26. When R¹=oxazol-5-yl, this group can be incorporated using a three-step procedure. The bromide of 26 is converted to a vinyl group via a palladium catalyzed coupling reaction with 2,4,6-trivinyl-1,3,5,2,4,6-trioxatriborinane pyridine complex in the presence of a base such as sodium carbonate and a catalyst such as Pd(PPh₃)₄, in a solvent such as toluene, dichloroethane, THF, DMF, methanol, ethanol, water or a combination thereof at temperatures ranging from 20° C. to 150° C. Oxidation of the resultant olefin with osmium tetroxide and sodium periodate in the presence of 2,6-lutidine or with ozone followed by treatment of the resultant aldehyde with TosMIC in the presence of a base such as potassium carbonate in a solvent such as methanol affords compounds of formula 27, wherein R¹=oxazol-5-yl. When R¹=pyridyl or pyrazole, this group can be installed directly by coupling of compounds of formula 26 with a pyridyl boronic acid or a pyrazole boronic acid in the presence of a palladium catalyst such as Pd(PPh₃)₄ or Pd(OAc)₂ and a base such as sodium carbonate, cesium carbonate, potassium carbonate, or potassium phosphate in the presence or absence of a ligand such as SPhos or XPhos and in a solvent such as DME, DMF, toluene, THF, dioxane, methanol, ethanol, n-butanol, or water or a combination thereof at temperatures ranging from 20° C. to 150° C. to furnish compounds of formula 27. The coupling reaction is carried out by heating the reaction mixture using standard laboratory methods or by heating the reaction mixture in a microwave. When R⁴ contains a protected amine, the protecting group can be removed as described in Scheme 2.

Compounds of formula (I) wherein R³=methoxy and R⁵═H may be prepared by treatment of compound 21 with NBS to form compounds of formula 24, wherein R⁵═H. This compound can be carried forward by the methods shown in Scheme 6 to convert 24 to compounds of formula (I), wherein R⁵═H.

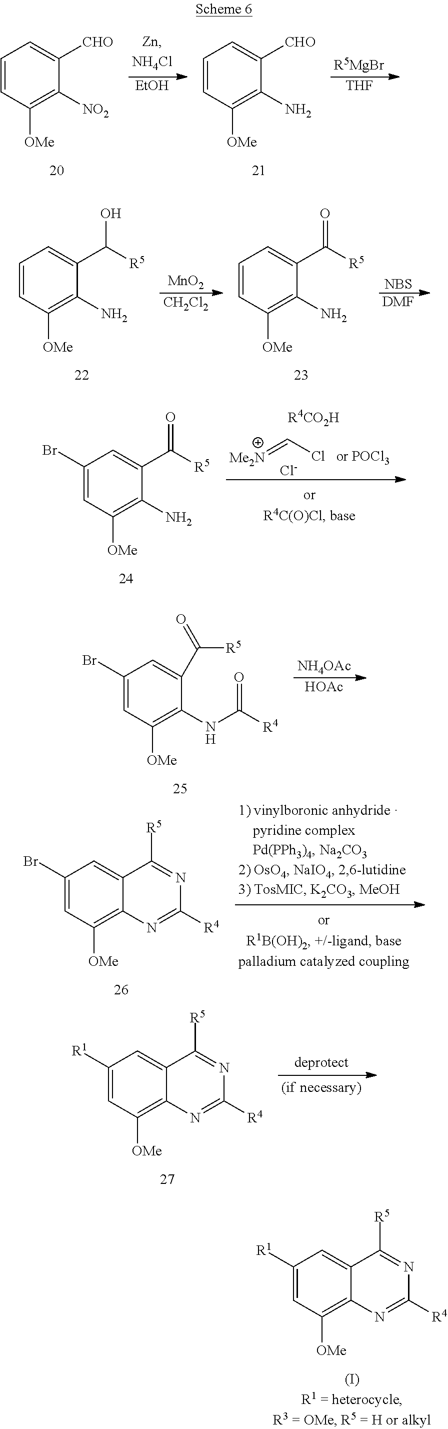

Compounds of formula (I), wherein R⁶=methoxy and R⁵═H, can be prepared by the methods shown in Scheme 7. Compound 28 can be treated with TMS diazomethane to form 29. Compound 29 can be coupled with a carboxylic acid (R⁴CO₂H) using standard peptide coupling reagents such as HATU, BOP, EDC, or TBTU in the presence of a base such as N,N-diisopropylethylamine and a solvent such as THF at temperatures ranging from 20° C. to 80° C. to form compounds of formula 30. Compound 29 can also be coupled with a carboxylic acid (R⁴CO₂H) under conditions wherein the acid is converted to an acid chloride in situ. Reaction of the acid with N-(chloromethylene)-N-methylmethanaminium, chloride salt, prepared as described by Jarrahpour et al. (Tetrahedron 2009, 65, 2927-2934), in the presence of triethylamine or reaction of the acid with phosphorus oxychloride in the presence of pyridine and compound 29 leads to the formation of compounds of formula 30. Alternatively, compound 29 can be coupled directly with an acid chloride (R⁴C(O)Cl) to form compounds of formula 30. Bromination of 30 with NBS in solvents such as DMF furnishes compounds of formula 31. When R¹=oxazol-5-yl, this group can be incorporated using a three-step procedure. The bromide of 31 is converted to a vinyl group via a palladium catalyzed coupling reaction with 2,4,6-trivinyl-1,3,5,2,4,6-trioxatriborinane pyridine complex in the presence of a base such as sodium carbonate and a catalyst such as Pd(PPh₃)₄, in a solvent such as toluene, dichloroethane, THF, DMF, methanol, ethanol, water or a combination thereof at temperatures ranging from 20° C. to 150° C. Oxidation of the resultant olefin with osmium tetroxide and sodium periodate in the presence of 2,6-lutidine or with ozone followed by treatment of the resultant aldehyde with TosMIC in the presence of a base such as potassium carbonate in a solvent such as methanol affords compounds of formula 32, wherein R¹=oxazol-5-yl. When R¹=pyridyl or pyrazole, this group can be installed directly by coupling of compounds of formula 31 with a pyridyl boronic acid or pyrazole boronic acid in the presence of a palladium catalyst such as Pd(PPh₃)₄ or Pd(OAc)₂ and a base such as sodium carbonate, cesium carbonate, potassium carbonate, or potassium phosphate in the presence or absence of a ligand such as SPhos or XPhos and in a solvent such as DME, DMF, toluene, THF, dioxane, methanol, ethanol, n-butanol, or water or a combination thereof at temperatures ranging from 20° C. to 150° C. to furnish compounds of formula 32. The coupling reaction is carried out by heating the reaction mixture using standard laboratory methods or by heating the reaction mixture in a microwave. Reduction of the ester in compounds of formula 32 with a reducing agent such as DIBAL followed by oxidation of the resultant alcohol to the aldehyde using conditions such as a Swern oxidation provides compounds of formula 34. Heating compounds of formula 34 in the presence of ammonium acetate and acetic acid at temperatures ranging from 50 to 120° C. furnishes compounds of formula 35. When R⁴ contains a protected amine, the protecting group can be removed as described in Scheme 2.

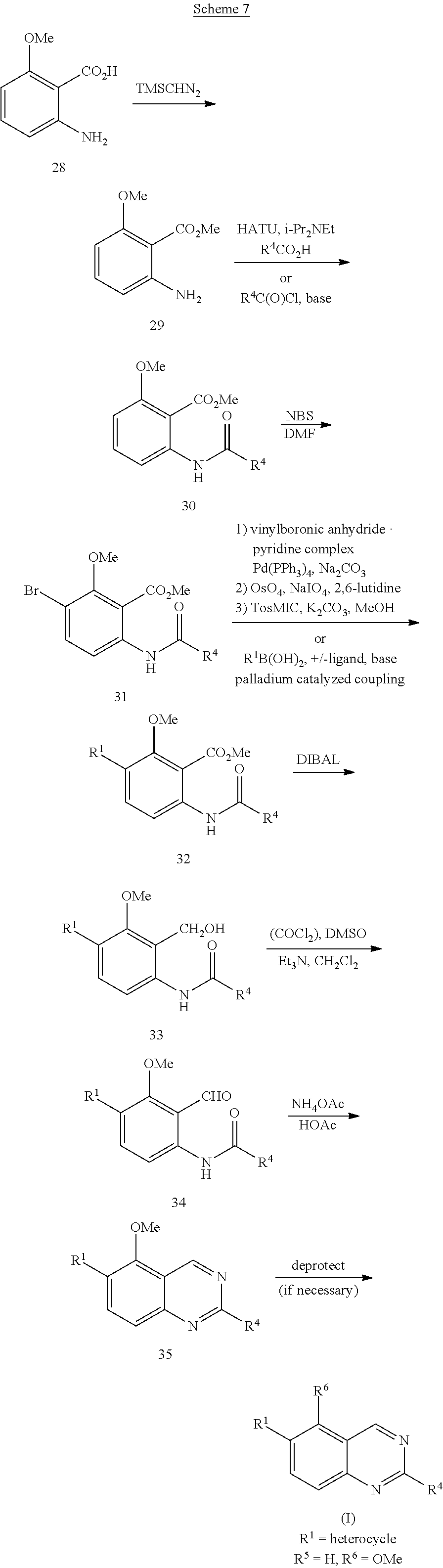

Compounds of formula (I), wherein R⁵=carbonyl, can be prepared by the methods shown in Scheme 8. Compounds of formula 36 can be coupled with a carboxylic acid (R⁴CO₂H) under conditions wherein the acid is converted to an acid chloride in situ. Reaction of the acid with N-(chloromethylene)-N-methylmethanaminium, chloride salt, prepared as described by Jarrahpour et al. (Tetrahedron 2009, 65, 2927-2934), in the presence of triethylamine or reaction of the acid with phosphorus oxychloride in the presence of pyridine and compounds of formula 36 leads to the formation of compounds of formula 37. Alternatively, compounds of formula 36 can be coupled directly with an acid chloride (R⁴C(O)Cl) to form compounds of formula 37. Compounds of formula 37 can be heated in a solvent such as ethanol in the presence of a base such as sodium carbonate at temperatures ranging from 50 to 120° C. to furnish compounds of formula 38. When R¹=oxazol-5-yl, this group can be incorporated using a three-step procedure. The bromide of 38 is converted to a vinyl group via a palladium catalyzed coupling reaction with 2,4,6-trivinyl-1,3,5,2,4,6-trioxatriborinane pyridine complex in the presence of a base such as sodium carbonate and a catalyst such as Pd(PPh₃)₄, in a solvent such as toluene, dichloroethane, THF, DMF, methanol, ethanol, water or a combination thereof at temperatures ranging from 20° C. to 150° C. Oxidation of the resultant olefin with osmium tetroxide and sodium periodate in the presence of 2,6-lutidine or with ozone followed by treatment of the resultant aldehyde with TosMIC in the presence of a base such as potassium carbonate in a solvent such as methanol affords compounds of formula 39, wherein R¹=oxazol-5-yl. When R¹=pyridyl or pyrazole, this group can be installed directly by coupling of compounds of formula 38 with a pyridyl boronic acid or pyrazole boronic acid in the presence of a palladium catalyst such as Pd(PPh₃)₄ or Pd(OAc)₂ and a base such as sodium carbonate, cesium carbonate, potassium carbonate, or potassium phosphate in the presence or absence of a ligand such as SPhos or XPhos and in a solvent such as DME, DMF, toluene, THF, dioxane, methanol, ethanol, n-butanol, or water or a combination thereof at temperatures ranging from 20° C. to 150° C. to furnish compounds of formula 39. The coupling reaction is carried out by heating the reaction mixture using standard laboratory methods or by heating the reaction mixture in a microwave. When R⁴ contains a protected amine, the protecting group can be removed as described in Scheme 2.

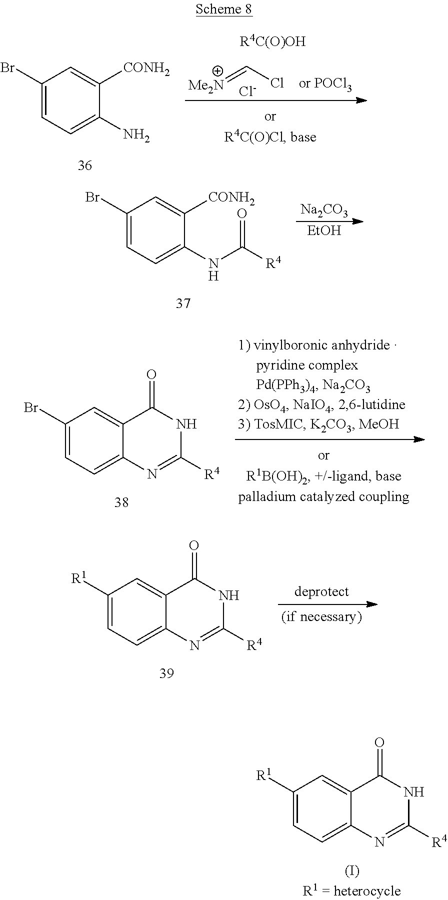

Compounds of formula (I), wherein R⁵═NR⁸R⁹, OR⁸ or CN, can be prepared by the methods shown in Scheme 9. Compounds of formula 39 can be converted to compounds of formula 40 using conditions described by Wan et al. (J. Org. Chem. 2007, 72, 10194-10210). When R⁵═NR⁸R⁹ or OR⁸, compound 39 is treated with the desired mono or disubstituted amine or the desired alcohol in the presence of BOP and DBU in DMF to furnish compounds of formula 40, wherein R⁵═NR⁸R⁹ or OR⁸, respectively. When R⁵═CN, compound 39 is treated with sodium cyanide and 18-crown-6 in the presence of BOP and DBU in acetonitrile to furnish compounds of formula 40, wherein R⁵═CN. When R⁴ contains a protected amine, the protecting group can be removed as described in Scheme 2.

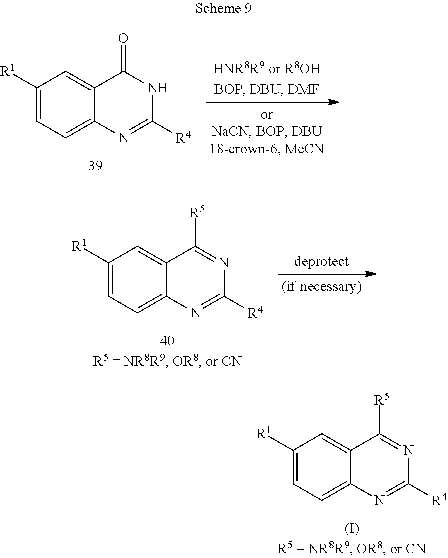

Alternatively, as shown in Scheme 10, compounds of formula 39, wherein R⁵═OR⁸, can be alkylated by deprotonating 39 with a base such as sodium hydride followed by addition of an alkylating agent such as an alkyl halide or an alkyl group with another suitable leaving group to give compounds of formula 40. The alkylation reaction produces a mixture of O and N alkylated products. When R⁴ contains a protected amine, the protecting group can be removed as described in Scheme 2.

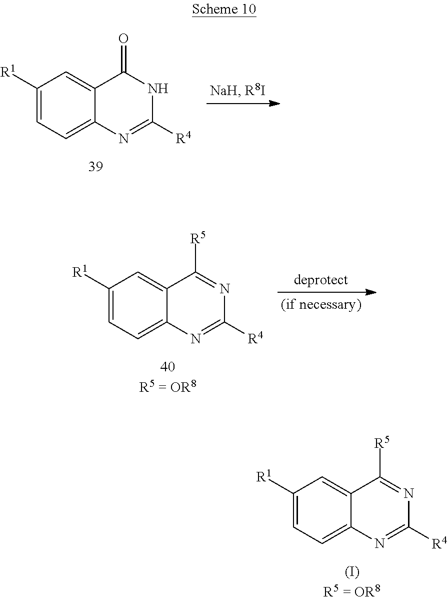

Alternatively, compounds of formula (I), wherein R⁵═NR⁸R⁹, OR⁸ or CN, can be prepared by the methods shown in Scheme 11. Compounds of formula 39 can be treated with a chlorinating agent such as phosphorous oxychloride. The resultant aryl chloride can be treated with an amine, an alcohol, or a cyanide, such as sodium cyanide, to furnish compounds of formula 40. When R⁴ contains a protected amine, the protecting group can be removed as described in Scheme 2.

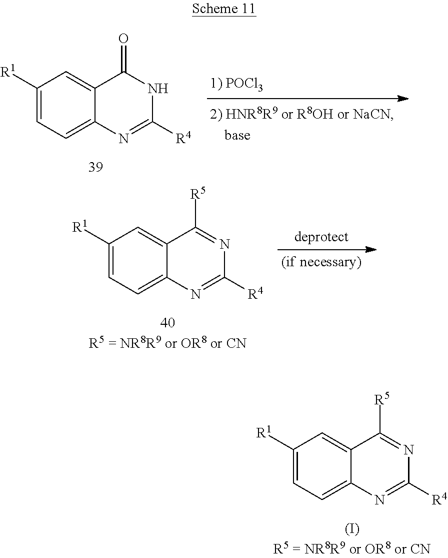

Compounds of formula (I) wherein R²═OMe and R⁵=carbonyl, NR⁸R⁹, OR⁸ or CN can be prepared by the methods shown in Scheme 12. Bromination of 41 with NBS in solvents such as DMF furnishes compound 42. Treatment of 42 with EDC in combination with HOBt followed by the addition of ammonium hydroxide affords compound 43. Compound 43 can be converted to compounds of formula (I), wherein R²═OMe and R⁵=carbonyl, NR⁸R⁹, OR⁸ or CN using the appropriate steps shown in Schemes 8 and 9.

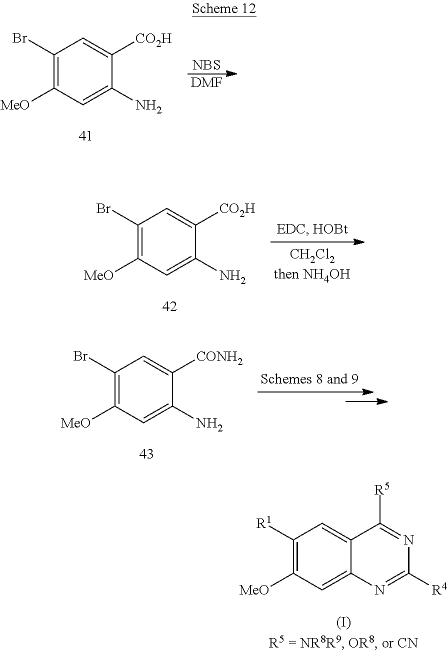

Compounds of formula (I), wherein R²═OMe and R⁵═H or alkyl, can be prepared by the methods shown in Scheme 13. The carboxylic acid in 42 (prepared as described in Scheme H) may be reduced with lithium aluminum hydride. Subsequent oxidation of the resultant alcohol with oxidizing agents or conditions such as Swern oxidation, TPAP, manganese dioxide, Dess-Martin periodinane or chromium trioxide may be used to furnish compound 44. Compound 44 can be coupled with a carboxylic acid (R⁴CO₂H) using standard peptide coupling reagents such as HATU, BOP, EDC, or TBTU in the presence of a base such as N,N-diisopropylethylamine and a solvent such as THF at temperatures ranging from 20° C. to 80° C. to form compounds of formula 45. Compounds of formula 44 can also be coupled with a carboxylic acid (R⁴CO₂H) under conditions wherein the acid is converted to an acid chloride in situ. Reaction of the acid with N-(chloromethylene)-N-methylmethanaminium, chloride salt, prepared as described by Jarrahpour et al. (Tetrahedron 2009, 65, 2927-2934), in the presence of triethylamine or reaction of the acid with phosphorus oxychloride in the presence of pyridine and compounds of formula 44 leads to the formation of compounds of formula 45. Alternatively, compounds of formula 44 can be coupled directly with an acid chloride (R⁴C(O)Cl) to form compounds of formula 45. Alternatively if R⁵=alkyl, compounds of formula 45 may be treated with a Grignard reagent in a solvent such as THF or diethyl ether. Subsequent oxidation of the resultant alcohol with oxidizing agents or conditions such as Swern oxidation, TPAP, manganese dioxide, Dess-Martin periodinane or chromium trioxide may be used to furnish compounds of formula 46. Heating compounds of formula 46 in the presence of ammonium acetate and acetic acid at temperatures ranging from 50 to 120° C. furnishes compounds of formula 47.

If R⁵═H, compounds of formula 45 may be directly converted to compounds of formula 47 using the conditions described for the conversion of compounds of formula 46 to compounds of formula 47. Compounds of formula 47 may be converted to compounds of formula 48 using a palladium catalyzed coupling reaction. When R¹=oxazol-5-yl, this group can be incorporated using a three-step procedure. The bromide of 47 is converted to a vinyl group via a palladium catalyzed coupling reaction with 2,4,6-trivinyl-1,3,5,2,4,6-trioxatriborinane pyridine complex in the presence of a base such as sodium carbonate and a catalyst such as Pd(PPh₃)₄, in a solvent such as toluene, dichloroethane, THF, DMF, methanol, ethanol, water or a combination thereof at temperatures ranging from 20° C. to 150° C. Oxidation of the resultant olefin with osmium tetroxide and sodium periodate in the presence of 2,6-lutidine or with ozone followed by treatment of the resultant aldehyde with TosMIC in the presence of a base such as potassium carbonate in a solvent such as methanol affords compounds of formula 48, wherein R¹=oxazol-5-yl. When R¹=pyridyl or pyrazole, this group can be installed directly by coupling of compounds of formula 47 with a pyridyl boronic acid or pyrazole boronic acid in the presence of a palladium catalyst such as Pd(PPh₃)₄ or Pd(OAc)₂ and a base such as sodium carbonate, cesium carbonate, potassium carbonate, or potassium phosphate in the presence or absence of a ligand such as SPhos or XPhos and in a solvent such as DME, DMF, toluene, THF, dioxane, methanol, ethanol, n-butanol, or water or a combination thereof at temperatures ranging from 20° C. to 150° C. to furnish compounds of formula 48. The coupling reaction is carried out by heating the reaction mixture using standard laboratory methods or by heating the reaction mixture in a microwave. When R⁴ contains a protected amine, the protecting group can be removed as described in Scheme 2.

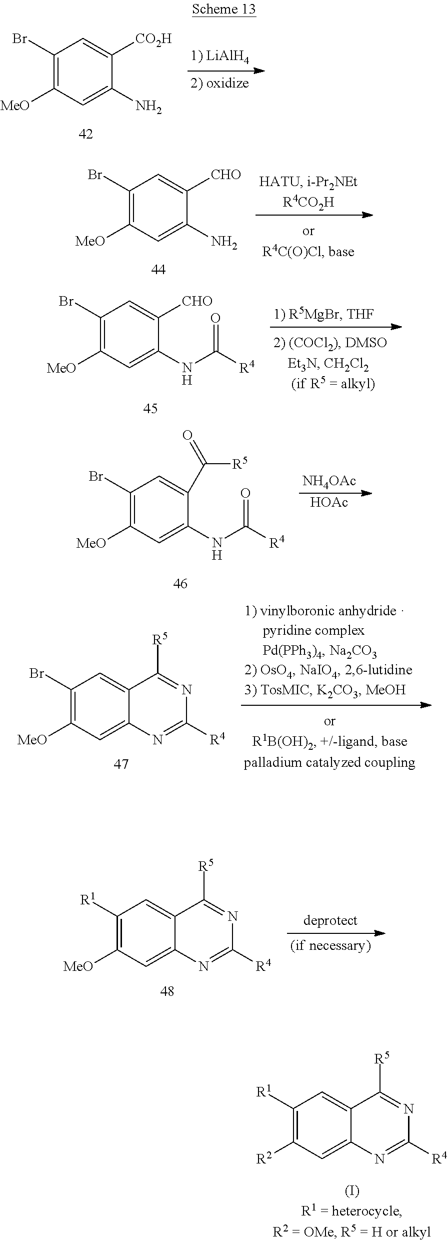

Compounds of formula (I), wherein R²═H or OMe and R⁵=alkyl or aryl, can be prepared by the methods shown in Scheme 14. The amide 50 can be prepared as described in Scheme 7. The compounds of formula 49 can also be coupled with a carboxylic acid (R⁴CO₂H) under conditions wherein the acid is converted to an acid chloride in situ. Reaction of the acid with N-(chloromethylene)-N-methylmethanaminium, chloride salt, prepared as described by Jarrahpour et al. (Tetrahedron 2009, 65, 2927-2934), in the presence of triethylamine or reaction of the acid with phosphorus oxychloride in the presence of pyridine and amine leads to the formation of compounds of formula 50. Compounds of formula 50 may be converted to compounds of formula 51 using a palladium catalyzed coupling reaction. When R¹=oxazol-5-yl, this group can be incorporated using a three-step procedure. The bromide of 50 is converted to a vinyl group via a palladium catalyzed coupling reaction with 2,4,6-trivinyl-1,3,5,2,4,6-trioxatriborinane pyridine complex in the presence of a base such as sodium carbonate and a catalyst such as Pd(PPh₃)₄, in a solvent such as toluene, dichloroethane, THF, DMF, methanol, ethanol, water or a combination thereof at temperatures ranging from 20° C. to 150° C. Oxidation of the resultant olefin with osmium tetroxide and sodium periodate in the presence of 2,6-lutidine or with ozone followed by treatment of the resultant aldehyde with TosMIC in the presence of a base such as potassium carbonate in a solvent such as methanol affords compounds of formula 51, wherein R¹=oxazol-5-yl. When R¹=pyridyl or pyrazole this group can be installed directly by coupling of compounds of formula 50 with a pyridyl boronic acid or pyrazole boronic acid in the presence of a palladium catalyst such as Pd(PPh₃)₄ or Pd(OAc)₂ and a base such as sodium carbonate, cesium carbonate, potassium carbonate, or potassium phosphate in the presence or absence of a ligand such as SPhos or XPhos and in a solvent such as DME, DMF, toluene, THF, dioxane, methanol, ethanol, n-butanol, or water or a combination thereof at temperatures ranging from 20° C. to 150° C. to furnish compounds of formula 51. The coupling reaction is carried out by heating the reaction mixture using standard laboratory methods or by heating the reaction mixture in a microwave. When R⁵=alkyl or aryl, compounds of formula 51 may be treated with a Grignard reagent in a solvent such as THF or diethyl ether. When R⁴ contains a protected amine, the protecting group can be removed as described in Scheme 2.

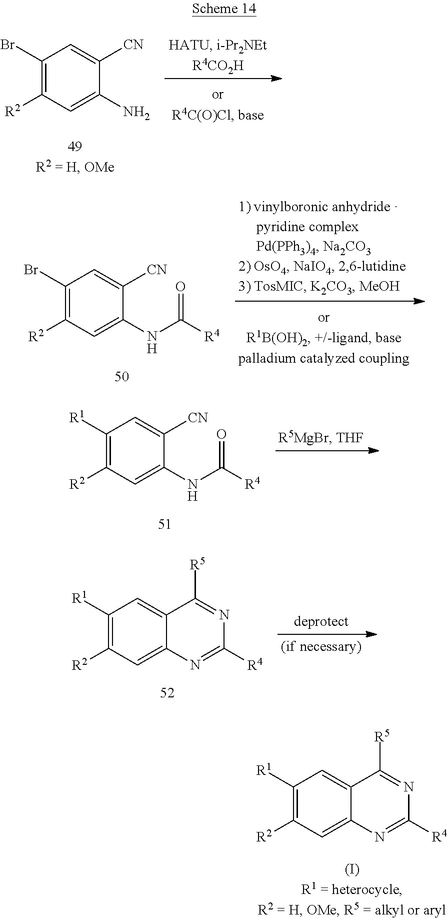

Compounds of formula (I), wherein R⁵═CONH₂, CN, or CONMe₂ and R¹=aryl, can be prepared by the methods shown in Scheme 15. Compounds of formula 54 can be prepared by treatment with a carboxylic acid (R⁴CO₂H). Reaction of the acid with N-(chloromethylene)-N-methylmethanaminium, chloride salt, prepared as described by Jarrahpour et al. (Tetrahedron 2009, 65, 2927-2934), in the presence of triethylamine or reaction of the acid with phosphorus oxychloride in the presence of pyridine and amine leads to the formation of compounds of formula 54. Compounds of formula 54 may be converted to compounds of formula 55 and 56 by treatment with ethanolic ammonia. When R¹=pyridyl, this group can be installed directly by coupling of compounds of formula 56 with a pyridyl boronic acid in the presence of a palladium catalyst such as Pd(PPh₃)₄ or Pd(OAc)₂ and a base such as sodium carbonate, cesium carbonate, potassium carbonate, or potassium phosphate in the presence or absence of a ligand such as SPhos or XPhos and in a solvent such as DME, DMF, toluene, THF, dioxane, methanol, ethanol, n-butanol, or water or a combination thereof at temperatures ranging from 20° C. to 150° C. to furnish compounds of formula (I) wherein R⁵═CONH₂. The coupling reaction is carried out by heating the reaction mixture using standard laboratory methods or by heating the reaction mixture in a microwave. Alternatively, compounds of formula (I) wherein R⁵═CONH₂ may be treated with trifluoroacetic anhydride in a solvent such as dioxane in presence of base such as pyridine to furnish compounds of formula (I) wherein R⁵═CN. Compounds of formula 55 can be treated with TMS diazomethane in solvent such as diethyl ether to afford compounds of formula 57. The compounds of formula 57 can be treated with diethyl amine in solvents such as THF at temperatures of about 100° C. to furnish compounds of formula 58. When R¹=pyridyl, this group can be installed directly by coupling of compounds of formula 58 with a pyridyl boronic acid in the presence of a palladium catalyst such as Pd(PPh₃)₄ or Pd(OAc)₂ and a base such as sodium carbonate, cesium carbonate, potassium carbonate, or potassium phosphate in the presence or absence of a ligand such as SPhos or XPhos and in a solvent such as DME, DMF, toluene, THF, dioxane, methanol, ethanol, n-butanol, or water or a combination thereof at temperatures ranging from 20° C. to 150° C. to furnish compounds of formula (I) wherein R⁵═CONMe₂. Alternatively, compounds of formula 57 can be treated with sodium borohydride in solvent such as THF or methanol to afford compounds of formula 59.

When R¹=pyridyl, this group can be installed directly by coupling of compounds of formula 59 with a pyridyl boronic acid in the presence of a palladium catalyst such as Pd(PPh₃)₄ or Pd(OAc)₂ and a base such as sodium carbonate, cesium carbonate, potassium carbonate, or potassium phosphate in the presence or absence of a ligand such as SPhos or XPhos and in a solvent such as DME, DMF, toluene, THF, dioxane, methanol, ethanol, n-butanol, or water or a combination thereof at temperatures ranging from 20° C. to 150° C. to furnish compounds of formula (I) wherein R⁵═CH₂OH.

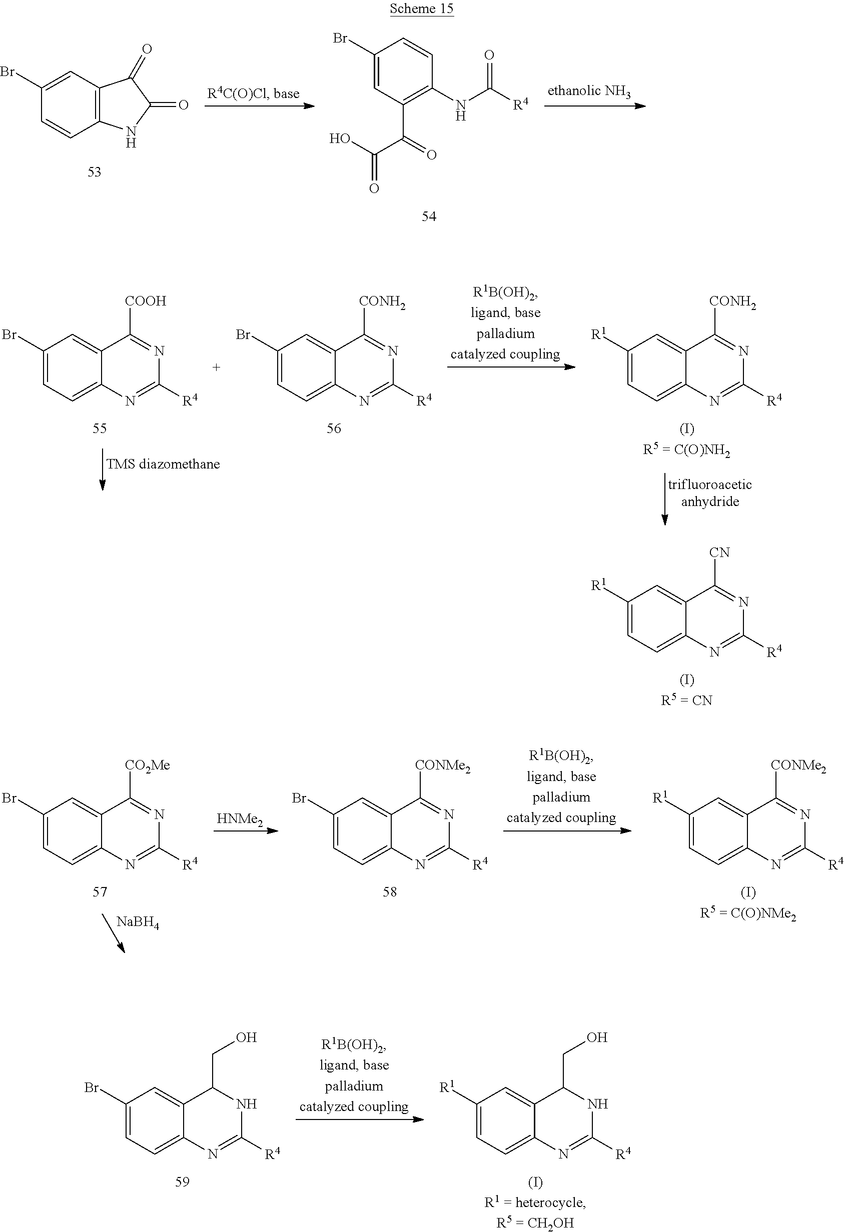

Various analogs synthesized using Schemes 1-15 are listed in Table 1.

TABLE 1
I

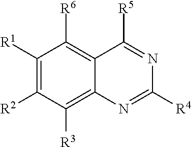

| Ex | R¹ | R² | R³ | R⁴ | R⁵ | R⁶ | (M + H)⁺ |
|---|---|---|---|---|---|---|---|
| 1 | 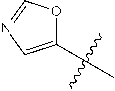 [SMILES: *c1cnco1] | H | H | 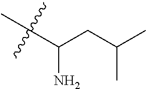 [SMILES: *C(N)CC(C)C] | H | H | 283.2 |
| 2 | 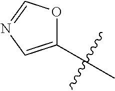 [SMILES: *c1cnco1] | H | H | 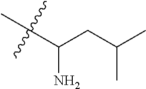 [SMILES: *C(N)CC(C)C] | Me | H | 297.2 |
| 3 | 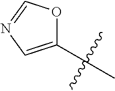 [SMILES: *c1cnco1] | H | H | 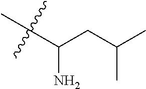 [SMILES: *C(N)CC(C)C] | Et | H | 311.2 |
| 4 | 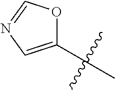 [SMILES: *c1cnco1] | H | H | 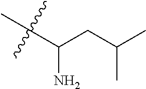 [SMILES: *C(N)CC(C)C] | Pr | H | 324.1 |
| 5 | 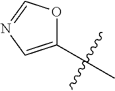 [SMILES: *c1cnco1] | H | OMe | 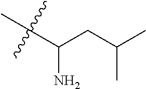 [SMILES: *C(N)CC(C)C] | Et | H | 341.1 |
| 6 | 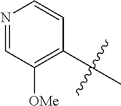 [SMILES: *c1ccncc1OC] | H | H | 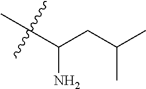 [SMILES: *C(N)CC(C)C] | Et | H | 351.1 |
| 7 | 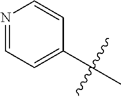 [SMILES: *c1ccncc1] | H | H | 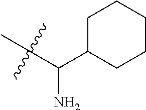 [SMILES: *C(N)C1CCCCC1] | Et | H | 347.2 |
| 8 | 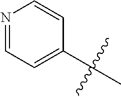 [SMILES: *c1ccncc1] | H | H | 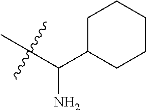 [SMILES: *C(N)C1CCCCC1] | Et | H | 347.2 |
| 9 | 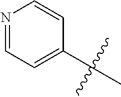 [SMILES: *c1ccncc1] | H | H | 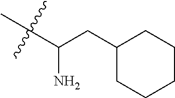 [SMILES: *C(N)CC1CCCCC1] | Et | H | 361.2 |
| 10 | 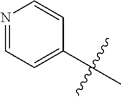 [SMILES: *c1ccncc1] | H | H | 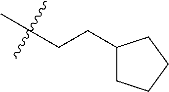 [SMILES: *CCC1CCCC1] | Et | H | 332.3 |
| 11 | 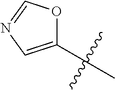 [SMILES: *c1cnco1] | H | H | 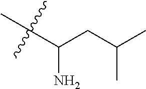 [SMILES: *C(N)CC(C)C] | H | OMe | 313.2 |
| 12 | 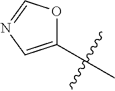 [SMILES: *c1cnco1] | H | H | 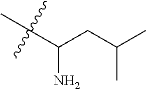 [SMILES: *C(N)CC(C)C] | O | H | 297.2 |
| 13 | 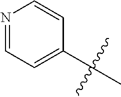 [SMILES: *c1ccncc1] | H | H | 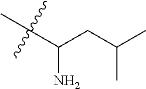 [SMILES: *C(N)CC(C)C] | O | H | 309.1 |
| 14 | 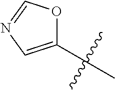 [SMILES: *c1cnco1] | H | H | 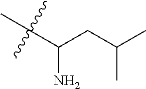 [SMILES: *C(N)CC(C)C] | OMe | H | 313.3 |
| 15 | 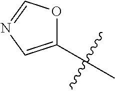 [SMILES: *c1cnco1] | H | H | 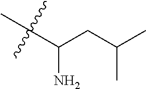 [SMILES: *C(N)CC(C)C] | OEt | H | 327.2 |
| 16 | 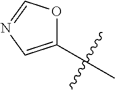 [SMILES: *c1cnco1] | H | H | 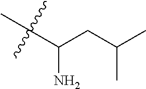 [SMILES: *C(N)CC(C)C] | NHMe | H | 312.2 |
| 17 | 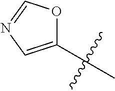 [SMILES: *c1cnco1] | H | H | 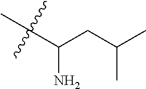 [SMILES: *C(N)CC(C)C] | NMe₂ | H | 326.2 |
| 18 | 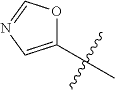 [SMILES: *c1cnco1] | H | H | 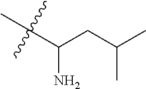 [SMILES: *C(N)CC(C)C] | NEt₂ | H | 354.4 |
| 19 | 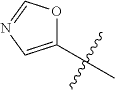 [SMILES: *c1cnco1] | H | H | 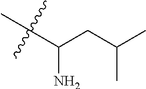 [SMILES: *C(N)CC(C)C] | 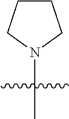 [SMILES: *N1CCCC1] | H | 352.2 |
| 20 | 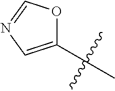 [SMILES: *c1cnco1] | H | H | 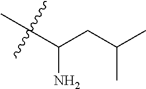 [SMILES: *C(N)CC(C)C] | NHEt | H | 326.3 |
| 21 | 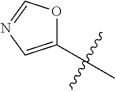 [SMILES: *c1cnco1] | H | H | 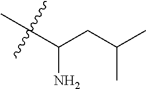 [SMILES: *C(N)CC(C)C] | 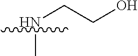 | H | 342.4 |
| 22 | 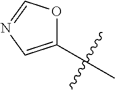 [SMILES: *c1cnco1] | H | H | 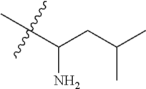 [SMILES: *C(N)CC(C)C] | 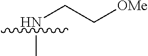 | H | 356.3 |
| 23 | 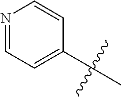 [SMILES: *c1ccncc1] | H | H | 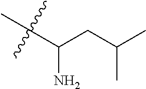 [SMILES: *C(N)CC(C)C] | NHMe | H | 322.3 |
| 24 | 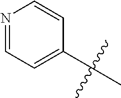 [SMILES: *c1ccncc1] | H | H | 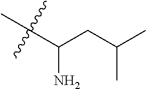 [SMILES: *C(N)CC(C)C] | NHEt | H | 336.4 |
| 25 | 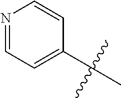 [SMILES: *c1ccncc1] | H | H | 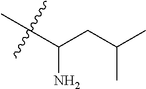 [SMILES: *C(N)CC(C)C] | NEt₂ | H | 364.3 |
| 26 | 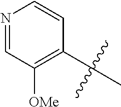 [SMILES: *c1ccncc1OC] | H | H | 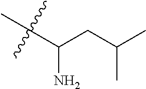 [SMILES: *C(N)CC(C)C] | NMe₂ | H | 366.3 |
| 27 | 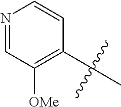 [SMILES: *c1ccncc1OC] | H | H | 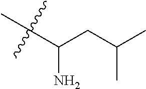 [SMILES: *C(N)CC(C)C] | NEt₂ | H | 394.4 |
| 28 | 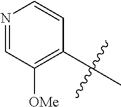 [SMILES: *c1ccncc1OC] | H | H | 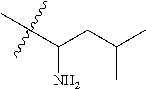 [SMILES: *C(N)CC(C)C] | NHEt | H | 366.3 |
| 29 | 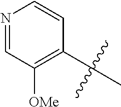 [SMILES: *c1ccncc1OC] | H | H | 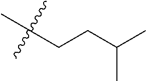 [SMILES: *CCC(C)C] | NMe₂ | H | 351.2 |
| 30 | 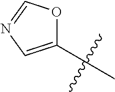 [SMILES: *c1cnco1] | H | H | 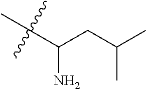 [SMILES: *C(N)CC(C)C] | CN | H | 399.1 |
| 31 | 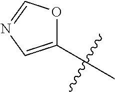 [SMILES: *c1cnco1] | H | H | 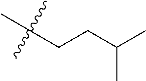 [SMILES: *CCC(C)C] | CN | H | 293.1 |
| 32 | 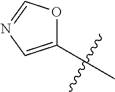 [SMILES: *c1cnco1] | OMe | H | 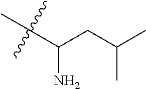 [SMILES: *C(N)CC(C)C] | CN | H | 338.1 |
| 33 | 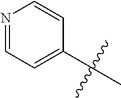 [SMILES: *c1ccncc1] | H | H | 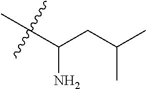 [SMILES: *C(N)CC(C)C] | CN | H | 318.3 |
| 34 | 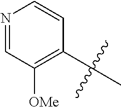 [SMILES: *c1ccncc1OC] | H | H | 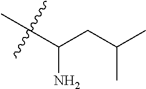 [SMILES: *C(N)CC(C)C] | CN | H | 348.3 |
| 35 | 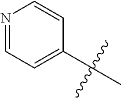 [SMILES: *c1ccncc1] | OMe | H | 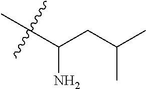 [SMILES: *C(N)CC(C)C] | CN | H | 348.1 |
| 36 | 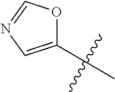 [SMILES: *c1cnco1] | OMe | H | 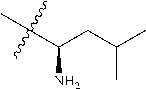 [SMILES: *[C@H](N)CC(C)C] | H | H | 313.2 |
| 37 | 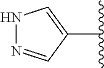 [SMILES: *c1cn[nH]c1] | H | H | 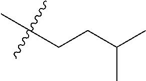 [SMILES: *CCC(C)C] | OMe | H | 296.37 |
| 38 | 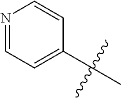 [SMILES: *c1ccncc1] | H | H | 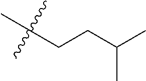 [SMILES: *CCC(C)C] | OMe | H | 307.40 |
| 39 | 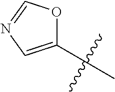 [SMILES: *c1cnco1] | H | H | 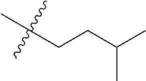 [SMILES: *CCC(C)C] | NHMe | H | 296.37 |
| 40 | 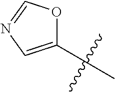 [SMILES: *c1cnco1] | H | H | 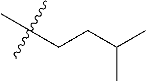 [SMILES: *CCC(C)C] | NMe₂ | H | 310.40 |
| 41 | 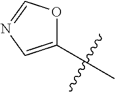 [SMILES: *c1cnco1] | H | H | 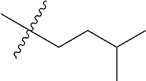 [SMILES: *CCC(C)C] | OMe | H | 297.36 |
| 42 | 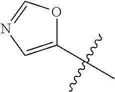 [SMILES: *c1cnco1] | H | H | 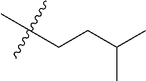 [SMILES: *CCC(C)C] | O | H | 283.33 |
| 43 | 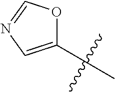 [SMILES: *c1cnco1] | H | H | 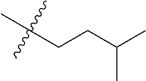 [SMILES: *CCC(C)C] | SMe | H | 313.42 |
| 44 | 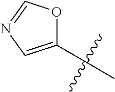 [SMILES: *c1cnco1] | H | H | 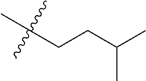 [SMILES: *CCC(C)C] | 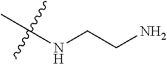 [SMILES: *NCCN] | H | 325.41 |
| 45 | 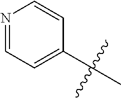 [SMILES: *c1ccncc1] | H | H | 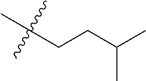 [SMILES: *CCC(C)C] | Et | H | 305.42 |
| 46 | 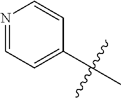 [SMILES: *c1ccncc1] | H | H | 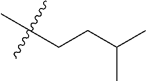 [SMILES: *CCC(C)C] | i-Pr | H | 319.45 |
| 47 | 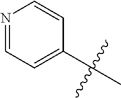 [SMILES: *c1ccncc1] | H | H | 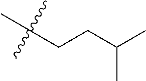 [SMILES: *CCC(C)C] | 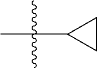 [SMILES: *C1CC1] | H | 317.43 |
| 48 | 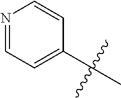 [SMILES: *c1ccncc1] | H | H | 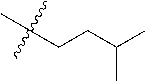 [SMILES: *CCC(C)C] | 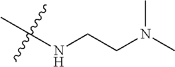 [SMILES: *NCCN(C)C] | H | 363.51 |
| 49 | 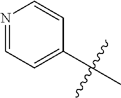 [SMILES: *c1ccncc1] | H | H | 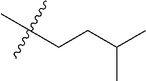 [SMILES: *CCC(C)C] | O | H | 293.37 |
| 50 | 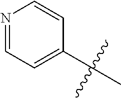 [SMILES: *c1ccncc1] | H | H | 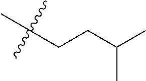 [SMILES: *CCC(C)C] | 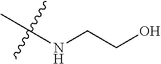 [SMILES: *NCCO] | H | 336.44 |
| 51 | 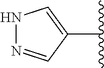 [SMILES: *c1cn[nH]c1] | H | H | 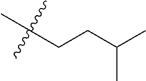 [SMILES: *CCC(C)C] | O | H | 282.35 |
| 52 | 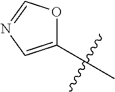 [SMILES: *c1cnco1] | OMe | H | 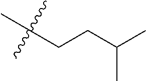 [SMILES: *CCC(C)C] | Et | H | 325.41 |
| 53 | 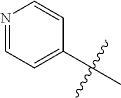 [SMILES: *c1ccncc1] | H | H | 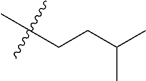 [SMILES: *CCC(C)C] | CONH₂ | H | 320.39 |
| 54 | 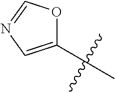 [SMILES: *c1cnco1] | OEt | H | 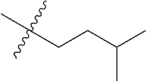 [SMILES: *CCC(C)C] | Et | H | 339.44 |
| 55 | 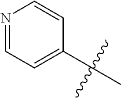 [SMILES: *c1ccncc1] | H | H | 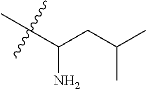 [SMILES: *C(N)CC(C)C] | OMe | H | 322.41 |
| 56 | 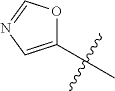 [SMILES: *c1cnco1] | H | H | 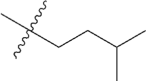 [SMILES: *CCC(C)C] | S | H | 299.40 |
| 57 | 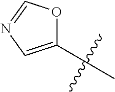 [SMILES: *c1cnco1] | H | H | 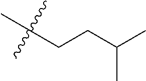 [SMILES: *CCC(C)C] | Et | H | 295.38 |
| 58 | 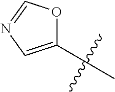 [SMILES: *c1cnco1] | OMe | H | 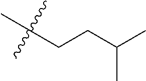 [SMILES: *CCC(C)C] | O | H | 313.36 |
| 59 | 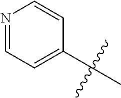 [SMILES: *c1ccncc1] | H | H | 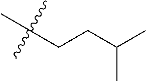 [SMILES: *CCC(C)C] | 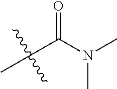 [SMILES: *C(=O)N(C)C] | H | 348.45 |
| 60 | 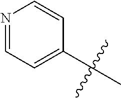 [SMILES: *c1ccncc1] | H | H | 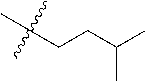 [SMILES: *CCC(C)C] | CN | H | 302.38 |
| 61 | 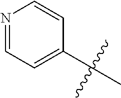 [SMILES: *c1ccncc1] | H | H | 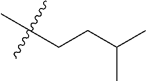 [SMILES: *CCC(C)C] | CH₂OH | H | 309.41 |
| 62 | 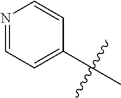 [SMILES: *c1ccncc1] | H | H | 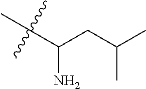 [SMILES: *C(N)CC(C)C] | Et | H | 320.44 |
| 63 | 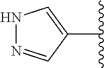 [SMILES: *c1cn[nH]c1] | H | H | 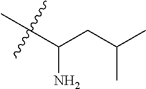 [SMILES: *C(N)CC(C)C] | Et | H | 309.42 |
| 64 | 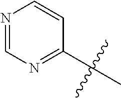 [SMILES: *c1ccncn1] | H | H | 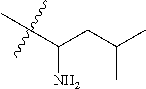 [SMILES: *C(N)CC(C)C] | Et | H | 321.43 |
| 65 | 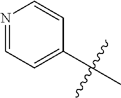 [SMILES: *c1ccncc1] | H | H | 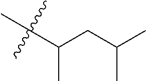 [SMILES: *C(C)CC(C)C] | Et | H | 319.45 |
| 66 | 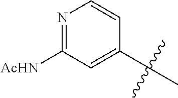 [SMILES: *c1ccnc(NC(C)=O)c1] | H | H | 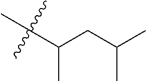 [SMILES: *C(C)CC(C)C] | Et | H | 376.50 |
| 67 | 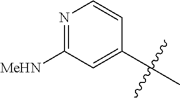 [SMILES: *c1ccnc(NC)c1] | H | H | 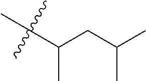 [SMILES: *C(C)CC(C)C] | Et | H | 348.49 |
| 68 | 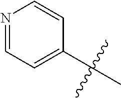 [SMILES: *c1ccncc1] | H | H | 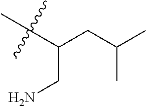 [SMILES: *C(CN)CC(C)C] | Et | H | 334.46 |
| 69 | 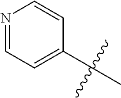 [SMILES: *c1ccncc1] | H | H | 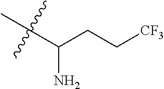 [SMILES: *C(N)CCC(F)(F)F] | Et | H | 360.38 |
| 70 | 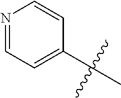 [SMILES: *c1ccncc1] | H | H | 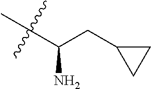 [SMILES: *[C@H](N)CC1CC1] | Et | H | 318.42 |
| 71 | 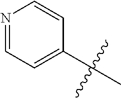 [SMILES: *c1ccncc1] | H | H | 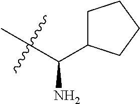 [SMILES: *[C@H](N)C1CCCC1] | Et | H | 330.20 |
| 72 | 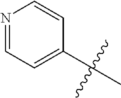 [SMILES: *c1ccncc1] | H | H | 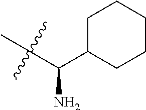 [SMILES: *[C@H](N)C1CCCCC1] | Et | H | 346.48 |
| 73 | 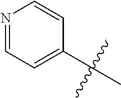 [SMILES: *c1ccncc1] | H | H | 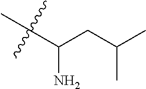 [SMILES: *C(N)CC(C)C] | 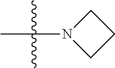 [SMILES: *N1CCC1] | H | 347.46 |
| 74 | 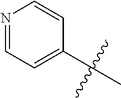 [SMILES: *c1ccncc1] | H | H | 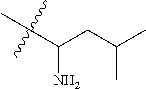 [SMILES: *C(N)CC(C)C] | 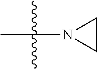 [SMILES: *N1CC1] | H | 333.44 |
| 75 | 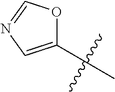 [SMILES: *c1cnco1] | OMe | H | 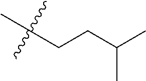 [SMILES: *CCC(C)C] | Me | H | 311.38 |
| 76 | 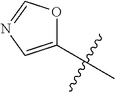 [SMILES: *c1cnco1] | H | H | 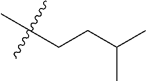 [SMILES: *CCC(C)C] | 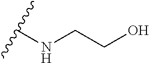 [SMILES: *NCCO] | H | 327.2 |
| 77 | 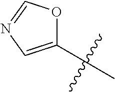 [SMILES: *c1cnco1] | H | H | 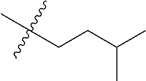 [SMILES: *CCC(C)C] | 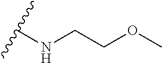 [SMILES: *NCCOC] | H | 341.2 |
| 78 | 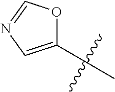 [SMILES: *c1cnco1] | H | H | 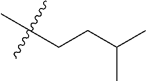 [SMILES: *CCC(C)C] | 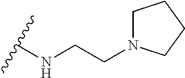 [SMILES: *NCCN1CCCC1] | H | 380.2 |
| 79 | 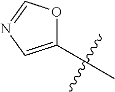 [SMILES: *c1cnco1] | H | H | 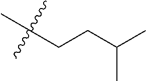 [SMILES: *CCC(C)C] | 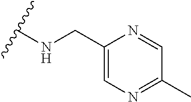 [SMILES: *NCc1cnc(C)cn1] | H | 389.2 |
| 80 | 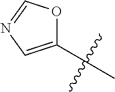 [SMILES: *c1cnco1] | H | H | 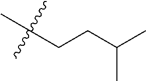 [SMILES: *CCC(C)C] | 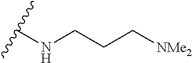 [SMILES: *NCCCN(C)C] | H | 368.2 |
| 81 | 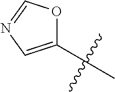 [SMILES: *c1cnco1] | H | H | 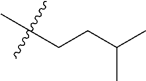 [SMILES: *CCC(C)C] | 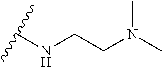 [SMILES: *NCCN(C)C] | H | 354.2 |
| 82 | 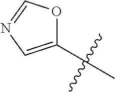 [SMILES: *c1cnco1] | H | H | 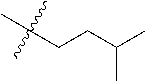 [SMILES: *CCC(C)C] | 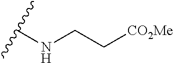 [SMILES: *NCCC(=O)OC] | H | 369.2 |
| 83 | 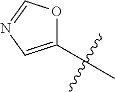 [SMILES: *c1cnco1] | H | H | 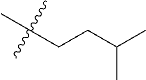 [SMILES: *CCC(C)C] | 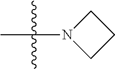 [SMILES: *N1CCC1] | H | 323.2 |
| 84 | 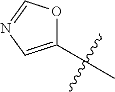 [SMILES: *c1cnco1] | H | H | 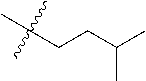 [SMILES: *CCC(C)C] | 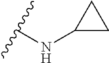 [SMILES: *NC1CC1] | H | 323.2 |
| 85 | 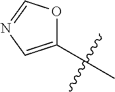 [SMILES: *c1cnco1] | OMe | H | 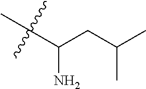 [SMILES: *C(N)CC(C)C] | Et | H | 340.43 |
| 86 | 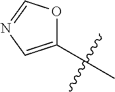 [SMILES: *c1cnco1] | OMe | H | 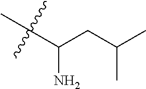 [SMILES: *C(N)CC(C)C] | Et | H | 340.43 |
| 87 | 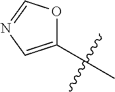 [SMILES: *c1cnco1] | OMe | H | 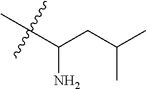 [SMILES: *C(N)CC(C)C] | Et | H | 340.43 |
| 88 | 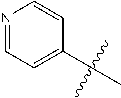 [SMILES: *c1ccncc1] | OMe | H | 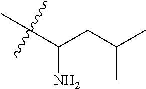 [SMILES: *C(N)CC(C)C] | Et | H | 350.46 |

In the following examples, proton NMR spectra were recorded on either a Bruker 400 or 500 MHz NMR spectrometer. Chemical shifts are reported in 8 values relative to tetramethylsilane. Liquid chromatography (LC)/mass spectra were run on a Shimadzu LC coupled to a Waters Micromass ZQ.

### LC-MS Methods:

LC/MS Method A=Column: PUROSPHER@star RP-18 (4×55 mm), 3 μm; Buffer: 20 mM NH₄OAC IN WATER; Mphase A: Buffer+ACN (90+10); Mphase B: Buffer+MeCN (10+90); Flow: 2.5 mL/min.
LC/MS Method B=Column: ZORBAX SB C18 (4.6×50 mm), 5 μm; Positive mode Mphase A: 10% MeOH—90% H₂O—0.1% TFA; Mphase B: 90% MeOH—10% H₂O—0.1% TFA; Flow: 5 mL/min.
LC/MS Method C=Column−Ascentis Express C8 (5×2.1 mm), 2.7 μm; Mphase A: 2% MeCN—98% H₂O—10 mM NH₄COOH; Mphase B: 98% ACN—2% H₂O—10 mM NH₄COOH; Flow: 1 mL/min.
LC/MS Method D=Column—ACQUITY UPLC BEH C18 (2.1×50 mm), 1.7 μm; Mphase A: 0.1% TFA in water; Mphase B: ACN; Flow: 1 mL/min.
LC/MS Method E=Column−Ascentis Express C18 (4.6×50 mm), 2.7 m; Mphase A: 5% MeCN—95% H₂O—10 mM NH₄OAC; Mphase B: 95% ACN—5% H₂O—10 mM NH₄OAC; Flow: 4 mL/min.
LC/MS Method F=Column−X Bridge Phe (4.6×30 mm), 3.5 m; Mphase A: 2% MeCN—98% H₂O—10 mM NH₄COOH; Mphase B: 98% ACN—2% H₂O—10 mM NH₄COOH; Flow: 1.8 mL/min.
LC/MS Method G=Column−Ascentis Express C18 (5×2.1 mm), 2.7 μm; Mphase A: 2% MeCN—98% H₂O—10 mM NH₄COOH; Mphase B: 98% ACN—2% H₂O—10 mM NH₄COOH; Flow: 1 mL/min.
LC/MS Method H=Column−ZORBAX Eclipse plus C18 (4.6×100 mm), 5 m; Mphase A: 20 mM NH₄OAc in H₂O 90%, MeCN 10%; Mphase B: 20 mM NH₄OAc in H₂O 10%, MeCN 90% Flow: 1 mL/min.
LC/MS Method I=Column−ZORBAX SB AQ (4.6×50 mm), 3.5 m; Mphase A: 0.1% HCOOH; Mphase B: MeCN Flow: 1 mL/min.

### GCMS Methods:

### Method A:

Column: DB-5MS, 30 m×0.25 mm ID×0.25u Film thickness.
Column flow: 0.9 mL/min at constant flow of Helium

### Carrier gas: Helium

Column temperature gradient:

| Rate (° C./min) | Temperature (° C.) | Hold Time (min) |
|---|---|---|
| — | 50 | 1 |
| 25 | 300 | 5 |

Injector temperature: 250° C.
Injection volume: 1 ul
Split ratio: 1:20

Mass detector:

| | |
|---|---|
| Source Temp: | 230° C. |
| Quadra pole Temp: | 150° C. |

### Chiral HPLC Methods:

Method A: CHIRALPAK AD-H (250×4.6) mm 5 micron

Mob. Phase: 0.2% DEA in n-hexane: IPA (80:20)

Method B: CHIRALPAK-ASH (250×4.6) mm 5 micron

Mob. Phase: 0.2% DEA in n-hexane: ethanol (70:30)

Method C: CHIRALPAK IC (250×4.6) mm 5 micron

Mob. Phase: 0.1% TFA in n-hexane: ethanol (40:60)

Method D: CHIRALPAK IA (250×4.6) mm 5 micron

Mob. Phase: 0.1% TFA in hexane: ethanol (50:50)

Method E: CHIRALPAK IC (250×4.6) mm 5 micron

Mob. Phase: 0.05% TFA in H₂O: acetonitrile (80:20)

Method F: CHIRALCEL ODH (250×4.6) mm 5 micron

Mob. phase: 0.2% DEA in n-hexane: ethanol (30:70)

### Chiral SFC Methods:

Method A1: Column: CHIRALPAK IC; Co Solvent: 0.5% DEA in Methanol; Co Solvent %: 50; Total flow: 3 g/min; Column Temperature: 35.4; Back pressure: 93 bar; Instrument: THAR SFC
Method A2: Column: CHIRALPAK IC; Co Solvent: 0.5% DEA in Methanol; Co Solvent %: 50; Total flow: 3 g/min; Column Temperature: 40.5; Back pressure: 100 bar; Instrument: THAR SFC
Method A3: Column: CHIRALPAK IC; Co Solvent: 0.5% DEA in Methanol; Co Solvent %: 40; Total flow: 3 g/min; Column Temperature: 34.7; Back pressure: 101 bar; Instrument: THAR SFC
Method A4: Column: CHIRALPAK IC; Co Solvent: 0.5% DEA in Methanol; Co Solvent %: 40; Total flow: 3 g/min; Column Temperature: 35.9; Back pressure: 101 bar; Instrument: THAR SFC
Method B1: Column: CHIRALCEL OD H; Co Solvent: 0.5% DEA in Methanol; Co Solvent %: 30; Total flow: 3 g/min; Column Temperature: 34.8; Back pressure: 100 bar; Instrument: THAR SFC
Method C1: Column: CHIRALPAK AD H; Co Solvent: 0.5% DEA in Methanol; Co Solvent %: 20; Total flow: 3 g/min; Column Temperature: 36.2; Back pressure: 100 bar; Instrument: THAR SFC
Method C2: Column: CHIRALPAK AD H; Co Solvent: 0.5% DEA in Methanol; Co Solvent %: 20; Total flow: 3 g/min; Column Temperature: 34.7; Back pressure: 99 bar; Instrument: THAR SFC
Method C3: Column: CHIRALPAK AD H; Co Solvent: 0.5% DEA in Methanol; Co Solvent %: 40; Total flow: 3 g/min; Column Temperature: 32; Back pressure: 97 bar; Instrument: THAR SFC
Method C₄: Column: CHIRALPAK AD H; Co Solvent: 0.5% DEA in Methanol; Co Solvent %: 30; Total flow: 3 g/min; Column Temperature: 34.8; Back pressure: 102 bar; Instrument: THAR SFC

### Analytical HPLC Methods:

Method A: Waters analytical C18 Sunfire column (4.6×150 mm, 3.5 m); mobile phase:
Buffer: 0.05% TFA in H₂O pH=2.5 adjusted with ammonia
A=buffer and acetonitrile (95:5), B=acetonitrile and buffer (95:5); 0-15 min, 0% B→50% B; 15-18 min, 50% B→100% B; 18-23 min, 100% B; flow rate=1 mL/min; λ=254 nm and 220 nm; run time=28 min.
Method B: Waters analytical phenyl xbridge column (4.6×150 mm, 3.5 μm), mobile phase:
Buffer: 0.05% TFA in H₂O pH=2.5 adjusted with ammonia
A=buffer and acetonitrile (95:5), B=acetonitrile and buffer (95:5); 0-15 min, 0% B→50% B; 15-18 min, 50% B→100% B; 18-23 min, 100% B; flow rate=1 mL/min; λ=254 nm and 220 nm; run time=28 min.
Method C: Waters analytical C18 sunfire column (4.6×150 mm, 3.5 m); mobile phase:
Buffer: 0.05% TFA in H₂O pH=2.5 adjusted with ammonia
A=buffer and acetonitrile (95:5), B=acetonitrile and buffer (95:5); 0-12 min, 10% B→100% B; 12-15 min, 100% B; flow rate=1 mL/min; λ=254 nm and 220 nm; run time=17 min.
Method D: Waters analytical phenyl xbridge column (4.6×150 mm, 3.5 m), mobile phase:
Buffer: 0.05% TFA in H₂O pH=2.5 adjusted with ammonia
A=buffer and acetonitrile (95:5), B=acetonitrile and buffer (95:5); 0-12 min, 10% B→100% B; 12-15 min, B→100% B; flow rate=1 mL/min; λ=254 nm and 220 nm; run time=17 min.
Method E: Waters analytical phenyl xbridge column (4.6×150 mm, 3.5 m), mobile phase: A=10 m M NH₄HCO₃ in H₂O pH=9.5 adjusted with ammonia, B=methanol; 0-12 min, 10% B→100% B; 12-20 min, B→100% B; flow rate=1 mL/min; λ=254 nm and 220 nm; run time=23 min.
Method F: Waters analytical C18 Sunfire column (4.6×150 mm, 3.5 m); mobile phase:
Buffer: 0.05% TFA in H₂O pH=2.5 adjusted with ammonia
A=buffer and acetonitrile (95:5), B=acetonitrile and buffer (95:5); 0-25 min, 10% B→100% B; 25-30 min, 100% B; flow rate=1 mL/min; λ=254 nm and 220 nm; run time=32 min.
Method G: ECLIPSE XDB C18 (4.6×150 mm, 3.5 μm); mobile phase A=20 mM NH₄OAc in H₂O, B=acetonitrile; 0-12 min, 10% B→100% B; 12-15 min, 100% B; flow rate=1 mL/min; λ=254 nm and 220 nm; run time=18 min.
Method H: Waters analytical phenyl xbridge column (4.6×150 mm, 3.5 μm); mobile phase: Buffer: 0.05% TFA in H₂O pH=2.5 adjusted with ammonia
A=buffer and acetonitrile (95:5), B=acetonitrile and buffer (95:5); 0-25 min, 10% B→100% B; 25-30 min, 100% B; flow rate=1 mL/min; λ=254 nm and 220 nm; run time=32 min.
Method I: Waters analytical phenyl xbridge column (4.6×150 mm, 3.5 μm), mobile phase: A=10 m M NH₄HCO₃ in H₂O pH=9.5 adjusted with ammonia, B=methanol; 0-25 min, 10% B→100% B; 25-30 min, B→100% B; flow rate=1 mL/min; λ=254 nm and 220 nm; run time=30 min.
Method J: ECLIPSE XDB C18 (4.6×150 mm, 5 μm); mobile phase A=20 mM NH₄OAc in H₂O, B=acetonitrile; 0-25 min, 10% B→100% B; 25-30 min, 100% B; flow rate=1 mL/min; λ=254 nm and 220 nm; run time=30 min.
Method K: Waters analytical phenyl xbridge column (4.6×150 mm, 3.5 μm), mobile phase: A=10 mM NH₄HCO₃ in H₂O pH=9.5 adjusted with ammonia, B=methanol; 0-15 min, 0% B→50% B; 15-18 min, 50%→100% B; 18-23 min, 100% B; flow rate=1 mL/min; λ=254 nm and 220 nm; run time=25 min.
Method L: ECLIPSE XDB C18 (4.6×150 mm, 5 μm); mobile phase:
A=20 mM NH₄OAc in H₂O, B=acetonitrile; 0-15 min, 0% B→50% B; 15-18 min, 50%-100% B; 18-23 min, 100% B; flow rate=1 mL/min; λ=254 nm and 220 nm; run time=25 min.
Method M: Waters analytical phenyl xbridge C18 column (4.6×150 mm, 3.5 μm), mobile phase: A=20 m M NH₄OAc in H₂O, B=acetonitrile; 0-25 min, 10% B→100% B; 25-30 min, B→100% B; flow rate=1 mL/min; λ=254 nm and 220 nm; run time=30 min.
Method N: Waters analytical phenyl xbridge C18 column (4.6×150 mm, 3.5 μm), mobile phase: A=20 mM NH₄OAc in H₂O, B=acetonitrile; 0-12 min, 10% B→100% B; 12-15 min, B→100% B; flow rate=1 mL/min; λ=254 nm and 220 nm; run time=20 min.
Method O: Waters analytical C18 Sunfire column (4.6×150 mm, 3.5 m); mobile phase: A=H₂O with 0.1% TFA, B=acetonitrile with 0.1% TFA; 1-15 min, 10% B→95% B; 15-18 min, 95% B; flow rate=1 mL/min; λ=254 nm; run time=18 min.
Method P: Waters analytical Phenyl Xbridge column (4.6×150 mm, 3.5 m), mobile phase: A=H₂O with 0.1% TFA, B=acetonitrile with 0.1% TFA, 1-15 min, 10% B→95% B; 15-18 min, 95% B; flow rate=1 mL/min; λ=254 nm; run time=18 min.

The following abbreviations are used: THF (tetrahydrofuran), MeOH (methanol), DMF (N,N-dimethylformamide), EtOH (ethanol), MeCN (acetonitrile), DCE (dichloroethane), DCM (dichloromethane), TFA (trifluoroacetic acid), HCl (hydrochloric acid), TosMIC (tosylmethyl isocyanide), DIPEA (diisopropylethyl amine), DMAP (dimethylaminopyridine), n-BuLi (n-butyllithium), NBS (N-bromosuccinimide), AIBN (azobisisobutyronitrile), DIPEA (N,N-diisopropylethylamine), DIBAL-H or DIBAL (diisobutylaluminum hydride), CDI (1,1′-carbonyldiimidazole), HATU (O-(7-azabenzotriazol-1-yl)-N,N,N′,N′-tetramethyluronium hexafluorophosphate), TBTU (N,N,N′,N′-tetramethyl-O-(benzotriazol-1-yl)uronium tetrafluoroborate), TLC (thin layer chromatography), NMR (nuclear magnetic resonance), LC/MS or LCMS (liquid chromatography/mass spectrometry), HPLC (high pressure liquid chromatography), GC (gas chromatography).

### Example 1

### 3-Methyl-1-(6-(oxazol-5-yl)quinazolin-2-yl)butan-1-amine

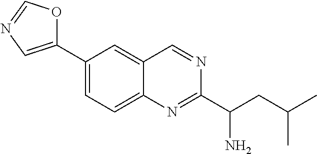 [SMILES: CC(C)CC(N)c1ncc2cc(-c3cnco3)ccc2n1]

### Part A. 5-(3-Bromo-4-nitrophenyl)oxazole

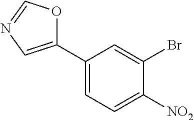 [SMILES: O=[N+]([O-])c1ccc(-c2cnco2)cc1Br]

To a solution of 3-bromo-4-nitrobenzaldehyde (3.70 g, 16.09 mmol) (Rao et al. Tetrahedron Lett., 1992, 33, 4799-4802) and TosMIC (3.77 g, 19.30 mmol) in MeOH (60 mL) was added potassium carbonate (2.78 g, 20.11 mmol). The reaction mixture was heated at reflux for 2.5 h. The reaction mixture was cooled to room temperature and was transferred to a separatory funnel containing saturated aqueous NaHCO₃ solution (150 mL). The aqueous layer was extracted with ethyl acetate (3×150 mL). The combined organic layers were washed with brine (100 mL), dried over MgSO₄, filtered, and concentrated. The residue was purified by column chromatography on silica gel (40%-60% ethyl acetate in hexanes) to afford 5-(3-bromo-4-nitrophenyl)oxazole (3.10 g, 72% yield) as a colorless solid: ¹H NMR (400 MHz, CDCl₃) δ 8.06 (d, J=2.0 Hz, 1H), 8.04 (s, 1H), 7.99 (d, J=8.6 Hz, 1H), 7.74 (dd, J=8.4, 1.9 Hz, 1H), 7.58 (s, 1H).

### Part B. 5-(4-Nitro-3-vinylphenyl)oxazole

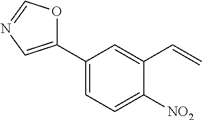 [SMILES: C=Cc1cc(-c2cnco2)ccc1[N+](=O)[O-]]

To a solution of 5-(3-bromo-4-nitrophenyl)oxazole (2.80 g, 10.41 mmol) in toluene (60 mL) and ethanol (15 mL) was added 2,4,6-trivinyl-1,3,5,2,4,6-trioxatriborinane pyridine complex (1.681 g, 10.41 mmol) and sodium carbonate (2 N, aq) (12.49 mL, 12.49 mmol). The solution was degassed with N₂ for 10 min with a glass frit. Pd(PPh₃)₄ (0.601 g, 0.520 mmol) was added to the reaction mixture and the mixture was heated at 95° C. for 2.5 h. The reaction mixture was cooled to room temperature and transferred to a separatory funnel containing saturated aqueous NaHCO₃ solution (75 mL). The aqueous layer was extracted with ether (3×100 mL). The combined organic layers were washed with brine (50 mL), dried over MgSO₄, filtered, and concentrated. The residue was purified by column chromatography on silica gel (40%-60% ethyl acetate in hexanes) to afford 5-(4-nitro-3-vinylphenyl)oxazole (2.04 g, 91% yield) as a yellow solid: ¹H NMR (400 MHz, CDCl₃) δ 8.08 (d, J=8.6 Hz, 1H), 8.04 (s, 1H), 7.89 (d, J=1.8 Hz, 1H), 7.70 (dd, J=8.6, 2.0 Hz, 1H), 7.57 (s, 1H), 7.27 (dd, J=17.4, 11.1 Hz, 1H), 5.84 (dd, J=17.4, 0.8 Hz, 1H), 5.59 (dd, J=11.0, 0.6 Hz, 1H); MS (ESI) m/e 258.1 [(M+H+CH₃CN)⁺, calcd for C₁₃H₁₂N₃O₃ 258.1].

### Part C. 4-(Oxazol-5-yl)-2-vinylaniline

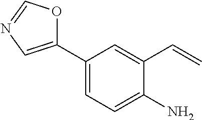

To a solution of 5-(4-nitro-3-vinylphenyl)oxazole (1.00 g, 4.63 mmol) in ethanol (35 mL) was added ammonium chloride (2.97 g, 55.5 mmol) and zinc powder (4.23 g, 64.8 mmol). The reaction mixture was heated at 70° C. for 4 h. The reaction mixture was cooled to room temperature and was filtered through a pad of Celite. The filtrate was transferred to a separatory funnel containing saturated aqueous NaHCO₃ solution (50 mL). The aqueous layer was extracted with CH₂Cl₂ (3×50 mL). The combined organic layers were washed with brine (50 mL), dried over MgSO₄, filtered and concentrated. The residue was purified by column chromatography on silica gel (20%-50% ethyl acetate in hexanes) to afford 4-(oxazol-5-yl)-2-vinylaniline (788 mg, 91% yield) as a colorless solid: ¹H NMR (400 MHz, CDCl₃) δ 7.87 (s, 1H), 7.58 (d, J=2.0 Hz, 1H), 7.41 (dd, J=8.3, 2.0 Hz, 1H), 7.21 (s, 1H) 6.84-6.72 (m, 2H), 5.73 (dd, J=17.4, 1.3 Hz, 1H), 5.43 (dd, J=11.1, 1.5 Hz, 1H), 3.95 (br. s., 2H); MS (ESI) m/e 187.1 [(M+H)⁺, calcd for C₁₁H₁₁N₂O 187.1].

### Part D. (R)-Benzyl 4-methyl-1-(4-(oxazol-5-yl)-2-vinylphenylamino)-1-oxopentan-2-ylcarbamate

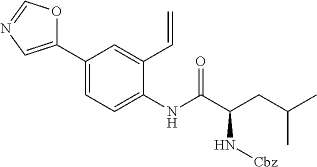 [SMILES: C=Cc1cc(-c2cnco2)ccc1NC(=O)[C@@H](CC(C)C)NC(=O)OCc1ccccc1]

To 4-(oxazol-5-yl)-2-vinylaniline (390 mg, 2.094 mmol) was added via cannula a solution of (R)-2-(benzyloxycarbonylamino)-4-methylpentanoic acid (667 mg, 2.51 mmol) in DCE (15 mL) (+2 mL rinse). N,N-Diisopropylethylamine (1.83 mL, 10.5 mmol) was then added followed by HATU (2.39 g, 6.28 mmol). The reaction mixture was stirred at room temperature for 10 min and was then heated at 40° C. for 18 h. The reaction mixture was transferred to a separatory funnel containing saturated aqueous NH₄Cl solution (50 mL). The aqueous layer was extracted with ethyl acetate (3×50 mL). The combined organic layers were washed with saturated aqueous NaHCO₃ solution (10 mL) and brine (50 mL), dried over MgSO₄, filtered, and concentrated. The residue was purified via column chromatography on silica gel (20%-50% ethyl acetate in hexanes) to afford (R)-benzyl 4-methyl-1-(4-(oxazol-5-yl)-2-vinylphenylamino)-1-oxopentan-2-ylcarbamate (773 mg, 85% yield) as a pale yellow solid: ¹H NMR (400 MHz, CDCl₃) δ 8.19 (br. s., 1H), 8.03 (d, J=8.6 Hz, 1H), 7.93 (s, 1H), 7.68 (s, 1H), 7.56 (dd, J=8.4, 2.1 Hz, 1H), 7.40-7.32 (m, 5H), 6.86-6.74 (m, 1H), 5.73 (d, J=17.6 Hz, 1H), 5.47 (d, J=11.1 Hz, 1H), 5.27 (d, J=5.8 Hz, 1H), 5.17 (s, 2H), 4.36 (d, J=5.3 Hz, 1H), 1.92-1.72 (m, 2H), 1.66-1.58 (m, 1H), 1.04-0.97 (m, 6H); MS (ESI) m/e 434.2 [(M+H)⁺, calcd for C₂₅H₂₈N₃O₄ 434.2].

### Part E. (R)-Benzyl 1-(2-formyl-4-(oxazol-5-yl)phenylamino)-4-methyl-1-oxopentan-2-ylcarbamate

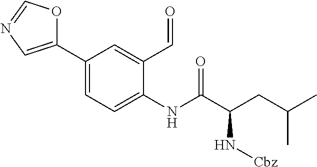 [SMILES: CC(C)C[C@@H](NC(=O)OCc1ccccc1)C(=O)Nc1ccc(-c2cnco2)cc1C=O]

To a solution of (R)-benzyl 4-methyl-1-(4-(oxazol-5-yl)-2-vinylphenylamino)-1-oxopentan-2-ylcarbamate (700 mg, 1.615 mmol) in dioxane (20 mL) and water (5 mL) at 0° C. was added 2,6-lutidine (0.376 mL, 3.23 mmol), osmium tetroxide (2.5% in 2-methyl-2-propanol) (0.405 mL, 0.032 mmol), and sodium periodate (1.38 g, 6.46 mmol). The cooling bath was removed and the reaction mixture was allowed to warm up to room temperature while stirring for 2.5 h. The reaction mixture was transferred to a separatory funnel containing water (5 mL) and saturated aqueous NaHCO₃ solution (5 mL). The aqueous layer was extracted with ethyl acetate (3×10 mL). The combined organic layers were washed with brine (5 mL), dried over MgSO₄, filtered, and concentrated. The residue was purified by column chromatography on silica gel (30%-60% ethyl acetate in hexanes) to afford (R)-benzyl 1-(2-formyl-4-(oxazol-5-yl)phenylamino)-4-methyl-1-oxopentan-2-ylcarbamate (589 mg, 84% yield) as an off-white solid: ¹H NMR (400 MHz, CDCl₃) δ 11.67 (br. s., 1H), 10.00 (s, 1H), 8.85 (d, J=8.8 Hz, 1H), 8.00-7.95 (m, 2H), 7.88 (dd, J=8.8, 2.3 Hz, 1H), 7.47-7.33 (m, 5H), 5.36 (d, J=7.3 Hz, 1H), 5.19 (s, 2H), 4.48-4.38 (m, 1H), 1.91-1.72 (m, 2H), 1.67-1.57 (m, 1H), 1.07-0.97 (m, 6H); MS (ESI) m/e 436.2 [(M+H)⁺, calcd for C₂₄H₂₆N₃O₅ 436.2].

### Part F. Benzyl 3-methyl-1-(6-(oxazol-5-yl)quinazolin-2-yl)butylcarbamate

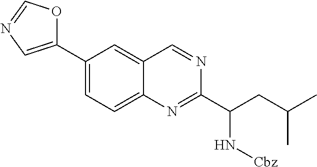 [SMILES: CC(C)CC(NC(=O)OCc1ccccc1)c1ncc2cc(-c3cnco3)ccc2n1]

To a solution of (R)-benzyl 1-(2-formyl-4-(oxazol-5-yl)phenylamino)-4-methyl-1-oxopentan-2-ylcarbamate (175 mg, 0.402 mmol) in acetic acid (7 mL) was added ammonium acetate (310 mg, 4.02 mmol). The reaction mixture was heated at 70° C. for 2.5 h. The mixture was cooled to room temperature and was concentrated. The reaction mixture was transferred to a separatory funnel containing saturated aqueous NaHCO₃ solution (5 mL). The aqueous layer was extracted with ethyl acetate (3×10 mL). The combined organic layers were washed with brine (5 mL), dried over MgSO₄, filtered, and concentrated. The residue was purified by column chromatography on silica gel (30%-60% ethyl acetate in hexanes) to afford benzyl 3-methyl-1-(6-(oxazol-5-yl)quinazolin-2-yl)butylcarbamate (130 mg, 78% yield) as a colorless foam: ¹H NMR (400 MHz, CDCl₃) δ 9.42 (s, 1H), 8.20 (s, 1H), 8.19-8.15 (m, 1H), 8.07 (d, J=8.8 Hz, 1H), 8.04 (s, 1H), 7.57 (s, 1H), 7.44-7.30 (m, 5H), 5.96 (d, J=8.6 Hz, 1H), 5.30-5.22 (m, 1H), 5.15 (d, J=2.5 Hz, 2H), 1.88-1.72 (m, 3H), 1.06 (d, J=5.8 Hz, 3H), 0.98 (d, J=5.8 Hz, 3H); MS (ESI) m/e 417.3 [(M+H)⁺, calcd for C₂₄H₂₅N₄O₃ 417.2].

### Part G. 3-Methyl-1-(6-(oxazol-5-yl)quinazolin-2-yl)butan-1-amine

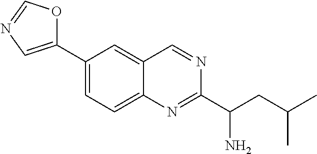 [SMILES: CC(C)CC(N)c1ncc2cc(-c3cnco3)ccc2n1]

To a solution of benzyl 3-methyl-1-(6-(oxazol-5-yl)quinazolin-2-yl)butylcarbamate (85 mg, 0.204 mmol) in CH₂Cl₂ (3 mL) at 0° C. was added boron tribromide (0.612 mL, 0.612 mmol). The reaction mixture was stirred at 0° C. for 10 min. The reaction was quenched by the addition of triethylamine. The reaction mixture was concentrated and the product was purified by reverse phase HPLC (5% MeCN:95% water:0.1% TFA→95% MeCN:5% water:0.1% TFA). The organic solvent was removed on the rotovapor and the aqueous mixture was transferred to separatory funnel containing saturated aqueous K₂CO₃. The aqueous layer was extracted with ethyl acetate (4×10 mL). The combined organic layers were washed with brine (5 mL), dried over MgSO₄, filtered and concentrated. The residue was taken up in acetonitrile/water and was frozen and placed on the lyophilizer to afford 3-methyl-1-(6-(oxazol-5-yl)quinazolin-2-yl)butan-1-amine (14.4 mg, 24% yield) as a colorless amorphous solid: ¹H NMR (400 MHz, DMSO-d₆) δ 9.69 (s, 1H), 8.60 (s, 1H), 8.47 (d, J=1.8 Hz, 1H), 8.38 (dd, J=8.9, 1.9 Hz, 1H), 8.07 (d, J=9.1 Hz, 1H), 7.95 (s, 1H), 4.19 (t, J=6.9 Hz, 1H), 1.77-1.57 (m, 3H), 0.92 (d, J=6.0 Hz, 3H), 0.89 (d, J=6.0 Hz, 3H); MS (ESI) m/e 283.2 [(M+H)⁺, calcd for C₁₆H₁₉N₄O 283.2]. HPLC retention time (Method O): t_{R}=7.91 min; HPLC retention time (Method P): t_{R}=7.69 min.

### Example 2

### 3-Methyl-1-(4-methyl-6-(oxazol-5-yl)quinazolin-2-yl)butan-1-amine

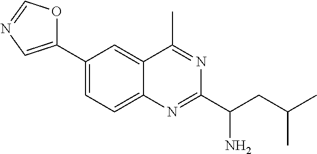 [SMILES: Cc1nc(C(N)CC(C)C)nc2ccc(-c3cnco3)cc12]

### Part A. (R)-tert-Butyl 4-methyl-1-(4-(oxazol-5-yl)-2-vinylphenylamino)-1-oxopentan-2-ylcarbamate

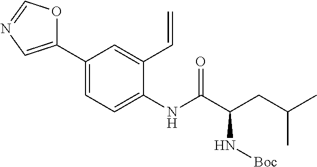 [SMILES: C=Cc1cc(-c2cnco2)ccc1NC(=O)[C@@H](CC(C)C)NC(=O)OC(C)(C)C]

4-(Oxazol-5-yl)-2-vinylaniline (150 mg, 0.806 mmol) (prepared as described in Example 1, Parts A-C), (R)-2-(tert-butoxycarbonylamino)-4-methylpentanoic acid (559 mg, 2.417 mmol) and dichloroethane (4 mL) were combined in a round bottom flask. N,N-Diisopropylethylamine (0.703 mL, 4.03 mmol) was then added followed by HATU (919 mg, 2.417 mmol). The reaction mixture was stirred at room temperature for 10 min and was then heated at 40° C. for 18 h. The reaction mixture was transferred to a separatory funnel containing saturated aqueous NH₄Cl solution (50 mL). The aqueous layer was extracted with ethyl acetate (3×20 mL). The combined organic layers were washed with saturated aqueous NaHCO₃ solution (20 mL) and brine (20 mL), dried over MgSO₄, filtered, and concentrated. The residue was purified by column chromatography on silica gel (10%-40% ethyl acetate in hexanes) to afford (R)-tert-butyl 4-methyl-1-(4-(oxazol-5-yl)-2-vinylphenylamino)-1-oxopentan-2-ylcarbamate (234 mg, 73% yield) as a pale yellow solid: ¹H NMR (400 MHz, CDCl₃) δ 8.36 (br. s., 1H), 8.09 (d, J=8.6 Hz, 1H), 7.92 (s, 1H), 7.70 (d, J=2.0 Hz, 1H), 7.57 (dd, J=8.4, 1.9 Hz, 1H), 7.34 (s, 1H), 6.86 (dd, J=17.2, 11.2 Hz, 1H), 5.77 (dd, J=17.4, 0.8 Hz, 1H), 5.51 (d, J=11.6 Hz, 1H), 4.94 (d, J=5.0 Hz, 1H), 4.27 (d, J=6.0 Hz, 1H), 1.92-1.83 (m, 1H), 1.82-1.73 (m, 1H), 1.60 (ddd, J=13.8, 8.9, 5.5 Hz, 1H), 1.50 (s, 8H), 1.02 (d, J=6.5 Hz, 3H), 0.99 (d, J=6.5 Hz, 3H); MS (ESI) m/e 400.3 [(M+H)⁺, calcd for C₂₂H₃₀N₃O₄ 400.2].

### Part B. (R)-tert-Butyl 1-(2-formyl-4-(oxazol-5-yl)phenylamino)-4-methyl-1-oxopentan-2-ylcarbamate

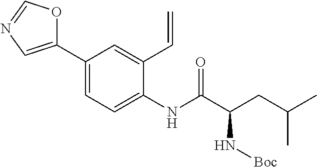 [SMILES: C=Cc1cc(-c2cnco2)ccc1NC(=O)[C@@H](CC(C)C)NC(=O)OC(C)(C)C]

To a solution of (R)-tert-butyl 4-methyl-1-(4-(oxazol-5-yl)-2-vinylphenylamino)-1-oxopentan-2-ylcarbamate (170 mg, 0.426 mmol) in dioxane (5 mL) and water (1.25 mL) at 0° C. was added 2,6-lutidine (0.099 mL, 0.851 mmol), osmium tetroxide (2.5% in 2-methyl-2-propanol) (0.107 mL, 8.51 μmol), and sodium periodate (364 mg, 1.702 mmol). The cooling bath was removed and the reaction mixture was allowed to warm up to room temperature while stirring for 2.5 h. The reaction mixture was transferred to a separatory funnel containing water (5 mL) and saturated aqueous NaHCO₃ solution (5 mL). The aqueous layer was extracted with ethyl acetate (3×10 mL). The combined organic layers were washed with brine (5 mL), dried over MgSO₄, filtered, and concentrated. The residue was purified by column chromatography on silica gel (30%-60% ethyl acetate in hexanes) to afford (R)-tert-butyl 1-(2-formyl-4-(oxazol-5-yl)phenylamino)-4-methyl-1-oxopentan-2-ylcarbamate (145 mg, 85% yield) as an off-white solid: ¹H NMR (400 MHz, CDCl₃) δ 11.69 (s, 1H), 10.02 (s, 1H), 8.88 (d, J=8.6 Hz, 1H), 7.98 (d, J=1.8 Hz, 1H), 7.97 (s, 1H), 7.89 (dd, J=8.8, 2.0 Hz, 1H), 7.41 (s, 1H), 5.04 (d, J=6.8 Hz, 1H), 4.35 (br. s., 1H), 1.88-1.75 (m, 2H), 1.64-1.56 (m, 1H), 1.50 (s, 9H), 1.03 (d, J=3.3 Hz, 3H), 1.01 (d, J=3.5 Hz, 3H); MS (ESI) m/e 302.1 [(M+H—C₅H₈O₂), calcd for C₁₆H₂₀N₃O₃ 302.2].

### Part C. tert-Butyl-1-(2-(1-hydroxyethyl)-4-(oxazol-5-yl)phenylamino)-4-methyl-1-oxopentan-2-ylcarbamate

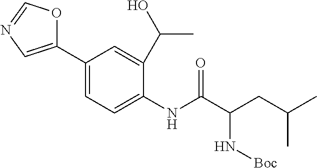 [SMILES: CC(C)CC(NC(=O)OC(C)(C)C)C(=O)Nc1ccc(-c2cnco2)cc1C(C)O]

To a solution of (R)-tert-butyl 1-(2-formyl-4-(oxazol-5-yl)phenylamino)-4-methyl-1-oxopentan-2-ylcarbamate (95 mg, 0.237 mmol) in THF (2 mL) at −78° C. was added methylmagnesium bromide (0.087 mL, 0.260 mmol, 3 M). The mixture was stirred at −78° C. for 30 min then warmed up to 0° C. and was stirred at 0° C. for 1 h. Additional methylmagnesium bromide (0.50 mL, 1.50 mmol, 3 M) was added and the mixture was stirred for 1 h at 0° C. The reaction was not complete. Additional methylmagnesium bromide (0.50 mL, 1.50 mmol, 3 M) was added and the mixture was stirred for an additional 30 min at 0° C. Nearly all the starting material was consumed. The reaction was quenched by the addition of saturated aqueous NaHCO₃ solution. The mixture was filtered through a pad of Celite and the mixture was transferred to a separatory funnel containing saturated aqueous NaHCO₃ solution (5 mL). The aqueous layer was extracted with ethyl acetate (3×10 mL). The combined organic layers were washed with brine (5 mL), dried over MgSO₄, filtered, and concentrated. The residue was purified by column chromatography on silica gel (20%-50% ethyl acetate in hexanes) to afford tert-butyl-1-(2-(1-hydroxyethyl)-4-(oxazol-5-yl)phenylamino)-4-methyl-1-oxopentan-2-ylcarbamate (40 mg, 41% yield) as a colorless foam: ¹H NMR (400 MHz, CDCl₃) δ 9.68 (br. s., 1H), 8.28 (d, J=4.5 Hz, 1H), 7.91 (s, 1H), 7.59 (dd, J=8.6, 1.8 Hz, 1H), 7.47 (s, 1H), 7.32 (s, 1H), 5.12-5.02 (m, 1H), 4.96 (br. s., 1H), 4.28 (br. s., 1H), 1.91-1.73 (m, 2H), 1.63 (d, J=6.5 Hz, 3H), 1.58-1.54 (m, 1H), 1.49 (s, 9H), 1.02 (d, J=3.5 Hz, 3H), 1.01 (d, J=3.5 Hz, 3H); MS (ESI) m/e 418.3 [(M+H)⁺, calcd for C₂₂H₃₂N₃O₅ 418.2].

### Part D. tert-Butyl 1-(2-acetyl-4-(oxazol-5-yl)phenylamino)-4-methyl-1-oxopentan-2-ylcarbamate

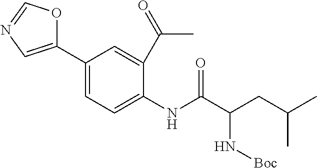 [SMILES: CC(=O)c1cc(-c2cnco2)ccc1NC(=O)C(CC(C)C)NC(=O)OC(C)(C)C]

Oxalyl chloride (0.147 mL, 0.293 mmol) was added to a solution of DMSO (0.036 mL, 0.503 mmol) in CH₂Cl₂ (1 mL) at −78° C. After stirring for 10 min, tert-butyl-1-(2-(1-hydroxyethyl)-4-(oxazol-5-yl)phenylamino)-4-methyl-1-oxopentan-2-ylcarbamate (35 mg, 0.084 mmol) dissolved in CH₂Cl₂ (1 mL) as added via cannula. The solution was stirred at −78° C. for 45 min. Triethylamine (0.140 mL, 1.006 mmol) was added dropwise via syringe and the reaction mixture was stirred at −78° C. for 15 min and was then allowed to warm up to room temperature over 30 min. The reaction mixture was transferred to a separatory funnel containing saturated aqueous NaHCO₃ solution (5 mL). The aqueous layer was extracted with ethyl acetate (3×10 mL). The combined organic layers were washed with brine (5 mL), dried over MgSO₄, filtered, and concentrated. The residue was purified by column chromatography on silica gel (30%-50% ethyl acetate in hexanes) to afford tert-butyl 1-(2-acetyl-4-(oxazol-5-yl)phenylamino)-4-methyl-1-oxopentan-2-ylcarbamate (25 mg, 72% yield) as a colorless solid: ¹H NMR (400 MHz, CDCl₃) δ 12.22 (br s, 1H), 8.90 (d, J=8.8 Hz, 1H), 8.18 (d, J=1.8 Hz, 1H), 7.96 (s, 1H), 7.83 (dd, J=8.8, 1.8 Hz, 1H), 7.39 (s, 1H), 5.03 (d, J=7.1 Hz, 1H), 4.34 (br s, 1H), 2.75 (s, 3H), 1.86-1.74 (m, 2H), 1.63-1.56 (m, 1H), 1.50 (s, 9H), 1.02 (t, J=5.9 Hz, 6H); MS (ESI) m/e 316.2 [(M+H—C₅H₈O₂)⁺, calcd for C₁₇H₂₂N₃O₃ 316.2].

### Part E. 3-Methyl-1-(4-methyl-6-(oxazol-5-yl)quinazolin-2-yl)butan-1-amine

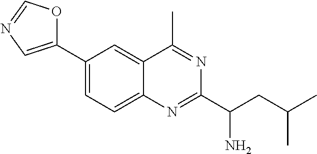 [SMILES: Cc1nc(C(N)CC(C)C)nc2ccc(-c3cnco3)cc12]

To a solution of tert-butyl 1-(2-acetyl-4-(oxazol-5-yl)phenylamino)-4-methyl-1-oxopentan-2-ylcarbamate (25 mg, 0.060 mmol) in acetic acid (1 mL) was added ammonium acetate (46.4 mg, 0.602 mmol). The reaction mixture was heated at 70° C. for 3 h. Additional ammonium acetate (46 mg) was added and the mixture was heated at 70° C. for 2 h. No reaction took place. The mixture was then heated at 85° C. for 16 h. All of the starting material was consumed. A mixture of products had formed (observed by LC/MS) consisting of the cyclized product with the Boc group still intact and the cyclized product wherein the Boc group was not present. The mixture was cooled to room temperature and was concentrated. The product was purified by reverse phase HPLC (5% MeCN:95% water:0.1% TFA→95% MeCN:5% water:0.1% TFA). Both the product with the Boc group still intact and the product wherein the Boc group was not present were isolated. The product with the Boc group still intact was taken up in CH₂Cl₂ (2 mL) and was cooled to 0° C. TFA (0.5 mL) was added. The cooling bath was removed, and the mixture was stirred at room temperature for 30 min. The mixture was concentrated and the product was purified by reverse phase HPLC (5% MeCN:95% water:0.1% TFA→95% MeCN:5% water:0.1% TFA). The product without the Boc group from both purifications was combined and the organic solvent was removed on the rotovapor and the aqueous mixture was frozen and placed on the lyophilizer to afford 3-methyl-1-(4-methyl-6-(oxazol-5-yl)quinazolin-2-yl)butan-1-amine (7 mg, 38% yield) as a TFA salt: ¹H NMR (500 MHz, CDCl₃) δ 8.37 (s, 1H), 8.17 (d, J=8.9 Hz, 1H), 8.08 (s, 1H), 8.06 (d, J=8.9 Hz, 1H), 7.59 (s, 1H), 4.82-4.74 (m, 1H), 3.03 (s, 2H), 2.19-2.08 (m, 1H), 2.04-1.94 (m, 1H), 1.94-1.83 (m, 1H), 1.06 (d, J=6.4 Hz, 6H); MS (ESI) m/e 297.2 [(M+H)⁺, calcd for C₁₇H₂₁N₄O 297.2]. HPLC retention time (Method O): t_{R}=8.31 min; HPLC retention time (Method P): t_{R}=8.26 min.

### Example 3

### 1-(4-Ethyl-6-(oxazol-5-yl)quinazolin-2-yl)-3-methylbutan-1-amine

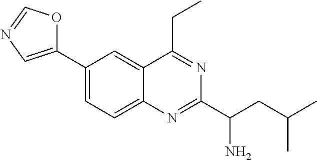 [SMILES: CCc1nc(C(N)CC(C)C)nc2ccc(-c3cnco3)cc12]

### Part A. tert-Butyl-1-(2-(1-hydroxypropyl)-4-(oxazol-5-yl)phenylamino)-4-methyl-1-oxopentan-2-ylcarbamate

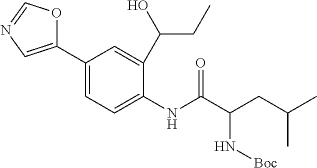 [SMILES: CCC(O)c1cc(-c2cnco2)ccc1NC(=O)C(CC(C)C)NC(=O)OC(C)(C)C]

To a solution of (R)-tert-butyl 1-(2-formyl-4-(oxazol-5-yl)phenylamino)-4-methyl-1-oxopentan-2-ylcarbamate (200 mg, 0.498 mmol), prepared as described in Example 2, Parts A-B, in THF (4 mL) at −78° C. was added ethylmagnesium bromide (0.996 mL, 0.996 mmol, 1 M). The mixture was stirred at −78° C. for 30 min then warmed up to 0° C. and was stirred at 0° C. for 1 h. Additional ethylmagnesium bromide (1.00 mL, 1.00 mmol, 1 M) was added and the mixture was stirred for 30 min at 0° C. The reaction was not complete. Additional ethylmagnesium bromide (3.0 mL, 3.00 mmol, 1 M) was added and the mixture was stirred for an additional 30 min at 0° C. The reaction was quenched by the addition of saturated aqueous NaHCO₃ solution. The mixture was filtered through a pad of Celite and the reaction mixture was transferred to a separatory funnel containing saturated aqueous NaHCO₃ solution (10 mL). The aqueous layer was extracted with ethyl acetate (3×20 mL). The combined organic layers were washed with brine (10 mL), dried over MgSO₄, filtered, and concentrated. The residue was purified by column chromatography on silica gel (20%-50% ethyl acetate in hexanes) to afford tert-butyl-1-(2-(1-hydroxypropyl)-4-(oxazol-5-yl)phenylamino)-4-methyl-1-oxopentan-2-ylcarbamate (113 mg, 53% yield) as a colorless foam: ¹H NMR (400 MHz, CDCl₃) δ 9.73 (br. s., 1H), 8.39-8.29 (m, 1H), 7.90 (s, 1H), 7.58 (dt, J=8.6, 2.0 Hz, 1H), 7.40 (d, J=2.0 Hz, 1H), 7.30 (s, 1H), 5.10 (br. s., 1H), 4.74 (td, J=7.1, 3.5 Hz, 1H), 4.20-4.10 (m, 1H), 2.13-2.03 (m, 1H), 2.00-1.81 (m, 2H), 1.62-1.51 (m, 1H), 1.48 (s, 9H), 1.26-1.15 (m, 1H), 1.07-0.94 (m, 9H); MS (ESI) m/e 432.3 [(M+H)⁺, calcd for C₂₃H₃₄N₃O₅ 432.2].

### Part B. tert-Butyl 4-methyl-1-(4-(oxazol-5-yl)-2-propionylphenylamino)-1-oxopentan-2-ylcarbamate

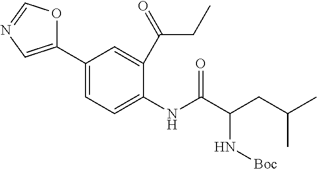 [SMILES: CCC(=O)c1cc(-c2cnco2)ccc1NC(=O)C(CC(C)C)NC(=O)OC(C)(C)C]

Oxalyl chloride (0.414 mL, 0.827 mmol) was added to a solution of DMSO (0.10 mL, 1.42 mmol) in CH₂Cl₂ (1.5 mL) at −78° C. After stirring for 10 min, tert-butyl-1-(2-(1-hydroxypropyl)-4-(oxazol-5-yl)phenylamino)-4-methyl-1-oxopentan-2-ylcarbamate (102 mg, 0.236 mmol) dissolved in CH₂Cl₂ (1 mL) was added via cannula. The solution was stirred at −78° C. for 45 min. Triethylamine (0.395 mL, 2.84 mmol) was then added dropwise via syringe and the reaction mixture was stirred at −78° C. for 15 min and was then allowed to warm up to room temperature over 30 min. The reaction mixture was transferred to a separatory funnel containing saturated aqueous NaHCO₃ solution (5 mL). The aqueous layer was extracted with ethyl acetate (3×10 mL). The combined organic layers were washed with brine (5 mL), dried over MgSO₄, filtered, and concentrated. The residue was purified by column chromatography on silica gel (30%-50% ethyl acetate in hexanes) to afford tert-butyl 4-methyl-1-(4-(oxazol-5-yl)-2-propionylphenylamino)-1-oxopentan-2-ylcarbamate (83 mg, 82% yield) as a colorless solid: ¹H NMR (400 MHz, CDCl₃) δ 12.26 (br. s., 1H), 8.91 (d, J=9.1 Hz, 1H), 8.23 (d, J=1.8 Hz, 1H), 7.95 (s, 1H), 7.82 (dd, J=8.8, 1.8 Hz, 1H), 7.38 (s, 1H), 5.17 (d, J=8.1 Hz, 1H), 4.29 (dd, J=7.6, 5.3 Hz, 1H), 3.16 (q, J=7.1 Hz, 2H), 2.13 (br. s., 1H), 1.51 (s, 9H), 1.48-1.41 (m, 1H), 1.27 (t, J=7.2 Hz, 3H), 1.25-1.14 (m, 1H), 1.05 (d, J=6.8 Hz, 3H), 0.96 (t, J=7.4 Hz, 3H); MS (ESI) m/e 330.3 [(M+H—C₅H₈O₂)⁺, calcd for C₁₈H₂₄N₃O₃ 330.2].

### Part C. 1-(4-Ethyl-6-(oxazol-5-yl)quinazolin-2-yl)-3-methylbutan-1-amine

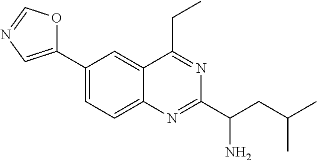 [SMILES: CCc1nc(C(N)CC(C)C)nc2ccc(-c3cnco3)cc12]

To a solution of tert-butyl 4-methyl-1-(4-(oxazol-5-yl)-2-propionylphenylamino)-1-oxopentan-2-ylcarbamate (72 mg, 0.168 mmol) in acetic acid (3 mL) was added ammonium acetate (258 mg, 3.35 mmol). The reaction mixture was heated at 95° C. for 17 h. The mixture was concentrated and the product was purified by reverse phase HPLC (5% MeCN:95% water:0.1% TFA→95% MeCN:5% water:0.1% TFA). The organic solvent was removed on the rotovapor and the aqueous mixture was frozen and placed on the lyophilizer to afford 1-(4-ethyl-6-(oxazol-5-yl)quinazolin-2-yl)-3-methylbutan-1-amine (23 mg, 44% yield) as a TFA salt: ¹H NMR (400 MHz, DMSO-d₆) δ 8.62 (s, 1H), 8.61 (d, J=1.5 Hz, 1H), 8.46 (d, J=6.5 Hz, 3H), 8.40 (dd, J=8.8, 1.0 Hz, 1H), 8.11 (dd, J=8.8, 1.3 Hz, 1H), 8.05 (s, 1H), 4.53-4.46 (m, 1H), 3.50-3.41 (m, 2H), 2.26-2.14 (m, 1H), 1.59-1.47 (m, 1H), 1.40 (t, J=7.3 Hz, 3H), 1.35-1.26 (m, 0.5H), 1.17-1.08 (m, 0.5H), 0.95-0.82 (m, 6H); MS (ESI) m/e 311.2 [(M+H)⁺, calcd for C₁₈H₂₃N₄O 311.2]. HPLC retention time (Method O): t_{R}=8.54 min; HPLC retention time (Method P): t_{R}=8.57 min.

### Example 4

### 3-Methyl-1-(6-(oxazol-5-yl)-4-propylquinazolin-2-yl)butan-1-amine

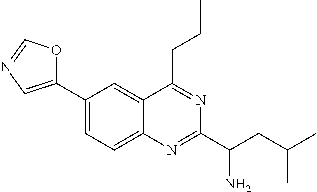 [SMILES: CCCc1nc(C(N)CC(C)C)nc2ccc(-c3cnco3)cc12]

Prepared in a similar fashion as described in Example 3 using propylmagnesium bromide in Part A to give 3-methyl-1-(6-(oxazol-5-yl)-4-propylquinazolin-2-yl)butan-1-amine (3 mg) as a TFA salt: ¹H NMR (500 MHz, CD₃OD) δ 8.85 (s, 1H), 8.75 (d, J=1.5 Hz, 1H), 8.41-8.37 (m, 1H), 8.17 (d, J=8.9 Hz, 1H), 8.14 (s, 1H), 4.59 (dd, J=4.7, 2.3 Hz, 1H), 3.48-3.41 (m, 2H), 2.43-2.28 (m, 1H), 2.03 (qd, J=7.4, 2.1 Hz, 2H), 1.63 (td, J=13.3, 5.8 Hz, 1H), 1.44-1.27 (m, 1H), 1.13 (td, J=7.5, 1.2 Hz, 3H), 1.07-0.96 (m, 6H); MS (ESI) m/e 324.1 [(M+H)⁺, calcd for C₂₀H₂₆N₃O 324.2]. HPLC retention time (Method O): t_{R}=7.08 min; HPLC retention time (Method P): t_{R}=7.14 min.

### Example 5

### 1-(4-Ethyl-8-methoxy-6-(oxazol-5-yl)quinazolin-2-yl)-3-methylbutan-1-amine

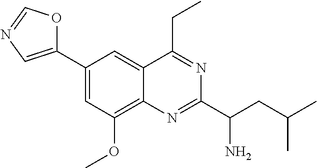 [SMILES: CCc1nc(C(N)CC(C)C)nc2c(OC)cc(-c3cnco3)cc12]

### Part A. 2-Amino-3-methoxybenzaldehyde

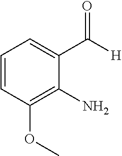

To a solution of 3-methoxy-2-nitrobenzaldehyde (15.0 g, 83 mmol) in ethanol (450 mL) was added ammonium chloride (31.0 g, 580 mmol) and zinc powder (43.3 g, 662 mmol). The reaction mixture was heated at 70° C. for 3 h. The reaction mixture was cooled to room temperature and was filtered through a pad of Celite. The filtrate was concentrated and the residue was purified by column chromatography on silica gel (20%-30% ethyl acetate in hexanes) to afford 2-amino-3-methoxybenzaldehyde (2.94 g, 24% yield) as a yellow oil: ¹H NMR (400 MHz, CDCl₃) δ 9.91 (s, 1H), 7.15 (dd, J=8.0, 1.2 Hz, 1H), 6.90 (dd, J=7.7, 0.9 Hz, 1H), 6.71 (t, J=7.9 Hz, 2H), 6.42 (br s, 2H), 3.91 (s, 3H); MS (ESI) m/e 152.1 [(M+H)⁺, calcd for C₈H₁₀NO₂ 152.1].

### Part B. 1-(2-Amino-3-methoxyphenyl)propan-1-ol

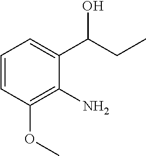

To a solution of 2-amino-3-methoxybenzaldehyde (2.80 g, 18.52 mmol) in THF (100 mL) at 0° C. was added ethylmagnesium bromide (185 mL, 185 mmol) slowly via syringe. The reaction mixture was stirred at 0° C. for 1 h. The reaction mixture was then cooled to −10° C. in an ice/acetone bath and was quenched by the dropwise addition of saturated aqueous NaHCO₃ solution. The mixture was filtered through a pad of Celite. The mixture was transferred to a separatory funnel containing saturated aqueous NaHCO₃ solution (25 mL). The aqueous layer was extracted with ethyl acetate (3×25 mL). The combined organic layers were washed with brine (20 mL), dried over MgSO₄, filtered, and concentrated. The residue was purified by column chromatography on silica gel (20%-40% ethyl acetate in hexanes) to afford 1-(2-amino-3-methoxyphenyl)propan-1-ol (1.65 g, 49% yield) as a pale yellow oil: ¹H NMR (400 MHz, CDCl₃) δ 6.80-6.65 (m, 3H), 4.65 (t, J=6.9 Hz, 1H), 4.41 (br. s., 2H), 3.88 (s, 3H), 1.96 (quin, J=7.3 Hz, 2H), 0.98 (t, J=7.4 Hz, 3H).

### Part C. 1-(2-Amino-3-methoxyphenyl)propan-1-one

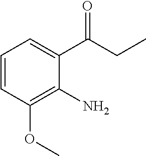

Manganese dioxide (3.79 g, 43.6 mmol) was added to a solution of 1-(2-amino-3-methoxyphenyl)propan-1-ol (1.58 g, 8.72 mmol) in CH₂Cl₂ (50 mL) and THF (10 mL). The mixture was stirred at room temperature for 16 h. The mixture was filtered through a pad of Celite and the filtrate was concentrated. The residue was purified by column chromatography on silica gel (10%-25% ethyl acetate in hexanes) to afford 1-(2-amino-3-methoxyphenyl)propan-1-one (1.11 g, 71% yield) as a yellow solid: ¹H NMR (400 MHz, CDCl₃) δ 7.40 (dd, J=8.4, 1.1 Hz, 1H), 6.86 (dd, J=7.8, 1.0 Hz, 1H), 6.63 (br. s., 2H), 6.60 (dd, J=8.3, 7.8 Hz, 1H), 3.89 (s, 3H), 3.00 (q, J=7.3 Hz, 2H), 1.23 (t, J=7.3 Hz, 3H); MS (ESI) m/e 180.1 [(M+H)⁺, calcd for C₁₀H₁₄NO₂ 180.1].

### Part D. 1-(2-Amino-5-bromo-3-methoxyphenyl)propan-1-one

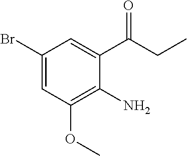

To a solution of 1-(2-amino-3-methoxyphenyl)propan-1-one (1.08 g, 6.03 mmol) in DMF (30 mL) at 0° C. was added NBS (1.180 g, 6.63 mmol). The cooling bath was removed and the reaction mixture was warmed up to room temperature and was stirred at room temperature for 1 h. The mixture was cooled to 0° C. and was treated with saturated sodium sulfite solution (5 mL) and was stirred for 5 min. The reaction mixture was transferred to a separatory funnel containing ether (50 mL). The aqueous layer was washed with water (5×5 mL). The combined organic layers were washed with brine (10 mL), dried over MgSO₄, filtered, and concentrated. The residue was purified by column chromatography on silica gel (10%-20% ethyl acetate in hexanes) to afford 1-(2-amino-5-bromo-3-methoxyphenyl)propan-1-one (1.26 g, 81% yield) as a yellow oil: ¹H NMR (400 MHz, CDCl₃) δ 7.50 (d, J=2.0 Hz, 1H), 6.91 (d, J=2.0 Hz, 1H), 6.65 (br. s., 2H), 3.88 (s, 3H), 2.95 (q, J=7.3 Hz, 2H), 1.21 (t, J=7.3 Hz, 3H); MS (ESI) m/e 257.9 [(M+H)⁺, calcd for C₁₀H₁₃BrNO₂ 258.0].

### Part E. Benzyl 1-(4-bromo-2-methoxy-6-propionylphenylamino)-4-methyl-1-oxopentan-2-ylcarbamate

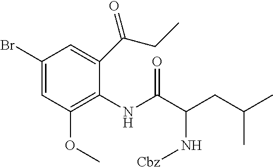 [SMILES: CCC(=O)c1cc(Br)cc(OC)c1NC(=O)C(CC(C)C)NC(=O)OCc1ccccc1]

To a solution of N-(chloromethylene)-N-methylmethanaminium, chloride salt (120 mg, 0.938 mmol) (Jarrahpour et al. Tetrahedron 2009, 65, 2927-2934) in CH₂Cl₂ (1.5 mL) at 0° C. was added 2-(benzyloxycarbonylamino)-4-methylpentanoic acid (213 mg, 0.804 mmol) in CH₂Cl₂ (1.5 mL) via cannula. The mixture was stirred at 0° C. for 15 min. A premixed solution of 1-(2-amino-5-bromo-3-methoxyphenyl)propan-1-one (173 mg, 0.670 mmol) and Et₃N (0.23 mL, 1.68 mmol) in CH₂Cl₂ (1 mL) was then added via cannula. The reaction mixture was stirred at 0° C. for 15 min followed by stirring at room temperature for 30 min. The reaction mixture was transferred to a separatory funnel containing saturated aqueous NaHCO₃ solution (5 mL). The aqueous layer was extracted with ethyl acetate (3×10 mL).

The combined organic layers were washed with brine (5 mL), dried over MgSO₄, filtered, and concentrated. The residue was purified by column chromatography on silica gel (20%-30% ethyl acetate in hexanes) to afford benzyl 1-(4-bromo-2-methoxy-6-propionylphenylamino)-4-methyl-1-oxopentan-2-ylcarbamate (74 mg, 21.9% yield) as a colorless foam. The product was carried forward directly to the next step.

### Part F. Benzyl 1-(6-bromo-4-ethyl-8-methoxyquinazolin-2-yl)-3-methylbutylcarbamate

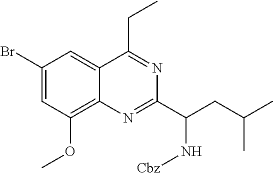 [SMILES: CCc1nc(C(CC(C)C)NC(=O)OCc2ccccc2)nc2c(OC)cc(Br)cc12]

To a solution of benzyl 1-(4-bromo-2-methoxy-6-propionylphenylamino)-4-methyl-1-oxopentan-2-ylcarbamate (74 mg, 0.146 mmol) in acetic acid (2.5 mL) was added ammonium acetate (226 mg, 2.93 mmol). The reaction mixture was heated at 95° C. for 14 h. The mixture was concentrated and the residue was transferred to a separatory funnel containing saturated aqueous NaHCO₃ solution (20 mL). The aqueous layer was extracted with ethyl acetate (3×20 mL). The combined organic layers were washed with brine (10 mL), dried over MgSO₄, filtered, and concentrated. The residue was purified by column chromatography on silica gel (20%-30% ethyl acetate in hexanes) to afford benzyl 1-(6-bromo-4-ethyl-8-methoxyquinazolin-2-yl)-3-methylbutylcarbamate (54 mg, 76% yield) as a colorless oil: ¹H NMR (400 MHz, CDCl₃) δ 7.81 (d, J=1.8 Hz, 1H), 7.43-7.30 (m, 5H), 7.26 (d, J=1.8 Hz, 1H), 6.06 (d, J=8.8 Hz, 1H), 5.29-5.21 (m, 1H), 5.18-5.09 (m, 2H), 4.06 (s, 3H), 3.22 (q, J=7.4 Hz, 2H), 1.82-1.71 (m, 3H), 1.43 (t, J=7.5 Hz, 3H), 1.03 (d, J=5.8 Hz, 3H), 0.96 (d, J=5.8 Hz, 3H); MS (ESI) m/e 486.0 [(M+H)⁺, calcd for C₂₄H₂₉BrN₃O₃ 486.1].

### Part G. Benzyl 1-(4-ethyl-8-methoxy-6-vinylquinazolin-2-yl)-3-methylbutylcarbamate

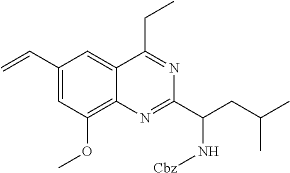 [SMILES: C=Cc1cc(OC)c2nc(C(CC(C)C)NC(=O)OCc3ccccc3)nc(CC)c2c1]

To a solution of benzyl 1-(6-bromo-4-ethyl-8-methoxyquinazolin-2-yl)-3-methylbutylcarbamate (90 mg, 0.185 mmol) in toluene (1 mL) and ethanol (0.25 mL) was added 2,4,6-trivinyl-1,3,5,2,4,6-trioxatriborinane pyridine complex (29.9 mg, 0.185 mmol) and sodium carbonate (2 N, aq) (0.222 mL, 0.222 mmol). The solution was degassed with N₂ for 10 min. Pd(PPh₃)₄ (21.38 mg, 0.019 mmol) was added to the reaction mixture and the mixture was heated at 95° C. for 2 h. The reaction mixture was cooled to room temperature and was transferred to a separatory funnel containing saturated aqueous NaHCO₃ solution (10 mL). The aqueous layer was extracted with ethyl acetate (3×15 mL). The combined organic layers were washed with brine (5 mL), dried over MgSO₄, filtered, and concentrated. The residue was purified by column chromatography on silica gel (20%-40% ethyl acetate in hexanes) to afford benzyl 1-(4-ethyl-8-methoxy-6-vinylquinazolin-2-yl)-3-methylbutylcarbamate (64 mg, 80% yield) as a colorless oil: ¹H NMR (400 MHz, CDCl₃) δ 7.56 (d, J=1.3 Hz, 1H), 7.43-7.30 (m, 6H), 6.88 (dd, J=17.4, 10.9 Hz, 1H), 6.12 (d, J=8.0 Hz, 1H), 5.90 (d, J=17.6 Hz, 1H), 5.45 (d, J=10.8 Hz, 1H), 5.30-5.22 (m, 1H), 5.15 (ABq, J_{AB}=12.5 Hz, Δυ=12.9 Hz, 2H), 4.10 (s, 3H), 3.27 (q, J=7.4 Hz, 2H), 1.84-1.72 (m, 3H), 1.44 (t, J=7.5 Hz, 3H), 1.04 (d, J=5.8 Hz, 3H), 0.96 (d, J=5.5 Hz, 3H); MS (ESI) m/e 434.1 [(M+H)⁺, calcd for C₂₆H₃₂N₃O₃ 434.2].

### Part H. Benzyl 1-(4-ethyl-6-formyl-8-methoxyquinazolin-2-yl)-3-methylbutylcarbamate

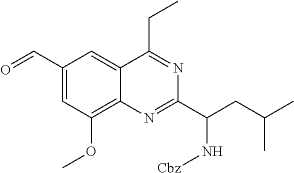 [SMILES: CCc1nc(C(CC(C)C)NC(=O)OCc2ccccc2)nc2c(OC)cc(C=O)cc12]

To a solution of benzyl 1-(4-ethyl-8-methoxy-6-vinylquinazolin-2-yl)-3-methylbutylcarbamate (63 mg, 0.145 mmol) in dioxane (2 mL) and water (0.5 mL) at 0° C. was added 2,6-lutidine (0.034 mL, 0.291 mmol), osmium tetroxide (2.5% in 2-methyl-2-propanol) (0.036 mL, 2.91 μmol), and sodium periodate (124 mg, 0.581 mmol). The cooling bath was removed and the reaction mixture was allowed to warm up to room temperature while stirring for 2 h. The reaction mixture was transferred to a separatory funnel containing a 1:1 mixture of water and saturated aqueous NaHCO₃ solution (5 mL). The aqueous layer was extracted with ethyl acetate (3×10 mL). The combined organic layers were washed with brine (5 mL), dried over MgSO₄, filtered and concentrated. The residue was purified by column chromatography on silica gel (20%-40% ethyl acetate in hexanes) to afford benzyl 1-(4-ethyl-6-formyl-8-methoxyquinazolin-2-yl)-3-methylbutylcarbamate (53 mg, 84% yield) as a colorless oil: ¹H NMR (400 MHz, CDCl₃) δ 10.15 (s, 1H), 8.17 (d, J=1.3 Hz, 1H), 7.65 (d, J=1.3 Hz, 1H), 7.44-7.30 (m, 5H), 7.27-7.23 (m, 1H), 5.34-5.26 (m, 1H), 5.15 (ABq, J_{AB}=12.3 Hz, Δυ=10.4 Hz, 2H), 4.14 (s, 3H), 3.37 (q, J=7.3 Hz, 2H), 1.84-1.74 (m, 3H), 1.49 (t, J=7.4 Hz, 3H), 1.05 (d, J=5.3 Hz, 3H), 0.98 (d, J=5.5 Hz, 3H); MS (ESI) m/e 436.0 [(M+H)⁺, calcd for C₂₅H₃₀N₃O₄ 436.2].

### Part I. Benzyl 1-(4-ethyl-8-methoxy-6-(oxazol-5-yl)quinazolin-2-yl)-3-methylbutylcarbamate

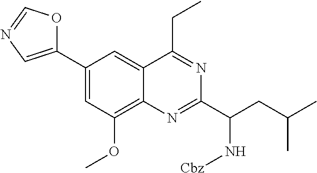 [SMILES: CCc1nc(C(CC(C)C)NC(=O)OCc2ccccc2)nc2c(OC)cc(-c3cnco3)cc12]

To a solution of benzyl 1-(4-ethyl-6-formyl-8-methoxyquinazolin-2-yl)-3-methylbutylcarbamate (50 mg, 0.115 mmol) and TosMIC (45.9 mg, 0.235 mmol) in methanol (2 mL) was added potassium carbonate (33.3 mg, 0.241 mmol). The reaction mixture was heated at reflux for 1.5 h. The reaction mixture was cooled to room temperature and was transferred to a separatory funnel containing saturated aqueous NaHCO₃ solution (5 mL). The aqueous layer was extracted with ethyl acetate (3×10 mL). The combined organic layers were washed with brine (5 mL), dried over MgSO₄, filtered, and concentrated. The residue was purified by column chromatography on silica gel (50%-90% ethyl acetate in hexanes) to afford benzyl 1-(4-ethyl-8-methoxy-6-(oxazol-5-yl)quinazolin-2-yl)-3-methylbutylcarbamate (35 mg, 64% yield) product as a colorless foam: ¹H NMR (400 MHz, CDCl₃) δ 8.03 (s, 1H), 7.94 (d, J=1.5 Hz, 1H), 7.54 (s, 1H), 7.44-7.30 (m, 6H), 6.12 (d, J=8.8 Hz, 1H), 5.32-5.24 (m, 1H), 5.15 (ABq, J_{AB}=12.5, Δυ=11.5 Hz, 2H), 4.14 (s, 3H), 3.34 (q, J=7.2 Hz, 2H), 1.85-1.74 (m, 3H), 1.48 (t, J=7.4 Hz, 3H), 1.05 (d, J=5.8 Hz, 3H), 0.98 (d, J=5.8 Hz, 3H); MS (ESI) m/e 475.0 [(M+H)⁺, calcd for C₂₇H₃₁N₄O₄ 475.2].

### Part J. 1-(4-Ethyl-8-methoxy-6-(oxazol-5-yl)quinazolin-2-yl)-3-methylbutan-1-amine

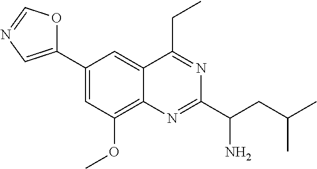 [SMILES: CCc1nc(C(N)CC(C)C)nc2c(OC)cc(-c3cnco3)cc12]

To a solution of benzyl 1-(4-ethyl-8-methoxy-6-(oxazol-5-yl)quinazolin-2-yl)-3-methylbutylcarbamate (28 mg, 0.059 mmol) in ethanol (1 mL) was added palladium on carbon (10%, Degussa type) (20 mg, 9.40 μmol). The reaction mixture was evacuated and filled with N₂ (3×) and then evacuated and filled with H₂ (3×). The reaction mixture was stirred under a H₂ atmosphere for 2 h. The mixture was filtered through a pad of Celite and was concentrated. The product was purified by reverse phase HPLC (5% MeCN:95% water:0.1% TFA→95% MeCN:5% water:0.1% TFA). The organic solvent was removed on the rotovapor and the aqueous mixture was frozen and placed on the lyophilizer to afford 1-(4-ethyl-8-methoxy-6-(oxazol-5-yl)quinazolin-2-yl)-3-methylbutan-1-amine (16.3 mg, 80% yield) as a TFA salt: ¹H NMR (400 MHz, DMSO-d₆) δ 8.62 (s, 1H), 8.50 (br. s., 3H), 8.14 (d, J=1.5 Hz, 1H), 8.10 (s, 1H), 7.80 (d, J=1.5 Hz, 1H), 4.60-4.51 (m, 1H), 4.11 (s, 3H), 3.42 (qd, J=7.4, 1.5 Hz, 2H), 1.93-1.77 (m, 2H), 1.71 (dt, J=13.4, 6.7 Hz, 1H), 1.41 (t, J=7.4 Hz, 3H), 0.96 (d, J=6.3 Hz, 3H), 0.93 (d, J=6.5 Hz, 3H); MS (ESI) m/e 341.1 [(M+H)⁺, calcd for C₁₉H₂₅N₄O₂ 341.2]. HPLC retention time (Method O): t_{R}=8.85 min; HPLC retention time (Method P): t_{R}=8.90 min.

### Example 6

### 1-(4-Ethyl-6-(3-methoxypyridin-4-yl)quinazolin-2-yl)-3-methylbutan-1-amine

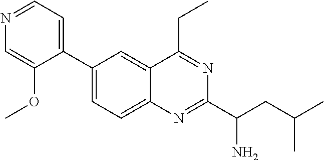 [SMILES: CCc1nc(C(N)CC(C)C)nc2ccc(-c3ccncc3OC)cc12]

### Part A. 1-(2-Amino-5-bromophenyl)propan-1-one

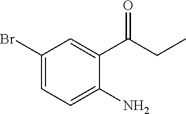

To a solution of 2-amino-5-bromobenzonitrile (2.50 g, 12.69 mmol) in THF (100 mL) at 0° C. was added ethylmagnesium bromide in ether (42.3 mL, 127 mmol). The cooling bath was removed and the reaction mixture was stirred at room temperature for 1.5 h. The mixture was cooled to 0° C. and 6 N HCl (21.2 mL, 127 mmol) was slowly added. The mixture was stirred at room temperature for 12 h. The reaction mixture was transferred to a separatory funnel containing saturated aqueous NaHCO₃ solution (250 mL). The aqueous layer was extracted with ethyl acetate (3×150 mL).

The combined organic layers were washed with brine (150 mL), dried over MgSO₄, filtered, and concentrated. The residue was purified by column chromatography on silica gel (10%-20% ethyl acetate in hexanes) to afford 1-(2-amino-5-bromophenyl)propan-1-one (2.09 g, 72% yield) as a yellow solid: ¹H NMR (400 MHz, CDCl₃) δ 7.86 (d, J=2.3 Hz, 1H), 7.34 (dd, J=8.9, 2.4 Hz, 1H), 6.60 (d, J=8.8 Hz, 1H), 6.38 (br. s., 2H), 2.97 (q, J=7.3 Hz, 2H), 1.22 (t, J=7.3 Hz, 3H); MS (ESI) m/e 228.0 [(M+H)⁺, calcd for C₉H₁₁BrNO 228.0].

### Part B. Benzyl 1-(4-bromo-2-propionylphenylamino)-4-methyl-1-oxopentan-2-ylcarbamate

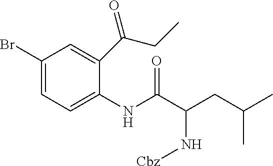 [SMILES: CCC(=O)c1cc(Br)ccc1NC(=O)C(CC(C)C)NC(=O)OCc1ccccc1]

To a solution of N-(chloromethylene)-N-methylmethanaminium, chloride salt (236 mg, 1.841 mmol) (Jarrahpour et al. Tetrahedron 2009, 65, 2927-2934) in CH₂Cl₂ (3.0 mL) at 0° C. was added 2-(benzyloxycarbonylamino)-4-methylpentanoic acid (419 mg, 1.578 mmol) in CH₂Cl₂ (3.0 mL) via cannula. The mixture was stirred at 0° C. for 15 min. A premixed solution of 1-(2-amino-5-bromophenyl)propan-1-one (300 mg, 1.315 mmol) and Et₃N (0.458 mL, 3.29 mmol) in CH₂Cl₂ (2 mL) was then added via cannula. The reaction mixture was stirred at 0° C. for 15 min followed by stirring at room temperature for 30 min. The reaction mixture was transferred to a separatory funnel containing saturated aqueous NaHCO₃ solution (10 mL). The aqueous layer was extracted with ethyl acetate (3×15 mL). The combined organic layers were washed with brine (10 mL), dried over MgSO₄, filtered, and concentrated. The residue was purified by column chromatography on silica gel (10%-20% ethyl acetate in hexanes) to afford benzyl 1-(4-bromo-2-propionylphenylamino)-4-methyl-1-oxopentan-2-ylcarbamate (299 mg, 48% yield) as a colorless foam: ¹H NMR (400 MHz, CDCl₃) δ 12.17 (br. s., 1H), 8.71 (d, J=9.0 Hz, 1H), 8.05 (d, J=1.8 Hz, 1H), 7.65 (dd, J=9.0, 2.3 Hz, 1H), 7.46-7.33 (m, 5H), 5.29 (d, J=7.5 Hz, 1H), 5.19 (ABq, J_{AB}=12.0, Δυ=10.3 Hz, 2H), 4.40 (dt, J=12.4, 4.7 Hz, 1H), 3.06 (q, J=7.3 Hz, 2H), 1.89-1.73 (m, 2H), 1.67-1.61 (m, 1H), 1.23 (t, J=7.3 Hz, 3H), 1.02 (d, J=6.5 Hz, 3H), 1.00 (d, J=6.5 Hz, 3H); MS (ESI) m/e 475.0 [(M+H)⁺, calcd for C₂₃H₂₈BrN₂O₄ 475.1].

### Part C. Benzyl 1-(6-bromo-4-ethylquinazolin-2-yl)-3-methylbutylcarbamate

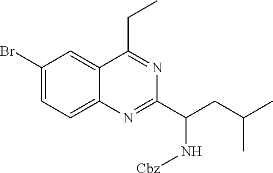 [SMILES: CCc1nc(C(CC(C)C)NC(=O)OCc2ccccc2)nc2ccc(Br)cc12]

To a solution of benzyl 1-(4-bromo-2-propionylphenylamino)-4-methyl-1-oxopentan-2-ylcarbamate (150 mg, 0.316 mmol) in acetic acid (5 mL) was added ammonium acetate (486 mg, 6.31 mmol). The reaction mixture was heated at 95° C. for 14 h.

The reaction was not complete. The reaction mixture was then heated at 110° C. for an additional 20 h. The mixture was cooled to room temperature and was concentrated. The residue was transferred to a separatory funnel containing saturated aqueous NaHCO₃ solution (20 mL). The aqueous layer was extracted with ethyl acetate (3×20 mL). The combined organic layers were washed with brine (10 mL), dried over MgSO₄, filtered, and concentrated. The residue was purified by column chromatography on silica gel (10%-20% ethyl acetate in hexanes) to afford benzyl 1-(6-bromo-4-ethylquinazolin-2-yl)-3-methylbutylcarbamate (117 mg, 81% yield) as a colorless oil: ¹H NMR (400 MHz, CDCl₃) δ 8.25 (d, J=1.8 Hz, 1H), 7.91-7.96 (m, 1H), 7.90-7.85 (m, 1H), 7.44-7.30 (m, 5H), 6.02 (d, J=8.5 Hz, 1H), 5.23-5.11 (m, 3H), 3.26 (q, J=7.5 Hz, 2H), 1.83-1.70 (m, 3H), 1.45 (t, J=7.5 Hz, 3H), 1.03 (d, J=5.8 Hz, 3H), 0.97 (d, J=6.0 Hz, 3H); MS (ESI) m/e 456.1 [(M+H)⁺, calcd for C₂₃H₂₇BrN₃O₂ 456.1].

### Part D. Benzyl 1-(4-ethyl-6-(3-methoxypyridin-4-yl)quinazolin-2-yl)-3-methylbutylcarbamate

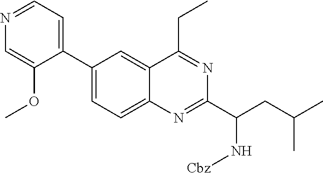 [SMILES: CCc1nc(C(CC(C)C)NC(=O)OCc2ccccc2)nc2ccc(-c3ccncc3OC)cc12]

To a mixture of 3-methoxypyridin-4-ylboronic acid (74.4 mg, 0.486 mmol), potassium phosphate (129 mg, 0.608 mmol), palladium acetate (5.46 mg, 0.024 mmol), and 2-dicyclohexylphosphino-2′,6′-dimethoxy-1,1′-biphenyl (SPhos) (19.97 mg, 0.049 mmol) under nitrogen in a vial sealed with a septa was added via cannula benzyl 1-(6-bromo-4-ethylquinazolin-2-yl)-3-methylbutylcarbamate (111 mg, 0.243 mmol) in n-butanol (2 mL), which was previously sonicated under nitrogen for several minutes. The septa was replaced with a screw cap, and the reaction mixture was heated at 100° C. for 4.5 h. The mixture was cooled to room temperature and was filtered through a pad of Celite with methanol rinsing and the filtrate was concentrated. The residue was purified by column chromatography on silica gel (1%→4% methanol in CH₂Cl₂) to afford benzyl 1-(4-ethyl-6-(3-methoxypyridin-4-yl)quinazolin-2-yl)-3-methylbutylcarbamate (102 mg, 87% yield) as a pale yellow oil: ¹H NMR (400 MHz, CDCl₃) δ 8.47 (s, 1H), 8.43 (d, J=4.8 Hz, 1H), 8.30 (s, 1H), 8.06 (s, 2H), 7.46-7.31 (m, 6H), 6.08 (d, J=8.5 Hz, 1H), 5.25-5.18 (m, 1H), 5.16 (s, 2H), 4.00 (s, 3H), 3.33 (q, J=7.3 Hz, 2H), 1.84-1.72 (m, 3H), 1.48 (t, J=7.5 Hz, 3H), 1.04 (d, J=6.0 Hz, 3H), 0.99 (d, J=5.0 Hz, 3H); MS (ESI) m/e 485.1 [(M+H)⁺, calcd for C₂₉H₃₃N₄O₃ 485.2].

### Part E. 1-(4-Ethyl-6-(3-methoxypyridin-4-yl)quinazolin-2-yl)-3-methylbutan-1-amine

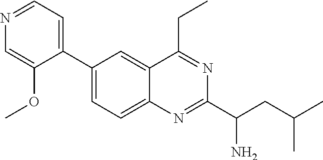 [SMILES: CCc1nc(C(N)CC(C)C)nc2ccc(-c3ccncc3OC)cc12]

To a solution of benzyl 1-(4-ethyl-6-(3-methoxypyridin-4-yl)quinazolin-2-yl)-3-methylbutylcarbamate (96 mg, 0.198 mmol) in ethanol (3 mL) was added palladium on carbon (10%, Degussa type) (65 mg, 0.031 mmol). The reaction mixture was evacuated and filled with N₂ (3×) and then evacuated and filled with H₂ (3×). The reaction mixture was stirred under a H₂ atmosphere for 2 h. The mixture was filtered through a pad of Celite and was concentrated. The product was purified by reverse phase HPLC (5% MeCN:95% water:0.1% TFA→95% MeCN:5% water:0.1% TFA). The organic solvent was removed on the rotovapor and the aqueous mixture was frozen and placed on the lyophilizer to afford 1-(4-ethyl-6-(3-methoxypyridin-4-yl)quinazolin-2-yl)-3-methylbutan-1-amine (46 mg, 66% yield) as a TFA salt: ¹H NMR (400 MHz, DMSO-d₆) δ 8.64 (s, 1H), 8.57 (d, J=1.5 Hz, 1H), 8.54 (br. s., 3H), 8.47 (d, J=5.0 Hz, 1H), 8.28 (dd, J=8.7, 1.9 Hz, 1H), 8.11 (d, J=8.8 Hz, 1H), 7.71 (d, J=5.0 Hz, 1H), 4.60 (q, J=6.0 Hz, 1H), 3.99 (s, 3H), 3.44 (qd, J=7.5, 2.8 Hz, 2H), 1.86-1.79 (m, 2H), 1.71 (dt, J=13.4, 6.7 Hz, 1H), 1.42 (t, J=7.4 Hz, 3H), 0.97 (d, J=6.5 Hz, 3H), 0.93 (d, J=6.5 Hz, 3H); MS (ESI) m/e 351.1 [(M+H)⁺, calcd for C₂₁H₂₇N₄O 351.2]. HPLC retention time (Method O): t_{R}=7.07 min; HPLC retention time (Method P): t_{R}=7.05 min.

### Example 7

### (−)-Cyclohexyl(4-ethyl-6-(pyridin-4-yl)quinazolin-2-yl)methanamine

### Example 8

### (+)-Cyclohexyl(4-ethyl-6-(pyridin-4-yl)quinazolin-2-yl)methanamine

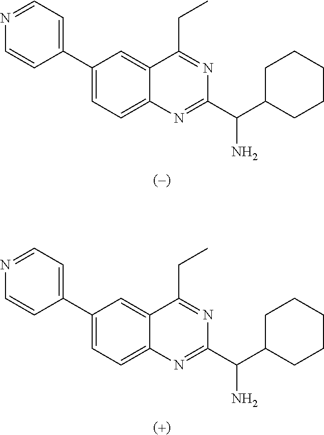 [SMILES: CCc1nc(C(N)C2CCCCC2)nc2ccc(-c3ccncc3)cc12 ; CCc1nc(C(N)C2CCCCC2)nc2ccc(-c3ccncc3)cc12]

### Part A. 1-(2-Amino-5-bromophenyl)propan-1-one

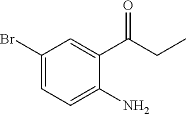

To a solution of ethylmagnesium bromide (3M in THF) (35.5 mL, 107 mmol) in a flask at 0° C. was added a mixture of 2-amino-5-bromobenzonitrile (3.00 g, 15.23 mmol) in THF (50 mL). The mixture was stirred at room temperature for 3 h. The mixture was cooled to 0° C. and 6 N HCl (17.8 mL, 107 mmol) was added slowly. The mixture was stirred at room temperature for 3 h. The mixture was made alkaline by the addition of NaHCO₃. The reaction mixture was transferred to a separatory funnel containing saturated aqueous NaHCO₃ solution (15 mL). The aqueous layer was extracted with ethyl acetate (3×15 mL). The combined organic layers were washed with brine (15 mL), dried over MgSO₄, filtered, and concentrated. The residue was purified by column chromatography on silica gel (20%-30% ethyl acetate in hexanes) to afford 1-(2-amino-5-bromophenyl)propan-1-one (2.00 g, 8.77 mmol, 58% yield) as a yellow solid: MS (ESI) m/e 228.0 [(M+H)⁺, calcd for C₉H₁₁BrNO 228.1].

### Part B. tert-Butyl 2-(4-bromo-2-propionylphenylamino)-1-cyclohexyl-2-oxoethylcarbamate

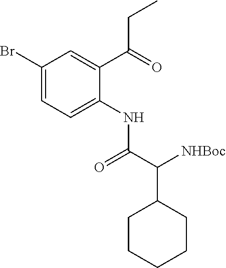 [SMILES: CCC(=O)c1cc(Br)ccc1NC(=O)C(NC(=O)OC(C)(C)C)C1CCCCC1]

To a suspension of 1-(2-amino-5-bromophenyl)propan-1-one (200 mg, 0.877 mmol) and (S)-2-(tert-butoxycarbonylamino)-2-cyclohexylacetic acid (248 mg, 0.965 mmol) in pyridine (3 mL) at −15° C. was added POCl₃ (0.098 mL, 1.052 mmol). The mixture was stirred at room temperature for 12 h. The mixture quenched with cold water and 1 N HCl (pH should not go below 4). The aqueous layer was extracted with ethyl acetate (3×20 mL). The combined organic layers were concentrated and the residue was purified by silica gel chromatography (30% ethyl acetate in hexanes) to afford tert-butyl 2-(4-bromo-2-propionylphenylamino)-1-cyclohexyl-2-oxoethylcarbamate (180 mg, 44% yield) as a yellow solid, which was used directly in the next step.

### Part C. tert-Butyl (6-bromo-4-ethylquinazolin-2-yl)(cyclohexyl)methylcarbamate

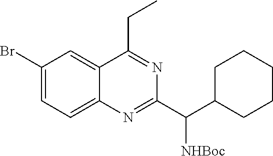 [SMILES: CCc1nc(C(NC(=O)OC(C)(C)C)C2CCCCC2)nc2ccc(Br)cc12]

A mixture of tert-butyl 2-(4-bromo-2-propionylphenylamino)-1-cyclohexyl-2-oxoethylcarbamate (180 mg, 0.385 mmol) and ammonium acetate (1.567 g, 20.33 mmol) in acetic acid (4 mL) was heated at 90° C. for 12 h. The solvent was concentrated and the mixture was purified by column chromatography on silica gel (20%-70% ethyl acetate in hexanes) to afford tert-butyl (6-bromo-4-ethylquinazolin-2-yl)(cyclohexyl)methylcarbamate (110 mg, 64% yield) as a yellow solid: MS (ESI) m/e 392.1 [(M+H-t-Bu)⁺, calcd for C₁₈H₂₃BrN₃O₂ 392.1].

### Part D. tert-Butyl cyclohexyl(4-ethyl-6-(pyridin-4-yl)quinazolin-2-yl)methylcarbamate

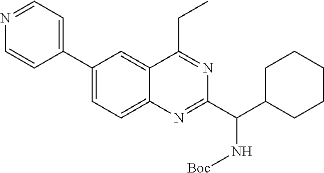 [SMILES: CCc1nc(C(NC(=O)OC(C)(C)C)C2CCCCC2)nc2ccc(-c3ccncc3)cc12]

A mixture of tert-butyl (6-bromo-4-ethylquinazolin-2-yl)(cyclohexyl)methylcarbamate (110 mg, 0.245 mmol), pyridin-4-ylboronic acid (60.3 mg, 0.491 mmol), potassium phosphate (130 mg, 0.613 mmol), palladium acetate (5.51 mg, 0.025 mmol), and 2-dicyclohexylphosphino-2′,6′-dimethoxy-1,1′-biphenyl (SPhos) (20.14 mg, 0.049 mmol) in n-butanol (2 mL) in a sealed vial was heated at 100° C. for 4.5 h. The mixture was cooled to room temperature and was filtered through a pad of Celite with methanol rinsing and the filtrate was concentrated. The residue was purified by column chromatography on silica gel (1%→4% methanol in CH₂Cl₂) to afford tert-butyl cyclohexyl(4-ethyl-6-(pyridin-4-yl)quinazolin-2-yl)methylcarbamate (80 mg, 73% yield) as a pale yellow oil: MS (ESI) m/e 447.3 [(M+H)⁺, calcd for C₂₇H₃₅N₄O₂ 447.3].

### Part E. Cyclohexyl(4-ethyl-6-(pyridin-4-yl)quinazolin-2-yl)methanamine

tert-Butyl cyclohexyl(4-ethyl-6-(pyridin-4-yl)quinazolin-2-yl)methylcarbamate (50 mg, 0.112 mmol) was dissolved in CH₂Cl₂ (2 mL) and cooled to 0° C. To this solution was added TFA (0.173 mL, 2.239 mmol). The reaction mixture was stirred at room temperature for 2 hours. The solvent was evaporated and the residue was purified by reverse phase HPLC (5% MeCN:95% water:0.1% TFA→95% MeCN:5% water:0.1% TFA) to afford cyclohexyl(4-ethyl-6-(pyridin-4-yl)quinazolin-2-yl)methanamine (55 mg, 86% yield) as a TFA salt.

The enantiomers were separated by chiral chromatography (ChiralPak IC-H column, 30×250 mm, 5 m, 30% methanol with 0.1% diethylamine/70% CO₂, 120 bar, 35° C., 70 mL/min, k=248 nm):

### Example 7 (Peak 1)

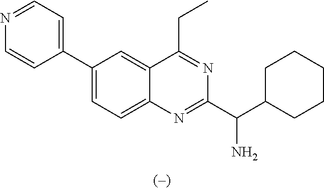 [SMILES: CCc1nc(C(N)C2CCCCC2)nc2ccc(-c3ccncc3)cc12]

[α]²²_{D}−7.07 (c 0.40, MeOH); ¹H NMR (400 MHz, CDCl₃) δ 8.80-8.74 (m, 2H), 8.34-8.31 (m, 1H), 8.14-8.08 (m, 2H), 7.66-7.60 (m, 2H), 4.00 (d, J=5.8 Hz, 1H), 3.38 (q, J=7.5 Hz, 2H), 1.90 (br. s., 2H), 1.80-1.63 (m, 6H), 1.50 (t, J=7.4 Hz, 3H), 1.28-1.13 (m, 5H); MS (ESI) m/e 347.2 [(M+H)⁺, calcd for C₂₂H₂₇N₄ 347.2]. HPLC retention time (Method O): t_{R}=6.05 min; HPLC retention time (Method P): t_{R}=7.12 min.

### Example 8 (Peak 2)

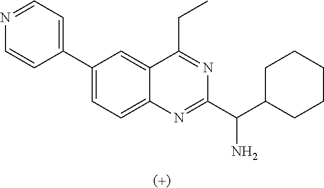 [SMILES: CCc1nc(C(N)C2CCCCC2)nc2ccc(-c3ccncc3)cc12]

[α]²²_{D}+9.29 (c 0.38, MeOH); ¹H NMR (400 MHz, CDCl₃) δ 8.81-8.74 (m, 2H), 8.32 (s, 1H), 8.17-8.08 (m, 2H), 7.67-7.60 (m, 2H), 4.00 (d, J=5.3 Hz, 1H), 3.38 (q, J=7.5 Hz, 2H), 1.90 (br. s., 2H), 1.83-1.60 (m, 6H), 1.50 (t, J=7.4 Hz, 3H), 1.27-1.13 (m, 5H); MS (ESI) m/e 347.2 [(M+H)⁺, calcd for C₂₂H₂₇N₄ 347.2]. HPLC retention time (Method O): t_{R}=5.97 min; HPLC retention time (Method P): t_{R}=4.83 min.

### Example 9

### 2-Cyclohexyl-1-(4-ethyl-6-(pyridin-4-yl)quinazolin-2-yl)ethanamine

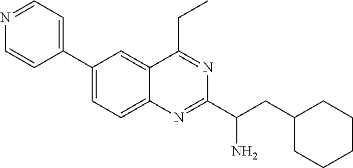 [SMILES: CCc1nc(C(N)CC2CCCCC2)nc2ccc(-c3ccncc3)cc12]

Prepared in a similar fashion as described in Example 7 using (S)-2-((tert-butoxycarbonyl)amino)-3-cyclohexylpropanoic acid in Part B to give 2-cyclohexyl-1-(4-ethyl-6-(pyridin-4-yl)quinazolin-2-yl)ethanamine (80 mg) as a TFA salt: MS (ESI) m/e 361.2 [(M+H)⁺, calcd for C₂₃H₂₉N₄ 361.2]; HPLC (Method O): t_{R}=7.79 min; HPLC (Method P): t_{R}=7.98 min.

### Example 10

### 2-(2-Cyclopentylethyl)-4-ethyl-6-(pyridin-4-yl)quinazoline

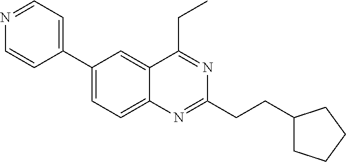 [SMILES: CCc1nc(CCC2CCCC2)nc2ccc(-c3ccncc3)cc12]

### Part A. N-(4-Bromo-2-propionylphenyl)-3-cyclopentylpropanamide

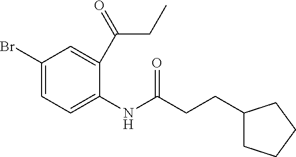 [SMILES: CCC(=O)c1cc(Br)ccc1NC(=O)CCC1CCCC1]

To a solution of 1-(2-amino-5-bromophenyl)propan-1-one (200 mg, 0.877 mmol) (Kapa et al. Org. Proc. Res. Dev. 2003, 7, 723-732.) in CH₂Cl₂ (5 mL) was added triethylamine (0.367 mL, 2.63 mmol) and DMAP (10.7 mg, 0.088 mmol). The mixture was cooled to 0° C. 3-Cyclopentylpropanoyl chloride (0.269 mL, 1.754 mmol) dissolved in CH₂Cl₂ (1 mL) was then added via cannula. The reaction mixture was stirred at 0° C. for 5 min and was then allowed to warm up to room temperature and was stirred at room temperature for 1 h. The reaction mixture was transferred to a separatory funnel containing saturated aqueous NaHCO₃ solution (5 mL). The aqueous layer was extracted with ethyl acetate (3×10 mL). The combined organic layers were washed with brine (5 mL), dried over MgSO₄, filtered, and concentrated. The residue was purified by column chromatography on silica gel (20%-40% ethyl acetate in hexanes) to afford N-(4-bromo-2-propionylphenyl)-3-cyclopentylpropanamide (180 mg, 58% yield) as a colorless solid: ¹H NMR (400 MHz, DMSO-d₆) δ 10.97 (s, 1H), 8.09 (d, J=8.8 Hz, 1H), 8.05 (d, J=2.3 Hz, 1H), 7.75 (dd, J=8.9, 2.4 Hz, 1H), 3.06 (q, J=7.1 Hz, 2H), 2.40-2.34 (m, 2H), 1.81-1.71 (m, 3H), 1.65-1.44 (m, 6H), 1.14-1.09 (m, 2H), 1.06 (t, J=7.0 Hz, 3H); MS (ESI) m/e 352.2 [(M+H)⁺, calcd for C₁₇H₂₃BrNO₂ 352.1].

### Part B. 6-Bromo-2-(2-cyclopentylethyl)-4-ethylquinazoline

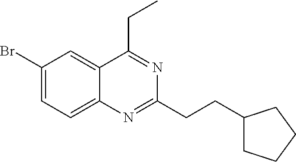 [SMILES: CCc1nc(CCC2CCCC2)nc2ccc(Br)cc12]

To a solution of N-(4-bromo-2-propionylphenyl)-3-cyclopentylpropanamide (180 mg, 0.511 mmol) in acetic acid (2 mL) was added ammonium acetate (394 mg, 5.11 mmol). The reaction mixture was heated at 85° C. for 14 h. The mixture was concentrated and the product was purified by reverse phase HPLC (5% MeCN:95% water:0.1% TFA→95% MeCN:5% water:0.1% TFA). The organic solvent was removed on the rotovapor and the aqueous mixture was frozen and placed on the lyophilizer to afford 6-bromo-2-(2-cyclopentylethyl)-4-ethylquinazoline (100 mg, 59% yield) as a pale yellow amorphous solid: ¹H NMR (400 MHz, CDCl₃) δ 8.21 (dd, J=2.0, 0.5 Hz, 1H), 7.87 (dd, J=8.8, 2.0 Hz, 1H), 7.84 (dd, J=9.0, 0.5 Hz, 1H), 3.23 (q, J=7.5 Hz, 2H), 3.11-3.04 (m, 2H), 1.96-1.77 (m, 5H), 1.66-1.48 (m, 4H), 1.44 (t, J=7.5 Hz, 3H), 1.24-1.13 (m, 2H); MS (ESI) m/e 333.1 [(M+H)⁺, calcd for C₁₇H₂₂BrN₂ 333.1].

### Part C. 2-(2-Cyclopentylethyl)-4-ethyl-6-(pyridin-4-yl)quinazoline

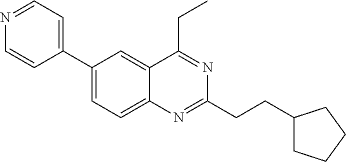 [SMILES: CCc1nc(CCC2CCCC2)nc2ccc(-c3ccncc3)cc12]

To a mixture of 6-bromo-2-(2-cyclopentylethyl)-4-ethylquinazoline (30 mg, 0.090 mmol) and 4-pyridylboronic acid (22.13 mg, 0.180 mmol) in THF (5 mL) was added sodium carbonate (2M in H₂O) (0.135 mL, 0.270 mmol). N₂ was bubbled through the reaction mixture for 2 min. Pd(PPh₃)Cl₂ (6.32 mg, 9.00 μmol) was then added and the reaction mixture was heated at 70° C. for 2 h. The reaction mixture was cooled to room temperature and was transferred to a separatory funnel containing saturated aqueous NaHCO₃ solution (10 mL). The aqueous layer was extracted with ethyl acetate (3×25 mL). The combined organic layers were washed with brine (10 mL), dried over MgSO₄, filtered and concentrated. The residue was purified by reverse phase HPLC (5% MeCN:95% water:0.1% TFA→95% MeCN:5% water:0.1% TFA) to afford 2-(2-cyclopentylethyl)-4-ethyl-6-(pyridin-4-yl)quinazoline (23 mg, 57% yield) as a TFA salt: ¹H NMR (400 MHz, CDCl₂ δ 8.79-8.74 (m, 2H), 8.32 (t, J=1.3 Hz, 1H), 8.09 (d, J=1.5 Hz, 2H), 7.64-7.61 (m, 2H), 3.36 (q, J=7.5 Hz, 2H), 3.15-3.10 (m, 2H), 2.01-1.91 (m, 3H), 1.90-1.81 (m, 2H), 1.69-1.59 (m, 2H), 1.59-1.51 (m, 2H), 1.50 (t, J=7.7 Hz, 3H), 1.28-1.16 (m, 2H); MS (ESI) m/e 332.3 [(M+H)⁺, calcd for C₂₂H₂₆N₃ 332.2]. HPLC retention time (Method O): t_{R}=7.81 min; HPLC retention time (Method P): t_{R}=8.02 min.

### Example 11

### 1-(5-Methoxy-6-(oxazol-5-yl)quinazolin-2-yl)-3-methylbutan-1-amine

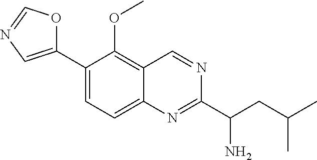 [SMILES: COc1c(-c2cnco2)ccc2nc(C(N)CC(C)C)ncc12]

### Part A. Methyl 2-amino-6-methoxybenzoate

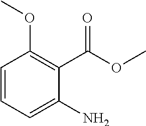

To a mixture of 2-amino-6-methoxybenzoic acid (2.00 g, 11.96 mmol) in Et₂O (30 mL) and methanol (30 mL) at 0° C. was added slowly TMS-diazomethane (12.0 mL, 23.9 mmol). The mixture was warmed to room temperature and was allowed to stir for 2 h. The solvent was evaporated to dryness and the residue was purified by column chromatography on silica gel (20%-30% ethyl acetate in hexanes) to afford methyl 2-amino-6-methoxybenzoate (1.20 g, 55% yield) as a colorless solid: ¹H NMR (400 MHz, DMSO-d₆) δ 7.08 (t, J=8.3 Hz, 1H), 6.34 (dd, J=8.3, 0.8 Hz, 1H), 6.20 (dd, J=8.2, 0.6 Hz, 1H), 5.66 (s, 2H), 3.76 (s, 3H), 3.70 (s, 3H).

### Part B. (R)-Methyl 2-(2-(tert-butoxycarbonylamino)-4-methylpentanamido)-6-methoxybenzoate

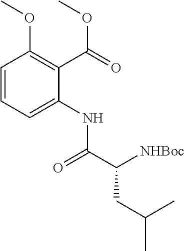 [SMILES: COC(=O)c1c(NC(=O)[C@@H](CC(C)C)NC(=O)OC(C)(C)C)cccc1OC]

To a mixture of methyl 2-amino-6-methoxybenzoate (1.20 g, 6.62 mmol) and (R)-2-(tert-butoxycarbonylamino)-4-methylpentanoic acid (3.06 g, 13.3 mmol) in CH₂Cl₂ (30 mL) was added N,N-diisopropylethylamine (5.78 mL, 33.1 mmol) and HATU (5.04 g, 13.25 mmol). The reaction mixture was stirred at 40° C. for 12 hours. The reaction mixture was transferred to a separatory funnel containing saturated aqueous NaHCO₃ (50 mL) solution. The aqueous layer was extracted with CH₂Cl₂ (3×30 mL). The combined organic layers were separated, concentrated and the residue was purified by column chromatography on silica gel (10-25% ethyl acetate in hexanes) to afford (R)-methyl 2-(2-(tert-butoxycarbonylamino)-4-methylpentanamido)-6-methoxybenzoate (1.30 g, 50% yield) as a yellow solid: ¹H NMR (400 MHz, DMSO-d₆) δ 9.54 (br. s., 1H), 7.46-7.38 (m, 1H), 7.24 (d, J=8.0 Hz, 1H), 6.99-6.90 (m, 2H), 4.02-3.94 (m, 1H), 3.79 (s, 3H), 3.78 (s, 3H), 1.77 (br. s., 1H), 1.50-1.44 (m, 1H), 1.41 (s, 9H), 1.22-1.09 (m, 1H), 0.92-0.80 (m, 6H); MS (ESI) m/e 295.2 [(M-Boc+H)⁺, calcd for C₁₅H₂₃N₂O₄ 295.2].

### Part C. (R)-Methyl 3-bromo-6-(2-(tert-butoxycarbonylamino)-4-methylpentanamido)-2-methoxybenzoate

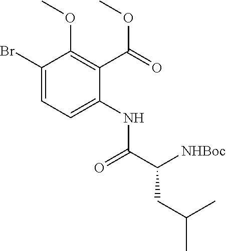 [SMILES: COC(=O)c1c(NC(=O)[C@@H](CC(C)C)NC(=O)OC(C)(C)C)ccc(Br)c1OC]

To a mixture of (R)-methyl 2-(2-(tert-butoxycarbonylamino)-4-methylpentanamido)-6-methoxybenzoate (570 mg, 1.45 mmol) in CH₂Cl₂ (10 mL) at 0° C. was added NBS (283 mg, 1.59 mmol). The mixture was stirred at room temperature for 12 h. LC/MS indicated consumption of the starting material and formation of the desired product.

The reaction mixture was transferred to a separatory funnel containing saturated aqueous NaHCO₃ solution (25 mL). The aqueous layer was extracted with methylene chloride (3×25 mL). The combined organic layers were washed with brine (25 mL), dried over MgSO₄, filtered, and concentrated. The residue was purified by column chromatography on silica gel (5%-15% ethyl acetate in hexanes) to afford (R)-methyl 3-bromo-6-(2-(tert-butoxycarbonylamino)-4-methylpentanamido)-2-methoxybenzoate (410 mg, 60% yield) as an off-white solid: ¹H NMR (400 MHz, DMSO-d₆) δ 9.79 (s, 1H), 7.75 (d, J=8.8 Hz, 1H), 7.35 (d, J=8.8 Hz, 1H), 6.98 (d, J=8.3 Hz, 1H), 4.03-3.96 (m, 1H), 3.83 (s, 3H), 3.81 (s, 3H), 1.76 (br. s., 1H), 1.50-1.44 (m, 1H), 1.41 (s, 9H), 1.22-1.12 (m, 1H), 0.91-0.82 (m, 6H); MS (ESI) m/e 373.1 [(M-Boc+H)⁺, calcd for C₁₅H₂₂BrN₂O₄ 373.1].

### Part D. (R)-Methyl 6-(2-(tert-butoxycarbonylamino)-4-methylpentanamido)-2-methoxy-3-vinylbenzoate

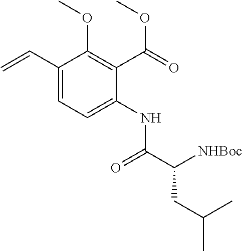 [SMILES: C=Cc1ccc(NC(=O)[C@@H](CC(C)C)NC(=O)OC(C)(C)C)c(C(=O)OC)c1OC]

To a solution of (R)-methyl 3-bromo-6-(2-(tert-butoxycarbonylamino)-4-methylpentanamido)-2-methoxybenzoate (400 mg, 0.845 mmol) in toluene (12 mL) and ethanol (3.0 mL) was added 2,4,6-trivinyl-1,3,5,2,4,6-trioxatriborinane pyridine complex (224 mg, 0.930 mmol) and sodium carbonate (2 N, aq) (1.27 mL, 2.54 mmol). The solution was degassed with N₂ for 5 min. Pd(PPh₃)₄ (98 mg, 0.085 mmol) was added to the reaction mixture and the mixture was heated at 95° C. for 2.5 h. The reaction mixture was cooled to room temperature and transferred to a separatory funnel containing saturated aqueous NaHCO₃ solution (75 mL). The aqueous layer was extracted with ether (3×100 mL). The combined organic layers were washed with brine (50 mL), dried over MgSO₄, filtered, and concentrated. The residue was purified by column chromatography on silica gel (20%-40% ethyl acetate in hexanes) to afford (R)-methyl 6-(2-(tert-butoxycarbonylamino)-4-methylpentanamido)-2-methoxy-3-vinylbenzoate (180 mg, 51% yield) as a tan solid: ¹H NMR (400 MHz, DMSO-d₆) δ 9.68 (br. s., 1H), 7.72 (d, J=8.5 Hz, 1H), 7.42 (d, J=8.8 Hz, 1H), 6.98 (d, J=8.0 Hz, 1H), 6.88 (dd, J=17.8, 11.0 Hz, 1H), 5.87 (dd, J=17.8, 1.3 Hz, 1H), 5.40-5.35 (m, 1H), 3.99 (t, J=7.9 Hz, 1H), 3.82 (s, 3H), 3.72 (s, 3H), 1.77 (br. s., 1H), 1.50-1.44 (m, 1H), 1.41 (s, 9H), 1.22-1.12 (m, 1H), 0.93-0.80 (m, 6H); MS (ESI) m/e 321.2 [(M-Boc+H)⁺, calcd for C₁₇H₂₅N₂O₄ 321.2].

### Part E. (R)-Methyl 6-(2-(tert-butoxycarbonylamino)-4-methylpentanamido)-3-formyl-2-methoxybenzoate

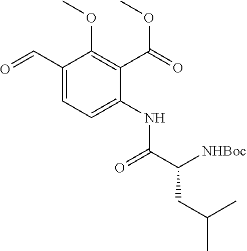 [SMILES: COC(=O)c1c(NC(=O)[C@@H](CC(C)C)NC(=O)OC(C)(C)C)ccc(C=O)c1OC]

To a solution of (R)-methyl 6-(2-(tert-butoxycarbonylamino)-4-methylpentanamido)-2-methoxy-3-vinylbenzoate (180 mg, 0.428 mmol) in dioxane (5 mL) and water (1.25 mL) at 0° C. was added 2,6-lutidine (0.100 mL, 0.856 mmol), osmium tetroxide (2.5% in 2-methyl-2-propanol) (0.161 mL, 0.013 mmol), and sodium periodate (366 mg, 1.71 mmol). The cooling bath was removed and the reaction mixture was allowed to warm up to room temperature while stirring for 2.5 h. The reaction mixture was transferred to a separatory funnel containing water (15 mL) and saturated aqueous NaHCO₃ solution (15 mL). The aqueous layer was extracted with ethyl acetate (3×15 mL). The combined organic layers were washed with brine (15 mL), dried over MgSO₄, filtered, and concentrated. The residue was purified by column chromatography on silica gel (30%-60% ethyl acetate in hexanes) to afford (R)-methyl 6-(2-(tert-butoxycarbonylamino)-4-methylpentanamido)-3-formyl-2-methoxybenzoate (150 mg, 83% yield) as an off-white solid: ¹H NMR (400 MHz, DMSO-d₆) δ 10.18 (d, J=0.5 Hz, 1H), 10.07 (br. s., 1H), 7.89 (d, J=8.5 Hz, 1H), 7.70 (d, J=8.5 Hz, 1H), 7.11 (d, J=7.8 Hz, 1H), 4.04 (t, J=7.3 Hz, 1H), 3.92 (s, 3H), 3.87 (s, 3H), 1.80 (br. s., 1H), 1.50-1.44 (m, 1H), 1.41 (s, 9H), 1.24-1.15 (m, 1H), 0.92-0.81 (m, 6H); MS (ESI) m/e 323.1 [(M+H)⁺, calcd for C₁₆H₂₃N₂O₅ 323.2].

### Part F. (R)-Methyl 6-(2-(tert-butoxycarbonylamino)-4-methylpentanamido)-2-methoxy-3-(oxazol-5-yl)benzoate

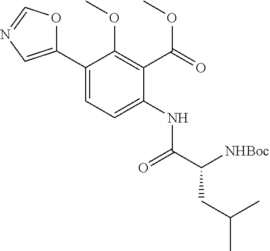 [SMILES: COC(=O)c1c(NC(=O)[C@@H](CC(C)C)NC(=O)OC(C)(C)C)ccc(-c2cnco2)c1OC]

To a solution of (R)-methyl 6-(2-(tert-butoxycarbonylamino)-4-methylpentanamido)-3-formyl-2-methoxybenzoate (150 mg, 0.355 mmol) and TosMIC (69.3 mg, 0.355 mmol) in methanol (10 mL) was added K₂CO₃ (51.5 mg, 0.373 mmol). The reaction mixture was heated at reflux for 1.5 h. The reaction mixture was cooled to room temperature and was transferred to a separatory funnel containing saturated aqueous NaHCO₃ solution (80 mL). The aqueous layer was extracted with ethyl acetate (3×50 mL). The combined organic layers were washed with brine (60 mL), dried over Na₂SO₄, filtered, and concentrated. The residue was purified by column chromatography on silica gel (20%-50% ethyl acetate in hexanes) to afford (R)-methyl 6-(2-(tert-butoxycarbonylamino)-4-methylpentanamido)-2-methoxy-3-(oxazol-5-yl)benzoate (140 mg, 85% yield) as a yellow solid: ¹H NMR (400 MHz, DMSO-d₆) δ 9.82 (s, 1H), 8.51 (s, 1H), 7.84 (d, J=8.8 Hz, 1H), 7.61-7.57 (m, 2H), 7.02 (d, J=7.8 Hz, 1H), 4.02 (t, J=7.4 Hz, 1H), 3.86 (s, 3H), 3.75 (s, 3H), 1.79 (br. s., 1H), 1.51-1.45 (m, 1H), 1.42 (s, 9H), 1.24-1.14 (m, 1H), 0.92-0.81 (m, 6H); MS (ESI) m/e 462.2 [(M+H)⁺, calcd for C₂₃H₃₂N₃O₇ 462.2].

### Part G. (R)-tert-Butyl 1-(2-(hydroxymethyl)-3-methoxy-4-(oxazol-5-yl)phenylamino)-4-methyl-1-oxopentan-2-ylcarbamate

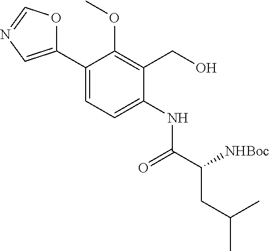 [SMILES: COc1c(-c2cnco2)ccc(NC(=O)[C@@H](CC(C)C)NC(=O)OC(C)(C)C)c1CO]

To a solution of (R)-methyl 6-(2-(tert-butoxycarbonylamino)-4-methylpentanamido)-2-methoxy-3-(oxazol-5-yl)benzoate (110 mg, 0.238 mmol) in CH₂Cl₂ (2 mL) at −78° C. was added DIBAL-H (1.19 mL, 1.19 mmol) dropwise via syringe. The reaction mixture was stirred at −78° C. for 1 h. Saturated Rochelle's salt solution (15 mL) was added to the reaction mixture and the mixture was allowed to stir at 0° C. for 2 h during which time the mixture became clear. The mixture was transferred to separatory funnel and was extracted with methylene chloride (3×15 mL). The combined organic layers were washed with brine (15 mL), dried over MgSO₄, filtered, and concentrated. The residue was purified by column chromatography on silica gel (50%-70% ethyl acetate in hexanes) to afford (R)-tert-butyl 1-(2-(hydroxymethyl)-3-methoxy-4-(oxazol-5-yl)phenylamino)-4-methyl-1-oxopentan-2-ylcarbamate (60 mg, 58% yield) as an off-white solid: ¹H NMR (400 MHz, CD₃OD) δ 8.26 (s, 1H), 7.88 (d, J=8.5 Hz, 1H), 7.73 (d, J=8.5 Hz, 1H), 7.55 (s, 1H), 4.84 (s, 3H), 4.82 (s, 2H), 4.07 (d, J=5.5 Hz, 1H), 3.33 (dt, J=3.3, 1.6 Hz, 1H), 2.03 (s, 1H), 1.66-1.54 (m, 1H), 1.38-1.28 (m, 1H), 1.05 (d, J=6.8 Hz, 3H), 0.97 (t, J=7.4 Hz, 3H); MS (ESI) m/e 434.2 [(M+H)⁺, calcd for C₂₂H₃₂N₃O₆ 434.2].

### Part H. (R)-tert-Butyl 1-(2-formyl-3-methoxy-4-(oxazol-5-yl)phenylamino)-4-methyl-1-oxopentan-2-ylcarbamate

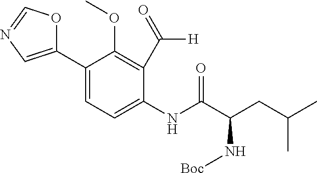 [SMILES: COc1c(-c2cnco2)ccc(NC(=O)[C@@H](CC(C)C)NC(=O)OC(C)(C)C)c1C=O]

Oxalyl chloride (0.041 mL, 0.468 mmol) was added to a solution of DMSO (0.057 mL, 0.803 mmol) in CH₂Cl₂ (1.5 mL) at −78° C. After stirring for 10 min, (R)-tert-butyl 1-(2-(hydroxymethyl)-3-methoxy-4-(oxazol-5-yl)phenylamino)-4-methyl-1-oxopentan-2-ylcarbamate (58 mg, 0.134 mmol) dissolved in CH₂Cl₂ (1 mL) as added via cannula. The solution was stirred at −78° C. for 45 min. Triethylamine (0.224 mL, 1.606 mmol) was then added dropwise via syringe and the reaction mixture was stirred at −78° C. for 15 min and was then allowed to warm up to room temperature over 30 min. The reaction mixture was transferred to a separatory funnel containing saturated aqueous NaHCO₃ solution (5 mL). The aqueous layer was extracted with ethyl acetate (3×10 mL). The combined organic layers were washed with brine (5 mL), dried over MgSO₄, filtered, and concentrated. The residue was purified by column chromatography on silica gel (30%-50% ethyl acetate in hexanes) to afford (R)-tert-butyl 1-(2-formyl-3-methoxy-4-(oxazol-5-yl)phenylamino)-4-methyl-1-oxopentan-2-ylcarbamate (30 mg, 52% yield) as a colorless solid: ¹H NMR (400 MHz, CD₃OD) δ 10.50 (d, J=0.8 Hz, 1H), 8.63 (dd, J=9.0, 0.5 Hz, 1H), 8.32 (s, 1H), 8.07 (d, J=9.0 Hz, 1H), 7.61 (s, 1H), 4.09 (d, J=5.3 Hz, 1H), 3.93 (s, 3H), 2.06 (br. s., 1H), 1.50 (s, 9H), 1.44-1.35 (m, 1H), 1.35-1.25 (m, 1H), 1.03-0.90 (m, 6H).

### Part I. tert-Butyl 1-(5-methoxy-6-(oxazol-5-yl)quinazolin-2-yl)-3-methylbutylcarbamate

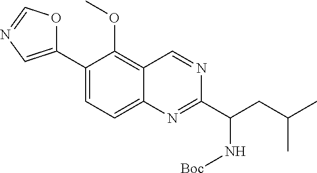 [SMILES: COc1c(-c2cnco2)ccc2nc(C(CC(C)C)NC(=O)OC(C)(C)C)ncc12]

To a solution of (R)-tert-butyl 1-(2-formyl-3-methoxy-4-(oxazol-5-yl)phenylamino)-4-methyl-1-oxopentan-2-ylcarbamate (30 mg, 0.070 mmol) in acetic acid (1 mL) was added ammonium acetate (53.6 mg, 0.695 mmol). The reaction mixture was heated at 85° C. for 14 h. The mixture was concentrated and the product was purified by reverse phase HPLC (5% MeCN:95% water:0.1% TFA→95% MeCN:5% water:0.1% TFA). The organic solvent was removed on the rotovapor and the aqueous mixture was frozen and placed on the lyophilizer to afford tert-butyl 1-(5-methoxy-6-(oxazol-5-yl)quinazolin-2-yl)-3-methylbutylcarbamate (20 mg, 70% yield) as a pale yellow amorphous solid: MS (ESI) m/e 413.3 [(M+H)⁺, calcd for C₂₂H₂₉N₄O₄ 413.2].

### Part J. 1-(5-Methoxy-6-(oxazol-5-yl)quinazolin-2-yl)-3-methylbutan-1-amine

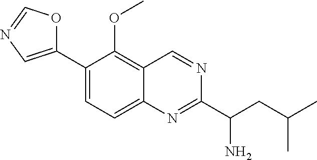 [SMILES: COc1c(-c2cnco2)ccc2nc(C(N)CC(C)C)ncc12]

tert-Butyl 1-(5-methoxy-6-(oxazol-5-yl)quinazolin-2-yl)-3-methylbutylcarbamate (20 mg, 0.048 mmol) was dissolved in CH₂Cl₂ (1 mL) and cooled to 0° C. To this solution was added TFA (0.037 mL, 0.485 mmol). The reaction mixture was stirred at room temperature for 2 h. The solvent was evaporated and the residue was purified by reverse phase HPLC (5% MeCN:95% water:0.1% TFA→95% MeCN:5% water:0.1% TFA) to afford 1-(5-methoxy-6-(oxazol-5-yl)quinazolin-2-yl)-3-methylbutan-1-amine (13 mg, 57% yield) as a TFA salt: ¹H NMR (400 MHz, DMSO-d₆) δ 9.89 (d, J=0.8 Hz, 1H), 8.66 (s, 1H), 8.53 (d, J=3.0 Hz, 3H), 8.44 (d, J=8.8 Hz, 1H), 7.97 (dd, J=9.0, 0.8 Hz, 1H), 7.90 (s, 1H), 4.57 (t, J=5.4 Hz, 1H), 4.08 (s, 3H), 2.26-2.15 (m, 1H), 1.62-1.51 (m, 1H), 1.39-1.27 (m, 1H), 0.94 (t, J=7.3 Hz, 3H), 0.87 (d, J=6.8 Hz, 3H); MS (ESI) m/e 313.2 [(M+H)⁺, calcd for C₁₇H₂₁N₄O₂ 313.2].

### Example 12

### 2-(1-Amino-3-methylbutyl)-6-(oxazol-5-yl)quinazolin-4-ol

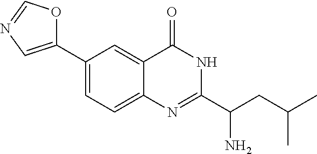 [SMILES: CC(C)CC(N)c1nc2ccc(-c3cnco3)cc2c(=O)[nH]1]

### Part A. Benzyl (1-((4-bromo-2-carbamoylphenyl)amino)-4-methyl-1-oxopentan-2-yl)carbamate

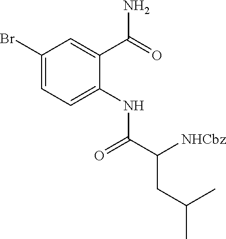 [SMILES: CC(C)CC(NC(=O)OCc1ccccc1)C(=O)Nc1ccc(Br)cc1C(N)=O]

To a solution of N-(chloromethylene)-N-methylmethanaminium, chloride salt (7.14 g, 55.8 mmol) (Jarrahpour et al. Tetrahedron 2009, 65, 2927-2934) in CH₂Cl₂ (80 mL) was added 2-(benzyloxycarbonylamino)-4-methylpentanoic acid (11.84 g, 44.6 mmol) at 0° C. The mixture was stirred at that temperature for 15 min. A premixed solution of 2-amino-5-bromobenzamide (8 g, 37.2 mmol) (Tobe et al. Bioorg. Med. Chem. 2003, 11, 383-392) and Et₃N (12.96 mL, 93 mmol) in CH₂Cl₂ (20 mL) was then added via cannula. The reaction mixture was stirred at 0° C. for 15 min and then at room temperature for 30 min. The reaction mixture was transferred to a separatory funnel containing saturated aqueous NaHCO₃ solution (150 mL). The aqueous layer was extracted with CH₂Cl₂ (3×100 mL). The organic layer was taken up in a separatory funnel and was washed with 6 N HCl (50 mL). The mixture was then washed with saturated NaHCO₃ solution (50 mL). The combined organic layers were washed with brine (5 mL), dried over MgSO₄, filtered, and concentrated to afford benzyl (1-((4-bromo-2-carbamoylphenyl)amino)-4-methyl-1-oxopentan-2-yl)carbamate. The solid was used without further purification. MS (ESI) m/e 445.1 [(M-NH₂+H), calcd for C₂₁H₂₂BrN₂O₄ 445.1].

### Part B. Benzyl (1-(6-bromo-4-oxo-3,4-dihydroquinazolin-2-yl)-3-methylbutyl)carbamate

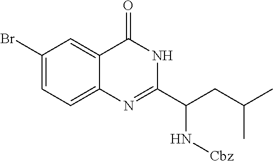 [SMILES: CC(C)CC(NC(=O)OCc1ccccc1)c1nc2ccc(Br)cc2c(=O)[nH]1]

Benzyl 1-(4-bromo-2-carbamoylphenylamino)-4-methyl-1-oxopentan-2-ylcarbamate (7.3 g, 15.8 mmol) and sodium carbonate (5.02 g, 47.4 mmol) in ethanol (20 mL) and water (20 mL) was heated at reflux for 2 h. The reaction mixture was acidified with citric acid. The reaction mixture was transferred to a separatory funnel and the aqueous layer was extracted with ethyl acetate (3×50 mL). The combined organic layers were washed with brine (50 mL), dried over MgSO₄, filtered, and concentrated to afford benzyl (1-(6-bromo-4-oxo-3,4-dihydroquinazolin-2-yl)-3-methylbutyl)carbamate. The product was used as is without further purification. MS (ESI) m/e 444.0 [(M+H)⁺, calcd for C₂₁H₂₃BrN₃O₃ 444.1].

### Part C. Benzyl 3-methyl-1-(4-oxo-6-vinyl-3,4-dihydroquinazolin-2-yl)butylcarbamate

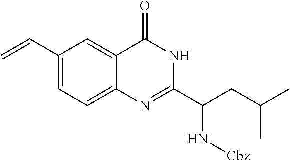 [SMILES: C=Cc1ccc2nc(C(CC(C)C)NC(=O)OCc3ccccc3)[nH]c(=O)c2c1]

To a solution of benzyl 1-(6-bromo-4-oxo-3,4-dihydroquinazolin-2-yl)-3-methylbutylcarbamate (7.00 g, 7.88 mmol) in toluene (50 mL) and ethanol (12.5 mL) was added 2,4,6-trivinyl-1,3,5,2,4,6-trioxatriborinane pyridine complex (1.90 g, 7.88 mmol) and sodium carbonate (2 N, aq) (11.82 mL, 23.63 mmol). The solution was degassed with N₂ for 5 min. Pd(PPh₃)₄ (0.455 g, 0.394 mmol) was added to the reaction mixture and the mixture was heated at 95° C. for 2.5 h. The reaction mixture was cooled to room temperature and transferred to a separatory funnel containing saturated aqueous NaHCO₃ solution (75 mL). The aqueous layer was extracted with ether (3×100 mL). The combined organic layers were washed with brine (50 mL), dried over MgSO₄, filtered, and concentrated. The residue was purified by column chromatography on silica gel (20%-50% ethyl acetate in hexanes) to afford benzyl 3-methyl-1-(4-oxo-6-vinyl-3,4-dihydroquinazolin-2-yl)butylcarbamate (1.50 g, 49% yield) as a colorless solid: ¹H NMR (400 MHz, DMSO-d₆) δ 12.32 (s, 1H), 8.11 (d, J=2.0 Hz, 1H), 8.00 (dd, J=8.5, 2.0 Hz, 1H), 7.72 (d, J=8.0 Hz, 1H), 7.61 (d, J=8.5 Hz, 1H), 7.39-7.29 (m, 5H), 6.91 (dd, J=17.6, 11.0 Hz, 1H), 5.98 (d, J=17.6 Hz, 1H), 5.38 (d, J=11.3 Hz, 1H), 5.10-4.97 (m, 2H), 4.62-4.51 (m, 1H), 1.75-1.64 (m, 2H), 1.57 (td, J=9.8, 5.5 Hz, 1H), 0.92 (d, J=2.8 Hz, 3H), 0.91 (d, J=2.8 Hz, 3H); MS (ESI) m/e 392.2 [(M+H)⁺, calcd for C₂₃H₂₆N₃O₃ 392.2].

### Part D. Benzyl 1-(6-formyl-4-oxo-3,4-dihydroquinazolin-2-yl)-3-methylbutylcarbamate

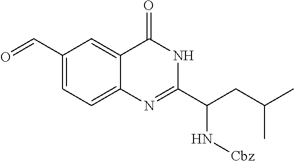 [SMILES: CC(C)CC(NC(=O)OCc1ccccc1)c1nc2ccc(C=O)cc2c(=O)[nH]1]

To a solution of benzyl 3-methyl-1-(4-oxo-6-vinyl-3,4-dihydroquinazolin-2-yl)butylcarbamate (1.5 g, 3.83 mmol) in dioxane (20 mL) and water (5 mL) at 0° C. was added 2,6-lutidine (0.893 mL, 7.66 mmol), osmium tetroxide (2.5% in 2-methyl-2-propanol) (0.962 mL, 0.077 mmol), and sodium periodate (3.28 g, 15.33 mmol).

The cooling bath was removed and the reaction mixture was allowed to warm up to room temperature while stirring for 2.5 h. The reaction mixture was transferred to a separatory funnel containing water (10 mL) and saturated aqueous NaHCO₃ solution (20 mL). The aqueous layer was extracted with ethyl acetate (3×25 mL). The combined organic layers were washed with brine (25 mL), dried over MgSO₄, filtered, and concentrated. The residue was purified by column chromatography on silica gel (30%-60% ethyl acetate in hexanes) to afford benzyl 1-(6-formyl-4-oxo-3,4-dihydroquinazolin-2-yl)-3-methylbutylcarbamate (1.2 g, 80% yield) as an off-white solid: ¹H NMR (400 MHz, DMSO-d₆) δ 12.66 (s, 1H), 10.14 (s, 1H), 8.67 (d, J=1.8 Hz, 1H), 8.23 (dd, J=8.5, 2.0 Hz, 1H), 7.82 (d, J=7.5 Hz, 1H), 7.76 (d, J=8.3 Hz, 1H), 7.38-7.28 (m, 5H), 5.10-4.99 (m, 2H), 4.63-4.54 (m, 1H), 1.77-1.66 (m, 2H), 1.63-1.53 (m, 1H), 0.93 (d, J=2.0 Hz, 2H), 0.91 (d, J=1.8 Hz, 2H); MS (ESI) m/e 394.2 [(M+H)⁺, calcd for C₂₂H₂₄N₃O₄ 394.2].

### Part E. Benzyl 3-methyl-1-(6-(oxazol-5-yl)-4-oxo-3,4-dihydroquinazolin-2-yl)butylcarbamate

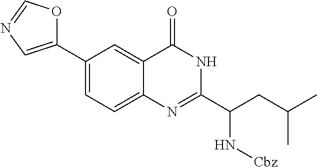 [SMILES: CC(C)CC(NC(=O)OCc1ccccc1)c1nc2ccc(-c3cnco3)cc2c(=O)[nH]1]

To a solution of benzyl 1-(6-formyl-4-oxo-3,4-dihydroquinazolin-2-yl)-3-methylbutylcarbamate (1.2 g, 3.05 mmol) and TosMIC (1.79 g, 9.15 mmol) in methanol (30 mL) was added K₂CO₃ (1.307 g, 9.46 mmol). The reaction mixture was heated at reflux for 1.5 h. The reaction mixture was cooled to room temperature and was transferred to a separatory funnel containing saturated aqueous NaHCO₃ solution (80 mL). The aqueous layer was extracted with ethyl acetate (3×50 mL).

The combined organic layers were washed with brine (60 mL), dried over Na₂SO₄, filtered and concentrated. The residue was purified by column chromatography on silica gel (20%-50% ethyl acetate in hexanes) to afford benzyl 3-methyl-1-(6-(oxazol-5-yl)-4-oxo-3,4-dihydroquinazolin-2-yl)butylcarbamate (700 mg, 53% yield) as a colorless solid: ¹H NMR (400 MHz, DMSO-d₆) δ 12.47 (s, 1H), 8.54 (s, 1H), 8.38 (d, J=1.8 Hz, 1H), 8.17 (dd, J=8.5, 1.8 Hz, 1H), 7.90 (s, 1H), 7.76 (d, J=8.0 Hz, 1H), 7.72 (d, J=8.5 Hz, 1H), 7.39-7.28 (m, 5H), 7.06 (br. s., 1H), 5.12-5.00 (m, 2H), 4.61-4.52 (m, 1H), 1.71 (t, J=9.8 Hz, 2H), 1.62-1.53 (m, 1H), 0.95-0.89 (m, 6H); MS (ESI) m/e 433.2 [(M+H)⁺, calcd for C₂₄H₂₅N₄O₄ 433.2].

### Part F. 2-(1-Amino-3-methylbutyl)-6-(oxazol-5-yl)quinazolin-4(3H)-one

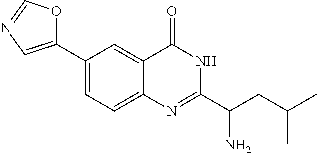 [SMILES: CC(C)CC(N)c1nc2ccc(-c3cnco3)cc2c(=O)[nH]1]

To a solution of benzyl 3-methyl-1-(6-(oxazol-5-yl)-4-oxo-3,4-dihydroquinazolin-2-yl)butylcarbamate (100 mg, 0.231 mmol) in DMF (4 mL) and ethanol (1 mL) at room temperature was added 10% palladium on carbon (49.2 mg, 0.046 mmol). The reaction mixture was stirred at room temperature under H₂ for 2 h. The solid was removed by filtration through a pad of Celite and the mixture was concentrated to afford yellow solid. This solid was triturated with hexanes and filtered to afford 2-(1-amino-3-methylbutyl)-6-(oxazol-5-yl)quinazolin-4(3H)-one (50 mg, 69% yield) as a yellow solid: ¹H NMR (400 MHz, DMSO-d₆) δ 8.53 (s, 1H), 8.38 (d, J=2.0 Hz, 1H), 8.14 (dd, J=8.5, 2.0 Hz, 1H), 7.97 (s, 1H), 7.88 (s, 1H), 7.73 (d, J=8.5 Hz, 1H), 5.67 (br. s., 2H), 3.74 (dd, J=8.4, 6.1 Hz, 1H), 1.72 (dq, J=13.6, 6.6 Hz, 1H), 1.64-1.46 (m, 2H), 0.93 (d, J=6.8 Hz, 3H), 0.90 (d, J=6.5 Hz, 3H); MS (ESI) m/e 297.2 [(M−H)⁻, calcd for C₁₆H₁₇N₄O₂ 297.1]. HPLC retention time (Method O): t_{R}=7.30 min; HPLC retention time (Method P): t_{R}=7.14 min.

### Example 13

### 2-(1-Amino-3-methylbutyl)-6-(pyridin-4-yl)quinazolin-4-ol

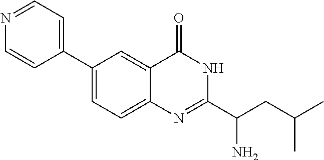 [SMILES: CC(C)CC(N)c1nc2ccc(-c3ccncc3)cc2c(=O)[nH]1]

### Part A. Benzyl (1-((4-bromo-2-carbamoylphenyl)amino)-4-methyl-1-oxopentan-2-yl)carbamate

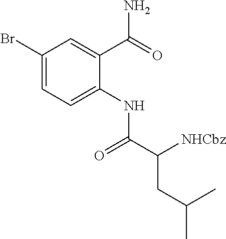 [SMILES: CC(C)CC(NC(=O)OCc1ccccc1)C(=O)Nc1ccc(Br)cc1C(N)=O]

To a solution of N-(chloromethylene)-N-methylmethanaminium, chloride salt (1.42 g, 11.1 mmol) (Jarrahpour et al. Tetrahedron 2009, 65, 2927-2934) in CH₂Cl₂ (20 mL) was added 2-(benzyloxycarbonylamino)-4-methylpentanoic acid (2.52 g, 9.49 mmol) at 0° C. The mixture was stirred at that temperature for 15 min. 2-Amino-5-bromobenzamide (1.7 g, 7.91 mmol) (Tobe et al. Bioorg. Med. Chem. 2003, 11, 383-392) dissolved in CH₂Cl₂ (2 mL) was then added via cannula. The reaction mixture was stirred at 0° C. for 15 min. The reaction mixture was transferred to a separatory funnel containing saturated aqueous NaHCO₃ solution (5 mL). The aqueous layer was extracted with methylene chloride (3×10 mL). The combined organic layers were washed with brine (5 mL), dried over MgSO₄, filtered, and concentrated to afford benzyl (1-((4-bromo-2-carbamoylphenyl)amino)-4-methyl-1-oxopentan-2-yl)carbamate (2.00 g, 55% yield) as a solid. The product was used without further purification. MS (ESI) m/e 462.0 [(M+H)⁺, calcd for C₂₁H₂₅BrN₃O₄ 462.1].

### Part B. Benzyl (1-(6-bromo-4-oxo-3,4-dihydroquinazolin-2-yl)-3-methylbutyl)carbamate

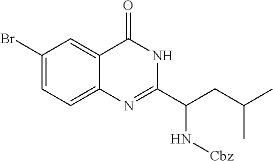 [SMILES: CC(C)CC(NC(=O)OCc1ccccc1)c1nc2ccc(Br)cc2c(=O)[nH]1]

A solution of benzyl 1-(4-bromo-2-carbamoylphenylamino)-4-methyl-1-oxopentan-2-ylcarbamate (2.00 g, 4.33 mmol) and sodium carbonate (1.38 g, 13.0 mmol) in ethanol (20 mL) and water (20 mL) was heated at reflux for 2 h. The reaction mixture was acidified with citric acid. The reaction mixture was transferred to a separatory funnel and the aqueous layer was extracted with ethyl acetate (3×50 mL).

The combined organic layers were washed with brine (50 mL), dried over MgSO₄, filtered, and concentrated. The residue was purified by column chromatography on silica gel (50%→70% ethyl acetate in hexanes) to afford benzyl 1-(6-bromo-4-oxo-3,4-dihydroquinazolin-2-yl)-3-methylbutylcarbamate (1.20 g, 63% yield) as a yellow solid: ¹H NMR (400 MHz, DMSO-d₆) δ 12.51 (s, 1H), 8.19 (d, J=2.3 Hz, 1H), 7.97 (dd, J=8.7, 2.4 Hz, 1H), 7.75 (d, J=7.8 Hz, 1H), 7.58 (d, J=8.8 Hz, 1H), 7.38-7.31 (m, 5H), 5.04 (d, J=4.8 Hz, 2H), 4.59-4.52 (m, 1H), 1.75-1.64 (m, 2H), 1.61-1.51 (m, 1H), 0.92 (d, J=2.5 Hz, 3H), 0.90 (d, J=2.5 Hz, 3H); MS (ESI) m/e 443.9 [(M+H)⁺, calcd for C₂₁H₂₃BrN₃O₃ 444.1].

### Part C. Benzyl 3-methyl-1-(4-oxo-6-(pyridin-4-yl)-3,4-dihydroquinazolin-2-yl)butylcarbamate

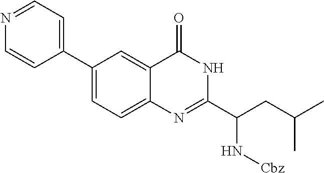 [SMILES: CC(C)CC(NC(=O)OCc1ccccc1)c1nc2ccc(-c3ccncc3)cc2c(=O)[nH]1]

To a mixture of benzyl 1-(6-bromo-4-oxo-3,4-dihydroquinazolin-2-yl)-3-methylbutylcarbamate (80 mg, 0.180 mmol) and 4-pyridylboronic acid (44.3 mg, 0.360 mmol) in THF (5 mL) was added sodium carbonate (2M in H₂O) (0.270 mL, 0.540 mmol). N₂ was bubbled through the reaction mixture for 2 min. Pd(PPh₃)Cl₂ (12.64 mg, 0.018 mmol) was then added and the reaction mixture was heated at 80° C. for 3 h. The reaction mixture was cooled to room temperature and was transferred to a separatory funnel containing saturated aqueous NaHCO₃ solution (10 mL). The aqueous layer was extracted with ethyl acetate (3×25 mL). The combined organic layers were washed with brine (10 mL), dried over MgSO₄, filtered and concentrated.

The residue was purified by column chromatography on silica gel (30%-70% ethyl acetate in hexanes) to afford benzyl 3-methyl-1-(4-oxo-6-(pyridin-4-yl)-3,4-dihydroquinazolin-2-yl)butylcarbamate (60 mg, 75% yield) as a yellow solid: ¹H NMR (400 MHz, CD₃OD) δ 8.66 (d, J=6.0 Hz, 2H), 8.59 (d, J=1.8 Hz, 1H), 8.23 (dd, J=8.4, 2.1 Hz, 1H), 7.88-7.82 (m, 3H), 7.42-7.28 (m, 5H), 5.12 (s, 2H), 4.70 (dd, J=9.7, 5.4 Hz, 1H), 1.86-1.71 (m, 3H), 1.02 (d, J=6.0 Hz, 6H); MS (ESI) m/e 443.3 [(M+H)⁺, calcd for C₂₆H₂₇N₄O₃ 443.2].

### Part D. 2-(1-Amino-3-methylbutyl)-6-(pyridin-4-yl)quinazolin-4(3H)-one

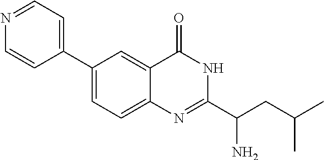 [SMILES: CC(C)CC(N)c1nc2ccc(-c3ccncc3)cc2c(=O)[nH]1]

Palladium on carbon (24.05 mg, 0.023 mmol) was added to a solution of benzyl 3-methyl-1-(4-oxo-6-(pyridin-4-yl)-3,4-dihydroquinazolin-2-yl)butylcarbamate (50 mg, 0.113 mmol) in DMF/EtOH (5/1.25 mL). The solution was purged with hydrogen and stirred under a hydrogen atmosphere (1 bar) for 2 h. The catalyst was filtered off and the filtrate was evaporated to dryness. The residue was purified by reverse phase HPLC (5% MeCN:95% water:0.1% TFA→95% MeCN:5% water:0.1% TFA) to afford 2-(1-amino-3-methylbutyl)-6-(pyridin-4-yl)quinazolin-4(3H)-one (15 mg, 25% yield) as a TFA salt: ¹H NMR (400 MHz, CD₃OD) δ 8.84 (d, J=6.3 Hz, 2H), 8.76 (d, J=2.0 Hz, 1H), 8.39 (dd, J=8.5, 2.3 Hz, 1H), 8.25 (d, J=6.5 Hz, 2H), 7.98 (d, J=8.8 Hz, 1H), 4.39 (t, J=7.2 Hz, 1H), 1.94 (t, J=7.2 Hz, 2H), 1.84 (dt, J=13.6, 6.8 Hz, 1H), 1.09 (d, J=6.3 Hz, 3H), 1.06 (d, J=6.5 Hz, 3H); MS (ESI) m/e 309.1 [(M+H)⁺, calcd for C₁₈H₂₁N₄O 309.2]. HPLC retention time (Method O): t_{R}=5.70 min; HPLC retention time (Method P): t_{R}=4.97 min.

### Example 14

### 1-(4-Methoxy-6-(oxazol-5-yl)quinazolin-2-yl)-3-methylbutan-1-amine

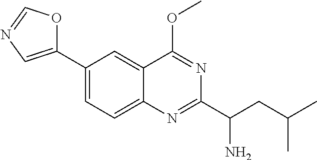 [SMILES: COc1nc(C(N)CC(C)C)nc2ccc(-c3cnco3)cc12]

### Part A. Benzyl (1-(4-methoxy-6-(oxazol-5-yl)quinazolin-2-yl)-3-methylbutyl)carbamate

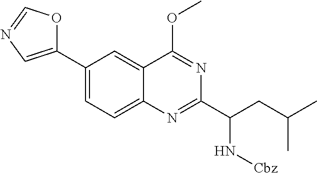 [SMILES: COc1nc(C(CC(C)C)NC(=O)OCc2ccccc2)nc2ccc(-c3cnco3)cc12]

To a solution of benzyl 3-methyl-1-(6-(oxazol-5-yl)-4-oxo-3,4-dihydroquinazolin-2-yl)butylcarbamate (25 mg, 0.058 mmol), prepared as described in Example 12, Parts A-E, and BOP (33.2 mg, 0.075 mmol) in DMF (1 mL) at room temperature was added DBU (0.013 mL, 0.087 mmol) and MeOH (3.51 μl, 0.087 mmol) (Wan et al.

J. Org. Chem., 2007, 72, 10,194-10,210). The mixture was stirred at room temperature for 2 h. The reaction mixture was transferred to a separatory funnel containing brine (20 mL). The aqueous layer was extracted with ethyl acetate (3×10 mL). The combined organic layers were dried over MgSO₄, filtered, and concentrated. The product was used in the next step without further purification. MS (ESI) m/e 447.3 [(M+H)⁺, calcd for C₂₅H₂₇N₄O₄ 447.2].

### Part B. 1-(4-Methoxy-6-(oxazol-5-yl)quinazolin-2-yl)-3-methylbutan-1-amine

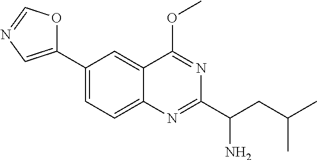 [SMILES: COc1nc(C(N)CC(C)C)nc2ccc(-c3cnco3)cc12]

A mixture of benzyl 1-(4-methoxy-6-(oxazol-5-yl)quinazolin-2-yl)-3-methylbutylcarbamate (20 mg, 0.045 mmol) and 10% palladium on carbon (9.5 mg, 0.009 mmol) in DMF (1 mL) and ethanol (1 mL) was stirred under a H₂ atmosphere under a balloon for 2 h. The mixture was filtered through a pad of Celite and was concentrated. The residue was purified by reverse phase HPLC (5% MeCN:95% water:0.1% TFA→95% MeCN:5% water:0.1% TFA) to afford 1-(4-methoxy-6-(oxazol-5-yl)quinazolin-2-yl)-3-methylbutan-1-amine (8 mg, 57% yield) as a yellow solid: ¹H NMR (400 MHz, CD₃OD) δ 8.58-8.53 (m, 1H), 8.38 (s, 1H), 8.33 (dd, J=8.8, 2.0 Hz, 1H), 8.05 (dd, J=8.8, 0.5 Hz, 1H), 7.78 (s, 1H), 4.56 (t, J=7.2 Hz, 1H), 4.31 (s, 3H), 2.13-1.99 (m, 1H), 1.90-1.79 (m, 2H), 1.10 (d, J=6.3 Hz, 3H), 1.06 (d, J=6.5 Hz, 3H); MS (ESI) m/e 313.3 [(M+H)⁺, calcd for C₁₇H₂₁N₄O₂ 313.2]. HPLC retention time (Method O): t_{R}=8.18 min; HPLC retention time (Method P): t_{R}=8.78 min.

### Example 15

### 1-(4-Ethoxy-6-(oxazol-5-yl)quinazolin-2-yl)-3-methylbutan-1-amine

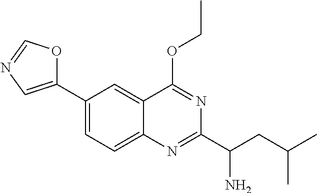 [SMILES: CCOc1nc(C(N)CC(C)C)nc2ccc(-c3cnco3)cc12]

### Part A. Benzyl (1-(4-ethoxy-6-(oxazol-5-yl)quinazolin-2-yl)-3-methylbutyl)carbamate

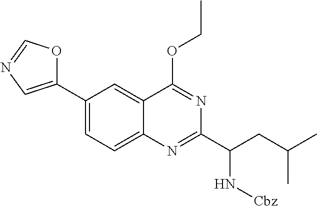 [SMILES: CCOc1nc(C(CC(C)C)NC(=O)OCc2ccccc2)nc2ccc(-c3cnco3)cc12]

To a solution of benzyl 3-methyl-1-(6-(oxazol-5-yl)-4-oxo-3,4-dihydroquinazolin-2-yl)butylcarbamate (50 mg, 0.116 mmol), prepared as described in Example 12, Parts A-E, in DMF (2 mL) at 0° C. was added NaH (60% in mineral oil) (5.09 mg, 0.127 mmol). The reaction mixture was stirred for 15 minutes. Iodoethane (9.34 μL, 0.116 mmol) was then added. The mixture was stirred at room temperature for 24 h. The reaction mixture was transferred to a separatory funnel containing brine (20 mL).

The aqueous layer was extracted with ethyl acetate (3×10 mL). The combined organic layers were dried over MgSO₄, filtered, and concentrated to give a mixture of N and O alkylated products. The residue was used without further purification in the next step: MS (ESI) m/e 461.3 [(M+H)⁺, calcd for C₂₆H₂₉N₄O₄ 461.2].

### Part B. 1-(4-Ethoxy-6-(oxazol-5-yl)quinazolin-2-yl)-3-methylbutan-1-amine

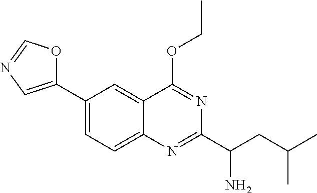 [SMILES: CCOc1nc(C(N)CC(C)C)nc2ccc(-c3cnco3)cc12]

To a solution of benzyl 1-(4-ethoxy-6-(oxazol-5-yl)quinazolin-2-yl)-3-methylbutylcarbamate (40 mg, 0.087 mmol) and benzyl 3-ethyl-1-(6-(oxazol-5-yl)-4-oxo-3,4-dihydroquinazolin-2-yl)butylcarbamate (37.6 mg, 0.087 mmol) in DMF (4 mL) and ethanol (1 mL) at room temperature was added 10% palladium on carbon (18.5 mg, 0.017 mmol). The reaction mixture was stirred at room temperature under H₂ for 2 h. The mixture was filtered and the residue was purified by reverse phase HPLC (5% MeCN:95% water:0.1% TFA→95% MeCN:5% water:0.1% TFA) to afford 2-(1-amino-3-methylbutyl)-3-ethyl-6-(oxazol-5-yl)quinazolin-4(3H)-one (10 mg, 25% yield) as a TFA salt as a byproduct and 1-(4-ethoxy-6-(oxazol-5-yl)quinazolin-2-yl)-3-methylbutan-1-amine (6 mg, 16% yield) as a TFA salt: ¹H NMR (400 MHz, DMSO-d₆) δ 8.59 (s, 1H), 8.46 (br. s., 3H), 8.41 (d, J=1.8 Hz, 1H), 8.37 (dd, J=8.8, 2.01 Hz, 1H), 8.04 (d, J=8.8 Hz, 1H), 8.00 (s, 1H), 4.74 (dtt, J=10.6, 7.1, 7.1, 3.6, 3.6 Hz, 2H), 4.44 (d, J=5.5 Hz, 1H), 1.98-1.85 (m, 1H), 1.84-1.67 (m, 2H), 1.52 (t, J=7.0 Hz, 3H,), 0.98 (d, J=6.0 Hz, 3H), 0.94 (d, J=6.3 Hz, 3H); MS (ESI) m/e 327.2 [(M+H)⁺, calcd for C18H₂₃N₄O₂ 327.2].

### Example 16

### 2-(1-Amino-3-methylbutyl)-N-methyl-6-(oxazol-5-yl)quinazolin-4-amine

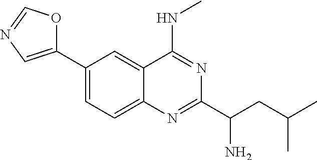 [SMILES: CNc1nc(C(N)CC(C)C)nc2ccc(-c3cnco3)cc12]

Prepared in a similar fashion as described in Example 14 using methylamine in Part A to give 2-(1-amino-3-methylbutyl)-N-methyl-6-(oxazol-5-yl)quinazolin-4-amine (25 mg) as a TFA salt: ¹H NMR (400 MHz, DMSO-d₆) δ 8.96 (br. s., 1H), 8.64 (d, J=2.01 Hz, 1H), 8.60 (s, 1H), 8.34 (br. s., 3H), 8.18 (dd, J=8.8, 1.8 Hz, 1H), 7.84-7.77 (m, 2H), 4.26 (d, J=5.3 Hz, 1H), 3.12 (d, J=4.5 Hz, 3H), 1.90-1.81 (m, 1H), 1.81-1.71 (m, 2H), 0.98 (d, J=6.02 Hz, 3H), 0.94 (d, J=6.27 Hz, 3H); MS (ESI) m/e 312.2 [(M+H)⁺, calcd for C₁₇H₂₂N₅O 312.2]. HPLC retention time (Method O): t_{R}=6.51 min; HPLC retention time (Method P): t_{R}=6.51 min.

### Example 17

### 2-(1-Amino-3-methylbut)-N,N-dimethyl-6-(oxazol-5-yl)quinazolin-4-amine

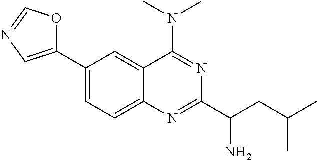 [SMILES: CC(C)CC(N)c1nc(N(C)C)c2cc(-c3cnco3)ccc2n1]

Prepared in a similar fashion as described in Example 14 using dimethylamine in Part A to give 2-(1-amino-3-methylbutyl)-N,N-dimethyl-6-(oxazol-5-yl)quinazolin-4-amine (10 mg) as a TFA salt: ¹H NMR (400 MHz, CD₃OD) δ 8.59 (d, J=1.8 Hz, 1H), 8.37 (s, 1H), 8.28 (dd, J=8.8, 2.0 Hz, 1H), 7.95-7.85 (m, 2H), 7.77 (s, 1H), 7.55-7.45 (m, 1H), 4.53 (dd, J=8.2, 6.4 Hz, 1H), 3.66 (s, 6H), 2.14-1.86 (m, 3H), 1.06 (d, J=6.53 Hz, 3H), 1.08 (d, J=6.53 Hz, 3H); MS (ESI) m/e 326.2 [(M+H)⁺, calcd for C18H₂₄N₅O 326.2]. HPLC retention time (Method O): t_{R}=6.26 min; HPLC retention time (Method P): t_{R}=6.32 min.

### Example 18

### 2-(1-Amino-3-methylbutyl)-N,N-diethyl-6-(oxazol-5-yl)quinazolin-4-amine

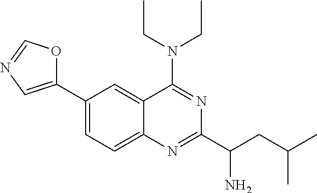 [SMILES: CCN(CC)c1nc(C(N)CC(C)C)nc2ccc(-c3cnco3)cc12]

Prepared in a similar fashion as described in Example 14 using diethylamine in Part A to give 2-(1-amino-3-methylbutyl)-N,N-diethyl-6-(oxazol-5-yl)quinazolin-4-amine (26 mg) as a TFA salt: ¹H NMR (400 MHz, DMSO-d₆) δ 8.56 (s, 1H), 8.31 (br. s., 2H), 8.28 (d, J=1.8 Hz, 1H), 8.20 (d, J=8.8 Hz, 1H), 7.92-7.81 (m, 2H), 4.26 (br. s., 1H), 3.84 (q, J=6.9 Hz, 4H), 1.90-1.81 (m, 1H), 1.78-1.68 (m, 2H), 1.41 (t, J=6.9 Hz, 5H), 0.97 (d, J=6.3 Hz, 3H), 0.91 (d, J=6.3 Hz, 3H); MS (ESI) m/e 354.4 [(M+H)⁺, calcd for C₂₀H₂₈N₅O 354.2]. HPLC retention time (Method O): t_{R}=7.06 min; HPLC retention time (Method P): t_{R}=7.15 min.

### Example 19

### 3-Methyl-1-(6-(oxazol-5-yl)-4-(pyrrolidin-1-yl)quinazolin-2-yl)butan-1-amine

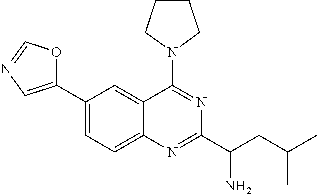 [SMILES: CC(C)CC(N)c1nc(N2CCCC2)c2cc(-c3cnco3)ccc2n1]

Prepared in a similar fashion as described in Example 14 using pyrrolidine in Part A to give 3-methyl-1-(6-(oxazol-5-yl)-4-(pyrrolidin-1-yl)quinazolin-2-yl)butan-1-amine (20 mg) as a TFA salt: ¹H NMR (400 MHz, DMSO-d₆) δ 8.55 (s, 2H), 8.33 (br. s., 3H), 8.19 (dd, J=8.5, 1.8 Hz, 1H), 7.89 (s, 1H), 7.83 (d, J=8.8 Hz, 1H), 4.26 (d, J=5.3 Hz, 1H), 4.03 (br. s., 4H), 2.13-1.99 (m, 4H), 1.90-1.82 (m, 1H), 1.80-1.70 (m, 2H), 0.98 (d, J=5.8 Hz, 3H), 0.92 (d, J=5.8 Hz, 3H); MS (ESI) m/e 352.2 [(M+H)⁺, calcd for C₂₀H₂₆N₅O 352.2]. HPLC retention time (Method O): t_{R}=6.96 min; HPLC retention time (Method P): t_{R}=6.93 min.

### Example 20

### 2-(1-Amino-3-methylbutyl)-N-ethyl-6-(oxazol-5-yl)quinazolin-4-amine

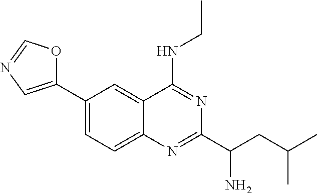 [SMILES: CCNc1nc(C(N)CC(C)C)nc2ccc(-c3cnco3)cc12]

Prepared in a similar fashion as described in Example 14 using ethylamine in Part A to give 2-(1-amino-3-methylbutyl)-N-ethyl-6-(oxazol-5-yl)quinazolin-4-amine (10 mg) as a TFA salt: ¹H NMR (400 MHz, CDCl₃) δ 8.00-7.90 (m, 3H), 7.89-7.74 (m, 1H), 7.46 (s, 1H), 5.87 (br. s., 1H), 4.02 (dd, J=7.9, 5.9 Hz, 1H), 3.84-3.68 (m, 2H), 1.86-1.71 (m, 2H), 1.69-1.58 (m, 1H), 1.40 (t, J=7.3 Hz, 3H), 1.00 (d, J=6.5 Hz, 3H), 0.97 (d, J=6.3 Hz, 3H); MS (ESI) m/e 326.3 [(M+H)⁺, calcd for C18H₂₄N₅O 326.2]. HPLC retention time (Method O): t_{R}=6.67 min; HPLC retention time (Method P): t_{R}=6.45 min.

### Example 21

### 2-((2-(1-Amino-3-methylbutyl)-6-(oxazol-5-yl)quinazolin-4-yl)amino)ethanol

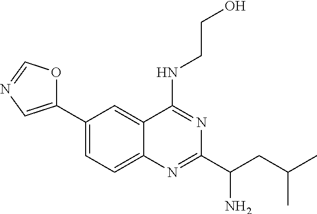 [SMILES: CC(C)CC(N)c1nc(NCCO)c2cc(-c3cnco3)ccc2n1]

Prepared in a similar fashion as described in Example 14 using 2-aminoethanol in Part A to give 2-((2-(1-amino-3-methylbutyl)-6-(oxazol-5-yl)quinazolin-4-yl)amino)ethanol (15 mg) as a TFA salt: ¹H NMR (400 MHz, DMSO-d₆) δ 8.89 (br. s., 1H), 8.72 (d, J=1.8 Hz, 1H), 8.60 (s, 1H), 8.30 (br. s., 2H), 8.22-8.07 (m, 1H), 7.83-7.75 (m, 1H), 4.23 (d, J=5.3 Hz, 1H), 3.82-3.60 (m, 4H), 1.88-1.79 (m, 1H), 1.79-1.66 (m, 2H), 0.97 (d, J=6.0 Hz, 3H), 0.93 (d, J=6.0 Hz, 3H); MS (ESI) m/e 342.4 [(M+H)⁺, calcd for C18H₂₄N₅O₂ 342.2]. HPLC retention time (Method O): t_{R}=6.10 min; HPLC retention time (Method P): t_{R}=6.28 min.

### Example 22

### 2-(1-Amino-3-methylbutyl)-N-(2-methoxyethyl)-6-(oxazol-5-yl)quinazolin-4-amine

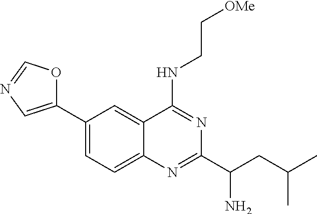 [SMILES: COCCNc1nc(C(N)CC(C)C)nc2ccc(-c3cnco3)cc12]

Prepared in a similar fashion as described in Example 14 using 2-methoxyethanamine in Part A to give 2-(1-amino-3-methylbutyl)-N-(2-methoxyethyl)-6-(oxazol-5-yl)quinazolin-4-amine (20 mg) as a TFA salt: ¹H NMR (400 MHz, DMSO-d₆) δ 8.91 (br. s., 1H), 8.71 (d, J=1.8 Hz, 1H), 8.60 (s, 1H), 8.29 (br. s., 3H), 8.17 (dd, J=8.8, 1.8 Hz, 1H), 7.82-7.79 (m, 2H), 4.23 (d, J=6.0 Hz, 1H), 3.84-3.77 (m, 2H), 3.63 (t, J=5.7 Hz, 2H), 3.41-3.24 (m, 3H), 1.87-1.80 (m, 1H), 1.77-1.69 (m, 2H), 0.98 (d, J=6.0 Hz, 3H), 0.93 (d, J=6.3 Hz, 3H); MS (ESI) m/e 356.3 [(M+H)⁺, calcd for C₁₉H₂₆N₅O₂ 356.2]. HPLC retention time (Method O): t_{R}=6.66 min; HPLC retention time (Method P): t_{R}=6.58 min.

### Example 23

### 2-(1-Amino-3-methylbutyl)-N-methyl-6-(pyridin-4-yl)quinazolin-4-amine

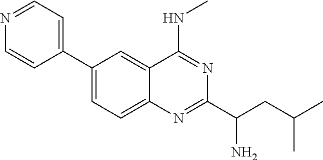 [SMILES: CNc1nc(C(N)CC(C)C)nc2ccc(-c3ccncc3)cc12]

### Part A. Benzyl (3-methyl-1-(4-(methylamino)-6-(pyridin-4-yl)quinazolin-2-yl)butyl)carbamate

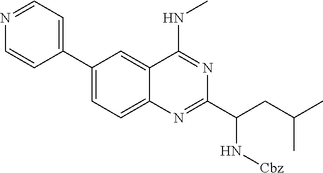 [SMILES: CNc1nc(C(CC(C)C)NC(=O)OCc2ccccc2)nc2ccc(-c3ccncc3)cc12]

To a solution of benzyl 3-methyl-1-(4-oxo-6-(pyridin-4-yl)-3,4-dihydroquinazolin-2-yl)butylcarbamate (35 mg, 0.079 mmol), prepared as described in Example 13, Parts A-C, and BOP (45.5 mg, 0.103 mmol) in DMF (1 mL) at room temperature was added DBU (0.024 mL, 0.158 mmol) and methylamine (2M in THF) (0.079 mL, 0.158 mmol) (Wan et al. J. Org. Chem., 2007, 72, 10,194-10,210). The mixture was stirred at room temperature for 2 h. The reaction mixture was transferred to a separatory funnel containing brine (20 mL). The aqueous layer was extracted with ethyl acetate (3×10 mL). The combined organic layers were dried over MgSO₄, filtered, and concentrated to furnish benzyl (3-methyl-1-(4-(methylamino)-6-(pyridin-4-yl)quinazolin-2-yl)butyl)carbamate (25 mg, 69% yield). The product was used in the next step without further purification. ¹H NMR (400 MHz, CDCl₃) δ 8.61 (d, J=5.5 Hz, 2H), 8.21 (s, 1H), 8.00 (s, 1H), 7.86 (d, J=8.5 Hz, 1H), 7.74 (d, J=8.5 Hz, 1H), 7.57 (d, J=5.5 Hz, 2H), 7.41-7.28 (m, 5H), 6.11 (d, J=8.5 Hz, 1H), 5.18-5.05 (m, 2H), 4.96-4.88 (m, 1H), 3.13 (d, J=4.3 Hz, 3H), 1.80-1.64 (m, 3H), 0.99 (d, J=5.3 Hz, 3H), 0.96 (d, J=5.5 Hz, 3H); MS (ESI) m/e 456.3 [(M+H)⁺, calcd for C₂₇H₃₀N₅O₂ 456.3].

### Part B. 2-(1-Amino-3-methylbutyl)-N-methyl-6-(pyridin-4-yl)quinazolin-4-amine

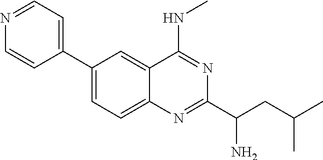 [SMILES: CNc1nc(C(N)CC(C)C)nc2ccc(-c3ccncc3)cc12]

To a solution of benzyl 3-methyl-1-(4-(methylamino)-6-(pyridin-4-yl)quinazolin-2-yl)butylcarbamate (25 mg, 0.055 mmol) in DMF (2 mL) and ethanol (2.0 mL) at room temperature was added 10% palladium on carbon (11.7 mg, 0.011 mmol). The reaction mixture was stirred at room temperature under H₂ for 2 h. The mixture was filtered and the residue was purified by reverse phase HPLC (5% MeCN:95% water:0.1% TFA→95% MeCN:5% water:0.1% TFA) to afford 2-(1-amino-3-methylbutyl)-N-methyl-6-(pyridin-4-yl)quinazolin-4-amine (20 mg, 66% yield) as a TFA salt: ¹H NMR (400 MHz, DMSO-d₆) δ 9.20 (br. s., 1H), 9.03-8.91 (m, 3H), 8.50-8.36 (m, 3H), 8.27 (d, J=5.8 Hz, 2H), 7.90 (d, J=8.8 Hz, 1H), 4.32 (br. s., 1H), 3.18 (d, J=4.5 Hz, 3H), 1.99-1.73 (m, 3H), 0.98 (d, J=6.0 Hz, 3H), 0.94 (d, J=6.0 Hz, 3H); MS (ESI) m/e 322.3 [(M+H)⁺, calcd for C₁₉H₂₄N₅ 322.2].

### Example 24

### 2-(1-Amino-3-methylbutyl)-N-ethyl-6-(pyridin-4-yl)quinazolin-4-amine

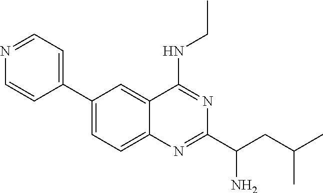 [SMILES: CCNc1nc(C(N)CC(C)C)nc2ccc(-c3ccncc3)cc12]

Prepared in a similar fashion as described in Example 23 using ethylamine in Part A to give 2-(1-amino-3-methylbutyl)-N-ethyl-6-(pyridin-4-yl)quinazolin-4-amine (35 mg) as a TFA salt: ¹H NMR (400 MHz, DMSO-d₆) δ 9.01 (br. s., 1H), 8.95-8.84 (m, 3H), 8.51-8.28 (m, 4H), 8.18 (d, J=6.3 Hz, 2H), 7.87 (d, J=8.8 Hz, 1H), 4.27 (br. s., 1H), 3.76-3.67 (m, 2H), 1.91-1.81 (m, 1H), 1.81-1.70 (m, 2H), 1.31 (t, J=7.2 Hz, 3H), 0.98 (d, J=5.8 Hz, 3H), 0.93 (d, J=6.0 Hz, 3H); MS (ESI) m/e 336.4 [(M+H)⁺, calcd for C₂₀H₂₆N₅ 336.3].

### Example 25

### 2-(1-Amino-3-methylbutyl)-N,N-diethyl-6-(pyridin-4-yl)quinazolin-4-amine

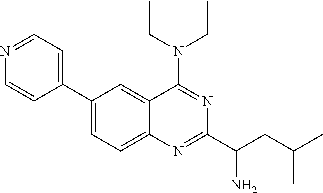 [SMILES: CCN(CC)c1nc(C(N)CC(C)C)nc2ccc(-c3ccncc3)cc12]

Prepared in a similar fashion as described in Example 23 using diethylamine in Part A to give 2-(1-amino-3-methylbutyl)-N,N-diethyl-6-(pyridin-4-yl)quinazolin-4-amine (35 mg) as a TFA salt: ¹H NMR (400 MHz, DMSO-d₆) δ 8.85 (d, J=6.0 Hz, 2H), 8.43-8.29 (m, 5H), 8.08 (d, J=6.0 Hz, 2H), 7.92 (d, J=8.8 Hz, 1H), 4.29 (d, J=5.3 Hz, 1H), 3.89 (q, J=7.0 Hz, 4H), 1.81-1.91 (m, 1H), 1.80-1.65 (m, 2H), 1.42 (t, J=6.9 Hz, 6H), 0.97 (d, J=6.3 Hz, 3H), 0.93 (d, J=6.3 Hz, 3H); MS (ESI) m/e 364.3 [(M+H)⁺, calcd for C₂₂H₃₀N₅ 364.3]. HPLC retention time (Method O): t_{R}=5.54 min; HPLC retention time (Method P): t_{R}=5.76 min.

### Example 26

### 2-(1-Amino-3-methylbutyl)-6-(3-methoxypyridin-4-yl)-N,N-dimethylquinazolin-4-amine

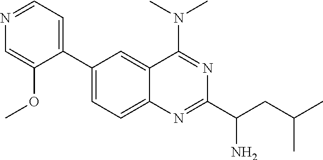 [SMILES: COc1cnccc1-c1ccc2nc(C(N)CC(C)C)nc(N(C)C)c2c1]

Prepared in a similar fashion as described in Example 23 using 2-(1-amino-3-methylbutyl)-6-(3-methoxypyridin-4-yl)quinazolin-4-ol and dimethylamine in Part A. 2-(1-Amino-3-methylbutyl)-6-(3-methoxypyridin-4-yl)quinazolin-4-ol was prepared as described in Example 13, Parts A-C using 3-methoxypyridylboronic acid in Part C. 2-(1-Amino-3-methylbutyl)-6-(3-methoxypyridin-4-yl)-N,N-dimethylquinazolin-4-amine (18 mg) was isolated as a TFA salt: ¹H NMR (400 MHz, CD₃OD) δ 8.76-8.70 (m, 2H), 8.57 (d, J=5.3 Hz, 1H), 8.27-8.23 (m, 1H), 8.06 (d, J=5.5 Hz, 1H), 7.96 (d, J=8.8 Hz, 1H), 4.57 (t, J=7.2 Hz, 1H), 4.12 (s, 3H), 3.69 (s, 6H), 2.06 (t, J=5.9 Hz, 1H), 1.95 (ddd, J=12.4, 8.2, 6.0 Hz, 2H), 1.08 (t, J=6.3 Hz, 6H); MS (ESI) m/e 366.3 [(M+H)⁺, calcd for C₂₁H₂₈N₅O 366.2]. HPLC retention time (Method O): t_{R}=7.36 min; HPLC retention time (Method P): t_{R}=6.92 min.

### Example 27

### 2-(1-Amino-3-methylbutyl)-N,N-diethyl-6-(3-methoxypyridin-4-yl)quinazolin-4-amine

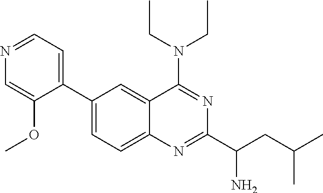 [SMILES: CCN(CC)c1nc(C(N)CC(C)C)nc2ccc(-c3ccncc3OC)cc12]

Prepared in a similar fashion as described in Example 23 using 2-(1-amino-3-methylbutyl)-6-(3-methoxypyridin-4-yl)quinazolin-4-ol and diethylamine in Part A. 2-(1-Amino-3-methylbutyl)-6-(3-methoxypyridin-4-yl)quinazolin-4-ol was prepared as described in Example 13, Parts A-C using 3-methoxypyridylboronic acid in Part C. 2-(1-Amino-3-methylbutyl)-N,N-diethyl-6-(3-methoxypyridin-4-yl)quinazolin-4-amine (20 mg) was isolated as a TFA salt: ¹H NMR (400 MHz, CD₃OD) δ 8.75 (s, 1H), 8.63-8.57 (m, 2H), 8.26 (dd, J=8.8, 2.0 Hz, 1H), 8.09 (d, J=5.5 Hz, 1H), 7.98 (d, J=8.8 Hz, 1H), 4.56 (dd, J=7.7, 6.9 Hz, 1H), 4.15 (s, 3H), 4.11-3.99 (m, 4H), 2.77-2.62 (m, 1H), 2.11-1.87 (m, 2H), 1.86-1.71 (m, 2H), 1.53 (t, J=6.0 Hz, 6H), 1.07 (d, J=6.0 Hz, 3H), 1.13-1.00 (m, 3H); MS (ESI) m/e 394.4 [(M+H)⁺, calcd for C₂₃H₃₂N₅O 394.3]. HPLC retention time (Method O): t_{R}=6.12 min; HPLC retention time (Method P): t_{R}=6.27 min.

### Example 28

### 2-(1-Amino-3-methylbutyl)-N-ethyl-6-(3-methoxypyridin-4-yl)quinazolin-4-amine

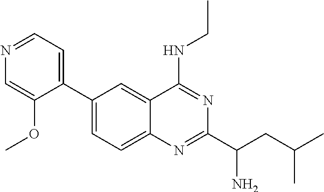 [SMILES: CCNc1nc(C(N)CC(C)C)nc2ccc(-c3ccncc3OC)cc12]

Prepared in a similar fashion as described in Example 23 using 2-(1-amino-3-methylbutyl)-6-(3-methoxypyridin-4-yl)quinazolin-4-ol and ethylamine in Part A. 2-(1-Amino-3-methylbutyl)-6-(3-methoxypyridin-4-yl)quinazolin-4-ol was prepared as described in Example 13, Parts A-C using 3-methoxypyridylboronic acid in Part C. 2-(1-Amino-3-methylbutyl)-N-ethyl-6-(3-methoxypyridin-4-yl)quinazolin-4-amine (13 mg) was isolated as a TFA salt: ¹H NMR (400 MHz, CD₃OD) δ 8.72-8.67 (m, 1H), 8.59 (d, J=1.5 Hz, 1H), 8.55 (br. s., 1H), 8.25-8.21 (m, 1H), 7.99-7.94 (m, 1H), 7.92-7.87 (m, 1H), 4.48 (dd, J=7.8, 6.5 Hz, 1H), 4.10 (s, 3H), 3.85 (q, J=7.1 Hz, 1H), 2.07-1.77 (m, 3H), 1.39 (t, J=7.3 Hz, 3H), 1.10-1.08 (m, 3H), 1.07-1.05 (m, 3H); MS (ESI) m/e 366.3 [(M+H)⁺, calcd for C₂₁H₂₈N₅O 366.2]. HPLC retention time (Method O): t_{R}=12.86 min; HPLC retention time (Method P): t_{R}=13.82 min.

### Example 29

### 2-Isopentyl-6-(3-methoxypyridin-4-yl)-N,N-dimethylquinazolin-4-amine

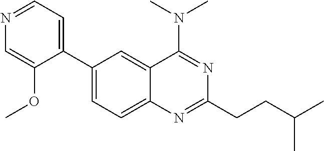 [SMILES: COc1cnccc1-c1ccc2nc(CCC(C)C)nc(N(C)C)c2c1]

Prepared in a similar fashion as described in Example 23 using 2-(1-amino-3-methylbutyl)-6-(3-methoxypyridin-4-yl)quinazolin-4-ol and dimethylamine in Part A. 2-(1-Amino-3-methylbutyl)-6-(3-methoxypyridin-4-yl)quinazolin-4-ol was prepared as described in Example 13, Parts A-C using 3-methoxypyridylboronic acid in Part C. 2-Isopentyl-6-(3-methoxypyridin-4-yl)-N,N-dimethylquinazolin-4-amine (10 mg) was isolated as a TFA salt: ¹H NMR (400 MHz, CDCl₃) δ 8.62 (s, 1H), 8.47 (d, J=4.8 Hz, 1H), 8.37-8.32 (m, 2H), 8.05 (d, J=8.5 Hz, 1H), 7.55 (d, J=5.0 Hz, 1H), 4.03 (s, 3H), 3.67 (br. s., 6H), 3.09-3.03 (m, 2H), 1.80-1.66 (m, 3H), 0.98 (d, J=6.5 Hz, 6H); MS (ESI) m/e 351.2 [(M+H)⁺, calcd for C₂₁H₂₇N₄O 351.2]. HPLC retention time (Method O): t_{R}=7.74 min; HPLC retention time (Method P): t_{R}=7.79 min.

### Example 30

### 2-(1-Amino-3-methylbutyl)-6-(oxazol-5-yl)quinazoline-4-carbonitrile

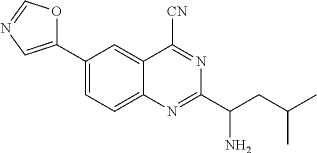 [SMILES: CC(C)CC(N)c1nc(C#N)c2cc(-c3cnco3)ccc2n1]

### Part A. tert-Butyl 3-methyl-1-(6-(oxazol-5-yl)-4-oxo-3,4-dihydroquinazolin-2-yl)butylcarbamate

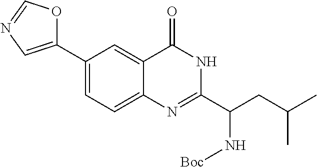 [SMILES: CC(C)CC(NC(=O)OC(C)(C)C)c1nc2ccc(-c3cnco3)cc2c(=O)[nH]1]

2-(1-Amino-3-methylbutyl)-6-(oxazol-5-yl)quinazolin-4(3H)-one (67 mg, 0.225 mmol), prepared as described in Example 12, was suspended in CH₂Cl₂ (3 mL) and was treated with triethylamine (0.20 mL, 1.44 mmol). The suspension became a solution. The mixture was cooled to 0° C. and was treated with di-tert-butyldicarbonate (0.078 mL, 0.337 mmol). The cooling bath was removed and the mixture was stirred at room temperature for 24 h. The mixture was concentrated and the residue was purified by column chromatography on silica gel (2%-5% methanol in CH₂Cl₂) to afford tert-butyl 3-methyl-1-(6-(oxazol-5-yl)-4-oxo-3,4-dihydroquinazolin-2-yl)butylcarbamate (57 mg, 64% yield) as an off-white foam: ¹H NMR (400 MHz, CDCl₃) δ 11.01 (br. s., 1H), 8.54 (d, J=1.8 Hz, 1H), 8.05 (dd, J=8.5, 2.0 Hz, 1H), 8.00 (s, 1H), 7.81 (d, J=8.5 Hz, 1H), 7.51 (s, 1H), 5.32 (br. s., 1H), 4.75 (br. s., 1H), 1.95-1.77 (m, 3H), 1.46 (s, 9H), 1.07 (d, J=6.3 Hz, 3H), 1.04 (d, J=6.3 Hz, 3H); MS (ESI) m/e 399.1 [(M+H)⁺, calcd for C₂₁H₂₇N₄O₄ 399.2].

### Part B. tert-Butyl 1-(4-cyano-6-(oxazol-5-yl)quinazolin-2-yl)-3-methylbutylcarbamate

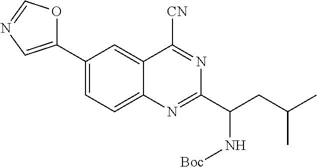 [SMILES: CC(C)CC(NC(=O)OC(C)(C)C)c1nc(C#N)c2cc(-c3cnco3)ccc2n1]

To a solution of tert-butyl 3-methyl-1-(6-(oxazol-5-yl)-4-oxo-3,4-dihydroquinazolin-2-yl)butylcarbamate (57 mg, 0.143 mmol) and BOP (82 mg, 0.186 mmol) in acetonitrile (1.2 mL) was added DBU (0.032 mL, 0.215 mmol). The mixture was stirred for 25 min at room temperature. Sodium cyanide (16.8 mg, 0.343 mmol) was added to the reaction mixture. After stirring the reaction mixture for an additional 5 min, 18-crown-6 (91 mg, 0.343 mmol) was added. The solution changed from pale yellow to dark red over 15 min. Stirring was continued for 36 h. The reaction mixture was transferred to a separatory funnel containing saturated aqueous NaHCO₃ solution (5 mL). The aqueous layer was extracted with CH₂Cl₂ (3×10 mL). The combined organic layers were washed with brine (5 mL), dried over MgSO₄, filtered, and concentrated. The residue was purified by column chromatography on silica gel (1%-4% methanol in CH₂Cl₂) to afford tert-butyl 1-(4-cyano-6-(oxazol-5-yl)quinazolin-2-yl)-3-methylbutylcarbamate (12.7 mg, 22% yield) as a yellow oil: 10 ¹H NMR (400 MHz, CDCl₃) δ 8.47 (d, J=1.3 Hz, 1H), 8.29 (dd, J=8.9, 1.4 Hz, 1H), 8.21-8.17 (m, 1H), 8.10 (s, 1H), 7.68 (s, 1H), 5.45 (d, J=8.5 Hz, 1H), 5.23-5.14 (m, 1H), 1.81-1.69 (m, 3H), 1.46 (s, 9H), 1.05 (d, J=6.3 Hz, 3H), 1.01 (d, J=6.3 Hz, 3H); MS (ESI) m/e 308.1 [(M-C₅H₈O₂+H)⁺, calcd for C₁₇H₁₈N₅O 308.2].

### Part C. 2-(1-Amino-3-methylbutyl)-6-(oxazol-5-yl)quinazoline-4-carbonitrile

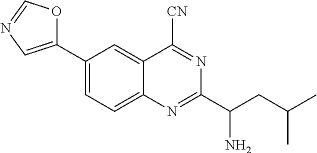 [SMILES: CC(C)CC(N)c1nc(C#N)c2cc(-c3cnco3)ccc2n1]

To a solution of tert-butyl 1-(4-cyano-6-(oxazol-5-yl)quinazolin-2-yl)-3-methylbutylcarbamate (12 mg, 0.029 mmol) in CH₂Cl₂ (1 mL) at 0° C. was added TFA (0.2 mL, 2.60 mmol). The cooling bath was removed and the reaction mixture was stirred at room temperature for 1 h. The reaction mixture was concentrated and the product was purified by reverse phase HPLC (5% MeCN:95% water:0.1% TFA→95% MeCN:5% water:0.1% TFA). The organic solvent was removed on the rotovapor and the aqueous mixture was frozen and placed on the lyophilizer to afford 2-(1-amino-3-methylbutyl)-6-(oxazol-5-yl)quinazoline-4-carbonitrile (6.8 mg, 75% yield) as a TFA salt: ¹H NMR (400 MHz, CDCl₃) δ 8.47 (d, J=1.3 Hz, 1H), 8.29 (dd, J=8.9, 1.4 Hz, 1H), 8.21-8.17 (m, 1H), 8.10 (s, 1H), 7.68 (s, 1H), 5.45 (d, J=8.5 Hz, 1H), 5.23-5.14 (m, 1H), 1.81-1.69 (m, 3H), 1.46 (s, 9H), 1.05 (d, J=6.3 Hz, 3H), 1.01 (d, J=6.3 Hz, 3H); MS (ESI) m/e 308.1 [(M-C₅H₈O₂+H)⁺, calcd for C₁₇H₁₈N₅O 308.2]. HPLC retention time (Method O): t_{R}=8.61 min; HPLC retention time (Method P): t_{R}=8.64 min.

### Example 31

### 2-Isopentyl-6-(oxazol-5-yl)quinazoline-4-carbonitrile

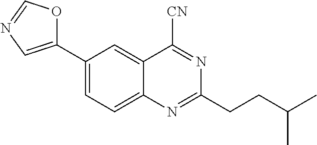 [SMILES: CC(C)CCc1nc(C#N)c2cc(-c3cnco3)ccc2n1]

To a solution of 2-isopentyl-6-(oxazol-5-yl)quinazolin-4(3H)-one (25 mg, 0.063 mmol), prepared in a similar fashion as described in Example 12, Parts A-E using 4-methylpentanoic acid in Part A, and BOP (36.2 mg, 0.082 mmol) in acetonitrile (2 mL) at 0° C. was added DBU (0.028 mL, 0.189 mmol). The mixture was stirred for 25 min at 0° C. Sodium cyanide (7.40 mg, 0.151 mmol) was added to the reaction mixture. After the reaction mixture was stirred for an additional 5 min, 18-crown-6 (39.9 mg, 0.151 mmol) was added. The solution changed from pale yellow to dark red over 15 min. Stirring was continued for 2 h. The reaction mixture was transferred to a separatory funnel containing saturated aqueous NaHCO₃ solution (5 mL). The aqueous layer was extracted with CH₂Cl₂ (3×5 mL). The combined organic layers were washed with brine (5 mL), dried over MgSO₄, filtered, and concentrated. The product was purified by reverse phase HPLC (5% MeCN:95% water:0.1% TFA→95% MeCN:5% water:0.1% TFA). The organic solvent was removed on the rotovapor and the aqueous mixture was frozen and placed on the lyophilizer to afford 2-isopentyl-6-(oxazol-5-yl)quinazoline-4-carbonitrile (3 mg, 12% yield) as a yellow solid: ¹H NMR (400 MHz, CDCl₃) δ 8.46 (d, J=1.8 Hz, 1H), 8.26 (dd, J=8.9, 1.9 Hz, 1H), 8.17-8.12 (m, 1H), 8.08 (s, 1H), 7.65 (s, 1H), 3.68-3.62 (m, 1H), 3.21-3.16 (m, 2H), 1.01 (d, J=6.8 Hz, 6H), 0.94-0.81 (m, 2H); MS (ESI) m/e 293.1 [(M+H)⁺, calcd for C₁₇H₁₇N₄O 293.1]. HPLC retention time (Method O): t_{R}=16.73 min; HPLC retention time (Method P): t_{R}=13.83 min.

### Example 32

### 2-(1-Amino-3-methylbutyl)-7-methoxy-6-(oxazol-5-yl)quinazoline-4-carbonitrile

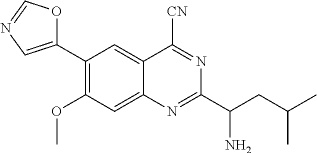 [SMILES: COc1cc2nc(C(N)CC(C)C)nc(C#N)c2cc1-c1cnco1]

### Part A. 2-Amino-5-bromo-4-methoxybenzoic

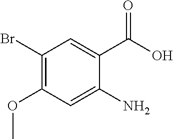

To a solution of 2-amino-4-methoxybenzoic acid (4.00 g, 23.93 mmol) in DMF (120 mL) at 0° C. was added NBS (4.68 g, 26.3 mmol). The cooling bath was removed and the reaction mixture was warmed up to room temperature and was stirred at room temperature for 1 h. The mixture was cooled to 0° C. and was treated with saturated sodium sulfite solution (25 mL) and was stirred for 5 min. The pH of the mixture was adjusted to pH=3 using conc. HCl (ca. 10-15 mL was added). The reaction mixture was transferred to a separatory funnel containing water (100 mL). The aqueous layer was extracted with ether (1×250 mL then 4×100 mL). The combined organic layers were washed with water (3×50 mL), brine (50 mL), dried over MgSO₄, filtered, and concentrated to afford 2-amino-5-bromo-4-methoxybenzoic acid (5.90 g, 100% yield) as a solid: ¹H NMR (400 MHz, DMSO-d₆) δ 7.78 (s, 1H), 6.43 (s, 1H), 3.81 (s, 3H), 2.90 (s, 1H), 2.74 (s, 1H); MS (ESI) m/e 228.0 [(M+H—H₂O)⁺, calcd for C₈H₇BrNO₂ 228.0].

### Part B. 2-Amino-5-bromo-4-methoxybenzamide

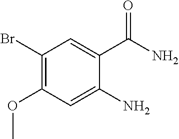

EDC (5.42 g, 28.3 mmol) was added to a solution of 2-amino-5-bromo-4-methoxybenzoic acid (5.80 g, 23.57 mmol) in CH₂Cl₂ (120 mL). HOBT hydrate (4.33 g, 28.3 mmol) was added and the reaction mixture was stirred for 1 h at room temperature. The reaction mixture was quenched with 30% ammonium hydroxide solution (58 mL) and stirring was continued for an additional 1 h. The reaction mixture was transferred to a separatory funnel containing saturated aqueous NaHCO₃ solution (150 mL). The aqueous layer was extracted with CH₂Cl₂ containing 5% MeOH (3×150 mL). The combined organic layers were washed with brine (150 mL), dried over MgSO₄, filtered, and concentrated to give 2-amino-5-bromo-4-methoxybenzamide (5.04 g, 87% yield) as a colorless solid: ¹H NMR (400 MHz, DMSO-d₆) δ 7.79 (s, 1H), 6.92 (s, 2H), 6.38 (s, 1H), 3.78 (s, 3H).

### Part C. Benzyl 1-(4-bromo-2-carbamoyl-5-methoxyphenylamino)-4-methyl-1-oxopentan-2-ylcarbamate

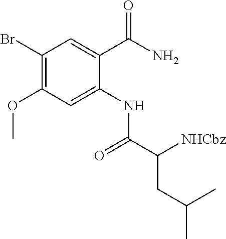 [SMILES: COc1cc(NC(=O)C(CC(C)C)NC(=O)OCc2ccccc2)c(C(N)=O)cc1Br]

To a suspension of N-(chloromethylene)-N-methylmethanaminium, chloride salt (627 mg, 4.90 mmol) (Jarrahpour et al. Tetrahedron 2009, 65, 2927-2934) in CH₂Cl₂ (8 mL) at 0° C. was added 2-(benzyloxycarbonylamino)-4-methylpentanoic acid (1.04 g, 3.92 mmol) in CH₂Cl₂ (4 mL) via cannula. The mixture was stirred at that temperature for 15 min. A premixed solution of 2-amino-5-bromo-4-methoxybenzamide (800 mg, 3.26 mmol) and Et₃N (1.14 mL, 8.16 mmol) in CH₂Cl₂/DMF (4 mL, 1:1) was then added via cannula. The reaction mixture was stirred at 0° C. for 3 h. The reaction mixture was transferred to a separatory funnel containing CH₂Cl₂ (200 mL). The organic layer was washed with 1 N HCl (2×50 mL), saturated NaHCO₃ (50 mL), brine (50 mL), dried over MgSO₄, filtered, and concentrated. The product was tritrated with ether/hexanes and the solid was collected in a Buchner funnel to give benzyl 1-(4-bromo-2-carbamoyl-5-methoxyphenylamino)-4-methyl-1-oxopentan-2-ylcarbamate (997 mg, 62% yield) as a solid. The product was used directly in the next step without further purification. ¹H NMR (400 MHz, DMSO-d₆) δ 12.72 (s, 1H), 8.48 (s, 1H), 8.27 (br. s., 1H), 8.14 (s, 1H), 7.94 (d, J=7.0 Hz, 1H), 7.67 (br. s., 1H), 7.45-7.35 (m, 4H), 7.35-7.31 (m, 1H), 5.08 (ABq, J_{AB}=12.5, Δυ=23.5 Hz, 2H), 4.03 (q, J=7.3 Hz, 1H), 3.89 (s, 3H), 1.73-1.65 (m, 1H), 1.58-1.64 (m, 2H), 0.91 (d, J=6.3 Hz, 3H), 0.88 (d, J=6.3 Hz, 3H).

### Part D. Benzyl (1-(6-bromo-7-methoxy-4-oxo-3,4-dihydroquinazolin-2-yl)-3-methylbutyl)carbamate

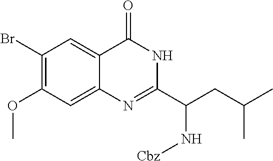 [SMILES: COc1cc2nc(C(CC(C)C)NC(=O)OCc3ccccc3)[nH]c(=O)c2cc1Br]

A mixture of benzyl 1-(4-bromo-2-carbamoyl-5-methoxyphenylamino)-4-methyl-1-oxopentan-2-ylcarbamate (1.47 g, 2.99 mmol) and sodium carbonate (0.949 g, 8.96 mmol) in ethanol (7 mL) and water (7 mL) was heated at reflux for 2 h. The reaction mixture was diluted with water and was acidified with citric acid (1.65 g) to ca. pH=6. The reaction mixture was transferred to a separatory funnel containing water (20 mL). The aqueous layer was extracted with CH₂Cl₂ (3×50 mL). The combined organic layers were washed with brine (50 mL). Some undissolved material was suspended in the organic layer. The mixture was diluted with CH₂Cl₂ and methanol and warmed to try to dissolve the suspended solids. Some of the solids dissolved, but some cloudiness remained. The mixture was dried over MgSO₄, filtered, and concentrated. The product, benzyl 1-(6-bromo-7-methoxy-4-oxo-3,4-dihydroquinazolin-2-yl)-3-methylbutylcarbamate (1.26 g, 89% yield), was isolated as a solid and was not purified further. ¹H NMR (400 MHz, DMSO-d₆) δ 12.33 (br. s., 1H), 8.19 (s, 1H), 7.67 (d, J=7.8 Hz, 1H), 7.42-7.25 (m, 5H), 7.18 (s, 1H), 5.04 (s, 2H), 4.58-4.49 (m, 1H), 4.00 (s, 3H), 1.74-1.62 (m, 2H), 1.61-1.49 (m, 1H), 0.91 (t, J=5.8 Hz, 6H); MS (ESI) m/e 474.1 [(M+H)⁺, calcd for C₂₂H₂₅BrN₃O₄ 474.1].

### Part E. Benzyl 1-(7-methoxy-4-oxo-6-vinyl-3,4-dihydroquinazolin-2-yl)-3-methylbutylcarbamate

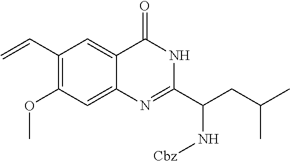 [SMILES: C=Cc1cc2c(=O)[nH]c(C(CC(C)C)NC(=O)OCc3ccccc3)nc2cc1OC]

To a solution of benzyl 1-(6-bromo-7-methoxy-4-oxo-3,4-dihydroquinazolin-2-yl)-3-methylbutylcarbamate (410 mg, 0.864 mmol) in toluene (8 mL) and ethanol (2 mL) was added 2,4,6-trivinyl-1,3,5,2,4,6-trioxatriborinane pyridine complex (140 mg, 0.864 mmol) and sodium carbonate (2 N, aq) (1.04 mL, 1.04 mmol). The solution was degassed by sonication for 5 min. Pd(PPh₃)₄ (100 mg, 0.086 mmol) was added to the reaction mixture and the mixture was heated at 95° C. for 2.5 h. The reaction mixture was cooled to room temperature and transferred to a separatory funnel containing saturated aqueous NaHCO₃ solution (25 mL). The aqueous layer was extracted with 5% methanol in dichloromethane (3×25 mL). The combined organic layers were washed with brine (25 mL), dried over MgSO₄, filtered, and concentrated. The residue was tritrated with ether and the solid was collected on a Buchner funnel to give benzyl 1-(7-methoxy-4-oxo-6-vinyl-3,4-dihydroquinazolin-2-yl)-3-methylbutylcarbamate (306 mg, 84% yield) as a pale yellow solid: ¹H NMR (400 MHz, DMSO-d₆) δ 12.18 (br. s., 1H), 8.15 (s, 1H), 7.65 (d, J=8.0 Hz, 1H), 7.39-7.30 (m, 5H), 7.08 (s, 1H), 7.00 (dd, J=17.8, 11.3 Hz, 1H), 5.93 (d, J=17.6 Hz, 1H), 5.41-5.36 (m, 1H), 5.05 (s, 2H), 4.59-4.51 (m, 1H), 3.96 (s, 3H), 1.74-1.65 (m, 2H), 1.62-1.52 (m, 1H), 0.92 (t, J=5.6 Hz, 6H); MS (ESI) m/e 422.1 [(M+H)⁺, calcd for C₂₄H₂₈N₃O₄ 422.2].

### Part F. Benzyl 1-(6-formyl-7-methoxy-4-oxo-3,4-dihydroquinazolin-2-yl)-3-methylbutylcarbamate

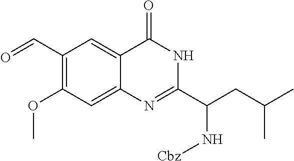 [SMILES: COc1cc2nc(C(CC(C)C)NC(=O)OCc3ccccc3)[nH]c(=O)c2cc1C=O]

To a solution of benzyl 1-(7-methoxy-4-oxo-6-vinyl-3,4-dihydroquinazolin-2-yl)-3-methylbutylcarbamate (402 mg, 0.954 mmol) in dioxane (10 mL) and water (2.5 mL) at 0° C. was added 2,6-lutidine (0.22 mL, 1.91 mmol), osmium tetroxide (2.5% in 2-methyl-2-propanol) (0.24 mL, 0.02 mmol), and sodium periodate (816 mg, 3.82 mmol). The cooling bath was removed and the reaction mixture was allowed to warm up to room temperature while stirring for 2.5 h. The reaction mixture was transferred to a separatory funnel containing a 1:1 mixture of water and saturated aqueous NaHCO₃ solution (20 mL). The aqueous layer was extracted with 5% methanol in dichloromethane (3×20 mL). The combined organic layers were washed with brine (20 mL), dried over MgSO₄, filtered and concentrated. The residue was purified by column chromatography on silica gel (1%-4% methanol in CH₂Cl₂) to afford benzyl 1-(6-formyl-7-methoxy-4-oxo-3,4-dihydroquinazolin-2-yl)-3-methylbutylcarbamate (231 mg, 57% yield) as an off-white solid: ¹H NMR (400 MHz, CDCl₃) δ 12.00 (br. s., 1H), 10.45 (s, 1H), 8.72 (s, 1H), 7.28 (s, 5H), 7.19 (s, 1H), 5.88-5.80 (m, 1H), 5.16-5.07 (m, 2H), 4.90-4.82 (m, 1H), 4.07 (s, 3H), 1.88-1.77 (m, 3H), 1.10-1.01 (m, 6H); MS (ESI) m/e 424.1 [(M+H)⁺, calcd for C₂₃H₂₆N₃O₅ 424.2].

### Part G. Benzyl 1-(7-methoxy-6-(oxazol-5-yl)-4-oxo-3,4-dihydroquinazolin-2-yl)-3-methylbutylcarbamate

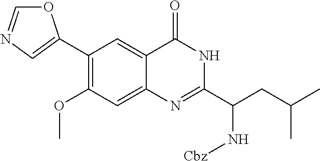 [SMILES: COc1cc2nc(C(CC(C)C)NC(=O)OCc3ccccc3)[nH]c(=O)c2cc1-c1cnco1]

To a solution of benzyl 1-(6-formyl-7-methoxy-4-oxo-3,4-dihydroquinazolin-2-yl)-3-methylbutylcarbamate (230 mg, 0.543 mmol) and TosMIC (323 mg, 1.657 mmol) in methanol (5 mL) was added potassium carbonate (233 mg, 1.684 mmol). The reaction mixture was heated at reflux for 4 h. The reaction mixture was cooled to room temperature and was transferred to a separatory funnel containing saturated aqueous NaHCO₃ solution (20 mL). The aqueous layer was extracted with CH₂Cl₂ (3×20 mL). The combined organic layers were washed with brine (20 mL), dried over MgSO₄, filtered, and concentrated. The residue was purified by column chromatography on silica gel (30%-65% ethyl acetate in hexanes) to afford benzyl 1-(7-methoxy-6-(oxazol-5-yl)-4-oxo-3,4-dihydroquinazolin-2-yl)-3-methylbutylcarbamate (65 mg, 26% yield) as an off-white solid: ¹H NMR (400 MHz, CDCl₃) δ 8.63 (s, 1H), 7.95 (s, 1H), 7.65 (s, 1H), 7.35-7.25 (m, 6H), 5.14 (ABq, J_{AB}=12.0, Δυ=27.2 Hz, 2H), 4.94-4.85 (m, 1H), 4.12 (s, 3H), 1.92-1.81 (m, 3H), 1.08 (d, J=5.5 Hz, 3H), 1.04 (d, J=6.0 Hz, 3H); MS (ESI) m/e 463.1 [(M+H)⁺, calcd for C₂₅H₂₇N₄O₅ 463.2].

### Part H. Benzyl 1-(4-cyano-7-methoxy-6-(oxazol-5-yl)quinazolin-2-yl)-3-methylbutylcarbamate

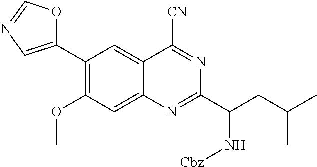 [SMILES: COc1cc2nc(C(CC(C)C)NC(=O)OCc3ccccc3)nc(C#N)c2cc1-c1cnco1]

To a solution of benzyl 1-(7-methoxy-6-(oxazol-5-yl)-4-oxo-3,4-dihydroquinazolin-2-yl)-3-methylbutylcarbamate (56 mg, 0.121 mmol) and BOP (80 mg, 0.182 mmol) in acetonitrile (1 mL) was added DBU (0.031 mL, 0.206 mmol). The mixture was stirred for 25 min at room temperature. Sodium cyanide (13.05 mg, 0.266 mmol) was added to the reaction mixture. After stirring the reaction mixture for an additional 5 min, 18-crown-6 (70.4 mg, 0.266 mmol) was added. The solution changed from pale yellow to dark red over 15 min. Stirring was continued for 3 h.

The reaction mixture was transferred to a separatory funnel containing saturated aqueous NaHCO₃ solution (20 mL). The aqueous layer was extracted with CH₂Cl₂ (3×25 mL). The combined organic layers were washed with brine (20 mL), dried over MgSO₄, filtered, and concentrated. The residue was purified by column chromatography on silica gel (40%-70% ethyl acetate in hexanes) to afford benzyl 1-(4-cyano-7-methoxy-6-(oxazol-5-yl)quinazolin-2-yl)-3-methylbutylcarbamate (36 mg, 63% yield) as an off-white foam. The product was purified further by reverse phase HPLC (5% MeCN:95% water:0.1% TFA→95% MeCN:5% water:0.1% TFA). ¹H NMR (400 MHz, CDCl₃) δ 8.57 (s, 1H), 8.10 (s, 1H), 7.82 (s, 1H), 7.50 (s, 1H), 7.42-7.32 (m, 5H), 5.77 (d, J=8.5 Hz, 1H), 5.27-5.18 (m, 1H), 5.14 (s, 2H), 4.21 (s, 3H), 1.81-1.70 (m, 3H), 1.05 (d, J=6.0 Hz, 3H), 1.00 (d, J=6.0 Hz, 3H); MS (ESI) m/e 472.1 [(M+H)⁺, calcd for C₂₆H₂₆N₅O₄ 472.2].

### Part I. 2-(1-Amino-3-methylbutyl)-7-methoxy-6-(oxazol-5-yl)quinazoline-4-carbonitrile

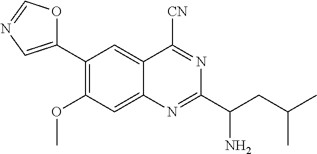 [SMILES: COc1cc2nc(C(N)CC(C)C)nc(C#N)c2cc1-c1cnco1]

To a solution of benzyl 1-(4-cyano-7-methoxy-6-(oxazol-5-yl)quinazolin-2-yl)-3-methylbutylcarbamate (16 mg, 0.034 mmol) in dichloromethane (1.0 mL) at room temperature under nitrogen was added methanesulfonic acid (0.077 mL, 1.19 mmol) and anisole (8.15 μl, 75 μmol). The reaction mixture was stirred for 16 h at room temperature under nitrogen. The reaction mixture was transferred to a separatory funnel containing saturated aqueous NaHCO₃ solution (2 mL). The aqueous layer was extracted with methylene chloride (3×5 mL). The combined organic layers were washed with brine (2 mL), dried over MgSO₄, filtered, and concentrated. The product was purified by reverse phase HPLC (5% MeCN:95% water:0.1% TFA→95% MeCN:5% water:0.1% TFA). The organic solvent was removed on the rotovapor and the aqueous mixture was frozen and placed on the lyophilizer to afford 2-(1-amino-3-methylbutyl)-7-methoxy-6-(oxazol-5-yl)quinazoline-4-carbonitrile (9.7 mg, 83% yield) as a colorless amorphous solid: ¹H NMR (500 MHz, DMSO-d₆) δ 8.71 (s, 1H), 8.60 (br. s., 3H), 8.45 (s, 1H), 7.99 (s, 1H), 7.68 (s, 1H), 4.64 (t, J=6.4 Hz, 1H), 4.26 (s, 3H), 1.94-1.80 (m, 2H), 1.70 (dt, J=13.2, 6.7 Hz, 1H), 0.98 (d, J=6.4 Hz, 3H), 0.94 (d, J=6.4 Hz, 3H); MS (ESI) m/e 338.1 [(M+H)⁺, calcd for C₁₈H₂₀N₅O₂ 338.2]. HPLC retention time (Method O): t_{R}=9.01 min; HPLC retention time (Method P): t_{R}=9.11 min.

### Example 33

### 2-(1-Amino-3-methylbutyl)-6-(pyridin-4-yl)quinazoline-4-carbonitrile

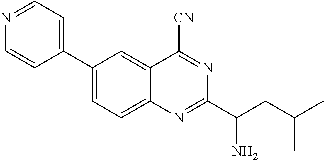 [SMILES: CC(C)CC(N)c1nc(C#N)c2cc(-c3ccncc3)ccc2n1]

### Part A. tert-Butyl 3-methyl-1-(4-oxo-6-(pyridin-4-yl)-3,4-dihydroquinazolin-2-yl)butylcarbamate

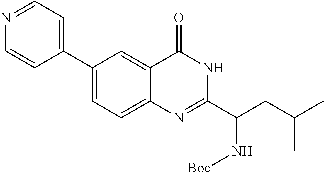 [SMILES: CC(C)CC(NC(=O)OC(C)(C)C)c1nc2ccc(-c3ccncc3)cc2c(=O)[nH]1]

2-(1-Amino-3-methylbutyl)-6-(pyridin-4-yl)quinazolin-4(3H)-one (75 mg, 0.243 mmol), prepared as described in Example 13, was suspended in CH₂Cl₂ (3 mL) and was treated with triethylamine (0.20 mL, 1.44 mmol). The suspension became a solution. The mixture was cooled to 0° C. and was treated with di-tert-butyldicarbonate (0.085 mL, 0.365 mmol). The cooling bath was removed and the mixture was stirred at room temperature for 24 h. The mixture was concentrated and the residue was purified by column chromatography on silica gel (2%-5% methanol in CH₂Cl₂) to afford tert-butyl 3-methyl-1-(4-oxo-6-(pyridin-4-yl)-3,4-dihydroquinazolin-2-yl)butylcarbamate (50 mg, 50% yield) as a colorless oil: MS (ESI) m/e 409.3 [(M+H)⁺, calcd for C₂₃H₂₉N₄O₃ 409.2].

### Part B. tert-Butyl 1-(4-cyano-6-(pyridin-4-yl)quinazolin-2-yl)-3-methylbutylcarbamate

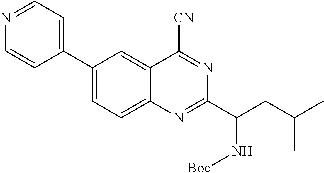 [SMILES: CC(C)CC(NC(=O)OC(C)(C)C)c1nc(C#N)c2cc(-c3ccncc3)ccc2n1]

To a solution of tert-butyl 3-methyl-1-(4-oxo-6-(pyridin-4-yl)-3,4-dihydroquinazolin-2-yl)butylcarbamate (50 mg, 0.122 mmol) and BOP (70.4 mg, 0.159 mmol) in acetonitrile (2 mL) was added DBU (0.028 mL, 0.184 mmol). The mixture was stirred for 25 min at room temperature. Sodium cyanide (14.40 mg, 0.294 mmol) was added to the reaction mixture. After stirring the reaction mixture for an additional 5 min, 18-crown-6 (78 mg, 0.294 mmol) was added. The solution changed from pale yellow to dark red over 15 min. Stirring was continued for 36 h.

The reaction mixture was transferred to a separatory funnel containing saturated aqueous NaHCO₃ solution (5 mL). The aqueous layer was extracted with CH₂Cl₂ (3×10 mL). The combined organic layers were washed with brine (5 mL), dried over MgSO₄, filtered, and concentrated. The residue was purified by reverse phase HPLC (5% MeCN:95% water:0.1% TFA→95% MeCN:5% water:0.1% TFA) to afford tert-butyl 1-(4-cyano-6-(pyridin-4-yl)quinazolin-2-yl)-3-methylbutylcarbamate (5 mg, 10% yield) as a red oil: ¹H NMR (400 MHz, CD₃OD) δ 8.96-8.93 (m, 2H), 8.72 (d, J=1.5 Hz, 1H), 8.62 (dd, J=9.0, 2.0 Hz, 1H), 8.42 (d, J=6.8 Hz, 2H), 8.37 (d, J=9.0 Hz, 1H), 4.32 (d, J=1.0 Hz, 1H), 1.85-1.75 (m, 3H), 1.46 (s, 9H), 1.08-1.02 (m, 6H); MS (ESI) m/e 418.2 [(M+H)⁺, calcd for C₂₄H₂₈N₅O₂ 418.2].

### Part C. 2-(1-Amino-3-methylbutyl)-6-(pyridin-4-yl)quinazoline-4-carbonitrile

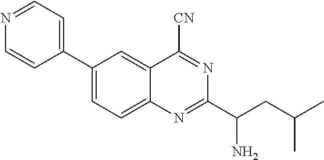 [SMILES: CC(C)CC(N)c1nc(C#N)c2cc(-c3ccncc3)ccc2n1]

To a solution of tert-butyl 1-(4-cyano-6-(pyridin-4-yl)quinazolin-2-yl)-3-methylbutylcarbamate (4 mg, 0.010 mmol) in CH₂Cl₂ (0.5 mL) in an icebath was added TFA (7.38 μl, 0.096 mmol). The mixture was stirred for 2 hours. The solvent was evaporated and the residue was purified by reverse phase HPLC (5% MeCN:95% water:0.1% TFA→95% MeCN:5% water:0.1% TFA) to afford 2-(1-amino-3-methylbutyl)-6-(pyridin-4-yl)quinazoline-4-carbonitrile (0.6 mg, 14% yield) as a red oil: ¹H NMR (400 MHz, CD₃OD) δ 8.89 (br. s., 1H), 8.72 (d, J=1.3 Hz, 1H), 8.68 (dd, J=8.8, 2.0 Hz, 1H), 8.45-8.40 (m, 1H), 8.33-8.25 (m, 1H), 8.23-8.09 (m, 2H), 3.65 (s, 1H), 2.17-2.04 (m, 1H), 1.99-1.88 (m, 1H), 1.81 (dt, J=13.7, 6.8 Hz, 1H), 1.14-1.09 (m, 3H), 1.09-1.05 (m, 3H); MS (ESI) m/e 318.3 [(M+H)⁺, calcd for C₁₉H₂₀N₅ 318.2]. HPLC retention time (Method O): t_{R}=6.62 min; HPLC retention time (Method P): t_{R}=6.63 min.

### Example 34

### 2-(1-Amino-3-methylbutyl)-6-(3-methoxypyridin-4-yl)quinazoline-4-carbonitrile

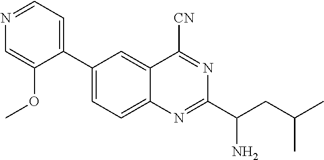 [SMILES: COc1cnccc1-c1ccc2nc(C(N)CC(C)C)nc(C#N)c2c1]

### Part A. Benzyl 1-(4-ethyl-6-(3-methoxypyridin-4-yl)quinazolin-2-yl)-3-methylbutylcarbamate

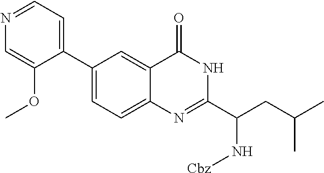 [SMILES: COc1cnccc1-c1ccc2nc(C(CC(C)C)NC(=O)OCc3ccccc3)[nH]c(=O)c2c1]

To a mixture of benzyl 1-(6-bromo-4-oxo-3,4-dihydroquinazolin-2-yl)-3-methylbutylcarbamate (500 mg, 1.125 mmol), prepared as described in Example 13, Parts A-B, potassium phosphate (597 mg, 2.81 mmol), Pd(OAc)₂ (25.3 mg, 0.113 mmol), and 2-dicyclohexylphosphino-2′,6′-dimethoxy-1,1′-biphenyl (SPhos) (92 mg, 0.225 mmol) under nitrogen in a vial was added 3-methoxypyridin-4-ylboronic acid (344 mg, 2.251 mmol). The mixture was heated at 100° C. for 2.5 h. The mixture was cooled to room temperature and was filtered through a pad of Celite with methanol rinsing and the filtrate was concentrated. The residue was purified by column chromatography on silica gel (1%-4% methanol in CH₂Cl₂) to afford benzyl 1-(6-(3-methoxypyridin-4-yl)-4-oxo-3,4-dihydroquinazolin-2-yl)-3-methylbutylcarbamate (200 mg, 38% yield) as a pale yellow solid: MS (ESI) m/e 473.4 [(M+H)⁺, calcd for C₂₇H₂₉N₄O₄ 473.2].

### Part B. Benzyl 1-(4-cyano-6-(3-methoxypyridin-4-yl)quinazolin-2-yl)-3-methylbutylcarbamate

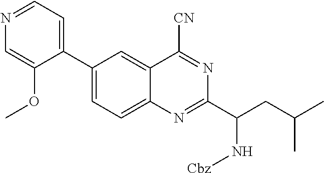 [SMILES: COc1cnccc1-c1ccc2nc(C(CC(C)C)NC(=O)OCc3ccccc3)nc(C#N)c2c1]

To a solution of benzyl 1-(6-(3-methoxypyridin-4-yl)-4-oxo-3,4-dihydroquinazolin-2-yl)-3-methylbutylcarbamate (100 mg, 0.212 mmol) in acetonitrile (2 mL) was added BOP (122 mg, 0.275 mmol). The mixture was stirred for 25 min at room temperature. Sodium cyanide (24.89 mg, 0.508 mmol) was added to the reaction mixture. After stirring the reaction mixture for an additional 5 min, 18-crown-6 (134 mg, 0.508 mmol) was added. The solution changed from pale yellow to dark red over 15 min. Stirring was continued for 36 h. The reaction mixture was transferred to a separatory funnel containing saturated aqueous NaHCO₃ solution (5 mL). The aqueous layer was extracted with CH₂Cl₂ (3×10 mL). The combined organic layers were washed with brine (5 mL), dried over MgSO₄, filtered, and concentrated. The residue was purified by column chromatography on silica gel (1%-4% methanol in CH₂Cl₂) to afford benzyl 1-(4-cyano-6-(3-methoxypyridin-4-yl)quinazolin-2-yl)-3-methylbutylcarbamate (45 mg, 44% yield): MS (ESI) m/e 482.2 [(M+H)⁺, calcd for C₂₈H₂₈N₅O₃ 482.2].

### Part C. 2-(1-Amino-3-methylbutyl)-6-(3-methoxypyridin-4-yl)quinazoline-4-carbonitrile

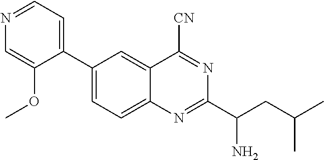 [SMILES: COc1cnccc1-c1ccc2nc(C(N)CC(C)C)nc(C#N)c2c1]

To a solution of benzyl 1-(4-cyano-6-(3-methoxypyridin-4-yl)quinazolin-2-yl)-3-methylbutylcarbamate (40 mg, 0.083 mmol) in dichloromethane (5 mL) at room temperature under nitrogen was added anisole (0.02 mL, 0.183 mmol) and methanesulfonic acid (0.108 mL, 1.661 mmol). The reaction mixture was stirred overnight at room temperature under nitrogen. The reaction was quenched with saturated aqueous Na₂CO₃. The mixture was transferred to a separatory funnel and was extracted with ethyl acetate (3×20 mL). The combined organic layers were concentrated. The residue was purified by reverse phase HPLC (5% MeCN:95% water:0.1% TFA→95% MeCN:5% water:0.1% TFA) to afford 2-(1-amino-3-methylbutyl)-6-(3-methoxypyridin-4-yl)quinazoline-4-carbonitrile (13 mg, 27% yield) as TFA salt: ¹H NMR (400 MHz, CDCl₃) δ 8.57 (s, 1H), 8.51-8.46 (m, 2H), 8.44-8.40 (m, 1H), 8.35-8.31 (m, 1H), 7.53 (d, J=4.8 Hz, 1H), 4.79 (dd, J=10.7, 4.6 Hz, 1H), 4.04 (s, 3H), 2.67 (s, 1H), 2.26-2.10 (m, 1H), 1.90-1.82 (m, 1H), 1.03 (d, J=6.5 Hz, 3H), 0.92 (d, J=6.5 Hz, 3H); MS (ESI) m/e 348.3 [(M+H)⁺, calcd for C₂₀H₂₂N₅O 348.2]. HPLC retention time (Method O): t_{R}=6.74 min; HPLC retention time (Method P): t_{R}=6.98 min.

### Example 35

### 2-(1-Amino-3-methylbutyl)-7-methoxy-6-(pyridin-4-yl)quinazoline-4-carbonitrile

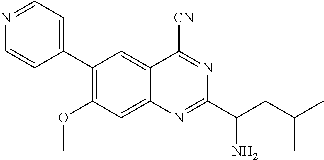 [SMILES: COc1cc2nc(C(N)CC(C)C)nc(C#N)c2cc1-c1ccncc1]

### Part A. Benzyl 1-(7-methoxy-4-oxo-6-(pyridin-4-yl)-3,4-dihydroquinazolin-2-yl)-3-methylbutylcarbamate

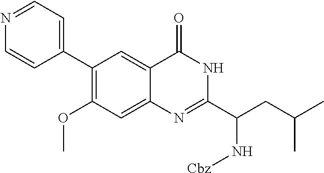 [SMILES: COc1cc2nc(C(CC(C)C)NC(=O)OCc3ccccc3)[nH]c(=O)c2cc1-c1ccncc1]

To a solution of benzyl 1-(6-bromo-7-methoxy-4-oxo-3,4-dihydroquinazolin-2-yl)-3-methylbutylcarbamate (590 mg, 1.244 mmol), prepared as described in Example 32, Parts A-D, in dioxane (10 mL) and water (2.5 mL) was added pyridin-4-ylboronic acid (229 mg, 1.87 mmol) and cesium carbonate (811 mg, 2.49 mmol). The solution was degassed by sonication under N₂ for 5 min. Pd(PPh₃)₄ (144 mg, 0.124 mmol) was then added and the mixture was heated at 95° C. for 3.5 h. The reaction mixture was cooled to room temperature and was diluted with CH₂Cl₂ and was transferred to a separatory funnel containing saturated aqueous NaHCO₃ solution (20 mL). The aqueous layer was extracted with CH₂Cl₂ (3×25 mL). The combined organic layers were washed with brine (20 mL), dried over MgSO₄, filtered and concentrated. The residue was purified by column chromatography on silica gel (1%-5% methanol in CH₂Cl₂) to afford benzyl 1-(7-methoxy-4-oxo-6-(pyridin-4-yl)-3,4-dihydroquinazolin-2-yl)-3-methylbutylcarbamate (392 mg, 67% yield) as a pale yellow solid: ¹H NMR (400 MHz, CDCl₃) δ 11.57 (br. s., 1H), 8.69 (d, J=6.0 Hz, 2H), 8.24 (s, 1H), 7.58 (d, J=6.3 Hz, 2H), 7.34-7.29 (m, 5H), 7.24 (s, 1H), 5.82 (d, J=8.0 Hz, 1H), 5.14 (ABq, J_{AB}=12.3, Δυ=14.2 Hz, 2H), 4.91-4.81 (m, 1H), 4.00 (s, 3H), 1.90-1.77 (m, 3H), 1.06 (d, J=4.8 Hz, 3H), 1.02 (d, J=5.8 Hz, 3H); MS (ESI) m/e 473.1 [(M+H)⁺, calcd for C₂₇H₂₉N₄O₄ 473.2].

### Part B. Benzyl 1-(4-cyano-7-methoxy-6-(pyridin-4-yl)quinazolin-2-yl)-3-methylbutylcarbamate

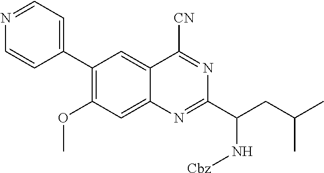 [SMILES: COc1cc2nc(C(CC(C)C)NC(=O)OCc3ccccc3)nc(C#N)c2cc1-c1ccncc1]

To a solution of benzyl 1-(7-methoxy-4-oxo-6-(pyridin-4-yl)-3,4-dihydroquinazolin-2-yl)-3-methylbutylcarbamate (174 mg, 0.368 mmol) and BOP (212 mg, 0.479 mmol) in acetonitrile (4 mL) was added DBU (0.083 mL, 0.552 mmol). The mixture was stirred for 25 min at room temperature. Sodium cyanide (36.1 mg, 0.736 mmol) was added to the reaction mixture. After stirring the reaction mixture for an additional 5 min, 18-crown-6 (195 mg, 0.736 mmol) was added. The solution changed from pale yellow to dark red over 15 min. Stirring was continued for 14 h.

The reaction mixture was transferred to a separatory funnel containing saturated aqueous NaHCO₃ solution (20 mL). The aqueous layer was extracted with CH₂Cl₂ (3×25 mL). The combined organic layers were washed with brine (20 mL), dried over MgSO₄, filtered, and concentrated. The residue was purified by column chromatography on silica gel (1%-3% methanol in CH₂Cl₂) to afford benzyl 1-(4-cyano-7-methoxy-6-(pyridin-4-yl)quinazolin-2-yl)-3-methylbutylcarbamate (104 mg, 59% yield) as an off-white foam: ¹H NMR (400 MHz, CDCl₃) δ 8.79 (d, J=5.8 Hz, 2H), 8.13 (s, 1H), 7.61 (d, J=6.0 Hz, 2H), 7.51 (s, 1H), 7.41-7.37 (m, 5H), 5.76 (d, J=8.8 Hz, 1H), 5.28-5.20 (m, 1H), 5.14 (s, 2H), 4.08 (s, 3H), 1.82-1.70 (m, 3H), 1.05 (d, J=6.0 Hz, 3H), 1.01 (d, J=6.0 Hz, 3H); MS (ESI) m/e 482.1 [(M+H)⁺, calcd for C₂₈H₂₈N₅O₃ 482.2].

### Part C. 2-(1-Amino-3-methylbutyl)-7-methoxy-6-(pyridin-4-yl)quinazoline-4-carbonitrile

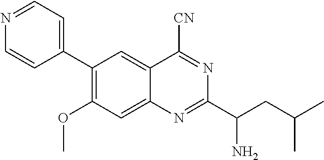 [SMILES: COc1cc2nc(C(N)CC(C)C)nc(C#N)c2cc1-c1ccncc1]

To a solution of benzyl 1-(4-cyano-7-methoxy-6-(pyridin-4-yl)quinazolin-2-yl)-3-methylbutylcarbamate (75 mg, 0.156 mmol) in CH₂Cl₂ (7 mL) at −15° C. (ice/acetone bath) was added boron tribromide (0.47 mL, 0.47 mmol). The reaction mixture was stirred at −15° C. for 20 min. The reaction was quenched by the addition of saturated aqueous NaHCO₃ solution. The reaction mixture was transferred to a separatory funnel containing saturated aqueous NaHCO₃ solution (5 mL). The aqueous layer was extracted with CH₂Cl₂ (3×10 mL). The combined organic layers were washed with brine (5 mL), dried over MgSO₄, filtered, and concentrated. The residue was purified by reverse phase HPLC (5% MeCN:95% water:0.1% TFA→95% MeCN:5% water:0.1% TFA). The organic solvent was removed on the rotovapor and the aqueous mixture was frozen and placed on the lyophilizer to afford 2-(1-amino-3-methylbutyl)-7-methoxy-6-(pyridin-4-yl)quinazoline-4-carbonitrile (7.3 mg) as a colorless amorphous solid: ¹H NMR (500 MHz, DMSO-d₆) δ 8.78 (d, J=5.5 Hz, 2H), 8.61 (br. s., 3H), 8.18 (s, 1H), 7.76 (d, J=5.5 Hz, 2H), 7.67 (s, 1H), 4.71-4.63 (m, 1H), 4.10 (s, 3H), 1.94-1.80 (m, 2H), 1.69 (dt, J=13.4, 6.6 Hz, 1H), 0.98 (d, J=6.7 Hz, 3H), 0.94 (d, J=6.4 Hz, 3H); MS (ESI) m/e 348.1 [(M+H)⁺, calcd for C₂₀H₂₂N₅O 348.1]. HPLC retention time (Method O): t_{R}=6.69 min; HPLC retention time (Method P): t_{R}=6.90 min.

### Example 36

### (R)-1-(7-Methoxy-6-(oxazol-5-yl)quinazolin-2-yl)-3-methylbutan-1-amine

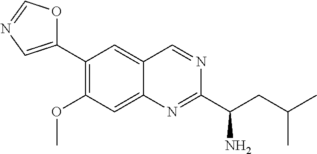 [SMILES: COc1cc2nc([C@H](N)CC(C)C)ncc2cc1-c1cnco1]

### Part A. Methyl 4-amino-2-methoxybenzoate

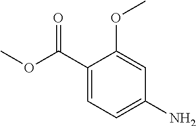

To a solution of methyl 2-methoxy-4-nitrobenzoate (10.0 g, 0.047 mol) in MeOH (100 mL) was added Pd/C (4.0 g, 10% w/w) under a nitrogen atmosphere in an autoclave and the reaction mixture was stirred at 45 psi (hydrogen atm) at room temperature for 12 h. The reaction mixture was passed through a Celite pad and the pad was washed with excess ethyl acetate. The filtrate was evaporated to dryness under reduced pressure to afford methyl 4-amino-2-methoxybenzoate (8.6 g, 0.048 mol, 99% yield) as a brown solid. ¹H NMR (400 MHz, DMSO-d₆) δ 7.52 (d, J=8.4 Hz, 1H), 6.32 (s, 1H), 6.23-6.25 (m, 1H), 5.6 (bs, 2H), 3.72 (s, 3H), 3.67 (s, 3H); LCMS (ESI) m/e 182.2 [(M+H)⁺, calcd for C₉H₁₂NO₃, 182.07]; LC/MS retention time (method B): t_{R}=0.87 min.

### Part B. Methyl 4-acetamido-2-methoxybenzoate

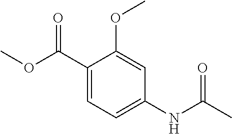 [SMILES: COC(=O)c1ccc(NC(C)=O)cc1OC]

To a solution of methyl 4-amino-2-methoxybenzoate (2.0 g, 0.011 mol) in DCM (50 mL) cooled to 0° C. was added triethylamine (3.0 mL, 0.022 mol) and the reaction mixture was stirred for 10 minutes. Acetyl chloride (1.3 g, 0.0165 mol) was added dropwise and the reaction was stirred for 3 h. Water (50 mL) was added and the aqueous layer was extracted with DCM (2×50 mL). The combined organic extracts were dried over sodium sulfate, filtered and volatiles were evaporated to dryness under reduced pressure to give methyl 4-acetamido-2-methoxybenzoate (2.3 g, 0.01 mol, 91% yield) as a light brown solid. ¹H NMR (400 MHz, DMSO-d₆) δ 10.22 (s, 1H), 7.66 (d, J=8.8 Hz, 1H), 7.47 (d, J=1.6 Hz, 1H), 7.20 (dd, J=8.4, 2.0 Hz, 1H), 3.74-3.79 (m, 6H), 2.07 (s, 3H); LCMS (ESI) m/e 224.2 [(M+H)⁺, calcd for C₁₁H₁₄NO₄, 224.08]; LC/MS retention time (method B): t_{R}=1.20 min.

### Part C. Methyl 4-acetamido-5-bromo-2-methoxybenzoate

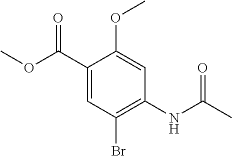 [SMILES: COC(=O)c1cc(Br)c(NC(C)=O)cc1OC]

To a solution of methyl 4-acetamido-2-methoxybenzoate (2.0 g, 8.9 mmol) in acetic acid (30 mL) cooled to 0° C. was added bromine (0.5 mL, 9.85 mmol) dropwise and the reaction mixture was stirred for 30 minutes. The volatiles were evaporated to dryness under reduced pressure and the pH of the reaction mixture was adjusted to pH 8 using saturated aq. sodium bicarbonate solution. The aqueous mixture was extracted with DCM (2×40 mL). The combined organic extracts were dried over sodium sulfate, filtered and volatiles were evaporated to dryness under reduced pressure to give methyl 4-acetamido-5-bromo-2-methoxybenzoate (2.0 g, 6.62 mmol, 63% yield) as a brown solid. ¹H NMR (400 MHz, DMSO-d₆) δ 9.44 (s, 1H), 7.86 (s, 1H), 7.65 (s, 1H), 3.75-3.77 (m, 6H), 2.13 (s, 3H); LCMS (ESI) m/e 302.0 [(M+H)⁺, calcd for C₁₁H₁₃BrNO₄, 301.99]; LC/MS retention time (method B): t_{R}=1.42 min.

### Part D. N-(2-Bromo-4-(hydroxymethyl)-5-methoxyphenyl)acetamide

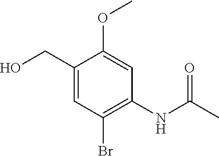

To a solution of methyl 4-acetamido-5-bromo-2-methoxybenzoate (2.0 g, 6.6 mmol) in THF (50 mL) cooled to −10° C. was added DIBAL-H (7.5 mL, 13.2 mmol, 25% solution in toluene) dropwise and the reaction mixture was allowed to warm to 0° C. and was stirred for 2 h. The reaction was quenched with saturated aq. ammonium chloride solution (80 mL) and extracted with ethyl acetate (3×30 mL). Combined organic extracts were evaporated to dryness under reduced pressure to give N-(2-bromo-4-(hydroxymethyl)-5-methoxyphenyl)acetamide (1.56 g, 5.71 mmol, 83% yield) as a light brown solid. ¹H NMR (400 MHz, DMSO-d₆) δ 9.39 (s, 1H), 7.51 (s, 1H), 7.23 (s, 1H), 5.15 (t, J=5.6 Hz, 1H), 4.44 (d, J=5.6 Hz, 2H), 3.74 (m, 3H), 2.07 (s, 3H).

### Part E. N-(2-Bromo-4-formyl-5-methoxyphenyl)acetamide

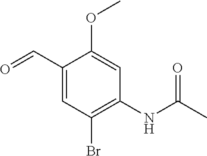

To a stirred solution of N-(2-bromo-4-(hydroxymethyl)-5-methoxyphenyl)acetamide (1.8 g, 3.6 mmol) in DCM (30 mL) cooled to 0° C. was added Dess-Martin periodinane (1.7 g, 4.0 mmol) under a nitrogen atmosphere and the reaction mixture was stirred at room temperature for 12 h. The reaction mixture was quenched with aq. sodium thiosulfate (25 mL) & aq. sodium bicarbonate (25 mL). The mixture was extracted with ethyl acetate (3×50 mL). The combined organic layers were washed with water (50 mL) and brine solution (50 mL), dried over sodium sulfate, and the volatiles were evaporated to dryness under reduced pressure to give N-(2-bromo-4-formyl-5-methoxyphenyl)acetamide (1.39 g, 5.1 mmol, 73% yield) as a yellow solid. ¹H NMR (400 MHz, DMSO-d₆) δ 10.18 (s, 1H), 9.51 (bs, 1H), 7.83 (d, J=8.8 Hz, 2H), 3.88 (s, 3H), 2.18 (s, 3H).

### Part F. N-(2-Bromo-5-methoxy-4-(oxazol-5-yl)phenyl)acetamide

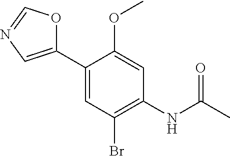 [SMILES: COc1cc(NC(C)=O)c(Br)cc1-c1cnco1]

To a stirred solution of N-(2-bromo-4-formyl-5-methoxyphenyl)acetamide (1.0 g, 3.6 mmol) in MeOH (50 mL) was added TosMIC (0.78 g, 3.6 mmol) and potassium carbonate (0.5 g, 3.6 mmol) under a nitrogen atmosphere and the reaction mixture was stirred at room temperature for 1 h. The volatiles were evaporated to dryness and the residue obtained was quenched with aq. sodium bicarbonate solution (50 mL). The aqueous mixture was extracted with DCM (2×25 mL). The combined organic extracts were dried over sodium sulfate and the volatiles were evaporated to dryness under reduced pressure to give N-(2-bromo-5-methoxy-4-(oxazol-5-yl)phenyl)acetamide (0.98 g, 3.14 mmol, 57% yield) as an off-white solid. ¹H NMR (400 MHz, DMSO-d₆) δ 9.49 (s, 1H), 8.45 (s, 1H), 7.86 (d, J=6.4 Hz, 1H), 7.55-7.59 (m, 2H), 3.91 (s, 3H), 2.13 (s, 3H); LCMS (ESI) m/e 313.0 (bromo pattern) [(M+H)⁺, calcd for C₁₂H₁₂BrN₂O₃, 311.00]; LC/MS retention time (method B): t_{R}=1.55 min.

### Part G. N-(5-Methoxy-4-(oxazol-5-yl)-2-vinylphenyl)acetamide

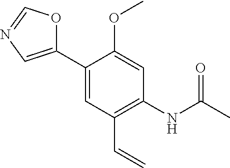 [SMILES: C=Cc1cc(-c2cnco2)c(OC)cc1NC(C)=O]

To a stirred solution of N-(2-bromo-5-methoxy-4-(oxazol-5-yl)phenyl)acetamide (60 mg, 0.19 mmol) in a mixture of toluene (10 mL) and EtOH (3 mL) was added vinyl boronic anhydride (0.05 g, 0.38 mmol) followed by sodium carbonate (0.08 g, 076 mmol). The reaction mixture was purged with nitrogen for 5 minutes followed by the addition of Pd(PPh₃)₄ (0.13 g, 0.011 mmol) and the reaction mixture was stirred at 95° C. for 4 h. Water (10 mL) was added and the mixture was extracted with ethyl acetate (2×15 mL). The combined organic extracts were washed with brine solution (20 mL) and the organic layer was dried over sodium sulfate. The volatiles were evaporated to dryness under reduced pressure and the residue was purified by Prep.

TLC using 40% ethyl acetate in hexanes. The required fractions were collected and concentrated to give N-(5-methoxy-4-(oxazol-5-yl)-2-vinylphenyl)acetamide (40 mg, 0.154 mmol, 22% yield) as an off-white solid. LCMS (ESI) m/e 259.2 [(M+H)⁺, calcd for C₁₄H₁₅N₂O₃, 259.10]; LC/MS retention time (method B): t_{R}=1.44 min.

### Part H. 5-Methoxy-4-(oxazol-5-yl)-2-vinylaniline

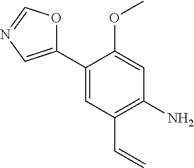 [SMILES: C=Cc1cc(-c2cnco2)c(OC)cc1N]

To a stirred solution of N-(5-methoxy-4-(oxazol-5-yl)-2-vinylphenyl)acetamide (40 mg, 0.15 mmol) in a mixture of EtOH (5 mL) and water (1 mL) was added KOH (8.7 mg, 1.5 mmol) and the reaction mixture was stirred at 65° C. for 12 h. The volatiles were evaporated to dryness under reduced pressure and water (10 mL) was added and the mixture was extracted with DCM (2×10 mL). The combined organic extracts were dried over sodium sulfate and the volatiles were evaporated to dryness under reduced pressure to give 5-methoxy-4-(oxazol-5-yl)-2-vinylaniline (20 mg, 0.092 mmol, 59% yield) as a brown solid. LCMS (ESI) m/e 217.2 [(M+H)⁺, calcd for C₁₂H₁₃N₂O₂, 217.09]; LC/MS retention time (method B): t_{R}=1.44 min.

### Part I. (R)-tert-Butyl 1-(5-methoxy-4-(oxazol-5-yl)-2-vinylphenylamino)-4-methyl-1-oxopentan-2-ylcarbamate

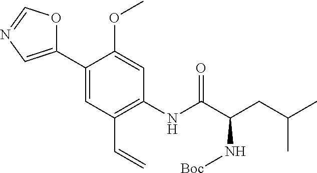 [SMILES: C=Cc1cc(-c2cnco2)c(OC)cc1NC(=O)[C@@H](CC(C)C)NC(=O)OC(C)(C)C]

A solution of 5-methoxy-4-(oxazol-5-yl)-2-vinylaniline (300 mg, 1.38 mmol), Boc-D-leucine (0.64 g, 2.7 mmol), HATU (1.04 g, 2.7 mmol) and DIPEA (0.5 mL, 5.2 mmol) in DCM (15 mL) was stirred at room temperature for 14 h. Water (10 mL) was added and the mixture was extracted with DCM (2×10 mL). The combined organic extracts were dried over sodium sulfate and the volatiles were evaporated to dryness under reduced pressure. The residue obtained was purified by prep. TLC using 40% ethyl acetate in hexanes. The required fractions were collected and concentrated to give (R)-tert-butyl 1-(5-methoxy-4-(oxazol-5-yl)-2-vinylphenylamino)-4-methyl-1-oxopentan-2-ylcarbamate (320 mg, 0.74 mmol, 11% yield). LCMS (ESI) m/e 430.2 [(M+H)⁺, calcd for C₂₃H₃₂N₃O₅, 430.23]; LC/MS retention time (method B): t_{R}=2.05 min.

### Part J. (R)-tert-Butyl 1-(2-formyl-5-methoxy-4-(oxazol-5-yl)phenylamino)-4-methyl-1-oxopentan-2-ylcarbamate

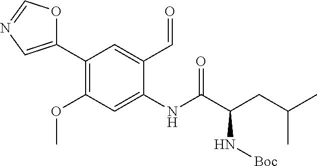 [SMILES: COc1cc(NC(=O)[C@@H](CC(C)C)NC(=O)OC(C)(C)C)c(C=O)cc1-c1cnco1]

A stirred solution of (R)-tert-butyl 1-(5-methoxy-4-(oxazol-5-yl)-2-vinylphenylamino)-4-methyl-1-oxopentan-2-ylcarbamate (200 mg, 0.46 mmol) in 1,4-dioxane (10 mL) and water (3 mL) was cooled to 0° C. and 2,6-lutidine, osmium tetraoxide (0.004 mL, 0.138 mmol, 2.5% solution in t-butanol) and sodium periodate (0.39 g, 1.8 mmol) were added and the reaction mixture was stirred at room temperature for 13 h. Water (10 mL) was added and the mixture was extracted with ethyl acetate (2×10 mL). The combined organic layers were dried over sodium sulfate and concentrated under reduced pressure. The residue obtained was purified by prep. TLC using 40% ethyl acetate in hexanes. The required fractions were collected and concentrated to give (R)-tert-butyl 1-(2-formyl-5-methoxy-4-(oxazol-5-yl)phenylamino)-4-methyl-1-oxopentan-2-ylcarbamate (150 mg, 0.34 mmol, 74% yield) as white solid. LCMS (ESI) m/e 432.2 [(M+H)⁺, calcd for C₂₂H₃₀N₃O₆, 432.21]; LC/MS retention time (method B): t_{R}=2.04 min.

### Part K. (R)-1-(7-Methoxy-6-(oxazol-5-yl)quinazolin-2-yl)-3-methylbutan-1-amine

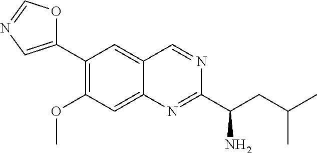 [SMILES: COc1cc2nc([C@H](N)CC(C)C)ncc2cc1-c1cnco1]

A stirred solution of (R)-tert-butyl 1-(2-formyl-5-methoxy-4-(oxazol-5-yl)phenylamino)-4-methyl-1-oxopentan-2-ylcarbamate (120 mg, 0.27 mmol) and ammonium acetate (0.41 g, 0.55 mmol) in AcOH (10 mL) was heated at 85° C. under a nitrogen atmosphere for 14 h. The completion of the reaction was judged by the disappearance of starting material by TLC (80% ethyl acetate in hexane). The volatiles were evaporated to dryness under reduced pressure and the residue obtained was basified by the addition of saturated aq. sodium bicarbonate solution (25 mL). The aqueous mixture was extracted with ethyl acetate (2×15 mL). The combined organic extracts were dried over sodium sulfate and the volatiles were evaporated to dryness under reduced pressure. The residue was purified by prep. HPLC (0.05% TFA in water and acetonitrile). The required fractions were collected and the volatiles were evaporated to dryness to give (R)-1-(7-methoxy-6-(oxazol-5-yl)quinazolin-2-yl)-3-methylbutan-1-amine (10 mg, 0.032 mmol, 11% yield) as brown solid which was isolated as TFA salt: ¹H NMR (400 MHz, CD₃OD) δ 9.50 (s, 1H), 8.56 (s, 1H), 8.40 (s, 1H), 7.80 (s, 1H), 7.59 (s, 1H), 4.66 (t, J=7.2 Hz, 1H), 4.23 (s, 3H), 2.03-2.10 (m, 1H), 1.86-1.93 (m, 1H), 1.73-1.80 (m, 1H), 1.04-1.08 (m, 6H); LCMS (ESI) m/e 313.2 [(M+H)⁺, calcd for C₁₇H₂₁N₄O₂, 313.16]; LC/MS retention time (method A): t_{R}=1.25 min; HPLC retention time (method B): t_{R}=11.50 min; HPLC retention time (method A): t_{R}=10.68 min; Chiral HPLC retention time (method E): t_{R}=11.46 min.

### Example 37

### 2-Isopentyl-4-methoxy-6-(1H-pyrazol-4-yl)quinazoline

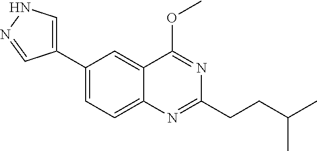 [SMILES: COc1nc(CCC(C)C)nc2ccc(-c3cn[nH]c3)cc12]

### Part A. 2-Amino-5-bromobenzoic acid

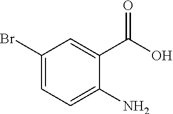 [SMILES: Nc1ccc(Br)cc1C(=O)O]

To a stirred solution of 5-bromoindoline-2,3-dione (20.0 g, 8.8 mmol) in aq. 3N NaOH (88 mL) stirred at 80° C., was added dropwise hydrogen peroxide (22.2 mL, 2.7 mmol, 35% in water) and the reaction mixture was stirred at 80° C. for 1 h. The reaction mixture was allowed to cool to room temperature. The reaction mixture was cooled to 0° C. and was quenched by addition of cone. HCl (100 mL) and the pH of the reaction was adjusted to pH 5. The volatiles were evaporated to dryness under reduced pressure to afford crude product as a brown solid. The solid was suspended in MeOH (150 mL) and stirred for 15 minutes, filtered and the filtrate was evaporated to dryness under reduced pressure to give 2-amino-5-bromobenzoic acid (18 g, 84.4 mmol, 96% yield) as brown solid. LCMS (ESI) m/e 216.0 [(M+H)⁺, calcd for C₇H₇BrNO₂, 216.0]; LC/MS retention time (method A): t_{R}=0.86 min.

### Part B. 2-Amino-5-bromobenzamide

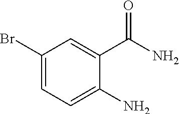 [SMILES: NC(=O)c1cc(Br)ccc1N]

To a stirred solution of 2-amino-5-bromobenzoic acid (14.0 g, 0.065 mol) dissolved in THF (230 mL) was added CDI (12.6 g, 0.077 mol). The reaction mixture was stirred for 2 h at rt. To this mixture ammonia (321 mL, 25% solution in water) was added and the mixture was stirred at room temperature for 14 h. The volatiles were evaporated to dryness; water (100 mL) was added and the mixture was extracted with ethyl acetate (2×150 mL). The combined organic extracts were concentrated under reduced pressure to afford a residue. The product was purified by silica gel column chromatography using 70% ethyl acetate in hexanes. The required fractions were collected and concentrated under reduced pressure to afford 2-amino-5-bromobenzamide (14.0 g, 0.065 mol, 82% yield) as a brown solid. LCMS (ESI) m/e 215.0 (bromo pattern) [(M+H)⁺, calcd for C₇H₈BrN₂O, 214.97]; LC/MS retention time (method B): t_{R}=0.93 min.

### Part C. 5-Bromo-2-(4-methylpentanamido)benzamide

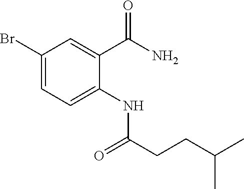 [SMILES: CC(C)CCC(=O)Nc1ccc(Br)cc1C(N)=O]

To a stirred solution of 2-amino-5-bromobenzamide (7.8 g, 0.036 mol), 4-methyl pentanoic acid (8.41 g, 0.072 mmol), and TBTU (17.46 g, 0.054 mol) in DMF (100 mL) was added DIPEA (25 mL, 0.154 mol) and the reaction mixture was stirred at room temperature for 14 h. The reaction mixture was diluted with water (100 mL) and extracted with ethyl acetate (2×100 mL). The combined organic layers were washed with aq. sodium bicarbonate solution (100 mL) and brine (100 mL). The organic layer was dried over sodium sulfate, and the volatiles were evaporated to dryness. The residue was purified by silica gel column chromatography using a gradient of 40% ethyl acetate in hexanes and the required fractions were collected and volatiles were evaporated to give 5-bromo-2-(4-methylpentanamido)benzamide (7.0 g, 0.022 mol, 55% yield) as a brown solid. LCMS (ESI) m/e 313.0 (bromo pattern) [(M+H)⁺, calcd for C₁₃H₁₈BrN₂O₂, 313.05]; LC/MS retention time (LC/MS Method=Column−YMC PACK TMS (3×50 mm), 3.0 μm; Mphase A: 2% MeCN—98% H₂O—10 mM NH₄OAc; Mphase B: 98% ACN—2% H₂O—10 mM NH₄OAC; Flow: 1.2 mL/min)): t_{R}=2.12 min.

### Part D. 6-Bromo-2-isopentylquinazolin-4(3H)-one

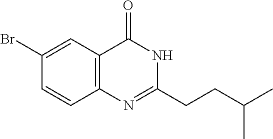 [SMILES: CC(C)CCc1nc2ccc(Br)cc2c(=O)[nH]1]

To a solution of 5-bromo-2-(4-methylpentanamido)benzamide (6.0 g, 0.019 mol) in EtOH (25.8 mL) was added 5% aq. NaOH solution (51.4 mL) and the reaction mixture was refluxed for 1.5 h. The reaction mixture was cooled to 0° C., acidified with acetic acid (100 mL) and ⅓ volume of solvent was evaporated. The solid obtained was collected, washed with water (100 mL), hexane (100 mL), and dried under vacuum give 6-bromo-2-isopentylquinazolin-4(3H)-one (5.7 g, 0.019, 86% yield) as a light brown solid. LCMS (ESI) m/e 295.0, 297.0 (bromo pattern) [(M+H)⁺, calcd for C₁₃H₁₆BrN₂O, 295.04]; LC/MS retention time (method D): t_{R}=0.93 min.

### Part E. 6-Bromo-2-isopentyl-4-methoxyquinazoline

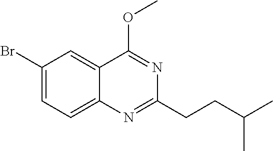 [SMILES: COc1nc(CCC(C)C)nc2ccc(Br)cc12]

6-Bromo-2-isopentylquinazolin-4(3H)-one (210 mg, 0.71 mmol) was dissolved in POCl₃ (0.5 mL) in a microwave vial and the reaction mixture was irradiated for 20 minutes at 90° C. The completion of reaction was judged by the disappearance of starting material by TLC (20% ethyl acetate in hexane). The volatiles were evaporated to dryness, and aq. saturated sodium bicarbonate solution (20 mL) was added. The aqueous mixture was extracted with ethyl acetate (2×15 mL). The combined organic extracts were dried over sodium sulfate, and the volatiles were evaporated to dryness under reduced pressure to afford 6-bromo-4-chloro-2-isopentylquinazoline (110 mg, 0.35 mmol, 52% yield) as an orange solid.

The product was dissolved in MeOH (2 mL) and sodium methoxide (0.019 mg, 0.37 mmol) was added under nitrogen atmosphere and the reaction mixture was stirred at room temperature for 30 minutes. The volatiles were evaporated to dryness, and water (10 mL) was added. The aqueous mixture was extracted with ethyl acetate (2×10 mL). The organic extracts were dried over sodium sulfate, and the volatiles were evaporated to dryness under reduced pressure to give 6-bromo-2-isopentyl-4-methoxyquinazoline (55 mg, 0.178 mmol, 50% yield) as an off white solid. LCMS (ESI) m/e 309.0 (bromo pattern) [(M+H)⁺, calcd for C₁₄H₁₈BrN₂O, 309.05]; LC/MS retention time (method A): t_{R}=2.78 min.

### Part F. 2-Isopentyl-4-methoxy-6-(1H-pyrazol-4-yl)quinazoline

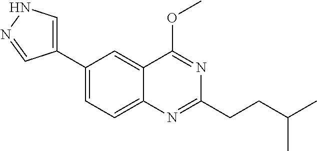 [SMILES: COc1nc(CCC(C)C)nc2ccc(-c3cn[nH]c3)cc12]

To a stirred solution of 6-bromo-2-isopentyl-4-methoxyquinazoline (110 mg, 0.357 mmol), pyrazole-4-boronic acid (0.142 mg, 0.53 mmol), and cesium carbonate (423 mg, 1.42 mmol) in a mixture of 1,4-dioxane (1 mL) and water (0.5 mL) under nitrogen was added Pd(PPh₃)₄ (18 mg, 0.014 mmol). The reaction mixture was heated at 90° C. for 3 h. The volatiles were evaporated to dryness. Water (10 mL) was added and the mixture was extracted with ethyl acetate (2×10 mL). The combined organic extracts were dried over sodium sulfate, and the volatiles were evaporated to dryness under reduced pressure. The residue was purified by silica gel column chromatography using a gradient of ethyl acetate in hexane. The required fractions were collected and concentrated to give 2-isopentyl-4-methoxy-6-(1H-pyrazol-4-yl)quinazoline (75 mg, 0.248 mmol, 68% yield) as an off white solid. ¹H NMR (400 MHz, CDCl₃) δ 8.22 (d, J=2 Hz, 1H), 7.94-7.98 (m, 3H), 7.88 (d, J=8.4 Hz, 1H), 4.20 (s, 3H), 2.94-2.98 (m, 2H), 1.77-1.82 (m, 2H), 1.67-1.75 (m, 1H), 0.99 (d, J=6.4 Hz, 6H); LCMS (ESI) m/e 297.2 [(M+H)⁺, calcd for C₁₇H₂₁N₄O, 297.16]; LC/MS retention time (method A): t_{R}=1.95 min; HPLC retention time (method C): t_{R}=7.20 min; HPLC retention time (method D): t_{R}=11.33 min.

### Example 38

### 2-Isopentyl-4-methoxy-6-(pyridin-4-yl)quinazoline

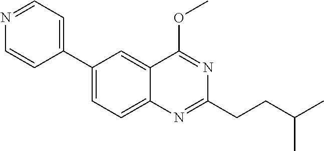 [SMILES: COc1nc(CCC(C)C)nc2ccc(-c3ccncc3)cc12]

A stirred solution of 6-bromo-2-isopentyl-4-methoxyquinazoline (110 mg, 0.324 mmol) (prepared as described in Example 37, Parts A-E), pyridine boronic acid (65 mg, 0.53 mmol), cesium carbonate (423 mg, 1.42 mmol), and Pd(PPh₃)₄ (18 mg, 0.014 mmol) in a mixture of 1,4-dioxane (1 mL) and water (0.5 mL) under nitrogen was heated at 90° C. for 3 h. The volatiles were evaporated to dryness; water (10 mL) was added and the mixture was extracted with ethyl acetate (2×10 mL). The combined organic extracts were dried over sodium sulfate, and the volatiles were evaporated to dryness under reduced pressure. The residue was purified by prep HPLC (10 mM ammonium acetate in water and acetonitrile). The required fractions were collected and concentrated to give 2-isopentyl-4-methoxy-6-(pyridin-4-yl)quinazoline (65 mg, 0.21 mmol, 59% yield) as pale yellow solid. ¹H NMR (400 MHz, CDCl₃) δ 8.71 (d, J=2 Hz, 2H), 8.39-8.40 (m, 1H), 8.05 (dd, J=8.8, 2 Hz, 1H), 7.96 (d, J=8.8 Hz, 1H), 7.61 (dd, J=4.8, 1.6 Hz, 2H), 4.20 (s, 3H), 2.96-3.00 (m, 2H), 1.77-1.82 (m, 2H), 1.67-1.73 (m, 1H), 0.99 (d, J=6.4 Hz, 6H); LCMS (ESI) m/e 308.2 [(M+H)⁺, calcd for C₁₉H₂₂N₃O, 308.17]; LC/MS retention time (method A): t_{R}=2.50 min; HPLC retention time (method D): t_{R}=7.63 min; HPLC retention time (method C): t_{R}=6.65 min.

### Example 39

### 2-Isopentyl-N-methyl-6-(oxazol-5-yl)quinazolin-4-amine

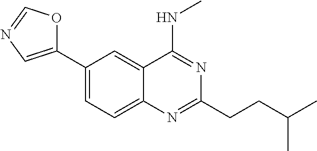 [SMILES: CNc1nc(CCC(C)C)nc2ccc(-c3cnco3)cc12]

### Part A. 6-Bromo-4-chloro-2-isopentylquinazoline

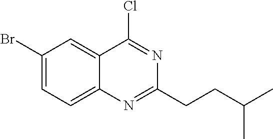 [SMILES: CC(C)CCc1nc(Cl)c2cc(Br)ccc2n1]

To a stirred solution of 6-bromo-2-isopentylquinazolin-4(3H)-one (0.50 g, 1.69 mmol) (prepared as described in Example 37, Parts A-D) in toluene (10 mL) at room temperature was added POCl₃(0.77 mL, 8.47 mmol) followed by the addition of DIPEA (0.3 mL, 1.69 mmol) (over 10 min). The mixture was stirred for 30 min at the same temperature. The reaction mixture was then heated at 110° C. for 5 h. The volatiles were evaporated to dryness under reduced pressure to give 6-bromo-4-chloro-2-isopentylquinazoline (0.5 g, 1.59 mmol, 94% yield) as a light brown solid.

The product was not stable. It was taken to next step without further purification and analysis.

### Part B. 6-Bromo-2-isopentyl-N-methylquinazolin-4-amine

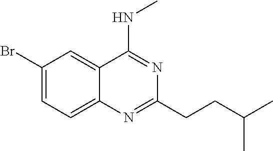 [SMILES: CNc1nc(CCC(C)C)nc2ccc(Br)cc12]

To a stirred solution of 6-bromo-4-chloro-2-isopentylquinazoline (500 mg, 1.59 mmol) in THF (10 mL) at 0° C. in a pressure tube was added methylamine (2M in THF, 3.18 mL, 6.37 mmol) followed by the addition of DIPEA (1.09 mL, 6.37 mmol). The vessel was sealed and the reaction mixture was heated at 65° C. for 16 h.

The completion of reaction was judged by the disappearance of starting material by TLC (30% ethyl acetate in hexanes). The reaction mixture was diluted with water (15 mL) and extracted with ethyl acetate (2×25 mL). The combined organic extracts were dried over sodium sulfate and the volatiles were evaporated to dryness under reduced pressure. The residue was purified by silica gel column chromatography using a gradient of 30% ethyl acetate in hexanes. The required fractions were collected and evaporated and the product was purified by silica gel column chromatography using a gradient of hexanes/ethyl acetate as mobile phase to afford 6-bromo-2-isopentyl-N-methylquinazolin-4-amine (90 mg, 0.29 mmol, 18% yield) as an off white solid. ¹H NMR (400 MHz, CDCl₃) δ 7.78-7.72 (m, 2H), 7.65 (d, J=8.8 Hz, 1H), 5.53 (bs, 1H), 3.20 (d, J=4.8 Hz, 3H), 2.87-2.83 (m, 2H), 1.78-1.73 (m, 2H), 1.69-1.66 (m, 1H), 0.97 (m, 6H).

### Part C. 2-Isopentyl-N-methyl-6-vinylquinazolin-4-amine

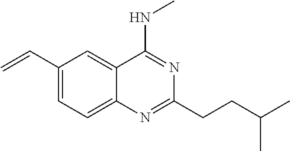 [SMILES: C=Cc1ccc2nc(CCC(C)C)nc(NC)c2c1]

To a stirred solution of 6-bromo-2-isopentyl-N-methylquinazolin-4-amine (90 mg, 0.29 mmol) in a toluene (2.16 mL) and ethanol (0.54 mL) mixture was added vinyl boronic anhydride pyridine complex (140 mg, 0.584 mmol) and 2N aq. sodium carbonate (61 mg, 0.584 mmol). N₂ gas was bubbled through the reaction mixture for 5 min. Pd(PPh₃)₄ (16 mg, 0.014 mmol) was then added and the reaction mixture was heated for 3 h at 95° C. The completion of the reaction was judged by the disappearance of starting material by TLC (30% ethyl acetate in hexane). The reaction mixture was allowed to cool to room temperature and was diluted with satd. aq. sodium bicarbonate solution (6 mL) and the mixture was extracted with ethyl acetate (2×5 mL). The combined organic extracts were washed with brine (5 mL) and dried over sodium sulfate. The volatiles were evaporated to dryness under reduced pressure and purified by Prep. TLC using 30% Ethyl acetate in hexane. The required fractions were collected and the volatiles were evaporated to dryness to afford 2-isopentyl-N-methyl-6-vinylquinazolin-4-amine (80 mg, 0.31 mmol, 98% yield) as a pale yellow solid. LCMS (ESI) m/e 256.2 [(M+H)⁺, calcd for C₁₆H₂₂N₃, 256.17]; LC/MS retention time (method A): t_{R}=1.75 min.

### Part D. 2-Isopentyl-4-(methyl amino) quinazoline-6-carbaldehyde

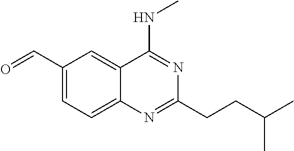 [SMILES: CNc1nc(CCC(C)C)nc2ccc(C=O)cc12]

To a stirred solution of 2-isopentyl-N-methyl-6-vinylquinazolin-4-amine (80 mg, 0.31 mmol) and 2,6-lutidine (67 mg, 0.626 mmol) in a dioxane (2.4 mL) and water (0.6 mL) mixture at 0° C. was added sodium meta periodate (268 mg, 1.25 mmol) followed by the addition of 2.5% solution of osmium tetroxide in t-butanol (0.078 ml, 0.006 mmol). The reaction mixture was allowed to warm to rt and stirred for 1 h.

The completion of reaction was judged by the disappearance of starting material by TLC (20% ethyl acetate in hexane). Then the reaction mixture was diluted with sat aqueous sodium bicarbonate (5 mL) solution and extracted with ethyl acetate (2×5 mL). The combined organic extracts were washed with brine (5 mL) and dried over sodium sulfate, and the volatiles were evaporated to dryness. The residue was purified by silica gel column chromatography using a gradient of 20% ethyl acetate in hexane. The required fractions were collected and evaporated to give 2-isopentyl-4-(methyl amino) quinazoline-6-carbaldehyde (40 mg, 0.15 mmol, 50% yield). LCMS (ESI) m/e 256.2 [(M−H)⁻, calcd for C₁₅H₁₈N₃O, 256.15]; LC/MS retention time (method A): t_{R}=1.82 min.

### Part E. 2-Isopentyl-N-methyl-6-(oxazol-5-yl) quinazolin-4-amine

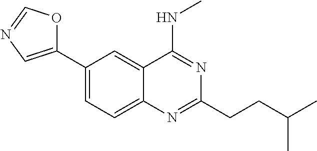 [SMILES: CNc1nc(CCC(C)C)nc2ccc(-c3cnco3)cc12]

To a stirred solution of 2-isopentyl-4-(methyl amino)quinazoline-6-carbaldehyde (40 mg, 0.15 mmol) in methanol (1.0 ml) at rt was added TosMIC (30 mg, 0.155 mmol) followed by the addition of K₂CO₃ (23 mg, 0.171 mmol). The mixture was stirred for 1 h. The completion of reaction was judged by the disappearance of starting material by TLC (40% ethyl acetate in hexane). The volatiles were evaporated to dryness under reduced pressure. To the residue was added 10% sat aq sodium bicarbonate (5 mL) was added and the mixture was extracted with ethyl acetate (2×5 mL). The combined organic extracts were washed with brine (5 mL) and dried over sodium sulfate. The volatiles were evaporated to dryness under reduced pressure and purified by Prep. TLC using a gradient of 40% ethyl acetate in hexane. The required fractions were collected and the volatiles were evaporated to dryness to give 2-isopentyl-N-methyl-6-(oxazol-5-yl) quinazolin-4-amine (20 mg, 0.06 mmol, 50% yield) as an off white solid. ¹H NMR (400 MHz, CD₃OD) δ 8.46 (d, J=1.6 Hz, 1H), 8.33 (s, 1H), 8.09 (dd, J=8.8, 2 Hz, 1H), 7.74 (d, J=8.4 Hz, 1H), 7.63 (s, 1H), 3.18 (s, 3H), 2.81-2.85 (m, 2H), 1.74-1.79 (m, 2H), 1.66-1.71 (m, 1H), 1.01 (d, J=6.4 Hz, 6H); LCMS (ESI) m/e 295.2 [(M−H)⁻, calcd for C₁₇H₁₉N₄O, 295.16]; LC/MS retention time (method A): t_{R}=1.81 min; HPLC retention time (method C): t_{R}=6.37 min; HPLC retention time (method D): t_{R}=7.99 min.

### Example 40

### 2-Isopentyl-N,N-dimethyl-6-(oxazol-5-yl)quinazolin-4-amine

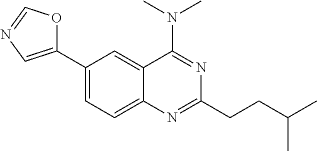 [SMILES: CC(C)CCc1nc(N(C)C)c2cc(-c3cnco3)ccc2n1]

### Part A. 6-Bromo-2-isopentyl-N,N-dimethylquinazolin-4-amine

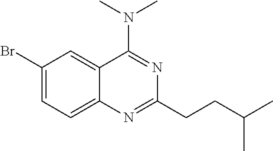 [SMILES: CC(C)CCc1nc(N(C)C)c2cc(Br)ccc2n1]

To a stirred solution of 6-bromo-4-chloro-2-isopentylquinazoline (500 mg, 1.59 mmol) (prepared as described in Example 37, Parts A-E) in THF (10 mL) at 0° C. was added dimethyl amine (2M in THF, 3.18 mL, 6.37 mmol) followed by the addition of DIPEA (1.09 mL, 6.37 mmol). The reaction mixture was allowed to warm to 65° C. and stirred for 16 h. The completion of reaction was judged by the disappearance of starting material by TLC (30% ethyl acetate in hexane). Then the reaction mixture was diluted with water (15 mL) and extracted with ethyl acetate (2×25 mL). The combined organic extracts were dried over sodium sulfate and volatiles were evaporated to dryness under reduced pressure. The residue was purified by silica gel column chromatography using 30% ethyl acetate in hexane. Required fractions were collected and evaporated under reduced pressure to give 6-bromo-2-isopentyl-N,N-dimethylquinazolin-4-amine (450 mg, 1.39 mmol, 88% yield) as a yellow solid.

LCMS (ESI) m/e 322.2 [(M+H)⁺, calcd for C₁₅H₂₁BrN₃, 322.08]; LC/MS retention time (method F): t_{R}=2.12 min.

### Part B. 2-Isopentyl-N,N-dimethyl-6-vinylquinazolin-4-amine

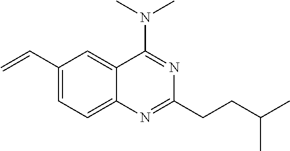 [SMILES: C=Cc1ccc2nc(CCC(C)C)nc(N(C)C)c2c1]

To a stirred solution of 6-bromo-2-isopentyl-N,N-dimethylquinazolin-4-amine (50 mg, 0.15 mmol) in a toluene (1.2 mL) and ethanol (0.3 mL) mixture was added vinyl boronic anhydride pyridine complex (74 mg, 0.310 mmol) and 2N aq. sodium carbonate (32 mg, 0.310 mmol) solution. Nitrogen gas was bubbled through the reaction mixture for 5 min. To the reaction mixture was added Pd(PPh₃)₄ (8 mg, 0.007 mmol) and the mixture was stirred for 3 h at 95° C. The reaction mixture was allowed to cool to rt and diluted with satd. aq. sodium bicarbonate solution (6 mL) and the mixture was extracted with ethyl acetate (2×10 mL). The combined organic extracts were washed with brine (5 mL) and dried over sodium sulfate. The volatiles were evaporated to dryness under reduced pressure and the residue was purified by prep. TLC using 30% ethyl acetate in hexane as a mobile phase. The required fractions were collected and the volatiles were evaporated to dryness under reduced pressure to give 2-isopentyl-N,N-dimethyl-6-vinylquinazolin-4-amine (35 mg, 0.108 mmol, 84% yield) as a pale yellow solid. ¹H NMR (400 MHz, CD₃OD) δ 8.06 (d, J=1.60 Hz, 1H), 7.90-7.94 (m, 1H), 7.61-7.68 (m, 1H), 6.87-6.94 (m, 1H), 5.89 (d, J=17.60 Hz, 1H), 5.34 (d, J=10.80 Hz, 1H), 3.42 (s, 6H), 2.79-2.83 (m, 2H), 1.72-1.76 (m, 2H), 1.59-1.71 (m, 1H), 1.10 (m, 6H).

### Part C. 4-(Dimethylamino)-2-isopentylquinazoline-6-carbaldehyde

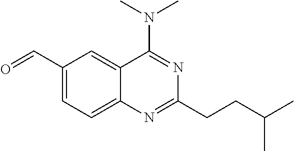 [SMILES: CC(C)CCc1nc(N(C)C)c2cc(C=O)ccc2n1]

To a stirred solution of 2-isopentyl-N,N-dimethyl-6-vinylquinazolin-4-amine (30 mg, 0.11 mmol) and 2,6-lutidine (23 mg, 0.222 mmol) in a dioxane (0.9 mL) and water (0.225 mL) mixture at 0° C. was added sodium metaperiodate (95 mg, 0.445 mmol) followed by the addition of 2.5% solution of osmium tetroxide in t-butanol (0.024 mL, 0.002 mmol). The reaction mixture was then allowed to warm to rt and stirred for 1 h. The completion of reaction was judged by the disappearance of starting material by TLC (20% ethyl acetate in hexane). The reaction mixture was then diluted with satd. aq. sodium bicarbonate (5 mL) solution and extracted with ethyl acetate (2×5 mL). The combined organic extracts were washed with brine (5 mL) and dried over sodium sulfate and the volatiles were evaporated to dryness. The residue was purified by silica gel column chromatography using a gradient of 20% ethyl acetate in hexane as a mobile phase. Required fractions were collected and concentrated under reduced pressure to give 4-(dimethylamino)-2-isopentylquinazoline-6-carbaldehyde (20 mg, 0.073 mmol, 67% yield) as a pale yellow solid. LCMS (ESI) m/e 272.2 [(M+H)⁺, calcd for C₁₆H₂₂N₃O, 272.17]; LC/MS retention time (method F): t_{R}=1.63 min.

### Part D. 2-Isopentyl-N,N-dimethyl-6-(oxazol-5-yl)quinazolin-4-amine

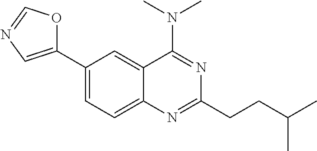 [SMILES: CC(C)CCc1nc(N(C)C)c2cc(-c3cnco3)ccc2n1]

To a stirred solution of 4-(dimethylamino)-2-isopentylquinazoline-6-carbaldehyde (20 mg, 0.074 mmol) in methanol (1.0 mL) at rt was added TosMIC (14 mg, 0.0738 mmol) followed by the addition of K₂CO₃ (11 mg, 0.0811 mmol). The reaction mixture was stirred for 1 h. The volatiles were evaporated to dryness under reduced pressure. To the residue was added 10% satd. aq. sodium bicarbonate solution (5 mL) and the mixture was extracted with ethyl acetate (2×5 mL). The combined organic extracts were washed with brine (5 mL) and dried over sodium sulfate. The volatiles were evaporated to dryness under reduced pressure and the residue so obtained was purified by prep. TLC using 30% ethyl acetate in hexane. The required fractions were collected and the volatiles were evaporated to dryness to give 2-isopentyl-N,N-dimethyl-6-(oxazol-5-yl)quinazolin-4-amine (15 mg, 0.047 mmol, 65% yield) as pale yellow sticky solid. ¹H NMR (400 MHz, CD₃OD) δ 8.47 (d, J=1.6 Hz, 1H), 8.32 (s, 1H), 8.09 (dd, J=8.8, 1.6 Hz, 1H), 7.79 (d, J=8.8 Hz, 1H), 7.66 (s, 1H), 3.49 (s, 6H), 2.82-2.86 (m, 2H), 1.73-1.79 (m, 2H), 1.63-1.69 (m, 1H), 1.00 (d, J=6.8 Hz, 6H); LCMS (ESI) m/e 310.8 [(M+H)⁺, calcd for C₁₈H₂₃N₄O, 311.18]; LC/MS retention time (method G): t_{R}=1.89 min; HPLC retention time (method D): t_{R}=8.27 min; HPLC retention time (method C): t_{R}=6.44 min.

### Example 41

### 5-(2-Isopentyl-4-methoxyquinazolin-6-yl)oxazole

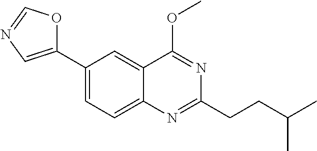 [SMILES: COc1nc(CCC(C)C)nc2ccc(-c3cnco3)cc12]

### Part A. 2-Isopentyl-4-methoxy-6-vinylquinazoline

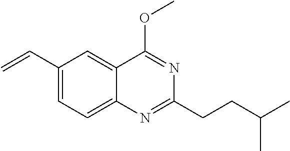 [SMILES: C=Cc1ccc2nc(CCC(C)C)nc(OC)c2c1]

A stirred solution of 6-bromo-2-isopentyl-4-methoxyquinazoline (310 mg, 1.0 mmol) (prepared as described in Example 37, Parts A-E), vinyl boronic anhydride pyridine complex (0.48 g, 2.0 mmol), and sodium carbonate (0.21 g, 2.0 mmol) in toluene (3.5 mL) and EtOH (1.5 mL) was purged with nitrogen for 5 minutes. Pd(PPh₃)₄ (0.13 g, 0.011 mmol) was then added and the reaction mixture was stirred at 95° C. for 3 h.

Water (10 mL) was added and the mixture was extracted with ethyl acetate (2×15 mL). The combined organic extracts were washed with brine solution (20 mL) and the organic layer was dried over sodium sulfate. The volatiles were evaporated to dryness under reduced pressure, and the residue was purified by prep. TLC using 20% ethyl acetate in hexanes as a mobile phase. The required fractions were collected and concentrated under reduced pressure to afford 2-isopentyl-4-methoxy-6-vinylquinazoline (85 mg, 0.33 mmol, 33% yield) as pale oil. LCMS (ESI) m/e 257.2 [(M+H)⁺, calcd for C₁₆H₂₁N₂O, 257.2]; LC/MS retention time (method F): t_{R}=1.85 min.

### Part B. 2-Isopentyl-4-methoxyquinazoline-6-carbaldehyde

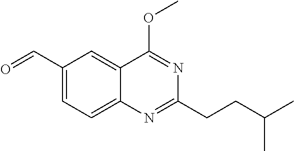 [SMILES: COc1nc(CCC(C)C)nc2ccc(C=O)cc12]

To a solution of 2-isopentyl-4-methoxy-6-vinylquinazoline (85 mg, 0.33 mmol) in 1,4-dioxane (2.5 mL) and water (0.5 mL) at 0° C. was added 2,6-lutidine (0.08 mL, 0.66 mmol), osmium tetroxide (0.070 mL, 0.066 mmol, 2.5% solution in t-butanol), and sodium periodate (0.3 g, 1.32 mmol). The reaction mixture was stirred at room temperature for 3 h. Water (10 mL) was added and the mixture was extracted with ethyl acetate (2×10 mL). The combined organic layers were dried over sodium sulfate and the mixture was concentrated under reduced pressure. The residue so obtained was purified by Prep. TLC using 30% ethyl acetate in hexanes. The required fractions were collected and concentrated under reduced pressure to afford 2-isopentyl-4-methoxyquinazoline-6-carbaldehyde (30 mg, 0.11 mmol 33.3% yield) as pale oil. LCMS (ESI) m/e 259.2 [(M+H)⁺, calcd for C₁₅H₁₉N₂O₂, 259.14]; LC/MS retention time (method F): t_{R}=1.74 min.

### Part C. 5-(2-Isopentyl-4-methoxyquinazolin-6-yl)oxazole

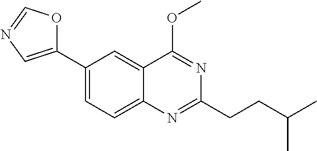 [SMILES: COc1nc(CCC(C)C)nc2ccc(-c3cnco3)cc12]

To a stirred solution of 2-isopentyl-4-methoxyquinazoline-6-carbaldehyde (30 mg, 0.1 mmol) in MeOH (1 mL) was added TosMIC (22 mg, 0.11 mmol) and potassium carbonate (0.5 g, 0.12 mmol) under a nitrogen atmosphere. The reaction mixture was stirred at room temperature for 2 h. The volatiles were evaporated to dryness, and the residue was diluted with aq. sodium bicarbonate solution (10 mL) and extracted with DCM (2×5 mL). The combined organic extracts were dried over sodium sulfate, and the volatiles were evaporated to dryness under reduced pressure to give 5-(2-isopentyl-4-methoxyquinazolin-6-yl)oxazole (15 mg, 0.05 mmol, 50% yield) as an off white solid. ¹H NMR (400 MHz, CDCl₃) δ 8.39 (d, J=2 Hz, 1H), 8.04 (dd, J=8.8, 2 Hz 1H), 7.98 (s, 1H), 7.91 (d, J=8.8 Hz, 1H), 7.48 (s, 1H), 4.21 (s, 3H), 2.95-2.99 (m, 2H), 1.77-1.82 (m, 2H), 1.65-1.75 (m, 1H), 0.99 (d, J=6.8 Hz, 6H); LCMS (ESI) m/e 298.2 [(M+H)⁺, calcd for C₁₇H₂₀N₃O₂, 298.15]; LC/MS retention time (method F): t_{R}=1.72 min; HPLC retention time (method D): t_{R}=9.21 min; HPLC retention time (method C): t_{R}=10.44 min.

### Example 42

### 2-Isopentyl-6-(oxazol-5-yl)quinazolin-4(3H)-one

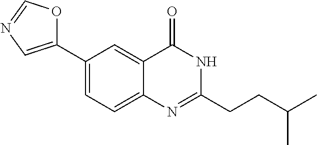 [SMILES: CC(C)CCc1nc2ccc(-c3cnco3)cc2c(=O)[nH]1]

### Part A. 2-Isopentyl-6-vinylquinazolin-4(3H)-one

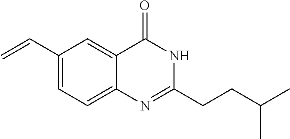 [SMILES: C=Cc1ccc2nc(CCC(C)C)[nH]c(=O)c2c1]

A stirred solution of 6-bromo-2-isopentylquinazolin-4(3H)-one (200 mg, 0.67 mmol) (prepared as described in Example 37, Parts A-D), vinyl boronic anhydride pyridine complex (0.32 g, 1.34 mmol), sodium carbonate (0.28 g, 2.68 mmol) and Pd(PPh₃)₄ (0.146 g, 0.04 mmol) in a mixture of toluene (10 mL), EtOH (2 mL) and water (1 mL) was stirred at 95° C. for 5 h under nitrogen. Water (15 mL) was added and the mixture was extracted with ethyl acetate (2×20 mL). The combined organic extracts were washed with brine solution (20 mL) and the volatiles were evaporated to dryness under reduced pressure. The residue was purified by silica gel column chromatography using a gradient of ethyl acetate in hexane. The required fractions were collected and concentrated to give 2-isopentyl-6-vinylquinazolin-4 (3H)-one (0.18 g, 0.74 mmol, 98.7% yield) as a yellow solid. ¹H NMR (400 MHz, DMSO-d₆) δ 12.19 (s, 1H), 8.08 (s, 1H), 7.94 (dd, J=8.4, 2.0 Hz, 1H), 7.68 (s, 1H), 6.9-6.51 (m, 1H), 5.94 (d, J=8.8 Hz, 1H), 5.35 (d, J=10.8 Hz 1H), 2.59-2.62 (m, 2H), 1.58-1.65 (m, 3H), 0.92 (d, J=6.0 Hz 6H); LCMS ESI) m/e 241.2 [(M−H)⁻, calcd for C₁₅H₁₇N₂O, 241.1]; LC/MS retention time (method F): t_{R}=1.53 min.

### Part B. 2-Isopentyl-4-oxo-3,4-dihydroquinazoline-6-carbaldehyde

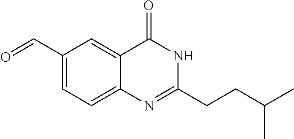 [SMILES: CC(C)CCc1nc2ccc(C=O)cc2c(=O)[nH]1]

To a stirred solution of 2-isopentyl-6-vinylquinazolin-4(3H)-one (180 mg, 0.74 mmol) in 1,4-dioxane (10 mL) and water (2 mL) at 0° C. was added 2,6-lutidine (0.159 g, 1.48 mmol), osmium tetroxide (0.007 mL, 0.022 mmol, 2.5% solution in t-butanol) and sodium periodate (0.63 g, 2.96 mmol). The reaction mixture was stirred at room temperature for 4 h. Water (10 mL) was added and the mixture was extracted with ethyl acetate (2×10 mL). The combined organic layers were dried over sodium sulfate and concentrated under reduced pressure. The residue so obtained was purified by silica gel column chromatography using a gradient of ethyl acetate in hexanes. The required fractions were collected and the volatiles were evaporated to dryness under reduced pressure to give 2-isopentyl-4-oxo-3,4-dihydroquinazoline-6-carbaldehyde (160 mg, 0.65 mmol, 72% yield) as a pale yellow solid. ¹H NMR (400 MHz, DMSO-d₆) δ 12.48 (s, 1H), 10.1 (s, 1H), 8.63 (d, J=2.0 Hz, 1H), 8.19 (dd, J=8.8, 2.0 Hz, 1H), 7.73 (d, J=8.4 Hz, 1H), 2.65 (d, J=7.6 Hz, 2H), 1.59-1.67 (m, 3H), 0.92 (d, J=6.0 Hz, 6H); LCMS (ESI) m/e 245.2 [(M+H)⁺, calcd for C₁₄H₁₇N₂O₂, 245.1]; LC/MS retention time (method B): t_{R}=1.68 min.

### Part C. 2-Isopentyl-6-(oxazol-5-yl)quinazolin-4(3H)-one

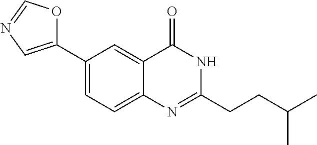 [SMILES: CC(C)CCc1nc2ccc(-c3cnco3)cc2c(=O)[nH]1]

To a stirred solution of 2-isopentyl-4-oxo-3,4-dihydroquinazoline-6-carbaldehyde (100 mg, 0.4 mmol) in MeOH (10 mL) was added TosMIC (0.079 g, 0.4 mmol) and potassium carbonate (0.05 g, 0.4 mmol) and the reaction mixture was stirred at 50° C. for 90 minutes. The volatiles were evaporated to dryness under reduced pressure; water (10 mL) was added and the mixture was extracted with DCM (2×20 mL).

The combined organic extracts were washed with water (10 mL) and brine solution (10 mL), dried over sodium sulfate and concentrated under reduced pressure. The residue obtained was purified by prep. HPLC (10 mM ammonium acetate in water and acetonitrile) to give 2-isopentyl-6-(oxazol-5-yl)quinazolin-4(3H)-one (17 mg, 0.06 mmol, 15% yield) as a white solid. ¹H NMR (400 MHz, CD₃OD) δ 8.52 (d, J=2 Hz, 1H), 8.34 (s, 1H), 8.17 (dd, J=8.8, 2 Hz, 1H), 7.75 (d, J=8.8 Hz, 1H), 7.68 (s, 1H), 2.75-2.71 (m, 2H), 1.74-1.70 (m, 3H), 1.02 (d, J=6.4 Hz, 6H); LCMS (ESI) m/e 284.2 [(M+H)⁺, calcd for C₁₆H₁₈N₃O₂, 284.13]; LC/MS retention time (method B): t_{R}=1.73 min; HPLC retention time (method D): t_{R}=7.29 min; HPLC retention time (method C): t_{R}=8.10 min.

### Example 43

### 5-(2-Isopentyl-4-(methylthio)quinazolin-6-yl)oxazole

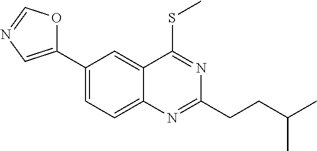 [SMILES: CSc1nc(CCC(C)C)nc2ccc(-c3cnco3)cc12]

### Part A. 6-Bromo-2-isopentylquinazoline-4(3H)-thione

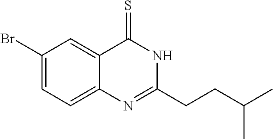 [SMILES: CC(C)CCc1nc2ccc(Br)cc2c(=S)[nH]1]

To a stirred solution of 6-bromo-2-isopentylquinazolin-4(3H)-one (100 mg, 0.34 mmol) (prepared as described in Example 37, Parts A-D) in anhydrous pyridine (1 mL) was added P₂S₅ (100 mg, 0.45 mmol) in the dark and the reaction mixture was refluxed for 1 h. After the completion of the reaction, as monitored by TLC, water (10 mL) was added and the mixture was stirred for 2 h. The volatiles were evaporated to dryness under reduced pressure. Saturated aq. ammonium chloride solution (10 mL) was added and the mixture was extracted with ethyl acetate (2×20 mL). The combined organic extracts were dried over sodium sulfate and concentrated under reduced pressure to afford 6-bromo-2-isopentylquinazoline-4(3H)-thione (90 mg, 0.29 mmol, 85% yield) as a yellow solid. ¹H NMR (400 MHz, DMSO-d₆) δ 13.92 (bs, 1H), 8.64 (d, J=2.4 Hz, 1H), 8.00 (dd, J=8.8, 2.4 Hz, 1H), 7.62 (d, J=8.8 Hz, 1H), 2.74 (m, 2H), 1.63 (m, 3H), 0.92 (d, J=6.4 Hz, 6H).

### Part B. 6-Bromo-2-isopentyl-4-(methylthio)quinazoline

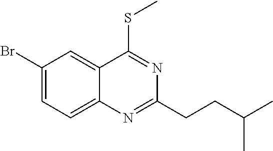 [SMILES: CSc1nc(CCC(C)C)nc2ccc(Br)cc12]

To a stirred solution of 6-bromo-2-isopentylquinazoline-4(3H)-thione (100 mg, 0.323 mmol) in EtOH (1 mL) was added 1N NaOH aq. solution (1 mL) and the reaction mixture was stirred at room temperature for 10 minutes. To this mixture, methyl iodide (0.025 mL, 0.361 mmol) was added slowly and the reaction mixture was stirred at room temperature for 1 h. The reaction mixture was then diluted with brine solution (10 mL) and the pH of the reaction mixture was adjusted to pH 4 using 10% citric acid. The mixture was extracted with ethyl acetate (2×15 mL). The organic extracts were dried over sodium sulfate and concentrated under reduced pressure to afford 6-bromo-2-isopentyl-4-(methylthio)quinazoline (80 mg, 0.246 mmol, 76% yield) as pale oily liquid. ¹H NMR (400 MHz, DMSO-d₆) δ 8.17 (d, J=1.6 Hz, 1H), 8.06-8.04 (m, 1H), 7.82 (d, J=9.2 Hz, 1H), 2.97-2.93 (m, 2H), 2.7 (s, 3H), 1.77-1.71 (m, 2H), 1.65-1.58 (m, 1H), 0.94 (d, J=6.8 Hz, 6H).

### Part C. 2-Isopentyl-4-(methylthio)-6-vinylquinazoline

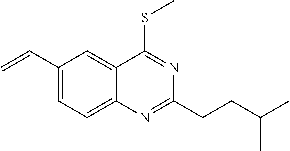 [SMILES: C=Cc1ccc2nc(CCC(C)C)nc(SC)c2c1]

To a stirred solution of 6-bromo-2-isopentyl-4-(methylthio)quinazoline (400 mg, 1.23 mmol) in a mixture of toluene (8 mL) and EtOH (2 mL) was added vinyl boronic anhydride (0.594 g, 2.46 mmol) and sodium carbonate (0.261 g, 2.46 mmol).

The reaction mixture was purged with nitrogen for 5 minutes followed by the addition of Pd(PPh₃)₄ (71 mg, 0.06 mmol). The reaction mixture was stirred at 95° C. for 3 h. The volatiles were evaporated to dryness under reduced pressure. Water (10 mL) was added and the mixture was extracted with ethyl acetate (2×15 mL). The combined organic extracts were washed with brine solution (20 mL) and organic layer was dried over sodium sulfate. The volatiles were evaporated to dryness under reduced pressure and the residue was purified by silica gel column chromatography using a gradient of ethyl acetate in hexanes. The required fractions were collected and concentrated under reduced pressure to give 2-isopentyl-4-(methylthio)-6-vinylquinazoline (190 mg, 0.69 mmol, 56.7% yield) as pale oil. LCMS (ESI) m/e 273.2 [(M+H)⁺, calcd for C₁₆H₂₁N₂S, 273.13]; LC/MS retention time (method F): t_{R}=1.93 min.

### Part D. 2-Isopentyl-4-(methylthio)quinazoline-6-carbaldehyde

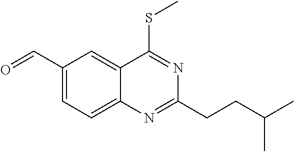 [SMILES: CSc1nc(CCC(C)C)nc2ccc(C=O)cc12]

2-Isopentyl-4-(methylthio)-6-vinylquinazoline (190 mg, 0.69 mmol) in 1,4-dioxane (5 mL) and water (1 mL) at 0° C. was treated with 2,6-lutidine (0.149 g 1.38 mmol), osmium tetroxide (0.16 mL, 0.013 mmol, 2.5% solution in t-butanol) and sodium periodate (0.597 g, 2.76 mmol). The reaction mixture was stirred at room temperature for 4 h. Saturated aq. sodium bicarbonate solution (10 mL) was added and the mixture was extracted with ethyl acetate (2×10 mL). The combined organic layers were dried over sodium sulfate and concentrated under reduced pressure. The residue so obtained was purified by prep. TLC using 30% ethyl acetate in hexanes.

The required fractions were collected and concentrated under reduced pressure to give 2-isopentyl-4-(methylthio)quinazoline-6-carbaldehyde (145 mg, 0.529 mmol, 77% yield) as light yellow solid. LCMS (ESI) m/e 275.2 [(M+H)⁺, calcd for C₁₅H₁₉N₂₀S, 275.11]; LC/MS retention time (method F): t_{R}=1.81 min.

### Part E. 5-(2-Isopentyl-4-(methylthio)quinazolin-6-yl)oxazole

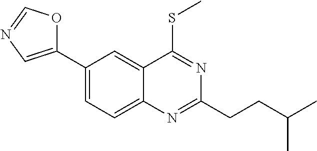 [SMILES: CSc1nc(CCC(C)C)nc2ccc(-c3cnco3)cc12]

To a stirred solution of 2-isopentyl-4-(methylthio)quinazoline-6-carbaldehyde (145 mg, 0.59 mmol) in MeOH (2 mL) was added potassium carbonate (83 mg, 0.58 mmol) and TosMIC (106 mg, 0.59 mmol) and the reaction mixture was heated to reflux for 2 h. The volatiles were evaporated to dryness, and water (10 mL) was added. The mixture was extracted with ethyl acetate (2×15 mL). The combined organic extracts were washed with brine solution (15 mL), and the organic layer was dried over sodium sulfate, filtered and the volatiles were evaporated to dryness under reduced pressure. The crude product was purified by preparative HPLC (10 mM ammonium acetate in water and acetonitrile) to give 5-(2-isopentyl-4-(methylthio)quinazolin-6-yl)oxazole (50 mg, 0.159 mmol, 27% yield) as a white solid. ¹H NMR (400 MHz, CD₃OD) δ 8.45 (d, J=1.6 Hz, 1H), 8.39 (s, 1H), 8.34 (dd, J=8.8, 1.6 Hz, 1H), 7.96 (d, J=8.8 Hz, 1H), 7.61 (s, 1H), 3.07-3.11 (m, 2H), 2.83 (s, 3H), 1.83-1.89 (m, 2H), 1.68-1.74 (m, 1H), 1.03 (d, J=6.4 Hz, 6H); LCMS (ESI) m/e 314.2 [(M+H)⁺, calcd for C₁₇H₂₀N₃OS, 314.12]; LC/MS retention time (method F): t_{R}=1.81 min; HPLC retention time (method H): t_{R}=14.54 min; HPLC retention time (method F): t_{R}=21.94 min.

### Example 44

### N¹-(2-Isopentyl-6-(oxazol-5-yl)quinazolin-4-yl)ethane-1,2-diamine

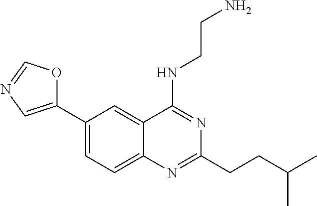 [SMILES: CC(C)CCc1nc(NCCN)c2cc(-c3cnco3)ccc2n1]

### Part A. 5-(4-Chloro-2-isopentylquinazolin-6-yl)oxazole

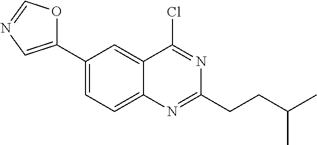 [SMILES: CC(C)CCc1nc(Cl)c2cc(-c3cnco3)ccc2n1]

To a stirred solution of 2-isopentyl-6-(oxazol-5-yl)quinazolin-4(3H)-one (100 mg, 0.35 mmol) (prepared as described in Example 42, Parts A-C) in toluene (0.16 mL) at rt was added POCl₃(0.160 mL, 1.766 mmol) followed by the addition of DIPEA (0.060 mL, 0.353 mmol). The reaction mixture was stirred for 3 h at 110° C. The completion of reaction was judged by the disappearance of starting material by TLC (30% ethyl acetate in hexanes). The reaction mixture was then allowed to cool to rt, and the volatiles were evaporated to dryness under reduced pressure to give 5-(4-chloro-2-isopentylquinazolin-6-yl)oxazole (100 mg, 94% crude yield). The crude product was not stable. It was taken to next step without purification.

### Part B. tert-Butyl 2-(2-isopentyl-6-(oxazol-5-yl)quinazolin-4-ylamino)ethylcarbamate

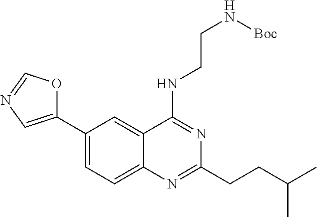 [SMILES: CC(C)CCc1nc(NCCNC(=O)OC(C)(C)C)c2cc(-c3cnco3)ccc2n1]

To a stirred solution of 5-(4-chloro-2-isopentylquinazolin-6-yl)oxazole (100 mg, 0.33 mmol) in THF (4 mL) at rt in a pressure tube was added N-Boc ethylenediamine (106 mg, 0.662 mmol) and DIPEA (0.236 mL, 1.325 mmol). The reaction mixture was stirred for 16 h at 65° C. The completion of reaction was judged by the disappearance of starting material by TLC (60% ethyl acetate in hexanes). The reaction mixture was then allowed to cool to rt, and the volatiles were evaporated to dryness under reduced pressure. The crude product was purified by preparative TLC using 60% ethyl acetate in hexane mobile phase to give tert-butyl 2-(2-isopentyl-6-(oxazol-5-yl)quinazolin-4-ylamino)ethylcarbamate (35 mg, 0.08 mmol, 25% yield). LCMS (ESI) m/e 426.0 [(M+H)⁺, calcd for C₂₃H₃₂N₅O₃, 426.24]; LC/MS retention time (method C): t_{R}=1.94 min.

### Part C. N-(2-Isopentyl-6-(oxazol-5-yl)quinazolin-4-yl)ethane-1,2-diamine

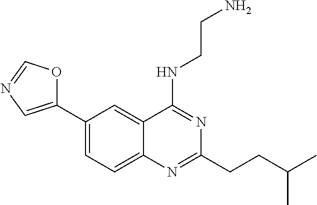 [SMILES: CC(C)CCc1nc(NCCN)c2cc(-c3cnco3)ccc2n1]

To a stirred solution of tert-butyl 2-(2-isopentyl-6-(oxazol-5-yl)quinazolin-4-ylamino)ethylcarbamate (35 mg, 0.082 mmol) in DCM (2 mL) at 0° C. was added TFA (0.031 ml, 0.419 mmol). The reaction mixture was allowed to warm the to rt and stirred for 2 h. The completion of the reaction was judged by the disappearance of starting material by TLC (10% MeOH in DCM). The volatiles were evaporated to dryness under reduced pressure. The residue so obtained was dissolved in water (5 mL) and was washed with ethyl acetate (8 mL). The aqueous layer was cooled to 0° C. and was adjusted to pH 8 with satd. aq. sodium carbonate solution (6 mL). The aqueous layer was extracted with dichloromethane (2×5 mL). The combined organic extracts were washed with brine, dried over sodium sulfate, and the volatiles were evaporated to dryness. The crude product was purified by prep. HPLC (10 mM ammonium acetate in water and acetonitrile) to afford N¹-(2-isopentyl-6-(oxazol-5-yl)quinazolin-4-yl)ethane-1,2-diamine (10 mg, 0.030 mmol, 37% yield) as pale yellow solid. ¹H NMR (400 MHz, CD₃OD) δ 8.52 (d, J=1.6 Hz, 1H), 8.34 (s, 1H), 8.11 (dd, J=4.8, 2 Hz 1H), 7.76 (s, 1H), 7.65 (s, 1H), 3.83 (t, J=6.46 Hz, 2H), 3.09 (t, J=6.0 Hz, 2H), 2.81-2.85 (m, 2H), 1.73-1.78 (m, 2H), 1.63-1.70 (m, 1H), 0.95-1.05 (m, 6H); LCMS (ESI) m/e 324.2 [(M−H)⁻, calcd for C₁₈H₂₂N₅O, 324.19]; LC/MS retention time (method A): t_{R}=1.27 min; HPLC retention time (method A): t_{R}=8.06 min; HPLC retention time (method B): t_{R}=9.17 min.

### Example 45

### 4-Ethyl-2-isopentyl-6-(pyridin-4-yl)quinazoline

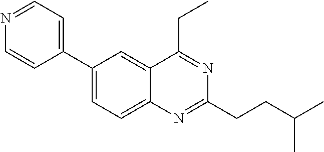 [SMILES: CCc1nc(CCC(C)C)nc2ccc(-c3ccncc3)cc12]

### Part A. N-(4-Bromo-2-cyanophenyl)-4-methylpentanamide

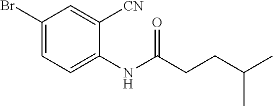 [SMILES: CC(C)CCC(=O)Nc1ccc(Br)cc1C#N]

Prepared in a similar fashion as described in Example 52, Part A, using 2-amino-5-bromobenzonitrile as the starting material to afford N-(4-bromo-2-cyanophenyl)-4-methylpentanamide (540 mg, 1.836 mmol, 36% yield) as pale yellow solid. LCMS (ESI) m/e 295.0 (bromo pattern) [(M+H)⁺, calcd for C₁₃H₁₆BrN₂O, 295.05]; LC/MS retention time (method B): t_{R}=1.81 min.

### Part B. N-(2-Cyano-4-(pyridin-4-yl)phenyl)-4-methylpentanamide

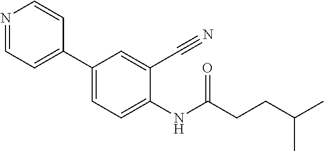 [SMILES: CC(C)CCC(=O)Nc1ccc(-c2ccncc2)cc1C#N]

Prepared in a similar fashion as described in Example 38, using N-(4-bromo-2-cyanophenyl)-4-methylpentanamide to afford N-(2-cyano-4-(pyridin-4-yl)phenyl)-4-methylpentanamide (130 mg, 0.443 mmol, 26% yield) as pale yellow solid. LCMS (ESI) m/e 294.2 [(M+H)⁺, calcd for C₁₈H₂₀N₃O, 294.15]; LC/MS retention time (method B): t_{R}=1.31 min.

### Part C. 4-Ethyl-2-isopentyl-6-(pyridin-4-yl)quinazoline

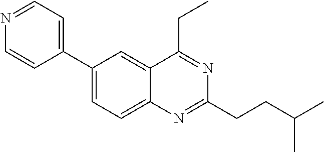 [SMILES: CCc1nc(CCC(C)C)nc2ccc(-c3ccncc3)cc12]

To a stirred solution of ethyl magnesium bromide (2.38 mL, 1M solution in THF, 2.3 mmol) in THF (10 mL) at 0° C. was added a solution of N-(2-cyano-4-(pyridin-4-yl)phenyl)-4-methylpentanamide (100 mg, 0.34 mmol) in THF (5 mL) dropwise under a nitrogen atmosphere. The reaction mixture was warmed to room temperature and was stirred at room temperature for 2 h. The completion of reaction was judged by LCMS. The reaction mixture was quenched with saturated aq. sodium bicarbonate solution (15 mL). The aqueous layer was extracted with ethyl acetate (2×15 mL). The combined organic extracts were washed with brine solution (10 mL), dried over sodium sulfate, and the volatiles were evaporated to dryness under reduced pressure. The product was purified by prep. HPLC (10 mM ammonium acetate in water and acetonitrile) to afford 4-ethyl-2-isopentyl-6-(pyridin-4-yl)quinazoline (6 mg, 6% yield) as a yellow solid. ¹H NMR (400 MHz, CD₃OD) δ 8.69 (d, J=4.8 Hz, 2H), 8.62 (d, J==1.6 Hz, 1H), 8.34 (dd, J==8.8 Hz, 2 Hz, 1H), 8.09 (d, J==8.4 Hz, 1H), 7.91 (m, 2H), 3.32-3.48 (m, 2H), 3.07-3.11 (m, 2H), 1.79-1.85 (m, 2H), 1.67-1.72 (m, 1H), 1.49 (t, J=7.2 Hz, 31H), 0.99-1.03 (m, 6H); LCMS (ESI) m/e 306.2 [(M+H)⁺, calcd for C₂₀H₂₄N₃, 306.19]; LC/MS retention time (method F): t_{R}=1.68 min; HPLC retention time (method E): t_{R}=17.21 min; HPLC retention time (method G): t_{R}=12.49 min.

### Example 46

### 2-Isopentyl-4-isopropyl-6-(pyridin-4-yl)quinazoline

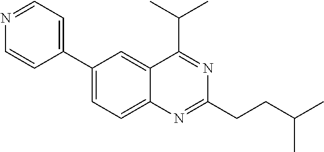 [SMILES: CC(C)CCc1nc(C(C)C)c2cc(-c3ccncc3)ccc2n1]

Prepared in a similar fashion as described in Example 45, Parts A-C, using N-(2-cyano-4-(pyridin-4-yl)phenyl)-4-methylpentanamide (100 mg, 0.34 mmol) and isopropylmagnesium bromide in Part C to afford 2-isopentyl-4-isopropyl-6-(pyridin-4-yl)quinazoline (10.68 mg, 10% yield) as a white solid. ¹H NMR (400 MHz, CD₃OD) δ 8.65-8.7 (m, 3H), 8.33 (dd, J=8.8 Hz, J=2.1 Hz, 1H), 8.08 (d, J=8.8 Hz, 1H), 7.91-7.93 (m, 2H), 4.14-4.21 (m, 1H), 3.08-3.12 (m, 21H), 1.82-1.86 (m, 2H), 1.62-1.72 (m, 1H), 1.47 (d, J=6.8 Hz, 6H), 0.99-1.02 (m, 6H); LCMS (ESI) m/e 320.2 [(M+H)⁺, calcd for C₂₁H₂₆N₃, 320.20]; LC/MS retention time (method F): t_{R}=1.80 min; HPLC retention time (method G): t_{R}=14.53 min; HPLC retention time (method C): t_{R}=8.66 min.

### Example 47

### 4-Cyclopropyl-2-isopentyl-6-(pyridin-4-yl)quinazoline

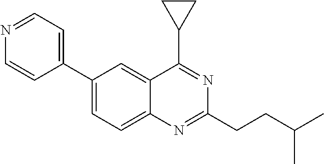 [SMILES: CC(C)CCc1nc(C2CC2)c2cc(-c3ccncc3)ccc2n1]

Prepared in a similar fashion as described previously in Example 45, Parts A-C, using N-(2-cyano-4-(pyridin-4-yl)phenyl)-4-methylpentanamide (100 mg, 0.34 mmol) and cyclopropylmagnesium bromide in Part C to afford 4-cyclopropyl-2-isopentyl-6-(pyridin-4-yl)quinazoline (17.63 mg, 16% yield) as a white solid. ¹H NMR (400 MHz, CD₃OD) δ 8.83 (d, J=2 Hz, 1H), 8.68 (m, 2H), 8.33 (dd, J==8.8 Hz, 2 Hz, 1H), 8.04 (d, J=8.8 Hz, 1H), 7.93-7.95 (m, 2H), 3.15 (m, 1H), 2.99 (m, 2H), 1.77 (m, 2H), 1.59-1.66 (m, 1H), 1.44 (m, 2H), 1.42 (m, 2H), 0.98-1.02 (m, 6H); LCMS (ESI) m/e 318.2 [(M+H)⁺, calcd for C₂₁H₂₄N₃, 318.19]; LC/MS retention time (method B): t_{R}=1.88 min; HPLC retention time (method D): t_{R}=8.40 min; HPLC retention time (method C): t_{R}=7.86 min.

### Example 48

### N-(2-Isopentyl-6-(pyridin-4-yl)quinazolin-4-yl)-N²,N²-dimethylethane-1,2-diamine

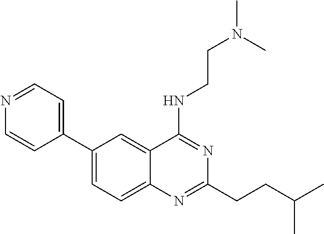 [SMILES: CC(C)CCc1nc(NCCN(C)C)c2cc(-c3ccncc3)ccc2n1]

### Part A. N′-(6-Bromo-2-isopentylquinazolin-4-yl)-N²,N²-dimethylethane-1,2-diamine

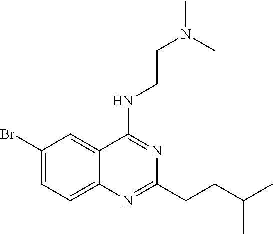 [SMILES: CC(C)CCc1nc(NCCN(C)C)c2cc(Br)ccc2n1]

To a stirred solution of 6-bromo-4-chloro-2-isopentylquinazoline (500 mg, 1.59 mmol) (prepared as described in Example 37, Parts A-E) in acetonitrile (10 mL) in a pressure tube was added DIPEA (1.11 mL, 6.377 mmol) and N,N-dimethylethylene diamine (0562 mg, 6.377 mmol) at rt. The reaction mixture was heated for 16 h at 100° C. The reaction mixture was allowed to cool to rt, and was concentrated under reduced pressure. The residue was diluted with water (25 mL) and the product was extracted with dichloromethane (3×25 mL). The organic layer was washed with H₂O (2×20 mL), dried over sodium sulfate, and concentrated under reduced pressure to give N′-(6-bromo-2-isopentylquinazolin-4-yl)-N²,N²-dimethylethane-1,2-diamine (500 mg, 86% yield). LCMS (ESI) m/e 365.2 (bromo pattern) [(M+H)⁺, calcd for C₁₇H₂₆BrN₄, 365.13]; LC/MS retention time (method A): t_{R}=1.64 min.

### Part B. N′-(2-Isopentyl-6-(pyridin-4-yl)quinazolin-4-yl)-N²,N²-dimethylethane-1,2-diamine

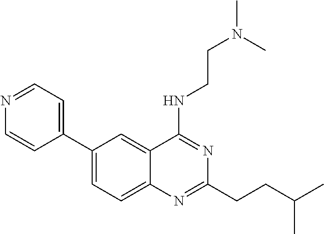 [SMILES: CC(C)CCc1nc(NCCN(C)C)c2cc(-c3ccncc3)ccc2n1]

Prepared in a similar fashion as described in Example 38, using N-(6-bromo-2-isopentylquinazolin-4-yl)-N²,N²-dimethylethane-1,2-diamine (100 mg, 0.27 mmol), to afford N′-(2-isopentyl-6-(pyridin-4-yl)quinazolin-4-yl)-N²,N²-dimethylethane-1,2-diamine (12 mg, 12% yield) as an off-white solid. The product was purified by preparative HPLC (10 mM ammonium acetate in water and acetonitrile). ¹H NMR (400 MHz, CD₃OD) δ 8.65-8.66 (m, 2H), 8.57 (d, J=1.6 Hz, 1H), 8.18 (dd, J=8.8 Hz, 2 Hz, 1H), 7.88-7.89 (m, 2H), 7.81 (d, J=8.8 Hz, 1H), 3.89 (t, J=6.4 Hz, 2H), 2.8-2.86 (m, 4H), 2.43 (s, 6H), 1.75-1.81 (m, 2H), 1.64-1.71 (m, 1H), 1.02 (m, 6H); LCMS (ESI) m/e 364.2 [(M+H)⁺, calcd for C₂₂H₃₀N₅, 364.24]; LC/MS retention time (method A): t_{R}=1.48 min; HPLC retention time (method B): t_{R}=8.29 min; HPLC retention time (method A): t_{R}=6.70 min.

### Example 49

### 2-Isopentyl-6-(pyridin-4-yl)quinazolin-4(3H)-one

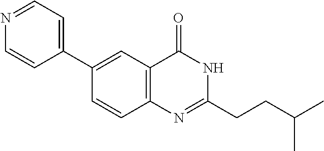 [SMILES: CC(C)CCc1nc2ccc(-c3ccncc3)cc2c(=O)[nH]1]

Prepared in a similar fashion as described in Example 38, using 6-bromo-2-isopentylquinazolin-4(3H)-one (0.1 g, 0.33 mmol) (prepared as described previously in Example 37, Parts A-D) to afford 2-isopentyl-6-(pyridin-4-yl)quinazolin-4(3H)-one (11.6 mg, 0.04 mmol, 12% yield) as a pale yellow solid. ¹H NMR (400 MHz, MeOD) δ 8.68 (m, J=1.60, 4.60 Hz, 2H), 8.45 (d, J=2.40 Hz, 1H), 8.21 (dd, J=2.00, 8.60 Hz, 1H), 7.81 (dd, J=1.60, 4.60 Hz, 2H), 7.73 (d, J=8.40 Hz, 1H), 2.51-2.66 (m, 2H), 1.60-1.68 (m, 3H), 0.94 (s, 6H); LCMS (ESI) m/e 294.2 [(M+H)⁺, calcd for C₁₈H₂₀N₃O, 294.2]; LC/MS retention time (method B): t_{R}=1.35 min; HPLC retention time (method C): t_{R}=5.13 min; HPLC retention time (method D): t_{R}=5.72 min.

### Example 50

### 2-(2-Isopentyl-6-(pyridin-4-yl)quinazolin-4-ylamino)ethanol

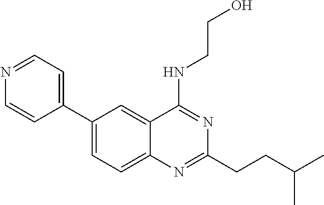 [SMILES: CC(C)CCc1nc(NCCO)c2cc(-c3ccncc3)ccc2n1]

To a solution of 2-isopentyl-6-(pyridin-4-yl)quinazolin-4(3H)-one (500 mg, 1.706 mmol) (prepared as described in Example 49) in toluene (10 mL) was added POCl₃ (0.8 mL, 8.532 mmol) and DIPEA (0.3 mL, 1.706 mmol). The reaction mixture was then refluxed at 110° C. for 2 h. The reaction progress was monitored through TLC, which showed complete consumption of starting materials. The reaction mixture was cooled to ambient temperature and concentrated under reduced pressure. The product was not stable and was taken to next step without further purification.

To a solution of 4-chloro-2-isopentyl-6-(pyridin-4-yl)quinazoline (200 mg, 0.644 mmol) in acetonitrile (2 mL) was added ethanolamine (156 mg, 2.564 mmol) and DIPEA (0.44 mL, 2.564 mmol). The mixture was heated at 100° C. overnight in a pressure tube. The reaction progress was monitored through LCMS, which showed complete consumption of starting materials. The reaction mixture was concentrated and the residue was transferred to a separatory funnel containing brine solution (20 mL). The aqueous layer was extracted with dichloromethane (3×10 mL). The combined organic layers were separated, dried over sodium sulfate, and concentrated under reduced pressure. The crude product was purified by preparative silica-gel TLC using 10% methanol-dichloromethane as mobile phase. It was then recrystallized from ethyl acetate-hexane mixture to give 2-(2-isopentyl-6-(pyridin-4-yl) quinazolin-4-ylamino)ethanol (30 mg, 14% yield) as white solid. ¹H NMR (400 MHz, DMSO-d₆) δ 8.75 (s, 1H), 8.69-8.75 (m, 2H), 8.43 (d, J=4.4 Hz, 1H), 8.17 (dd, J=8.8, 2.0 Hz, 1H), 7.87-7.88 (m, 2H), 7.72 (d, J=8.8 Hz, 1H), 4.85 (t, J=5.2 Hz, 1H), 3.65-3.69 (m, 4H), 2.70-2.74 (m, 2H), 1.59-1.71 (m, 3H), 0.94 (d, J=6.4 Hz, 6H); LCMS (ESI) m/e 337.2 [(M+H)⁺, calcd for C₂₀H₂₅N₄O, 337.2]; LC/MS retention time (method C): t_{R}=1.67 min; HPLC retention time (method B): t_{R}=9.68 min; HPLC retention time (method A): t_{R}=8.44 min.

### Example 51

### 2-Isopentyl-6-(1H-pyrazol-4-yl)quinazolin-4(3H)-one

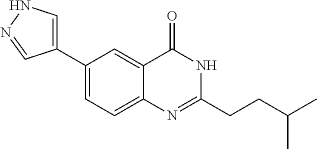 [SMILES: CC(C)CCc1nc2ccc(-c3cn[nH]c3)cc2c(=O)[nH]1]

To a solution of 6-bromo-2-isopentylquinazolin-4(3H)-one (100 mg, 0.338 mmol) (prepared as described in Example 37, Parts A-D) in dioxane (3 mL) and water (1 mL) was added pyrazole boronic acid (143 mg, 0.677 mmol) and Cs₂CO₃ (441 mg, 1.355 mmol). Nitrogen gas was bubbled through the reaction mixture and Pd(PPh₃)₄ (19 mg, 0.016 mmol) was added. The reaction mixture was heated at 90° C. overnight. The reaction mixture was cooled to ambient temperature and was diluted with 10 mL of water and extracted with ethyl acetate (3×10 mL). The organic layer was separated, dried over sodium sulfate and concentrated under reduced pressure.

The crude product was recrystallized with dichloromethane and hexane to give 2-isopentyl-6-(1H-pyrazol-4-yl) quinazolin-4(3H)-one (55 mg, 53% yield) as brownish solid. ¹H NMR (400 MHz, DMSO-d₆) δ 13.03 (bs, 1H), 12.14 (bs, 1H), 8.35 (s, 1H), 8.24 (d, J=2 Hz, 1H), 8.03 (m, 2H), 7.58 (d, J=8.4 Hz, 1H), 2.60 (t, J=7.2 Hz, 2H), 1.61-1.65 (m, 3H), 0.92 (m, 6H); LCMS (ESI) m/e 283.2 [(M+H)⁺, calcd for C₁₆H₁₉N₄O, 283.15]; LC/MS retention time (method C): t_{R}=1.61 min; HPLC retention time (method C): t_{R}=6.38 min; HPLC retention time (method D): t_{R}=6.17 min.

### Example 52

### 5-(4-Ethyl-2-isopentyl-7-methoxyquinazolin-6-yl)oxazole

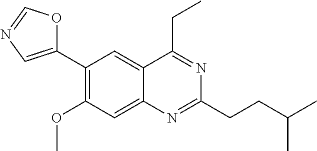 [SMILES: CCc1nc(CCC(C)C)nc2cc(OC)c(-c3cnco3)cc12]

### Part A. N-(4-Bromo-5-methoxy-2-propionylphenyl)-4-methylpentanamide

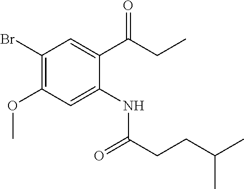 [SMILES: CCC(=O)c1cc(Br)c(OC)cc1NC(=O)CCC(C)C]

To a solution of 1-(2-amino-5-bromo-4-methoxyphenyl) propan-1-one (2.7 g, 0.010 mmol) (prepared as described in Example 85, Parts A-B) in dichloroethane (14 mL) was added DMAP (0.161 g, 1.319 mmol) followed by 4-methylpentanoyl chloride (2.8 mL, 0.020 mmol). The mixture was stirred for 10 min. DIPEA (0.3 mL, 2.325 mmol) was added and the mixture was heated at 45° C. overnight. The reaction mixture was diluted with saturated aq. NaHCO₃ solution (50 mL) and dichloromethane (100 mL). The organic layer was separated, dried over sodium sulfate, and concentrated under reduced pressure to afford N-(4-bromo-5-methoxy-2-propionylphenyl)-4-methylpentanamide (2.9 g, 45% yield) as light yellow solid.

LCMS (ESI) m/e 358.0 (bromo pattern) [(M+H)⁺, calcd for C₁₆H₂₃BrNO₃, 356.08]; LC/MS retention time (method A): t=2.53 min.

### Part B. 6-Bromo-4-ethyl-2-isopentyl-7-methoxyquinazoline

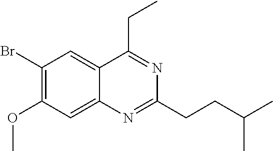 [SMILES: CCc1nc(CCC(C)C)nc2cc(OC)c(Br)cc12]

To a solution of N-(4-bromo-5-methoxy-2-propionylphenyl)-4-methylpentanamide (2.9 g, 8.169 mmol) in acetic acid (90 mL) was added ammonium acetate (13.5 g, 0.221 mol). The mixture was heated at reflux overnight. The reaction mixture was concentrated under reduced pressure and the residue was diluted with water (100 mL) and ethyl acetate (100 mL). The organic layer was separated, dried over sodium sulfate and concentrated under reduced pressure to afford 6-bromo-4-ethyl-2-isopentyl-7-methoxyquinazoline (1.85 g, 64% yield) as an off-white solid. ¹H NMR (400 MHz, CDCl₃) 8.26 (s, 1H), 7.30 (s, 1H), 4.04 (s, 3H), 3.14-3.20 (m, 2H), 2.99-3.04 (m, 2H), 1.65-1.81 (m, 3H), 1.35-1.45 (m, 3H), 0.9-1.0 (m, 6H).

### Part C. 4-Ethyl-2-isopentyl-7-methoxy-6-vinylquinazoline

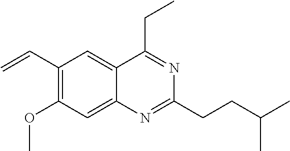 [SMILES: C=Cc1cc2c(CC)nc(CCC(C)C)nc2cc1OC]

To a solution of 6-bromo-4-ethyl-2-isopentyl-7-methoxyquinazoline (1.85 g, 5.022 mmol) in toluene (40 mL) and water (9 mL) was added vinyl boronic acid (2.65 g, 11.01 mmol), Na₂CO₃ (1.17 g, 11.03 mmol), and Pd(PPh₃)₄ (0.318 g, 0275 mmol).

The reaction mixture was heated at 90° C. overnight. The reaction mixture was concentrated under reduced pressure and diluted with water (50 mL) and ethyl acetate (80 mL). The organic layer was separated, dried over sodium sulfate, and concentrated under reduced pressure to afford 4-ethyl-2-isopentyl-7-methoxy-6-vinylquinazoline (1.28 g, 65% yield) as solid. LCMS (ESI) m/e 285.2 [(M+H)⁺, calcd for C₁₈H₂₅N₂O, 285.2]; LC/MS retention time (method C): t_{R}=2.21 min.

### Part D. 4-Ethyl-2-isopentyl-7-methoxyquinazoline-6-carbaldehyde

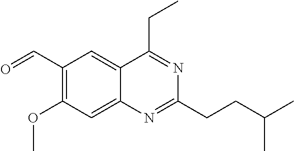 [SMILES: CCc1nc(CCC(C)C)nc2cc(OC)c(C=O)cc12]

To a solution of 4-ethyl-2-isopentyl-7-methoxy-6-vinylquinazoline (1.28 g, 4.507 mmol) in dioxane (38 mL) and water (9.6 mL) at 0° C. was added lutidine (1.07 g, 9.985 mmol) and OsO₄ (0.99 mL, 3.894 mmol). The mixture was stirred for 10 min. NaIO₄ (3.85 g, 18.0 mmol) was then added to the reaction mixture. The reaction mixture was then warmed to rt and was stirred for 3 h. The reaction mixture was diluted with saturated aq. NaHCO₃ solution (25 mL) and extracted with ethyl acetate (3×25 mL). The organic layer was separated, dried over sodium sulfate, and concentrated under reduced pressure to afford 4-ethyl-2-isopentyl-7-methoxyquinazoline-6-carbaldehyde (850 mg, 85% yield) as white solid. LCMS (ESI) m/e 287.0 [(M+H)⁺, calcd for C₁₇H₂₃N₂O₂, 287.17]; LC/MS retention time (method C): t_{R}=2.05 min.

### Part E. 5-(4-Ethyl-2-isopentyl-7-methoxyquinazolin-6-yl)oxazole

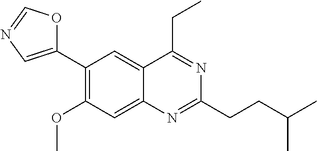 [SMILES: CCc1nc(CCC(C)C)nc2cc(OC)c(-c3cnco3)cc12]

To a solution of 4-ethyl-2-isopentyl-7-methoxyquinazoline-6-carbaldehyde (850 mg, 2.972 mmol) in MeOH (10 ml) was added K₂CO₃ (492 mg, 3.056 mmol) followed by TosMIC (0.58 g, 2.972 mmol). The mixture was heated to reflux for 3 h. MeOH was removed under reduced pressure. The reaction was diluted with saturated aq.

NaHCO₃ solution (25 mL) and extracted with ethyl acetate (3×25 mL). The organic layer was separated, dried over sodium sulfate, and concentrated under reduced pressure to afford 5-(4-ethyl-2-isopentyl-7-methoxyquinazolin-6-yl) oxazole (800 mg, 83% yield) as pale yellow solid. ¹H NMR (400 MHz, MeOD) δ 8.49 (s, 1H), 8.36 (s, 1H), 7.70 (s, 1H), 7.37 (s, 1H), 4.16 (s, 3H), 3.33 (m, 2H), 3.01 (m, 2H), 1.78 (m, 2H), 1.66 (m, 1H), 1.45 (t, J=7.60 Hz, 3H), 1.00 (m, 6H); LCMS (ESI) m/e 326.2 [(M+H)⁺, calcd for C₁₉H₂₄N₃O₂, 326.18]; LC/MS retention time (method C): t_{R}=2.04 min; HPLC retention time (method G): t_{R}=12.55 min; HPLC retention time (method E): t_{R}=13.54 min.

### Example 53

### 2-Isopentyl-6-(pyridin-4-yl)quinazoline-4-carboxamide

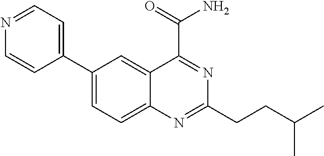 [SMILES: CC(C)CCc1nc(C(N)=O)c2cc(-c3ccncc3)ccc2n1]

### Part A. 2-(5-Bromo-2-(4-methylpentanamido)phenyl)-2-oxoacetic acid

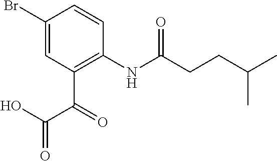 [SMILES: CC(C)CCC(=O)Nc1ccc(Br)cc1C(=O)C(=O)O]

To a solution of 5-bromoindoline-2,3-dione (2.0 g, 8.848 mmol) in DCM (20 mL) was added DIPEA (7.7 mL, 44.28 mmol) followed by 4-methylpentyl chloride (2.37 mL, 17.686 mmol). The mixture was allowed to stir at RT overnight. To the reaction mixture was added water (50 mL) followed by ethyl acetate (80 mL). The organic layer was separated, dried over sodium sulfate, and concentrated under reduced pressure to afford 2-(5-bromo-2-(4-methylpentanamido)phenyl)-2-oxoacetic acid (3.0 g, 72% yield). LCMS (ESI) m/e 342.0 [(M+H)⁺ calcd for C₁₄H₁₇BrNO₄, 342.0]; LC/MS retention time (method A): t_{R} 1.34 min.

### Part B. 6-Bromo-2-isopentylquinazoline-4-carboxamide

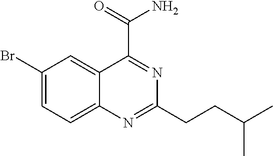 [SMILES: CC(C)CCc1nc(C(N)=O)c2cc(Br)ccc2n1]

To a solution of 2-(5-bromo-2-(4-methylpentanamido)phenyl)-2-oxoacetic acid (2.0 g, 5.84 mmol) in ethanol (20 mL) at −78° C. was slowly added ethanolic ammonia (15 mL). The reaction mixture was warmed to rt. The reaction mixture was then heated in an autoclave at 100° C. overnight. The reaction mixture was cooled to rt and concentrated under reduced pressure. The reaction mixture was diluted with water (20 mL) and ethyl acetate (50 mL). The organic layer was separated, dried over sodium sulfate, and concentrated under reduced pressure to afford 6-bromo-2-isopentylquinazoline-4-carboxylic acid (200 mg) and 6-bromo-2-isopentylquinazoline-4-carboxamide (500 mg, 27% yield). 6-Bromo-2-isopentylquinazoline-4-carboxylic acid: LCMS (ESI) m/e 323.0, [(M+H) calcd for C₁₄H₁₆BrN₂O₂, 323.0]; LC/MS retention time (method A): t_{R}=1.24 min. 6-Bromo-2-isopentylquinazoline-4-carboxamide: LCMS (ESI) m/e 322.0 [(M+H)⁺ calcd for C₁₄H₁₇BrN₃O, 322.1]; LC/MS retention time (method A): t_{R}=1.24 min.

### Part C. 2-Isopentyl-6-(pyridin-4-yl)quinazoline-4-carboxamide

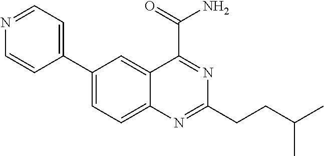 [SMILES: CC(C)CCc1nc(C(N)=O)c2cc(-c3ccncc3)ccc2n1]

To a solution of 6-bromo-2-isopentylquinazoline-4-carboxamide (0.2 g, 0.621 mmol) in 1, 4-dioxane (4 mL) and water (2 mL) was added pyridine 4-boronic acid (0.115 g, 0.934 mmol), Cs₂CO₃ (0.607 g, 1.861 mmol) and Pd(PPh₃)₄ (0.07 g, 0.060 mmol).

The reaction mixture was heated at 90° C. overnight. The reaction mixture was concentrated under reduced pressure and diluted with water (50 mL) and ethyl acetate (80 mL). The organic layer was separated, dried over sodium sulfate, and concentrated under reduced pressure to afford 2-isopentyl-6-(pyridin-4-yl) quinazoline-4-carboxamide (120 mg, 61% yield) as white solid. ¹H NMR (400 MHz, CD₃OD) δ 9.4 (d, J=2 Hz, 1H), 8.69-8.71 (m, 2H), 8.41 (dd, J=8.8 Hz, 2 Hz, 1H), 8.17 (d, J=8.8 Hz, 1H), 7.89 (m, 2H), 3.18-3.22 (m, 2H), 1.85-1.91 (m, 2H), 1.68-1.75 (m, 1H), 1.04 (d, J=6.4 Hz, 6H); LCMS (ESI) m/e 319.2 [(M−H)⁻, calcd for C₁₉H₁₉N₄O, 319.16]; LC/MS retention time (method A): t_{R}=1.78 min; HPLC retention time (method C): t_{R}=6.21 min; HPLC retention time (method D): t_{R}=6.75 min.

### Example 54

### 5-(7-Ethoxy-4-ethyl-2-isopentylquinazolin-6-yl) oxazole

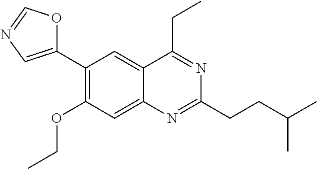 [SMILES: CCOc1cc2nc(CCC(C)C)nc(CC)c2cc1-c1cnco1]

### Part A. 4-Ethyl-2-isopentyl-6-(oxazol-5-yl)quinazolin-7-ol

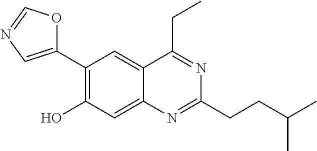 [SMILES: CCc1nc(CCC(C)C)nc2cc(O)c(-c3cnco3)cc12]

In a pressure tube, a solution of 5-(4-ethyl-2-isopentyl-7-methoxyquinazolin-6-yl) oxazole (800 mg, 2.461 mmol) (prepared as described in Example 52, Parts A-E) in DCM (8 mL) was cooled at 0° C. To this solution was added BBr₃ (0.5 mL, 2.024 mmol) and the mixture was heated at 80° C. for 2 h. The reaction mixture was diluted with satd. aq. NaHCO₃ solution (50 mL) and was extracted with dichloromethane (3×25 mL). The combined organic layers were separated, dried over sodium sulfate, and concentrated under reduced pressure to afford 4-ethyl-2-isopentyl-6-(oxazol-5-yl)quinazolin-7-ol (350 mg, 45% yield) as brown solid. LCMS (ESI) m/e 312.0 [(M+H)⁺, calcd for C₁₈H₂₂N₃O₂, 312.16]; LC/MS retention time (method F): t_{R}=1.55 min.

### Part B. 5-(7-Ethoxy-4-ethyl-2-isopentylquinazolin-6-yl)oxazole

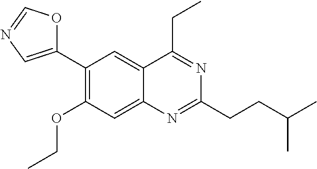 [SMILES: CCOc1cc2nc(CCC(C)C)nc(CC)c2cc1-c1cnco1]

To a suspension of NaH (0.01 mg, 0.416 mmol) in THF (1 mL) at 0° C. was added a solution of 4-ethyl-2-isopentyl-6-(oxazol-5-yl)quinazolin-7-ol (40 mg, 0.128 mmol) in THF (5 mL). The reaction mixture was stirred for 10 min and then warmed to rt and stirred for 2 h. To this mixture, ethyl iodide (0.012 ml, 0.076 mmol) was added dropwise and the mixture was stirred at ambient temperature for 16 h. The reaction mixture was quenched with water (20 mL) and extracted with dichloromethane (3×20 mL). The combined organic layers were separated, dried over sodium sulfate, and concentrated under reduced pressure. The residue was purified by prep HPLC (10 mM ammonium acetate in acetonitrile) to afford 5-(7-ethoxy-4-ethyl-2-isopentylquinazolin-6-yl)oxazole (10 mg, 28% yield) as grey solid. ¹H NMR (400 MHz, CD₃OD) δ 8.54 (s, 1H), 8.38 (s, 1H), 7.73 (s, 1H), 7.37 (s, 1H), 4.39-4.44 (m, 2H), 3.28-3.33 (m, 2H), 3.0 (t, J=8 Hz, 2H), 1.76-1.82 (m, 2H), 1.63-1.72 (m, 4H), 1.45 (t, J=7.6 Hz, 3H), 1.02 (d, J=6.4 Hz, 6H); LCMS (ESI) m/e 340.2 [(M+H)⁺, calcd for C₂₀H₂₆N₃O₂, 340.19]; LC/MS retention time (method F): t_{R}=1.76 min; HPLC retention time (method G): t_{R}=13.76 min; HPLC retention time (method E): t_{R}=13.32 min.

### Example 55

### 1-(4-Methoxy-6-(pyridin-4-yl)quinazolin-2-yl)-3-methylbutan-1-amine

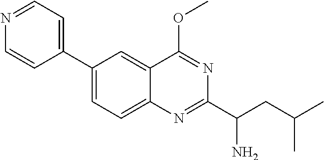 [SMILES: COc1nc(C(N)CC(C)C)nc2ccc(-c3ccncc3)cc12]

### Part A. tert-Butyl 1-(6-bromo-4-oxo-3,4-dihydroquinazolin-2-yl)-3-methylbutylcarbamate

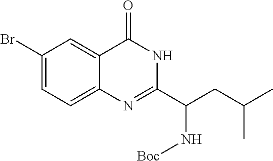 [SMILES: CC(C)CC(NC(=O)OC(C)(C)C)c1nc2ccc(Br)cc2c(=O)[nH]1]

Prepared in a similar fashion as described in Example 37, Parts A-D, 2-((tert-butoxycarbonyl)amino)-4-methylpentanoic acid in Part C to give tert-butyl 1-(6-bromo-4-oxo-3,4-dihydroquinazolin-2-yl)-3-methylbutylcarbamate (2.5 g). The crude product was taken to next step without further purification. LCMS (ESI) m/e 410.2 [(M+H)⁺, calcd for C₁₈H₂₅BrN₃O₃, 410.10]; LC/MS retention time (method D): t_{R}=0.79 min.

### Part B. tert-Butyl 3-methyl-1-(4-oxo-6-(pyridin-4-yl)-3,4-dihydroquinazolin-2-yl)butylcarbamate

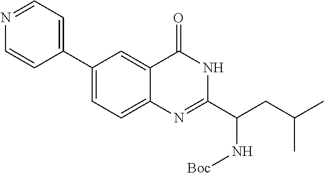 [SMILES: CC(C)CC(NC(=O)OC(C)(C)C)c1nc2ccc(-c3ccncc3)cc2c(=O)[nH]1]

Prepared in a similar fashion as described in Example 38, using tert-butyl 1-(6-bromo-4-oxo-3,4-dihydroquinazolin-2-yl)-3-methylbutylcarbamate (1.0 g, 2.439 mmol) to give tert-butyl 3-methyl-1-(4-oxo-6-(pyridin-4-yl)-3,4-dihydroquinazolin-2-yl)butylcarbamate (0.8 g, 80% yield). The crude product was purified by silica gel column chromatography using a gradient of ethyl acetate in hexanes as a mobile phase. LCMS (ESI) m/e 409.2 [(M+H)⁺, calcd for C₂₃H₂₉N₄O₃, 409.22]; LC/MS retention time (method A): t_{R}=1.76 min.

### Part C. tert-Butyl 1-(4-methoxy-6-(pyridin-4-yl) quinazolin-2-yl)-3-methylbutylcarbamate

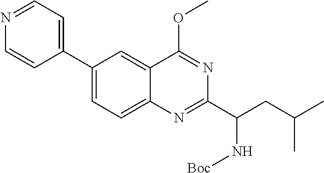 [SMILES: COc1nc(C(CC(C)C)NC(=O)OC(C)(C)C)nc2ccc(-c3ccncc3)cc12]

To a stirred solution of tert-butyl 3-methyl-1-(4-oxo-6-(pyridin-4-yl)-3,4-dihydroquinazolin-2-yl)butylcarbamate (20 mg, 0.049 mmol) in MeOH (0.5 mL), was added BOP (44 mg, 0.099 mmol) and DBU (0.095 mL, 0.625 mmol) at room temperature. It was stirred for 20 min, cooled to 0° C. and NaOMe (5 mg, 0.092 mmol) was added. The reaction was quenched with ice cold water and diluted with ethyl acetate (30 mL). The organic layer was collected, dried over Na₂SO₄ and concentrated to give tert-butyl 1-(4-methoxy-6-(pyridin-4-yl) quinazolin-2-yl)-3-methylbutylcarbamate (20 mg, 97% yield). LCMS (ESI) m/e 423.2 [(M+H)⁺ calcd for C₂₄H₃₁N₄O₃, 423.2]; LC/MS retention time (method B): t_{R}=1.89 min.

### Part D. 1-(4-Methoxy-6-(pyridin-4-yl) quinazolin-2-yl)-3-methylbutan-1-amine

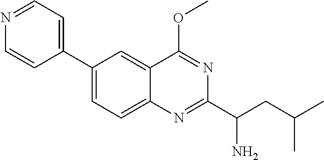 [SMILES: COc1nc(C(N)CC(C)C)nc2ccc(-c3ccncc3)cc12]

To a stirred solution of tert-butyl 3-methyl-1-(4-oxo-6-(pyridin-4-yl)-3,4-dihydroquinazolin-2-yl)butylcarbamate (50 mg, 0.118 mmol) in dichloromethane (2 mL) at 0° C., was added trifluoroacetic acid (0.14 mL, 1.228 mmol). The mixture was then stirred for 2 h at room temperature. The mixture was concentrated and the residue purified by reverse phase HPLC to give 1-(4-methoxy-6-(pyridin-4-yl) quinazolin-2-yl)-3-methylbutan-1-amine (20 mg, 100%) as a brown solid. ¹H NMR (400 MHz, CD₃OD) δ 8.66-8.68 (m, 2H), 8.55 (d, J=1.6 Hz, 1H), 8.30 (dd, J=8.8 Hz, 2 Hz, 1H), 8.04 (d, J=8.8 Hz, 1H), 7.85 (d, J=6.4 Hz, 2H), 4.3 (s, 3H), 4.18-4.21 (m, 1H), 1.9-1.97 (m, 1H), 1.69-1.75 (m, 2H), 1.05 (d, J=6.4 Hz, 3H), 0.99 (d, J=6.4 Hz, 3H); LCMS (ESI) m/e 323.2 [(M+H)⁺, calcd for C₁₉H₂₃N₄O, 323.2]; LC/MS retention time (method B): t_{R}=1.18 min; HPLC retention time (method B): t_{R}=8.59 min; HPLC retention time (method A): t_{R}=7.49 min; Chiral HPLC retention time (method A): t_{R}=11.91, 16.68 min.

### Example 56

### 2-Isopentyl-6-(oxazol-5-yl)quinazoline-4(3H)-thione

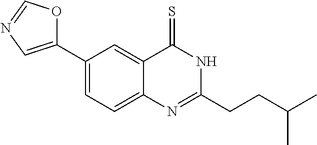 [SMILES: CC(C)CCc1nc2ccc(-c3cnco3)cc2c(=S)[nH]1]

To a solution of 2-isopentyl-6-(oxazol-5-yl)quinazolin-4(3H)-one (100 mg, 0.352 mmol) (prepared as described in Example 42, parts A-C) in pyridine (2 mL) at 115° C. in the dark, was added P₂S₅(104 mg, 0.469 mmol). The mixture was heated at reflux for 1 h. The reaction mixture was cooled to RT and 0.5 mL of water was added. The mixture was stirred for 2 h and then 25 mL of aq. ammonium chloride was added followed by 10 mL of ethyl acetate. The organic layer was separated, dried over sodium sulfate, and concentrated under reduced pressure. The crude product was purified by preparative HPLC to give 2-isopentyl-6-(oxazol-5-yl)quinazoline-4(3H)-thione (18 mg, 17% yield). ¹H NMR (400 MHz, DMSO-d₆) δ 13.87 (s, 1H), 8.79 (d, J=2.4 Hz, 1H), 8.54 (s, 1H), 8.21-8.24 (m, 1H), 7.88 (s, 1H), 7.76 (d, J=8.4 Hz, 1H), 2.74-2.79 (m, 2H), 1.63-1.67 (m, 3H), 0.94 (d, J=6.4 Hz, 6H); LCMS (ESI) m/e 298.0 [(M−H)⁻, calcd for C₁₆H₁₆N₃OS, 298.11]; LC/MS retention time (method F): t_{R}=1.62 min; HPLC retention time (method C): t_{R}=10.69 min; HPLC retention time (method D): t_{R}=9.46 min.

### Example 57

### 5-(4-Ethyl-2-isopentylquinazolin-6-yl)oxazole

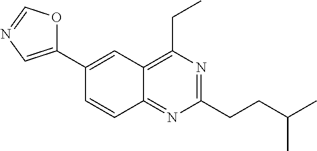 [SMILES: CCc1nc(CCC(C)C)nc2ccc(-c3cnco3)cc12]

Prepared in a similar fashion as described in Example 52, Parts A-E, using 1-(2-amino-5-bromo)propan-1-one in Part A to afford 5-(4-ethyl-2-isopentylquinazolin-6-yl)oxazole (280 mg) as a pale yellow solid. The crude product was purified by silica gel column chromatography using the gradient of ethyl acetate in pet ether as a mobile phase. ¹H NMR (400 MHz, CD₃OD) δ 8.56 (d, J=2 Hz, 1H), 8.38 (s, 1H), 8.30 (dd, J: 8.8 Hz, 2 Hz, 1H), 8.03 (d, J: 8.8 Hz, 1H), 7.8 (s, 1H), 3.38-3.44 (m, 2H), 3.06-3.1 (m, 2H), 1.78-1.84 (m, 2H), 1.65-1.73 (m, 1H), 1.48 (t, J=7.6 Hz, 3H), 1.00-1.11 (m, 6H); LCMS (ESI) m/e 296.2 [(M+H)⁺, calcd for C₁₈H₂₂N₃O, 296.2]; LC/MS retention time (method F): t_{R}=1.68 min; HPLC retention time (method E): t_{R}=15.73 min; HPLC retention time (method G): t_{R}=12.39 min.

### Example 58

### 2-Isopentyl-7-methoxy-6-(oxazol-5-yl)quinazolin-4(3H)-one

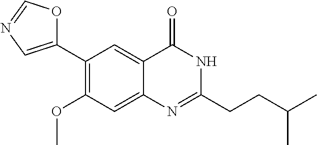 [SMILES: COc1cc2nc(CCC(C)C)[nH]c(=O)c2cc1-c1cnco1]

### Part A. N-(4-Bromo-2-cyano-5-methoxyphenyl)-4-methylpentanamide

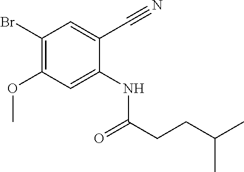 [SMILES: COc1cc(NC(=O)CCC(C)C)c(C#N)cc1Br]

To a solution of 2-amino-5-bromo-4-methoxybenzonitrile (3.0 g, 0.013 mol) (prepared as described in Example 85, Part A) in dichloromethane (60 mL) was added DIPEA (2.76 mL, 0.0158 mmol) followed by 4-methylpentanoyl chloride (1.95 g, 0.0145 mmol). The reaction mixture was stirred at rt for 2 h. The reaction mass was diluted with water (100 mL) and extracted with dichloromethane (3×25 mL). The organic layer was separated, dried over sodium sulfate, and concentrated under reduced pressure. The crude product was purified by silica-gel column chromatography using a gradient of hexanes and ethyl acetate as mobile phase to give N-(4-bromo-2-cyano-5-methoxyphenyl)-4-methylpentanamide (1.8 g, 42% yield). LCMS (ESI) m/e 325.0 (bromo pattern) [(M+H)⁺, calcd for C₁₄H₁₈BrN₂O₂, 325.05]; LC/MS retention time (method G): t_{R}=1.85 min.

### Part B. 6-Bromo-2-isopentyl-7-methoxyquinazolin-4(3H)-one

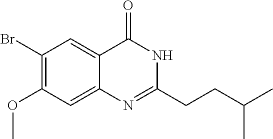 [SMILES: COc1cc2nc(CCC(C)C)[nH]c(=O)c2cc1Br]

To a solution of N-(4-bromo-2-cyano-5-methoxyphenyl)-4-methylpentanamide (1.77 g, 5.44 mmol) in ethanol (17.7 mL) and water (3.5 mL) was added sodium hydroxide (0.37 g, 9.252 mmol) followed by 30% hydrogen peroxide solution (4.31 mL, 38.10 mmol). The reaction mixture was heated at 50° C. for 1 h. After cooling, the solution was acidified with 1.5N hydrochloric acid solution (aq). The precipitate that formed was filtered, washed with water and dried under vacuum to give 6-bromo-2-isopentyl-7-methoxyquinazolin-4(3H)-one (900 mg, 51% yield). LCMS (ESI) m/e 325.0 (bromo pattern) [(M+H)⁺, calcd for C₁₄H₁₈BrN₂O₂, 325.05]; LC/MS retention time (method A): t_{R}=1.80 min.

### Part C. 2-Isopentyl-7-methoxy-6-vinylquinazolin-4(3H)-one

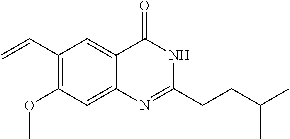 [SMILES: C=Cc1cc2c(=O)[nH]c(CCC(C)C)nc2cc1OC]

To a solution of 6-bromo-2-isopentyl-7-methoxyquinazolin-4(3H)-one (700 mg, 2.15 mmol) in toluene (14 mL), water (2 mL) and ethanol (3.5 mL) was added vinyl boronic acid (1.03 g, 4.305 mmol), Na₂CO₃ (456 mg, 4.305 mmol), and Pd(PPh₃)₄ (124 mg, 0.107 mmol). The reaction mixture was stirred for 10 min and was then heated at 90° C. overnight. The reaction mixture was concentrated under reduced pressure and diluted with water (50 mL) and extracted with ethyl acetate (2×20 mL).

The organic layer was separated, dried over sodium sulfate, and concentrated under reduced pressure. The crude product was purified by column chromatography using a gradient of hexanes and ethyl acetate as mobile phase to give 2-isopentyl-7-methoxy-6-vinylquinazolin-4(3H)-one (320 mg, 55% yield). LCMS (ESI) m/e 273.2 [(M+H)⁺, calcd for C₁₆H₂₁N₂O₂, 273.15]; LC/MS retention time (method A): t_{R}=1.82 min.

### Part D. 2-Isopentyl-7-methoxy-4-oxo-3,4-dihydroquinazoline-6-carbaldehyde

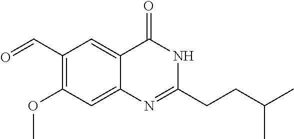 [SMILES: COc1cc2nc(CCC(C)C)[nH]c(=O)c2cc1C=O]

To a solution of 2-isopentyl-7-methoxy-6-vinylquinazolin-4(3H)-one (320 mg, 1.17 mmol) in dioxane (9.6 mL) and water (2.4 mL) was added lutidine (0.251 g, 2.350 mmol) and OsO₄ (0.28 mL, 0.023 mmol). The mixture was stirred for 10 min at RT and was then cooled to 0° C. NaIO₄ (1.0 g, 4.70 mmol) was then added and the reaction mixture was stirred at RT for 3 h. The reaction mixture was quenched with saturated aq. NaHCO₃ solution and extracted with ethyl acetate (3×20 mL). The combined organic layers were separated, dried over sodium sulfate, and concentrated under reduced pressure. The crude product was purified by column chromatography using a gradient of hexanes and ethyl acetate as mobile phase to give 2-isopentyl-7-methoxy-4-oxo-3,4-dihydroquinazoline-6-carbaldehyde (110 mg, 34% yield). LCMS (ESI) m/e 273.2 [(M−H)⁻, calcd for C₁₅H₁₇N₂O₃, 273.13]; LC/MS retention time (method A): t_{R}=1.56 min.

### Part E. 2-Isopentyl-7-methoxy-6-(oxazol-5-yl) quinazolin-4(3H)-one

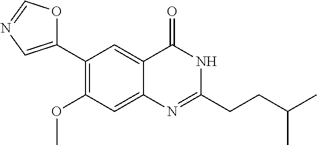 [SMILES: COc1cc2nc(CCC(C)C)[nH]c(=O)c2cc1-c1cnco1]

To a solution of 2-isopentyl-7-methoxy-4-oxo-3,4-dihydroquinazoline-6-carbaldehyde (110 mg, 0.36 mmol) in MeOH (2 mL) was added K₂CO₃ (55 mg, 0.401 mmol) and TosMIC (71 mg, 0.364 mmol). The reaction mixture was heated at 75° C. for 5 h. MeOH was removed under reduced pressure. The mixture was diluted with saturated aq. NaHCO₃ solution (25 mL) and extracted with ethyl acetate (3×25 mL). The combined organic layers were separated, dried over sodium sulfate, and concentrated under reduced pressure. The crude product was purified by preparative HPLC to give 2-isopentyl-7-methoxy-6-(oxazol-5-yl) quinazolin-4(3H)-one (50 mg, 40% yield). ¹H NMR (400 MHz, CD₃OD) δ 8.60 (s, 1H), 8.36 (s, 1H), 7.71 (s, 1H), 7.27 (s, 1H), 4.18 (s, 3H₁), 2.79-2.84 (m, 2H), 1.72-1.79 (m, 3H), 1.04 (d, J=6.4 Hz, 6H); LCMS (ESI) m/e 314.2 [(M+H)⁺, calcd for C₁₇H₂₀N₃O₃, 314.14]; LC/MS retention time (method A): t_{R}=1.59 min; HPLC retention time (method D): t_{R}=7.41 min; HPLC retention time (method C): t_{R}=8.09 min.

### Example 59

### 2-Isopentyl-N,N-dimethyl-6-(pyridin-4-yl)quinazoline-4-carboxamide

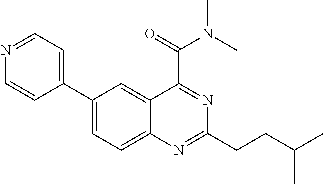 [SMILES: CC(C)CCc1nc(C(=O)N(C)C)c2cc(-c3ccncc3)ccc2n1]

### Part A. Methyl 6-bromo-2-isopentylquinazoline-4-carboxylate

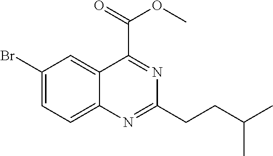 [SMILES: COC(=O)c1nc(CCC(C)C)nc2ccc(Br)cc12]

To a cooled solution of 6-bromo-2-isopentylquinazoline-4-carboxylic acid (100 mg, 0.31 mmol) (prepared as described in Example 53, Parts A-B) in diethyl ether (3 mL) at 0° C., was added TMS diazomethane (0.46 mL, 2M solution in ether) dropwise. The reaction mixture was allowed to warm to room temperature and was stirred for 4 h. LCMS analysis indicated the formation of the desired product. The reaction mixture was diluted with diethyl ether (10 mL) and was washed with water (10 mL) and brine (10 mL). The organic extract was dried over sodium sulfate, filtered, and concentrated under reduced pressure. The residue was purified by silica gel column chromatography using the gradient of ethyl acetate in hexanes to afford methyl 6-bromo-2-isopentylquinazoline-4-carboxylate (50 mg, 0.148 mmol, 48% yield) as a light brown solid. LCMS (ESI) m/e 337.2 (bromo pattern) [(M+H)⁺, calcd for C₁₅H₁₈BrN₂O₂, 337.05]; LC/MS retention time (method A): t_{R}=2.44 min.

### Part B. 6-Bromo-2-isopentyl-N,N-dimethylquinazoline-4-carboxamide

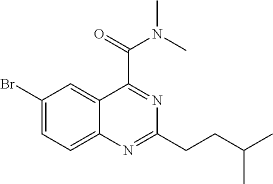 [SMILES: CC(C)CCc1nc(C(=O)N(C)C)c2cc(Br)ccc2n1]

To a stirred solution of methyl 6-bromo-2-isopentylquinazoline-4-carboxylate (300 mg, 0.89 mmol) in dry THF (2 ml) in a pressure tube, was added dimethyl amine (6.67 mL, 13.09 mmol, 2M solution in THF). The reaction vessel cap was sealed tightly and the mixture was heated at 100° C. for 18 h. The reaction mixture was allowed to cool to rt and was concentrated under reduced pressure. The residue was purified through silica gel column chromatography using gradient of ethyl acetate in hexanes as a mobile phase to afford 6-bromo-2-isopentyl-N,N-dimethylquinazoline-4-carboxamide (150 mg, 0.428 mmol, 48% yield) as an off-white solid. ¹H NMR (400 MHz, CDCl₃) δ 8.11 (d, J=2 Hz, 1H), 7.95 (dd, J=9.2, 2 Hz, 1H), 7.88 (d, J=8.8 Hz, 1H), 3.27 (s, 3H), 3.09-3.13 (m, 2H), 2.92 (s, 3H), 1.76-1.82 (m, 2H), 1.61-1.71 (m, 1H), 0.97-0.99 (m, 6H).

### Part C. 2-Isopentyl-N,N-dimethyl-6-(pyridin-4-yl) quinazoline-4-carboxamide

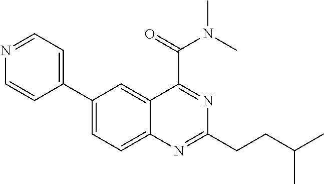 [SMILES: CC(C)CCc1nc(C(=O)N(C)C)c2cc(-c3ccncc3)ccc2n1]

Prepared as described in Example 38 from 6-bromo-2-isopentyl-N,N-dimethylquinazoline-4-carboxamide (130 mg, 0.37 mmol). The crude product was purified by silica gel column chromatography using gradient of ethyl acetate in pet ether as a mobile phase to afford 2-isopentyl-N,N-dimethyl-6-(pyridin-4-yl)quinazoline-4-carboxamide (70 mg, 0.2008 mmol, 54% yield) as an off white solid. ¹H NMR (400 MHz, CD₃OD) δ 8.69-8.70 (m, 2H), 8.42 (dd, J=8.8 Hz, 2 Hz, 2H), 8.27 (m, 1H), 8.18-8.20 (m, 2H), 7.87-7.88 (m, 2H), 3.31 (s, 3H), 3.15-3.19 (m, 2H), 2.97 (s, 3H), 1.82-1.88 (m, 2H), 1.65-1.72 (m, 1H), 1.02 (d, J=6.8 Hz, 6H); LCMS (ESI) m/e 349.2 [(M+H)⁺, calcd for C₂₁H₂₅N₄O, 349.20]; LC/MS retention time (method A): t_{R}=1.72 min; HPLC retention time (method D): t_{R}=6.78 min; HPLC retention time (method C): t_{R}=6.16 min.

### Example 60

### 2-Isopentyl-6-(pyridin-4-yl)quinazoline-4-carbonitrile

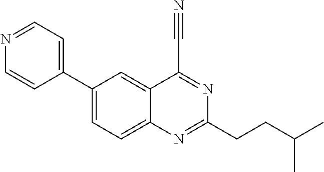 [SMILES: CC(C)CCc1nc(C#N)c2cc(-c3ccncc3)ccc2n1]

### Part A. 2-Isopentyl-6-(pyridin-4-yl) quinazoline-4-carboxamide

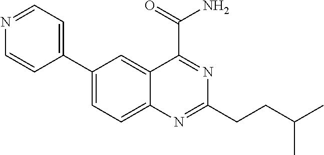 [SMILES: CC(C)CCc1nc(C(N)=O)c2cc(-c3ccncc3)ccc2n1]

Prepared in a similar fashion as described in Example 38, using 6-bromo-2-isopentylquinazoline-4-carboxamide (200 mg, 0.62 mmol) (prepared as described in Example 53, Parts A-B). The crude product was purified by silica gel column chromatography from a gradient of ethyl acetate in pet ether as the mobile phase to afford 2-isopentyl-6-(pyridin-4-yl) quinazoline-4-carboxamide (120 mg, 61% yield) as a white solid. LCMS (ESI) m/e 319.2 [(M−H)⁻, calcd for C₁₉H₁₉N₄O, 319.16]; LC/MS retention time (method A): t_{R}=1.78 min.

### Part B. 2-Isopentyl-6-(pyridin-4-yl)quinazoline-4-carbonitrile

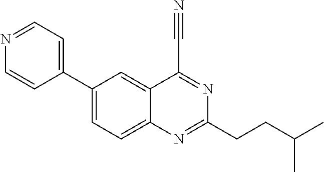 [SMILES: CC(C)CCc1nc(C#N)c2cc(-c3ccncc3)ccc2n1]

To a solution of 2-isopentyl-6-(pyridin-4-yl)quinazoline-4-carboxamide (100 mg, 0.313 mmol) in dioxane (2 mL) at 0° C. was added pyridine (0.05 mL, 0.375 mmol) followed by the slow addition of trifluoroacetic anhydride (0.05 mL, 0.625 mmol). The reaction mixture was stirred for 60 min. The reaction mixture was then warmed to room temperature and was stirred for 12 h. The pyridinium salt was removed by vacuum filtration and the filtrate was diluted with chloroform (10 mL). The organic layer was washed with water (10 ml) and brine solution (10 ml), dried over sodium sulfate, filtered, and concentrated under reduced pressure. The product was purified by preparative HPLC (10 mM ammonium acetate in water and acetonitrile) to afford 2-isopentyl-6-(pyridin-4-yl)quinazoline-4-carbonitrile (70 mg, 0.231 mmol, 72% yield) as an off white solid. ¹H NMR (400 MHz, CD₃OD) δ 8.74 (d, J=5.6 Hz, 2H), 8.50-8.53 (m, 2H), 8.24 (d, J=8.8 Hz, 1H), 7.95 (d, J=6 Hz, 2H), 3.2 (t, J=8 Hz, 2H), 1.83-1.89 (m, 2H), 1.65-1.75 (m, 1H), 1.03 (d, J=6.8 Hz, 6H); LCMS (ESI) m/e 303.0 [(M+H)⁺, calcd for C₁₉H₁₉N₄, 303.15]; LC/MS retention time (method F): t_{R}=1.78 min; HPLC retention time (method C): t_{R}=8.56 min; HPLC retention time (method D): t_{R}=8.65 min.

### Example 61

### (2-Isopentyl-6-(pyridin-4-yl)-3,4-dihydroquinazolin-4-yl)methanol

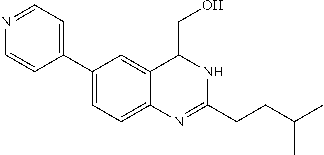 [SMILES: CC(C)CCC1=Nc2ccc(-c3ccncc3)cc2C(CO)N1]

### Part A. (6-Bromo-2-isopentyl-3,4-dihydroquinazolin-4-yl)methanol

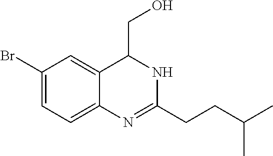 [SMILES: CC(C)CCC1=Nc2ccc(Br)cc2C(CO)N1]

To a suspension of NaBH₄ (0.2 g, 5.34 mmol) in THF (1.5 mL) was added a solution of methyl 6-bromo-2-isopentylquinazoline-4-carboxylate (0.3 g, 0.8 mmol) (prepared as described previously in Example 59, Part A) in THF (1.5 mL). To this mixture was added MeOH (1.3 mL) and the mixture was heated at 70° C. for 2 h. The reaction mixture was cooled to room temperature and was quenched with satd. aq. ammonium chloride solution and stirred for 1.5 h. The reaction mixture was filtered and the filtrate was extracted with ethyl acetate (2×10 mL). The combined organic extracts were washed with brine (10 mL), dried over sodium sulfate, and concentrated under reduced pressure to afford (6-bromo-2-isopentyl-3,4-dihydroquinazolin-4-yl)methanol (0.13 g, 52% yield) as an off-white solid. LCMS (ESI) m/e 309.2 [(M−H)⁻, calcd for C₁₄H₁₈BrN₂O, 309.1]; LC/MS retention time (method A): t_{R}=1.24 min.

### Part B. (2-Isopentyl-6-(pyridin-4-yl)-3,4-dihydroquinazolin-4-yl)methanol

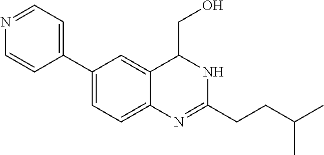 [SMILES: CC(C)CCC1=Nc2ccc(-c3ccncc3)cc2C(CO)N1]

Prepared as described previously in Example 38, using (6-bromo-2-isopentyl-3,4-dihydroquinazolin-4-yl)methanol (0.227 g, 0.7 mmol) to afford (2-isopentyl-6-(pyridin-4-yl)-3,4-dihydroquinazolin-4-yl)methanol (60 mg, 28% yield) as a white solid. ¹H NMR (400 MHz, CD₃OD): δ 8.74 (d, J=6.4 Hz, 2H), 8.03 (d, J=6.0 Hz, 2H), 7.93 (m, 1H), 7.86 (s, 1H), 7.27 (d, J=8.0 Hz, 1H), 5.06 (m, 1H), 3.92-3.84 (m, 2H), 2.72-2.67 (m, 2H), 1.76-1.73 (m, 3H), 1.05 (m, 6H); LCMS (ESI) m/e 310.2 [(M+H)⁺, calcd for C₁₉H₂₄N₃O, 310.2]; LC/MS retention time (method A): t_{R}=1.14 min; HPLC retention time (method B): t_{R}=7.59 min; HPLC retention time (method A): t_{R}=7.06 min.

### Example 62

### 1-(4-Ethyl-6-(pyridin-4-yl)quinazolin-2-yl)-3-methylbutan-1-amine

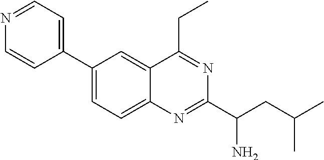 [SMILES: CCc1nc(C(N)CC(C)C)nc2ccc(-c3ccncc3)cc12]

### Part A. Benzyl 1-(6-bromo-4-ethylquinazolin-2-yl)-3-methylbutylcarbamate

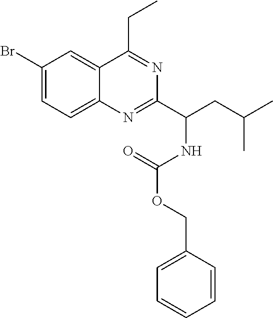 [SMILES: CCc1nc(C(CC(C)C)NC(=O)OCc2ccccc2)nc2ccc(Br)cc12]

Prepared as described in Example 6, Parts A-C to give benzyl 1-(6-bromo-4-ethylquinazolin-2-yl)-3-methylbutylcarbamate (450 mg, 91% purity by UV). LCMS (ESI) m/e 455.8 [(M+H)⁺, calcd for C₂₃H₂₇BrN₃O₂, 456.1]; LC/MS retention time (method H): t_{R}=2.32 min.

### Part B. Benzyl 1-(4-ethyl-6-(pyridin-4-yl)quinazolin-2-yl)-3-methylbutylcarbamate

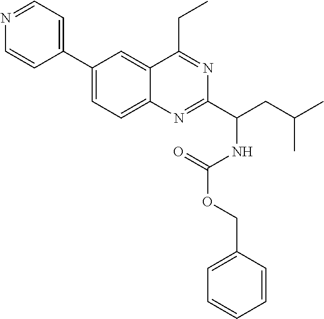 [SMILES: CCc1nc(C(CC(C)C)NC(=O)OCc2ccccc2)nc2ccc(-c3ccncc3)cc12]

Prepared in a similar fashion as described in Example 38, using benzyl 1-(6-bromo-4-ethylquinazolin-2-yl)-3-methylbutylcarbamate (200 mg, 0.438 mmol) to give benzyl 1-(4-ethyl-6-(pyridin-4-yl)quinazolin-2-yl)-3-methylbutylcarbamate (123 mg, 0.271 mmol, 62% yield). LCMS (ESI) m/e 455.2 [(M+H)⁺, calcd for C₂₈H₃₁N₄O₂, 455.24]; LC/MS retention time (method B): t_{R}=1.88 min.

### Part C. 1-(4-Ethyl-6-(pyridin-4-yl)quinazolin-2-yl)-3-methylbutan-1-amine

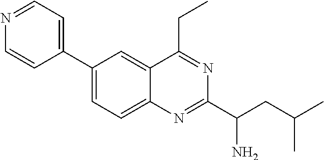 [SMILES: CCc1nc(C(N)CC(C)C)nc2ccc(-c3ccncc3)cc12]

Prepared in a similar fashion as described in Example 63, Part B, using benzyl 1-(4-ethyl-6-(pyridin-4-yl)quinazolin-2-yl)-3-methylbutylcarbamate (100 mg, 0.220 mmol) to give 1-(4-ethyl-6-(pyridin-4-yl)quinazolin-2-yl)-3-methylbutan-1-amine (30 mg, 38% yield) as a yellow solid which was isolated as an HCl salt. The crude product was purified by preparative HPLC (0.1% HCl in water and acetonitrile). ¹H NMR (400 MHz, CD₃OD) δ 8.97-9.02 (m, 3H), 8.66 (d, J=6.4 Hz, 2H), 8.58 (dd, J=8.88 Hz, 1.6 Hz, 1H), 8.31 (d, J=8.8 Hz, 1H), 4.73 (t, J=6.8 Hz, 1H), 2.06-2.13 (m, 1H), 1.89-1.96 (m, 1H), 1.79-1.86 (m, 1H), 1.55 (t, J=7.2 Hz, 3H), 1.00-1.15 (m, 6H); LCMS (ESI) m/e 321.2 [(M+H)⁺, calcd for C₂₀H₂₅N₄, 321.2]; LC/MS retention time (method B): t_{R}=1.21 min; HPLC retention time (method A): t_{R}=7.79 min.

### Example 63

### 1-(4-Ethyl-6-(1H-pyrazol-4-yl)quinazolin-2-yl)-3-methylbutan-1-amine

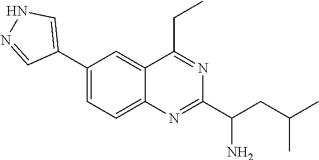 [SMILES: CCc1nc(C(N)CC(C)C)nc2ccc(-c3cn[nH]c3)cc12]

### Part A. Benzyl 1-(4-ethyl-6-(1H-pyrazol-4-yl) quinazolin-2-yl)-3-methylbutylcarbamate

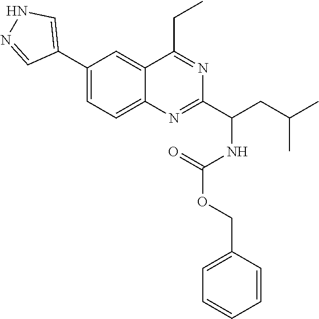 [SMILES: CCc1nc(C(CC(C)C)NC(=O)OCc2ccccc2)nc2ccc(-c3cn[nH]c3)cc12]

Prepared in a similar fashion as described in Example 37, Part F, using benzyl 1-(6-bromo-4-ethylquinazolin-2-yl)-3-methylbutylcarbamate (200 mg, 0.438 mmol) (prepared as described in Example 6, Parts A-C) and N-Boc-4-pyrazole boronate ester to afford benzyl 1-(4-ethyl-6-(1H-pyrazol-4-yl) quinazolin-2-yl)-3-methylbutylcarbamate (116 mg, 0.262 mmol, 60% yield) as a light brown solid. LCMS (ESI) m/e 444.2 [(M+H)⁺, calcd for C₂₆H₃₀N₅O₂, 444.23]; LC/MS retention time (method B): t_{R}=2.07 min.

### Part B. 1-(4-Ethyl-6-(1H-pyrazol-4-yl) quinazolin-2-yl)-3-methylbutan-1-amine

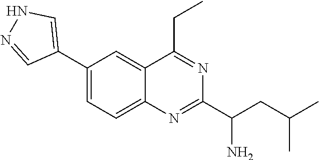 [SMILES: CCc1nc(C(N)CC(C)C)nc2ccc(-c3cn[nH]c3)cc12]

To a solution of benzyl 1-(4-ethyl-6-(1H-pyrazol-4-yl) quinazolin-2-yl)-3-methylbutylcarbamate (100 mg, 0.225 mmol) in acetic acid (4 ml) was added HBr in acetic acid (0.8 ml) at 0° C. The reaction mixture was warmed to RT and was stirred for 30 min. The reaction was worked up by washing with MTBE (10 mL). The mixture was concentrated and the crude product was purified by preparative HPLC (0.1% HCl in water and acetonitrile) to afford 1-(4-ethyl-6-(1H-pyrazol-4-yl) quinazolin-2-yl)-3-methylbutan-1-amine (20 mg, 0.064, 25% yield) as yellow solid, which was isolated as the HCl salt. ¹H NMR (400 MHz, CD₃OD) δ 8.51 (d, J=1.6 Hz, 1H), 8.37 (s, 2H), 8.31 (dd, J=8.8 Hz, 2.0 Hz, 1H), 8.08 (d, J=8.8 Hz, 1H), 4.65 (t, J=7.2 Hz, 1H), 3.46-3.52 (m, 2H), 2.04-2.11 (m, 1H), 1.86-1.93 (m, 1H), 1.76-1.82 (m, 1H), 1.45-1.55 (m, 3H), 1.00-1.15 (m, 6H); LCMS (ESI) m/e 310.2 [(M+H)⁺, calcd for C₁₈H₂₄N₅, 310.20]; LC/MS retention time (method B): t_{R}=1.64 min; HPLC retention time (method D): t_{R}=6.03 min; HPLC retention time (method C): t_{R}=5.42 min.

### Example 64

### 1-(4-Ethyl-6-(pyrimidin-4-yl)quinazolin-2-yl)-3-methylbutan-1-amine

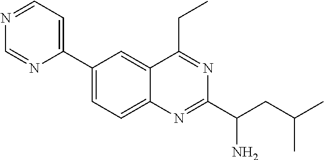 [SMILES: CCc1nc(C(N)CC(C)C)nc2ccc(-c3ccncn3)cc12]

### Part A. Benzyl (1-(4-ethyl-6-(4,4,5,5-tetramethyl-1,3,2-dioxaborolan-2-yl)quinazolin-2-yl)-3-methylbutyl)carbamate

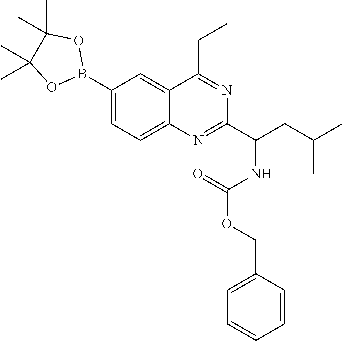 [SMILES: CCc1nc(C(CC(C)C)NC(=O)OCc2ccccc2)nc2ccc(B3OC(C)(C)C(C)(C)O3)cc12]

Prepared as described in Example 66, Part A, using benzyl 1-(6-bromo-4-ethylquinazolin-2-yl)-3-methylbutylcarbamate (500 mg, 1.096 mmol) (prepared as described in Example 6, Parts A-C) to afford benzyl (1-(4-ethyl-6-(4,4,5,5-tetramethyl-1,3,2-dioxaborolan-2-yl)quinazolin-2-yl)-3-methylbutyl)carbamate (680 mg, 0.621 mmol, 57% yield, 46% purity by UV) as a black solid. LCMS (ESI) m/e 504.2 [(M+H)⁺, calcd for C₂₉H₃₉BN₃O₄, 504.3]; LC/MS retention time (method C): t_{R}=2.48 min.

### Part B. Benzyl (1-(4-ethyl-6-(pyrimidin-4-yl)quinazolin-2-yl)-3-methylbutyl)carbamate

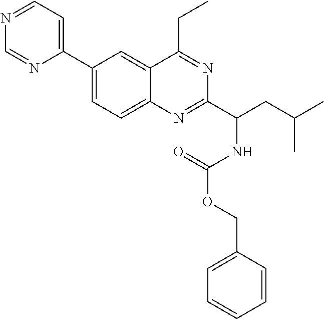 [SMILES: CCc1nc(C(CC(C)C)NC(=O)OCc2ccccc2)nc2ccc(-c3ccncn3)cc12]

To a stirred solution of benzyl (1-(4-ethyl-6-(4,4,5,5-tetramethyl-1,3,2-dioxaborolan-2-yl)quinazolin-2-yl)-3-methylbutyl)carbamate (500 mg, 0.994 mmol) in DMF (2 mL) was added a solution of 4-chloropyrimidine (111 mg, 0.97 mmol) in DMF (3 mL). To this mixture potassium phosphate (632 mg, 2.98 mmol) and Pd(PPh₃)₄ (91 mg, 0.078 mmol) were added, and nitrogen gas was bubbled through the reaction mixture for several minutes. The reaction mixture was heated at 95° C. overnight. The reaction mixture was diluted with water (20 mL) and filtered through a Celite plug. The plug was washed with excess ethyl acetate and the filtrate was extracted with ethyl acetate (2×20 mL). The combined organic extracts were dried over sodium sulfate, and the volatiles were evaporated to dryness under reduced pressure. The residue was purified by silica gel column chromatography using a gradient of ethyl acetate in hexanes. The required fractions were collected and concentrated to afford benzyl (1-(4-ethyl-6-(pyrimidin-4-yl)quinazolin-2-yl)-3-methylbutyl)carbamate (40 mg, 0.088 mmol, 9% yield) as a brown solid. LCMS (ESI) m/e 456.2 [(M+H)⁺, calcd for C₂₇H₃₀N₅O₂, 456.2]; LC/MS retention time (method C): t_{R}=2.11 min.

### Part C. 1-(4-Ethyl-6-(pyrimidin-4-yl)quinazolin-2-yl)-3-methylbutan-1-amine

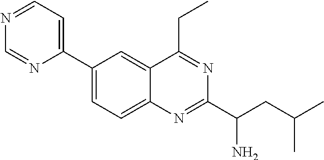 [SMILES: CCc1nc(C(N)CC(C)C)nc2ccc(-c3ccncn3)cc12]

Prepared as described in Example 63, Part B, using benzyl (1-(4-ethyl-6-(pyrimidin-4-yl)quinazolin-2-yl)-3-methylbutyl)carbamate (40 mg, 0.088 mmol) to afford 1-(4-ethyl-6-(pyrimidin-4-yl)quinazolin-2-yl)-3-methylbutan-1-amine (6 mg, 0.019 mmol, 21% yield) as a brown sticky solid as the HCl salt. The product was purified by prep. HPLC (0.1% HCl in water and acetonitrile). ¹H NMR (400 MHz, CD₃OD) δ 9.35 (s, 1H), 9.203 (d, J=1.6 Hz, 1H), 8.96 (d, J=5.2 Hz, 1H), 8.82 (dd, J=8.8, 2.0 Hz, 1H), 8.33 (d, J=1.6 Hz, 1H), 8.30 (m, 1H), 4.70 (m, 1H), 3.65-3.55 (m, 2H), 2.18-2.08 (m, 1H), 1.95-1.80 (m, 2H), 1.55 (t, J=7.6 Hz, 1H), 1.09 (d, J=6.8 Hz, 1H), 1.05 (d, J=6.8 Hz, 1H); LCMS (ESI) m/e 322.0 [(M+H)⁺, calcd for C₁₉H₂₄N₅, 322.2]; LC/MS retention time (method G): t_{R}=1.56 min. HPLC retention time (method G): t_{R}=7.32 min; HPLC retention time (method E): t_{R}=12.44 min.

### Example 65

### 4-Ethyl-2-(4-methylpentan-2-yl)-6-(pyridin-4-yl)quinazoline

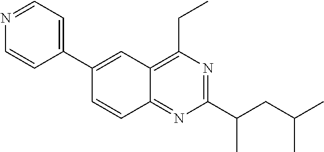 [SMILES: CCc1nc(C(C)CC(C)C)nc2ccc(-c3ccncc3)cc12]

### Part A. 2,4-Dimethylpentanoic acid

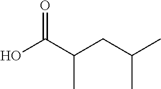

A solution of (E)-2,4-dimethylpent-2-enoic acid (100 mg, 0.780 mmol) in diethyl ether (5 mL) was purged with N₂ gas. To this solution was added platinum(IV) oxide (20 mg, 0.018 mmol) and the reaction mixture was evacuated and back-filled with hydrogen gas. The reaction mixture was stirred under hydrogen atmosphere using H₂ bladder for 4 h.

The reaction mixture was then filtered through a Celite plug and was washed with excess diethyl ether. The filtrate was concentrated under reduced pressure to afford 2,4-dimethylpentanoic acid (70 mg, 0.538 mmol, 68.9% yield). ¹H NMR (400 MHz, CDCl₃) δ 2.56-2.51 (m, 1H), 1.68-1.58 (m, 2H), 1.27-1.23 (m, 1H), 1.17 (d, J=6.8 Hz, 3H), 0.93-0.88 (m, 6H).

### Part B. N-(4-Bromo-2-propionylphenyl)-2,4-dimethylpentanamide

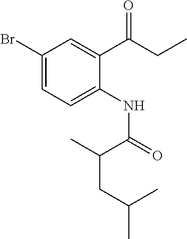 [SMILES: CCC(=O)c1cc(Br)ccc1NC(=O)C(C)CC(C)C]

To a solution of 1-(2-amino-5-bromophenyl)propan-1-one (600 mg, 2.63 mmol) (prepared in a similar fashion as described in Example 6, Part A) in pyridine (12 mL) was added 2,4-dimethylpentanoic acid (410 mg, 3.16 mmol). The mixture was cooled to −15° C. and POCl₃ (0.294 mL, 3.16 mmol) was added slowly over a period of 10 min. The reaction mixture was stirred at −15° C. for 15 min. The cold bath was then removed and the reaction mixture was allowed to warm to room temperature and was stirred for 30 min. The reaction mixture was concentrated under reduced pressure and the residue so obtained was diluted with water (15 mL). The pH of the solution was adjusted to 3 using 1.5N aq. HCl solution. The product was extracted with ethyl acetate (3×15 mL). The combined organic layers were washed with brine solution (15 mL), dried over sodium sulfate, and concentrated under reduced pressure to afford N-(4-bromo-2-propionylphenyl)-2,4-dimethylpentanamide (840 mg, 2.469 mmol, 94% yield). LCMS (ESI) m/e 340.0 [(M+H)⁺, calcd for C₁₆H₂₃BrNO₂, 340.1]; LC/MS retention time (method C): t_{R}=2.43 min.

### Part C. 6-Bromo-4-ethyl-2-(4-methylpentan-2-yl)quinazoline

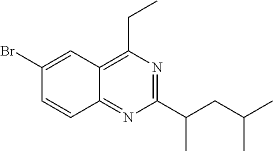 [SMILES: CCc1nc(C(C)CC(C)C)nc2ccc(Br)cc12]

To a solution of N-(4-bromo-2-propionylphenyl)-2,4-dimethylpentanamide (950 mg, 2.79 mmol) in acetic acid (20 mL) was added ammonium acetate (3.23 g, 41.9 mmol). The reaction mixture was heated at 100° C. for 12 h. The reaction mixture was then cooled to room temperature and was concentrated under reduced pressure. The residue was diluted with water. The product was extracted with ethyl acetate (3×25 mL). The combined organic layers were dried over sodium sulfate and concentrated under reduced pressure. The residue so obtained was purified by flash silica gel column chromatography using a gradient of ethyl acetate in pet ether to afford 6-bromo-4-ethyl-2-(4-methylpentan-2-yl)quinazoline (680 mg, 2.117 mmol, 76% yield). LCMS (ESI) m/e 321.2, 323.2 (bromo pattern) [(M+H)⁺, calcd for C₁₆H₂₂BrN₂, 321.1]; LC/MS retention time (method C): t_{R}=2.69 min.

### Part D. 4-Ethyl-2-(4-methylpentan-2-yl)-6-(pyridin-4-yl)quinazoline

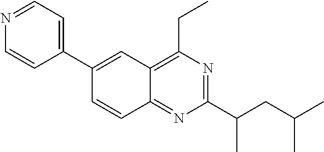 [SMILES: CCc1nc(C(C)CC(C)C)nc2ccc(-c3ccncc3)cc12]

Prepared as described in Example 38, using 6-bromo-4-ethyl-2-(4-methylpentan-2-yl)quinazoline (300 mg, 0.934 mmol). The crude product was purified by prep. HPLC (10 mM ammonium acetate in water and acetonitrile) to afford 4-ethyl-2-(4-methylpentan-2-yl)-6-(pyridin-4-yl)quinazoline (15 mg, 0.047 mmol, 5% yield) as an oil. ¹H NMR (400 MHz, CD₃OD) a 8.66 (d, J=6 Hz, 2H), 8.57 (d, J=2 Hz, 1H), 8.29 (dd, J=8.8 Hz, 2 Hz, 1H), 8.07 (d, J=8.8 Hz, 1H), 7.88-7.89 (m, 2H), 3.41-3.46 (m, 2H), 3.25-3.29 (m, 11-1), 1.93-2.00 (m, 1H), 1.36-1.55 (m, 8H), 0.93 (d, J=6.4 Hz, 3H), 0.86 (d, J=6.4 Hz, 3H); LCMS (ESI) m/e 320.2 [(M+H)⁺, calcd for C₂₁H₂₆N₃, 320.2]; LC/MS retention time (method C): t_{R}=2.27 min; HPLC retention time (method J): t_{R}=23.34 min; HPLC retention time (method M): t_{R}=17.48 min.

### Example 66

### N-(4-(4-Ethyl-2-(4-methylpentan-2-yl)quinazolin-6-yl)pyridin-2-yl)acetamide

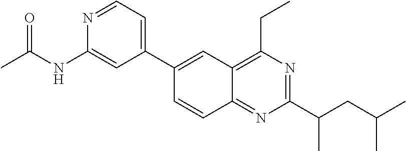 [SMILES: CCc1nc(C(C)CC(C)C)nc2ccc(-c3ccnc(NC(C)=O)c3)cc12]

### Part A. 4-Ethyl-2-(4-methylpentan-2-yl)-6-(4,4,5,5-tetramethyl-1,3,2-dioxaborolan-2-yl)quinazoline

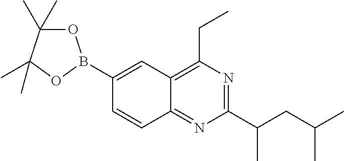 [SMILES: CCc1nc(C(C)CC(C)C)nc2ccc(B3OC(C)(C)C(C)(C)O3)cc12]

To a solution of 6-bromo-4-ethyl-2-(4-methylpentan-2-yl)quinazoline (0.477 g, 1.485 mmol) (prepared as described in Example 65, Parts A-C) and bis(pinacolato)diboron (1.131 g, 4.45 mmol) in dioxane (6 mL) was added Et₃N (0.621 mL, 4.45 mmol). Nitrogen gas was bubbled through the mixture for 10 min and [1,1-bis(diphenylphosphino)ferrocene]dichloropalladium(II) complex with dichloromethane (0.121 g, 0.148 mmol) was added. The reaction mixture was purged with nitrogen gas for 5 min and the reaction mixture was stirred at 90° C. for 14 h. The mixture was cooled to room temperature and water (20 mL) was added. The aqueous layer was extracted with ethyl acetate (3×20 mL). The combined organic layers were washed with brine solution (20 mL), dried over sodium sulfate, and concentrated under reduced pressure to afford a 4-ethyl-2-(4-methylpentan-2-yl)-6-(4,4,5,5-tetramethyl-1,3,2-dioxaborolan-2-yl)quinazoline (1.0 g, 1.222 mmol, 82% yield). The product was used without further purification in the next step. LCMS (ESI) m/e 369.4 [(M+H)⁺, calcd for C₂₂H₃₄BN₂O₂, 369.3]; LC/MS retention time (method C): t_{R}=2.80 min.

### Part B. N-(4-(4-Ethyl-2-(4-methylpentan-2-yl)quinazolin-6-yl)pyridin-2-yl)acetamide

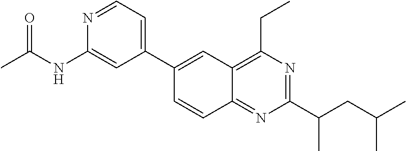 [SMILES: CCc1nc(C(C)CC(C)C)nc2ccc(-c3ccnc(NC(C)=O)c3)cc12]

Prepared in a similar fashion as described in Example 38, using 4-ethyl-2-(4-methylpentan-2-yl)-6-(4,4,5,5-tetramethyl-1,3-dioxolan-2-yl)quinazoline (0.155 g, 0.418 mmol) and N-(4-bromopyridin-2-yl)acetamide to afford N-(4-(4-ethyl-2-(4-methylpentan-2-yl)quinazolin-6-yl)pyridin-2-yl)acetamide (0.014 g, 0.037 mmol, 9% yield) as a green solid. The product was purified by reverse phase HPLC (10 mM ammonium acetate in water and acetonitrile). ¹H NMR (400 MHz, CD₃OD) δ 8.56-8.53 (m, 2H), 8.43 (d, J=5.2 Hz, 1H), 8.29 (dd, J=8.8 Hz, 1.6 Hz, 1H), 8.09 (d, J=8.8 Hz, 1H), 7.56 (dd, J=5.2 Hz, 1.6 Hz, 1H), 3.48-3.30 (m, 2H), 2.24 (s, 3H), 2.03-1.96 (m, 1H), 1.56 (d, J=6 Hz, 1H), 1.53 (d, J=7.6 Hz, 3H), 1.47-1.51 (m, 4H), 1.41-1.47 (m, 1H), 0.92-0.99 (d, J=6.4 Hz, 3H), 0.89-0.87 (d, J=6.4 Hz, 3H); LCMS (ESI) m/e 377.2 [(M+H)⁺, calcd for C₂₃H₂₉N₄O, 377.2]; LC/MS retention time (method A): t_{R}=2.29 min; HPLC retention time (method G): t_{R}=12.95 min; HPLC retention time (method N): t_{R}=10.70 min.

### Example 67

### 4-(4-Ethyl-2-(4-methylpentan-2-yl)quinazolin-6-yl)-N-methylpyridin-2-amine

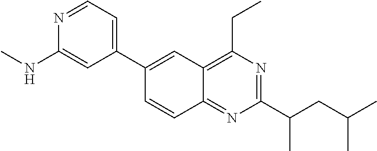 [SMILES: CCc1nc(C(C)CC(C)C)nc2ccc(-c3ccnc(NC)c3)cc12]

### Part A. tert-Butyl (4-(4-ethyl-2-(4-methylpentan-2-yl)quinazolin-6-yl)pyridin-2-yl)(methyl)carbamate

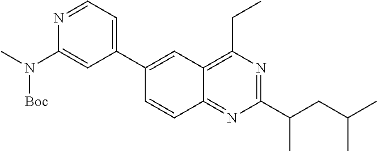 [SMILES: CCc1nc(C(C)CC(C)C)nc2ccc(-c3ccnc(N(C)C(=O)OC(C)(C)C)c3)cc12]

Prepared as described in Example 38, using 4-ethyl-2-(4-methylpentan-2-yl)-6-(4,4,5,5-tetramethyl-1,3-dioxolan-2-yl)quinazoline (0.19 g, 0.513 mmol) (prepared as described in Example 66, Part A) and tert-butyl (4-bromopyridin-2-yl)(methyl)carbamate (0.162 g, 0.564 mmol) to afford tert-butyl (4-(4-ethyl-2-(4-methylpentan-2-yl)quinazolin-6-yl)pyridin-2-yl)(methyl)carbamate (0.113 g, 0.252 mmol, 49% yield) as an oil. LCMS (ESI) m/e 449.4 [(M+H)⁺, calcd for C₂₇H₃₇N₄O₂, 449.3]; LC/MS retention time (method C): t_{R}=2.84 min.

### Part B. 4-(4-Ethyl-2-(4-methylpentan-2-yl)quinazolin-6-yl)-N-methylpyridin-2-amine

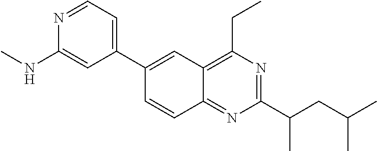 [SMILES: CCc1nc(C(C)CC(C)C)nc2ccc(-c3ccnc(NC)c3)cc12]

Prepared as described in Example 55, Part D, using tert-butyl (4-(4-ethyl-2-(4-methylpentan-2-yl)quinazolin-6-yl)pyridin-2-yl)(methyl)carbamate (0.06 g, 0.134 mmol). The crude product was purified by prep HPLC (10 mM ammonium acetate in water and acetonitrile) to afford 4-(4-ethyl-2-(4-methylpentan-2-yl)quinazolin-6-yl)-N-methylpyridin-2-amine (0.014 g, 0.039 mmol, 29.1% yield) as a light green solid. ¹H NMR (400 MHz, CD₃OD) δ 8.47 (s, 1H), 8.23 (dd, J=8.8 Hz, 1.6 Hz, 1H), 8.09-8.04 (m, 2H), 7.00 (dd, J=5.6 Hz, 2.0 Hz, 1H), 6.90 (s, 1H), 3.45 (m, 2H), 2.97 (s, 3H), 2.01-1.97 (m, 1H), 1.57-1.55 (m, 1H), 1.54-1.45 (m, 3H), 1.44-1.31 (m, 1H), 1.31-1.28 (m, 3H), 1.25 (m, 1H), 0.97-0.95 (d, J=6.8 Hz, 3H), 0.91-0.88 (d, J=6.8 Hz, 3H); LCMS (ESI) m/e 349.2 [(M+H)⁺, calcd for C₂₂H₂₉N₄, 349.2]; LC/MS retention time (method A): t_{R}=2.49 min; HPLC retention time (method N): t_{R}=11.08 min.

### Example 68

### 2-(4-Ethyl-6-(pyridin-4-yl)quinazolin-2-yl)-4-methylpentan-1-amine

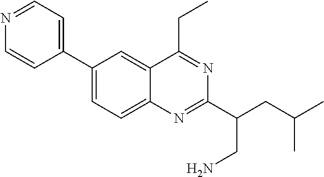 [SMILES: CCc1nc(C(CN)CC(C)C)nc2ccc(-c3ccncc3)cc12]

### Part A. 2-(Aminomethyl)-4-methylpentanoic acid

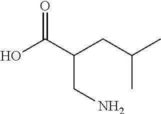

Prepared as described previously in WO2009/139834 A1, 2009.

### Part B. 2-((tert-Butoxycarbonylamino)methyl)-4-methylpentanoic acid

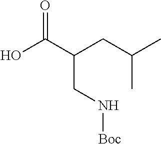 [SMILES: CC(C)CC(CNC(=O)OC(C)(C)C)C(=O)O]

Prepared as described previously (Greene, Wuts; 3rd ed., 1999, John Wiley & Sons, Inc.) from 2-(aminomethyl)-4-methylpentanoic acid hydrochloride salt (2.2 g, 15.172 mmol) to afford 2-((tert-butoxycarbonylamino)methyl)-4-methylpentanoic acid (2.5 g, 97% yield) as a pale yellow oil. The crude product was taken to next step without further purification. LCMS (ESI) m/e 244.2 [(M−H)⁻, calcd for Cl₂H₂₂NO₄, 244.16]; LC/MS retention time (method H): t_{R}=1.69 min.

### Part C. tert-Butyl 2-(4-bromo-2-propionylphenylcarbamoyl)-4-methylpentylcarbamate

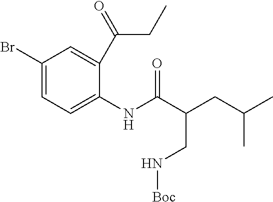 [SMILES: CCC(=O)c1cc(Br)ccc1NC(=O)C(CNC(=O)OC(C)(C)C)CC(C)C]

Prepared in a similar fashion as described in Example 8, Part B, using 1-(2-amino-5-bromophenyl)propan-1-one (453 mg, 1.98 mmol, 0.75 eq.) and 2-((tert-butoxycarbonylamino)methyl)-4-methylpentanoic acid (650 mg, 2.65 mmol, 1 eq.) to afford tert-butyl 2-(4-bromo-2-propionylphenylcarbamoyl)-4-methylpentylcarbamate (0.89 g, 48% yield) as a yellow solid. The crude product was taken to next step without further purification. LCMS (ESI) m/e 453.0 [(M−H)⁻, calcd for C₂₁H₃₀BrN₂O₄, 453.15]; LC/MS retention time (method G): t_{R}=2.29 min.

### Part D. 2-(6-Bromo-4-ethylquinazolin-2-yl)-4-methylpentan-1-amine

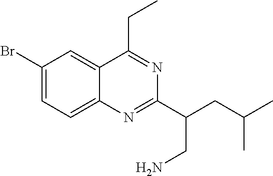 [SMILES: CCc1nc(C(CN)CC(C)C)nc2ccc(Br)cc12]

Prepared in a similar fashion as described in Example 8, Part C, using tert-butyl 2-(4-bromo-2-propionylphenylcarbamoyl)-4-methylpentylcarbamate (890 mg, 1.95 mmol) to afford 2-(6-bromo-4-ethylquinazolin-2-yl)-4-methylpentan-1-amine (200 mg, 23% yield) as a brown solid and tert-butyl 2-(6-bromo-4-ethylquinazolin-2-yl)-4-methylpentylcarbamate (300 mg, 46% yield) as light brown solid. The crude product was purified by silica gel column chromatography using pet ether:ethyl acetate mobile phase. 2-(6-Bromo-4-ethylquinazolin-2-yl)-4-methylpentan-1-amine: LCMS (ESI) m/e 336.2, 338.2 (bromo pattern) [(M+H)⁺, calcd for C₁₆H₂₃BrN₃, 336.10]; LC/MS retention time (method A): t_{R}=1.62 min. tert-Butyl 2-(6-bromo-4-ethylquinazolin-2-yl)-4-methylpentylcarbamate: LCMS (ESI) m/e 436.20, 438.2 (bromo pattern) [(M+H)⁺, calcd for C₂₁H₃₁BrN₃O₂, 436.15]; LC/MS retention time (method A): t_{R}=2.67 min.

### Part E. tert-Butyl 2-(6-bromo-4-ethylquinazolin-2-yl)-4-methylpentylcarbamate

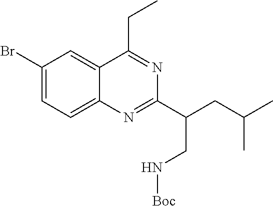 [SMILES: CCc1nc(C(CNC(=O)OC(C)(C)C)CC(C)C)nc2ccc(Br)cc12]

Prepared in a similar fashion as described in Greene, Wuts; 3rd ed., 1999, John Wiley & Sons, Inc., using 2-(6-bromo-4-ethylquinazolin-2-yl)-4-methylpentan-1-amine (200 mg, 0.595 mmol) to afford tert-butyl 2-(6-bromo-4-ethylquinazolin-2-yl)-4-methylpentylcarbamate (90 mg, 57% yield) as a light brown solid. The crude product was purified by preparative TLC using 20% ethyl acetate in pet ether as a mobile phase. LCMS (ESI) m/e 436.20, 438.2 (bromo pattern) [(M+H)⁺, calcd for C₂₁H₃₁BrN₃O₂, 436.15]; LC/MS retention time (method A): t_{R}=2.50 min.

### Part F. tert-Butyl 2-(4-ethyl-6-(pyridin-4-yl)quinazolin-2-yl)-4-methylpentylcarbamate

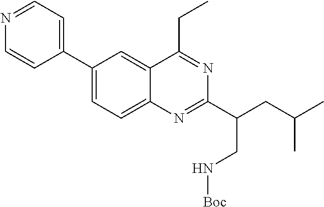 [SMILES: CCc1nc(C(CNC(=O)OC(C)(C)C)CC(C)C)nc2ccc(-c3ccncc3)cc12]

Prepared in a similar fashion as described in Example 38, using tert-butyl 2-(6-bromo-4-ethylquinazolin-2-yl)-4-methylpentylcarbamate (90 mg, 0.206 mmol) to afford tert-butyl 2-(4-ethyl-6-(pyridin-4-yl)quinazolin-2-yl)-4-methylpentylcarbamate (90 mg, quant. yield) as an off white solid. The crude product was taken to next step without further purification. LCMS (ESI) m/e 435.2 [(M+H)⁺, calcd for C₂₆H₃₅N₄O₂, 435.27]; LC/MS retention time (method A): t_{R}=2.32 min;

### Part G. 2-(4-Ethyl-6-(pyridin-4-yl)quinazolin-2-yl)-4-methylpentan-1-amine

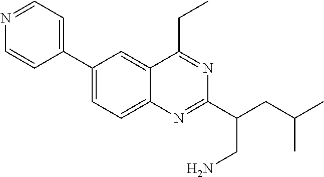 [SMILES: CCc1nc(C(CN)CC(C)C)nc2ccc(-c3ccncc3)cc12]

Prepared in a similar fashion as described in Example 55, Part D, using tert-butyl 2-(4-ethyl-6-(pyridin-4-yl)quinazolin-2-yl)-4-methylpentylcarbamate (90 mg, 0.206 mmol) to afford 2-(4-ethyl-6-(pyridin-4-yl)quinazolin-2-yl)-4-methylpentan-1-amine (30 mg, 38% yield) as yellow sticky solid which was isolated as the TFA salt. The crude product was purified by preparative HPLC using 0.05% TFA in water and acetonitrile as a mobile phase. ¹H NMR (400 MHz, MeOD) δ 8.82 (d, J=6.4 Hz, 2H), 8.77 (d, J=2 Hz, 1H), 8.44 (dd, J=8.8, 2.4 Hz, 1H), 8.20 (m, 3H), 3.61-3.51 (m, 4H), 3.39-3.37 (m, 1H), 2.05-1.88 (m, 1H), 1.65-1.56 (m, 1H), 1.55 (t, 3H), 1.03 (d, J=6.4 Hz, 3H), 0.95 (d, J=6.4 Hz, 3H); LCMS (ESI) m/e 335.2 [(M+H)⁺, calcd for C₂₁H₂₇N₄, 335.22]; LC/MS retention time (method H): t_{R}=1.67 min; HPLC retention time (method A): t_{R}=9.50 min; HPLC retention time (method B): t_{R}=10.81 min.

### Example 69

### 1-(4-Ethyl-6-(pyridin-4-yl)quinazolin-2-yl)-4,4,4-trifluorobutan-1-amine

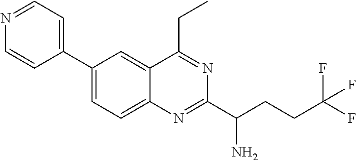 [SMILES: CCc1nc(C(N)CCC(F)(F)F)nc2ccc(-c3ccncc3)cc12]

### Part A. tert-Butyl 2-(diphenylmethyleneamino)-5,5,5-trifluoropentanoate

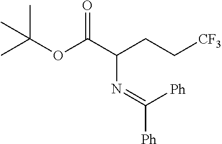 [SMILES: CC(C)(C)OC(=O)C(CCC(F)(F)F)N=C(c1ccccc1)c1ccccc1]

To a stirred solution of tert-butyl 2-((diphenylmethylene)amino)acetate (1 g, 3.39 mmol) in THF (20 mL) at −78° C. under nitrogen was added a 2M solution of LDA in THF (2.54 mL, 5.08 mmol) dropwise over 30 min. To this mixture was added 3,3,3-trifluoropropyl trifluoromethanesulfonate (1.083 g, 4.40 mmol). The reaction mixture was gradually warmed to rt and was stirred for 4 h. The reaction mixture was quenched by the addition of saturated aqueous ammonium chloride solution at 0° C. The reaction mixture was then extracted with ethyl acetate (3×10 mL). The combined organic extracts were washed with water (1×10 mL) and brine (1×10 mL), dried over sodium sulfate, and concentrated under reduced pressure. The crude oil was purified by silica gel column chromatography (2% ethyl acetate in hexane) to afford tert-butyl 2-((diphenylmethylene)amino)-5,5,5-trifluoropentanoate (800 mg, 2.02 mmol, 60% yield) as a yellow oil. LC/MS (ESI) m/e 391.9 [(M+H)⁺, calcd for C₂₂H₂₅F₃NO₂, 392.2]; LC/MS retention time (method E): t_{R}=2.49 min.

### Part B. 2-Amino-5,5,5-trifluoropentanoic acid (hydrochloride salt)

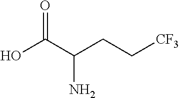 [SMILES: NC(CCC(F)(F)F)C(=O)O]

A stirred solution of tert-butyl 2-((diphenylmethylene)amino)-5,5,5-trifluoropentanoate (800 mg, 2.023 mmol) in 50% aqueous HCl (0.123 mL, 2.023 mmol) was heated at reflux at 100° C. for 8 h. The reaction mixture was cooled to rt and concentrated under reduced pressure to afford 2-amino-5,5,5-trifluoropentanoic acid hydrochloride (400 mg, 1.82 mmol, 90% yield, 78% pure by LC/MS) as a white solid. LC/MS (ESI) m/e 171.7 [(M+H)⁺, calcd for C₅H₉F₃NO₂, 172.1]; LC/MS retention time (method E): t_{R}=0.80 min.

### Part C. 2-(tert-Butoxycarbonylamino)-5,5,5-trifluoropentanoic acid

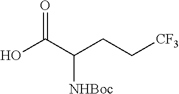 [SMILES: CC(C)(C)OC(=O)NC(CCC(F)(F)F)C(=O)O]

To a stirred solution of 2-amino-5,5,5-trifluoropentanoic acid hydrochloride (400 mg, 1.50 mmol, 78% by LC/MS) in THF (8 mL) and water (8 mL) at rt was added K₂CO₃ (831 mg, 6.01 mmol) and the solution stirred for 10 min. To this mixture was added Boc₂O (656 mg, 3.01 mmol). The reaction mixture was stirred for 8 h at rt then concentrated under reduced pressure. The aqueous layer was washed with ethyl acetate (3×5 mL). The aqueous layer was acidified with saturated citric acid solution (5 mL) and extracted with ethyl acetate (3×8 mL). The combined organic layers were washed with water (3×5 mL) followed by brine solution (1×10 mL), dried over sodium sulfate, and concentrated under reduced pressure to afford 2-((tert-butoxycarbonyl)amino)-5,5,5-trifluoropentanoic acid (500 mg, 1.84 mmol, 100% yield) as a colorless oil. The material was taken into the next step without further purification. ¹H NMR (400 MHz, CDCl₃) δ 5.04 (s, 1H), 4.38 (s, 1H), 2.15-2.28 (m, 2H), 1.91-1.95 (m, 2H), 1.46 (s, 9H).

### Part D. 1-(4-Ethyl-6-(pyridin-4-yl)quinazolin-2-yl)-4,4,4-trifluorobutan-1-amine

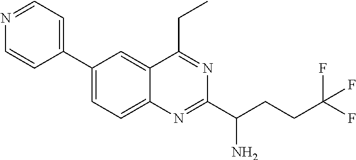 [SMILES: CCc1nc(C(N)CCC(F)(F)F)nc2ccc(-c3ccncc3)cc12]

Prepared in a similar fashion as described in Example 68, Parts C-G, using 2-((tert-butoxycarbonyl)amino)-5,5,5-trifluoropentanoic acid in Part C to give 1-(4-ethyl-6-(pyridin-4-yl)quinazolin-2-yl)-4,4,4-trifluorobutan-1-amine (35 mg) as an off-white solid. ¹H NMR (400 MHz, MeOD) δ 8.71-8.69 (m, 2H), 8.65 (d, J=2 Hz, 1H), 8.36 (dd, J=8.8, 2.0 Hz, 1H), 8.16 (d, J=8.8, 2.0 Hz, 1H), 7.94-7.93 (m, 2H), 4.26-4.23 (m, 1H), 3.50 (q, J=7.6 Hz, 2H), 2.41-2.36 (m, 3H), 2.15 (m, 1H), 1.52 (t, J=7.6 Hz, 3H); LCMS (ESI) m/e 361.2 [(M+H)⁺, calcd for C₁₉H₂₀F₃N₄, 361.16]; LC/MS retention time (method G): t_{R}=1.67 min; HPLC retention time (method B): t_{R}=9.11 min; HPLC retention time (method A): t_{R}=7.79 min.

### Example 70

### (R)-2-Cyclopropyl-1-(4-ethyl-6-(pyridin-4-yl)quinazolin-2-yl)ethanamine

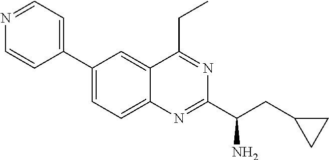 [SMILES: CCc1nc([C@H](N)CC2CC2)nc2ccc(-c3ccncc3)cc12]

Prepared in a similar fashion as described in Example 68, Parts C-G, using (R)-2-((tert-butoxycarbonyl)amino)-3-cyclopropylpropanoic acid in Part C to give (R)-2-cyclopropyl-1-(4-ethyl-6-(pyridin-4-yl)quinazolin-2-yl)ethanamine (90 mg, 69% yield) as a yellow solid which was isolated as HCl salt. The product was purified by prep HPLC using 0.1% HCl in water and acetonitrile as a mobile phase. ¹H NMR (400 MHz, MeOD) δ 9.00-8.97 (m, 3H), 8.66 (d, J=6.4 Hz, 2H), 8.58 (dd, J=8.8 Hz, 2.0 Hz, 1H), 8.31 (d, J=8.8 Hz, 1H), 4.78 (m, 1H), 3.50-3.60 (m, 2H), 2.02-2.20 (m, 2H), 1.54 (t, J=7.2 Hz, 3H), 0.84-0.89 (m, 1H), 0.51-0.55 (m, 2H), 0.1-0.14 (m, 2H); LCMS (ESI) m/e 319.2 [(M+H)⁺, calcd for C₂₀H₂₃N₄, 319.18]; LC/MS retention time (method G): t_{R}=1.56 min; HPLC retention time (method A): t_{R}=7.62 min; HPLC retention time (method B): t_{R}=8.01 min; Chiral SFC retention time (method C₃): t_{R}=2.15 min.

### Example 71

### (R)-Cyclopentyl(4-ethyl-6-(pyridin-4-yl)quinazolin-2-yl)methanamine

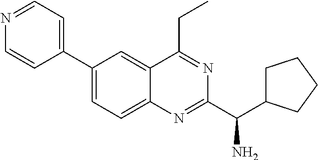 [SMILES: CCc1nc([C@H](N)C2CCCC2)nc2ccc(-c3ccncc3)cc12]

Prepared in a similar fashion as described in Example 68, Parts C-G, using (R)-2-((tert-butoxycarbonyl)amino)-2-cyclopentylacetic acid in Part C to give (R)-cyclopentyl(4-ethyl-6-(pyridin-4-yl)quinazolin-2-yl)methanamine (10 mg, 30% yield) as brown oil which was isolated as TFA salt. The crude product was purified by preparative HPLC using 0.1% TFA in water and acetonitrile as a mobile phase. ¹H NMR (400 MHz, MeOD) δ 8.83-8.80 (m, 3H), 8.48 (dd, J=8.8, 2.0 Hz, 1H), 8.26-8.20 (m, 3H), 4.51 (d, J=8.4 Hz, 1H), 3.59-3.53 (m, 2H), 2.61-2.55 (m, 1H), 1.99-1.94 (m, 1H), 1.69-1.52 (m, 10H); LCMS (ESI) m/e 333.2 [(M+H)⁺, calcd for C₂₁H₂₅N₄, 333.20]; LC/MS retention time (method A): t_{R}=1.41 min; HPLC retention time (method A): t_{R}=8.78 min; HPLC retention time (method B): t_{R}=9.45 min.

### Example 72

### (R)-Cyclohexyl(4-ethyl-6-(pyridin-4-yl)quinazolin-2-yl)methanamine

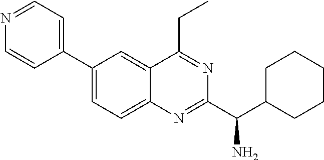 [SMILES: CCc1nc([C@H](N)C2CCCCC2)nc2ccc(-c3ccncc3)cc12]

Prepared in a similar fashion as described in Example 68, Parts C-G, using (R)-2-((tert-butoxycarbonyl)amino)-2-cyclohexylacetic acid in Part C to give (R)-cyclohexyl(4-ethyl-6-(pyridin-4-yl)quinazolin-2-yl)methanamine (10 mg, 30% yield) as brown oil which was isolated as TFA salt. The crude product was purified by preparative HPLC using 0.1% TFA in water and acetonitrile as a mobile phase. ¹H NMR (400 MHz, MeOD): δ 8.69-8.63 (m, 2H), 8.62 (s, 1H), 8.35 (dd, J=8.8, 2.0 Hz, 1H), 8.13 (d, J=8.8 Hz, 1H), 7.92 (m, 2H), 3.94 (d, J=6.4 Hz, 1H), 3.49 (q, J=7.6 Hz, 2H), 1.99-1.65 (m, 5H), 1.50 (t, J=7.6 Hz, 3H), 1.47-1.43 (m, 1H), 1.39-1.12 (m, 5H); LCMS (ESI) m/e 347.2 [(M+H)⁺, calcd for C₂₂H₂₇N₄, 347.2]; LC/MS retention time (method C): t_{R}=1.67 min; HPLC retention time (method B): t_{R}=10.15 min; HPLC retention time (method A): t_{R}=9.24 min.

### Example 73

### 1-(4-(Azetidin-1-yl)-6-(pyridin-4-yl)quinazolin-2-yl)-3-methylbutan-1-amine

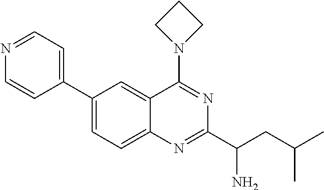 [SMILES: CC(C)CC(N)c1nc(N2CCC2)c2cc(-c3ccncc3)ccc2n1]

### Part A. tert-Butyl 3-methyl-1-(4-oxo-6-(pyridin-4-yl)-3,4-dihydroquinazolin-2-yl)butylcarbamate

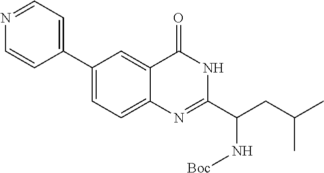 [SMILES: CC(C)CC(NC(=O)OC(C)(C)C)c1nc2ccc(-c3ccncc3)cc2c(=O)[nH]1]

Prepared in a similar fashion as described in Example 37, Parts A-D, using 2-((tert-butoxycarbonyl)amino)-4-methylpentanoic acid in Part C followed by coupling the resultant product with pyridin-4-yl boronic acid in a similar fashion as described in Example 38 to afford tert-butyl 3-methyl-1-(4-oxo-6-(pyridin-4-yl)-3,4-dihydroquinazolin-2-yl)butylcarbamate (800 mg, 38% yield). LCMS (ESI) m/e 409.2 [(M+H)⁺, calcd for C₂₃H₂₉N₄O₃, 409.22]; LC/MS retention time (method A): t_{R}=1.77 min.

### Part B. tert-Butyl 1-(4-(azetidin-1-yl)-6-(pyridin-4-yl)quinazolin-2-yl)-3-methylbutylcarbamate

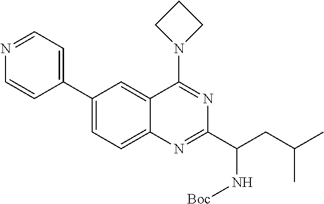 [SMILES: CC(C)CC(NC(=O)OC(C)(C)C)c1nc(N2CCC2)c2cc(-c3ccncc3)ccc2n1]

Prepared in a similar fashion as described in Example 14, Part A, using tert-butyl 3-methyl-1-(4-oxo-6-(pyridin-4-yl)-3,4-dihydroquinazolin-2-yl)butylcarbamate and azetidine to give tert-butyl 1-(4-(azetidin-1-yl)-6-(pyridin-4-yl)quinazolin-2-yl)-3-methylbutylcarbamate (100 mg). LCMS (ESI) m/e 449.0 [(M+H)⁺, calcd for C₂₆H₃₄N₅O₂, 448.26]; LC/MS retention time (method D): t_{R}=0.71 min.

### Part C. 1-(4-(Azetidin-1-yl)-6-(pyridin-4-yl)quinazolin-2-yl)-3-methylbutan-1-amine

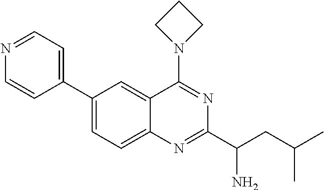 [SMILES: CC(C)CC(N)c1nc(N2CCC2)c2cc(-c3ccncc3)ccc2n1]

Prepared in a similar fashion as described in Example 55, Part D, using tert-butyl 1-(4-(azetidin-1-yl)-6-(pyridin-4-yl)quinazolin-2-yl)-3-methylbutylcarbamate to give 1-(4-(azetidin-1-yl)-6-(pyridin-4-yl)quinazolin-2-yl)-3-methylbutan-1-amine (25 mg) as pale yellow solid which was isolated as the TFA salt. The crude product was purified by preparative HPLC (0.05% TFA in water and acetonitrile). ¹H NMR (400 MHz, CD₃OD) δ 8.90 (m, 2H), 8.47-8.36 (m, 4H), 7.95 (d, J=8.8 Hz, 1H), 4.95 (m, 2H), 4.81 (m, 2H), 4.45 (m, 1H), 2.65 (m, 2H) 2.01-1.81 (m, 3H), 1.09 (m, 6H); LCMS (ESI) m/e 348.2 [(M+H)⁺, calcd for C₂₁H₂₆N₅, 348.21]; LC/MS retention time (method A): t_{R}=1.30 min; HPLC retention time (method B): t_{R}=7.44 min; HPLC retention time (method A): t_{R}=6.70 min.

### Example 74

### 1-(4-(Aziridin-1-yl)-6-(pyridin-4-yl)quinazolin-2-yl)-3-methylbutan-1-amine

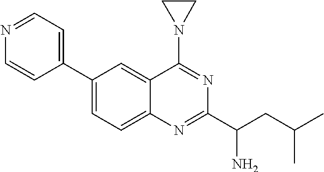 [SMILES: CC(C)CC(N)c1nc(N2CC2)c2cc(-c3ccncc3)ccc2n1]

### Part A. tert-Butyl 1-(4-(2-hydroxyethylamino)-6-(pyridin-4-yl)quinazolin-2-yl)-3-methylbutylcarbamate

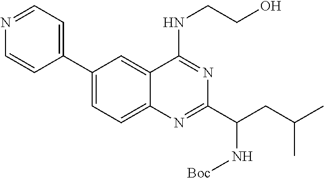 [SMILES: CC(C)CC(NC(=O)OC(C)(C)C)c1nc(NCCO)c2cc(-c3ccncc3)ccc2n1]

Prepared in a similar fashion as described in Example 13, Parts A-C, followed by the procedure in Example 23, Part A using 2-aminoethanol to give tert-butyl 1-(4-(2-hydroxyethylamino)-6-(pyridin-4-yl)quinazolin-2-yl)-3-methylbutylcarbamate (300 mg). The crude product was purified by silica gel column chromatography using pet ether: ethyl acetate mobile phase. LCMS (ESI) m/e 453.0 [(M+H)⁺, calcd for C₂₅H₃₄N₅O₃, 452.26]; LC/MS retention time (method D): t_{R}=0.67 min.

### Part B. tert-Butyl 1-(4-(aziridin-1-yl)-6-(pyridin-4-yl)quinazolin-2-yl)-3-methylbutylcarbamate

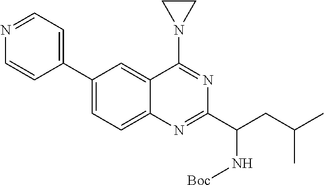 [SMILES: CC(C)CC(NC(=O)OC(C)(C)C)c1nc(N2CC2)c2cc(-c3ccncc3)ccc2n1]

A solution of PPh₃ (44 mg, 0.167 mmol) and DEAD (0.029 mL, 0.167 mmol) in anhydrous THF (1.5 mL) was cooled to 0° C. and was stirred for 30 min. The mixture was added to a solution of tert-butyl 1-(4-(2-hydroxyethylamino)-6-(pyridin-4-yl)quinazolin-2-yl)-3-methylbutylcarbamate (50 mg, 0.110 mmol) in THF (1.5 mL). The reaction mixture was warmed to rt and was heated at reflux for 4 h. The reaction mixture was allowed to cool to room temperature and was quenched by the addition of satd. aq. sodium bicarbonate solution. The product was extracted with ethyl acetate (2×10 mL). The combined organic extracts were washed with brine (10 mL), dried over sodium sulfate, and concentrated under reduced pressure. The residue was purified by silica gel column chromatography using a gradient of ethyl acetate and hexanes to afford tert-butyl 1-(4-(aziridin-1-yl)-6-(pyridin-4-yl)quinazolin-2-yl)-3-methylbutylcarbamate (30 mg, 63% yield). LCMS (ESI) m/e 434.2 [(M+H)⁺, calcd for C₂₅H₃₂N₅O₂, 434.25]; LC/MS retention time (method A): t_{R}=1.83 min.

### Part C. 1-(4-(Aziridin-1-yl)-6-(pyridin-4-yl)quinazolin-2-yl)-3-methylbutan-1-amine

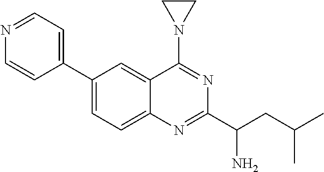 [SMILES: CC(C)CC(N)c1nc(N2CC2)c2cc(-c3ccncc3)ccc2n1]

Prepared in a similar fashion as described in Example 55, Part D, using tert-butyl 1-(4-(aziridin-1-yl)-6-(pyridin-4-yl)quinazolin-2-yl)-3-methylbutylcarbamate to give 1-(4-(aziridin-1-yl)-6-(pyridin-4-yl)quinazolin-2-yl)-3-methylbutan-1-amine (30 mg) as pale yellow solid which was isolated as the TFA salt. The crude product was purified by preparative HPLC (0.05% TFA in water and acetonitrile). ¹H NMR (400 MHz, CD₃OD): δ 8.92-8.90 (m, 2H), 8.86 (d, J=2.0 Hz, 1H), 8.62 (dd, J=8.8, 2.0 Hz, 1H), 8.27-8.25 (m, 2H), 8.18 (d, J=8.4 Hz, 1H), 5.05-4.95 (m, 1H), 4.95-4.76 (m, 2H), 4.46-4.41 (m, 2H), 1.97-1.96 (m, 3H), 1.18 (d, J=6.0 Hz, 3H), 1.11 (d, =6.0 Hz, 3H); LCMS (ESI) m/e 334.2 [(M+H)⁺, calcd for C₂₀H₂₄N₅, 334.2]; LC/MS retention time (method A): t_{R}=1.23 min; HPLC retention time (method K): t_{R}=20.05 min; HPLC retention time (method L): t_{R}=13.00 min.

### Example 75

### 5-(2-Isopentyl-7-methoxy-4-methylquinazolin-6-yl)oxazole

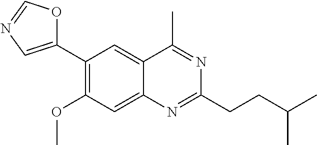 [SMILES: COc1cc2nc(CCC(C)C)nc(C)c2cc1-c1cnco1]

### Part A. 1-(2-Amino-5-bromo-4-methoxyphenyl)ethanone

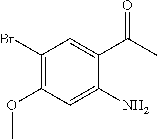

To a solution of 1.4M methyl magnesium bromide in THF (44.04 mL, 61.65 mmol) at 0° C., was added dropwise over 10 min a solution of 2-amino-5-bromo-4-methoxybenzonitrile (2.0 g, 8.80 mmol) in THF (100 mL). The cold bath was removed and the reaction mixture was warmed to RT and stirred for 5 min. The reaction mixture was heated to 55° C. for 16 h. The mixture was then cooled to 0° C. and 6N HCl (12.8 mL) was added. The mixture was stirred for 30 min. The reaction mixture was made basic by adding 10% aq. sodium bicarbonate solution. The aqueous layer was extracted with ethyl acetate (2×40 mL). The combined organic layers were washed with brine (20 mL), dried over Na₂SO₄, and concentrated under reduced pressure. The crude product was purified by column chromatography using a gradient of pet ether-ethyl acetate as mobile phase to afford 1-(2-amino-5-bromo-4-methoxyphenyl)ethanone (1.0 g, 4.09 mmol, 41% yield). LCMS (ESI) m/e 244.6 (bromo pattern) [(M+H)⁺, calcd for C₉H₁₁BrNO₂, 244.0]; LC/MS retention time (method G): t_{R}=1.65 min.

### Part B. N-(2-Acetyl-4-bromo-5-methoxyphenyl)-4-methylpentanamide

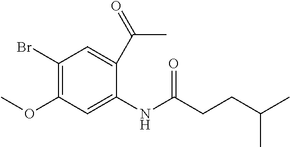 [SMILES: COc1cc(NC(=O)CCC(C)C)c(C(C)=O)cc1Br]

To a solution of 1-(2-amino-5-bromo-4-methoxyphenyl)ethanone (800 mg, 3.27 mmol) in DCM (16 mL) at 0° C. was added DIPEA (0.70 mL, 3.93 mmol) followed by 4-methylpentanoyl chloride (485 mg, 3.60 mmol). The reaction mixture was warmed to RT gradually and stirred for 16 h. The reaction mass was diluted with water (50 mL) and extracted with DCM (200 mL). The organic phase was washed with brine (50 mL), dried over Na₂SO₄, and concentrated under reduced pressure to afford N-(2-acetyl-4-bromo-5-methoxyphenyl)-4-methylpentanamide (1.12 g, 3.27 mmol, 70% yield). The product was taken to the next step without further purification. LCMS (ESI) m/e 342.2 (bromo pattern) [(M+H)⁺, calcd for C₁₅H₂₁BrNO₃, 342.06]; LC/MS retention time (method A): t_{R}=2.34 min.

### Part C. 6-Bromo-2-isopentyl-7-methoxy-4-methylquinazoline

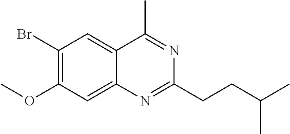 [SMILES: COc1cc2nc(CCC(C)C)nc(C)c2cc1Br]

To a solution of N-(2-acetyl-4-bromo-5-methoxyphenyl)-4-methylpentanamide (1.12 g, 3.27 mmol) in acetic acid (11 mL), was added ammonium acetate (6.31 g, 81.9 mmol) and the reaction mixture was heated to 110° C. for 12 h. The reaction completion was confirmed by LC-MS. The reaction mixture was cooled to RT and then acetic acid was removed under reduced pressure. The residue obtained was dissolved in water (100 mL) and solid sodium carbonate was added until the pH became basic. The aqueous layer was extracted with ethyl acetate (2×60 mL). The combined organic layers were washed with brine (50 mL), dried over Na₂SO₄, and concentrated under reduced pressure. The crude product was purified by column chromatography using a gradient of pet ether-ethyl acetate as the mobile phase to give 6-bromo-2-isopentyl-7-methoxy-4-methylquinazoline (710 mg, 2.196 mmol, 95% yield). LCMS (ESI) m/e 323.2 [(M+H)⁺, calcd for C₁₅H₂₀BrN₂O, 323.07]; LC/MS retention time (method A): t_{R}=2.32 min.

### Part D. 2-Isopentyl-7-methoxy-4-methyl-6-vinylquinazoline

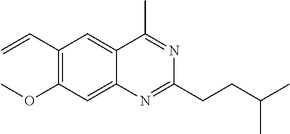 [SMILES: C=Cc1cc2c(C)nc(CCC(C)C)nc2cc1OC]

Prepared in a similar fashion as described in Example 39, Part C, using 6-bromo-2-isopentyl-7-methoxy-4-methylquinazoline (710 mg, 2.204 mmol). The crude product was purified by column chromatography using a gradient of pet ether-ethyl acetate as mobile phase to give 2-isopentyl-7-methoxy-4-methyl-6-vinylquinazoline (540 mg, 1.997 mmol, 65% yield). LCMS (ESI) m/e 271.2 [(M+H)⁺, calcd for C₁₇H₂₃N₂O, 271.17]; LC/MS retention time (method A): t_{R}=2.32 min.

### Part E. 2-Isopentyl-7-methoxy-4-methylquinazoline-6-carbaldehyde

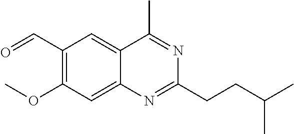 [SMILES: COc1cc2nc(CCC(C)C)nc(C)c2cc1C=O]

Prepared in a similar fashion as described in Example 39, Part D, using 2-isopentyl-7-methoxy-4-methyl-6-vinylquinazoline (540 mg, 1.99 mmol). The crude product was purified by column chromatography using a gradient of pet ether-ethyl acetate as the mobile phase to give 2-isopentyl-7-methoxy-4-methylquinazoline-6-carbaldehyde (100 mg, 0.366 mmol, 20% yield). LCMS (ESI) m/e 273.2 [(M+H)⁺, calcd for C₁₆H₂₁N₂O₂, 273.15]; LC/MS retention time (method A): t_{R}=2.00 min.

### Part F. 5-(2-Isopentyl-7-methoxy-4-methylquinazolin-6-yl)oxazole

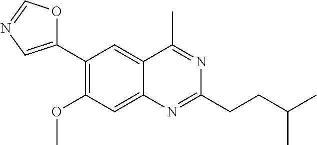 [SMILES: COc1cc2nc(CCC(C)C)nc(C)c2cc1-c1cnco1]

Prepared in a similar fashion as described in Example 39, Part E, using 2-isopentyl-7-methoxy-4-methylquinazoline-6-carbaldehyde (100 mg, 0.367 mmol). The crude product was purified by preparative TLC using 30% ethyl acetate in pet ether as the mobile phase to afford 5-(2-isopentyl-7-methoxy-4-methylquinazolin-6-yl)oxazole (30 mg, 0.096 mmol, 36% yield) as pale yellow solid. ¹H NMR (400 MHz, CD₃OD) δ 8.58 (s, 1H), 8.38 (s, 1H), 7.75 (s, 1H), 7.44 (s, 1H), 4.19 (s, 3H), 2.99-3.04 (m, 2H), 2.97 (s, 3H), 1.75-1.81 (m, 2H), 1.65-1.72 (m, 1H), 0.95-1.05 (m, 6H); LCMS (ESI) m/e 312.2 [(M+H)⁺, calcd for C₁₈H₂₂N₃O₂, 312.16]; LC/MS retention time (method B): t_{R}=1.80 min; HPLC retention time (method G): t_{R}=10.85 min; HPLC retention time (method D): t_{R}=7.62 min.

### Example 76

### 2-((2-Isopentyl-6-(oxazol-5-yl)quinazolin-4-yl)amino)ethanol

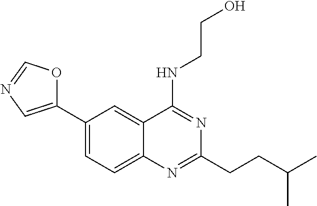 [SMILES: CC(C)CCc1nc(NCCO)c2cc(-c3cnco3)ccc2n1]

2-((2-Isopentyl-6-(oxazol-5-yl)quinazolin-4-yl)amino)ethanol was prepared as described in Example 42 followed by the method described in Example 50, using 2-aminoethanol. ¹H NMR (400 MHz. DMSO-d₆) δ 8.59 (d, J=1.6 Hz, 1H), 8.53 (s, 1H), 8.37 (bs, 1H), 8.05 (dd, J=8.4 Hz, 1.6 Hz, 1H), 7.72 (s, 1H), 7.67 (d, J=8.8 Hz, 1H), 4.83 (t, J=5.2 Hz, 1H), 3.63-3.69 (m, 4H), 2.68-2.72 (m, 2H), 1.58-1.69 (m, 3H), 0.90-1.00 (m, 6H); LCMS (ESI) m/e 327.2 [(M+H)⁺, calcd for C₁₈H₂₃N₄O₂, 327.2]; LC/MS retention time (method E): t_{R}=1.88 min.

### Example 77

### 2-Isopentyl-N-(2-methoxyethyl)-6-(oxazol-5-yl)quinazolin-4-amine

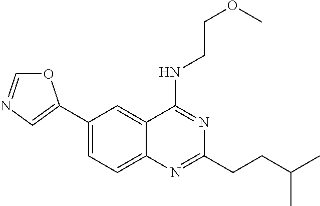 [SMILES: COCCNc1nc(CCC(C)C)nc2ccc(-c3cnco3)cc12]

2-Isopentyl-N-(2-methoxyethyl)-6-(oxazol-5-yl)quinazolin-4-amine was prepared as described in Example 42 followed by the method described in Example 50, using 2-methoxyethanamine. ¹H NMR (400 MHz, DMSO-d₆) δ 10.09 (bs, 1H), 8.8 (s, 1H), 8.63 (s, 1H), 8.30 (d, J=9.6 Hz, 1H), 7.78-7.83 (m, 2H), 3.87-3.91 (m, 2H), 3.66 (t, J=5.6 Hz, 2H), 3.33 (s, 3H), 2.83-2.87 (nm, 2H), 1.6-1.77 (m, 3H), 0.90-1.00 (m, 6H); LCMS (ESI) m/e 341.2 [(M+H)⁺, calcd for C₁₉H₂₅N₄O₂, 341.2]; LC/MS retention time (method E): t_{R}=2.33 min.

### Example 78

### 2-Isopentyl-6-(oxazol-5-yl)-N-(2-(pyrrolidin-1-yl)ethyl)quinazolin-4-amine

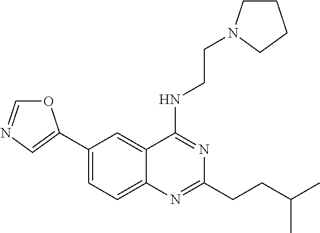 [SMILES: CC(C)CCc1nc(NCCN2CCCC2)c2cc(-c3cnco3)ccc2n1]

2-Isopentyl-6-(oxazol-5-yl)-N-(2-(pyrrolidin-1-yl)ethyl)quinazolin-4-amine was prepared as described in Example 42 followed by the method described in Example 50, using 2-(pyrrolidin-1-yl)ethanamine. ¹H NMR (400 MHz, DMSO-d₆) δ 8.55 (s, 1H), 8.520 (d, J=1.6 Hz, 1H), 8.45 (bs, 1H), 8.08 (dd, J=8.8 Hz, 2 Hz, 1H), 7.74 (s, 1H), 7.7 (d, J=8.8 Hz, 1H), 3.8 (s, 2H), 2.8-3.2 (m, 5H), 2.67-2.75 (m, 2H), 1.75-1.83 (m, 4H), 1.59-1.71 (m, 4H), 0.9-1.0 (m, 6H); LCMS (ESI) m/e 380.2 [(M+H)⁺, calcd for C₂₂H₃₀N₅O, 380.2]; LC/MS retention time (method E): t_{R}=2.60 min.

### Example 79

### 2-Isopentyl-6-(oxazol-5-yl)-N-(2-(pyrrolidin-1-yl)ethyl)quinazolin-4-amine

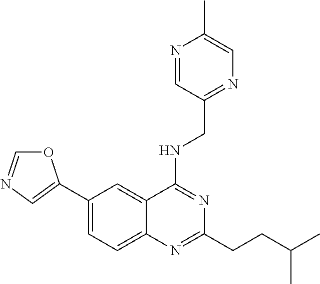 [SMILES: Cc1cnc(CNc2nc(CCC(C)C)nc3ccc(-c4cnco4)cc23)cn1]

2-Isopentyl-6-(oxazol-5-yl)-N-(2-(pyrrolidin-1-yl)ethyl)quinazolin-4-amine was prepared as described in Example 42 followed by the method described in Example 50, using (5-methylpyrazin-2-yl)methanamine. ¹H NMR (400 MHz, DMSO-d₆) δ 9.12 (bs, 1H), 8.67 (s, 1H), 8.57-8.55 (m, 2H), 8.47 (s, 1H), 8.11 (d, J=8 Hz, 1H), 7.74-7.70 (m, 2H), 4.85 (d, J=5.6 Hz, 2H), 2.68-2.63 (m, 2H), 2.46 (s, 3H), 1.50-1.40 (m, 3H), 0.82 (d, J=6.4 Hz, 6H); LCMS (ESI) m/e 389.2 [(M+H)⁺, calcd for C₂₂H₂₅N₆O, 389.2]; LC/MS retention time (method E): t_{R}=2.17 min.

### Example 80

### N¹-(2-Isopentyl-6-(oxazol-5-yl)quinazolin-4-yl)-N³,N³-dimethylpropane-1,3-diamine

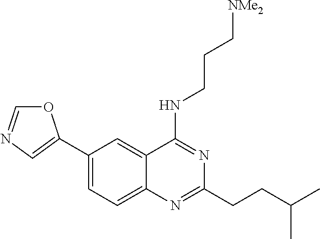 [SMILES: CC(C)CCc1nc(NCCCN(C)C)c2cc(-c3cnco3)ccc2n1]

N¹-(2-Isopentyl-6-(oxazol-5-yl)quinazolin-4-yl)-N³,N³-dimethylpropane-1,3-diamine was prepared as described in Example 42 followed by the method described in Example 50, using N¹,N¹-dimethylpropane-1,3-diamine. ¹H NMR (400 MHz, DMSO-d₆) δ 8.54 (s, 1H), 8.51 (d, J=2 Hz, 1H), 8.45 (s, 1H), 8.06 (dd, J=8.8 Hz, 2 Hz, 1H), 7.73 (s, 1H), 7.69 (d, J=8.8 Hz, 1H), 3.59-3.63 (m, 2H), 2.55-2.95 (m, 10H), 1.91-1.98 (m, 2H), 1.55-1.7 (m, 3H), 0.9-1.0 (m, 6H); LCMS (ESI) m/e 368.2 [(M+H)⁺, calcd for C₂₁H₃₀N₅O, 368.2]; LC/MS retention time (method E): t_{R}=2.49 min.

### Example 81

### N¹-(2-Isopentyl-6-(oxazol-5-yl)quinazolin-4-yl)-N²,N²-dimethylethane-1,2-diamine

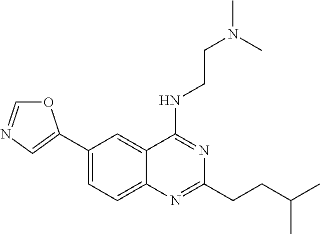 [SMILES: CC(C)CCc1nc(NCCN(C)C)c2cc(-c3cnco3)ccc2n1]

N¹-(2-Isopentyl-6-(oxazol-5-yl)quinazolin-4-yl)-N²,N²-dimethylethane-1,2-diamine was prepared as described in Example 42 followed by the method described in Example 50, using N¹,N¹-dimethylethane-1,2-diamine. ¹H NMR (400 MHz, DMSO-d₆) δ 8.52-8.53 (m, 2H), 8.32 (m, 1H), 8.05 (dd, J=8.8 Hz, 2 Hz, 1H), 7.71 (s, 1H), 7.67 (d, J=8.4 Hz, 1H), 3.66-3.7 (m, 2H), 2.71 (m, 2H), 2.51 (m, 2H), 2.27 (s, 6H), 1.71-1.59 (m, 31-H), 0.88-0.95 (m, 6H); LCMS (ESI) m/e 354.2 [(M+H)⁺, calcd for C₂₀H₂₈N₅O, 354.2]; LC/MS retention time (method E): t_{R}=1.82 min.

### Example 82

### Methyl 3-((2-isopentyl-6-(oxazol-5-yl)quinazolin-4-yl)amino)propanoate

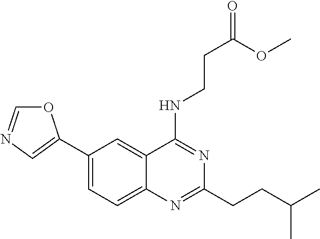 [SMILES: COC(=O)CCNc1nc(CCC(C)C)nc2ccc(-c3cnco3)cc12]

Methyl 3-((2-isopentyl-6-(oxazol-5-yl)quinazolin-4-yl)amino)propanoate was prepared as described in Example 42 followed by the method described in Example 50, using methyl 3-aminopropanoate. ¹H NMR (400 MHz, DMSO-d₆) δ 8.65 (s, 1H), 8.59 (s, 1H), 8.19 (d, J=6 Hz, 1H), 7.76 (m, 2H), 3.85-3.87 (m, 2H), 3.62 (s, 3H), 2.8 (t, J=6.8 Hz, 4H), 1.61-1.73 (m, 3H), 0.89-0.98 (m, 6H); LCMS (ESI) m/e 369.2 [(M+H)⁺, calcd for C₂₀H₂₅N₄O₃, 369.2]; LC/MS retention time (method E): t_{R}=2.35 min.

### Example 83

### 5-(4-(Azetidin-1-yl)-2-isopentylquinazolin-6-yl)oxazole

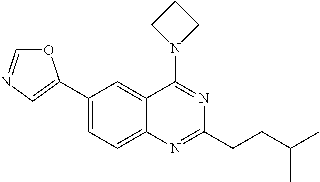 [SMILES: CC(C)CCc1nc(N2CCC2)c2cc(-c3cnco3)ccc2n1]

5-(4-(Azetidin-1-yl)-2-isopentylquinazolin-6-yl)oxazole was prepared as described in Example 42 followed by the method described in Example 50, using azetidine. ¹H NMR (400 MHz, DMSO-d₆) δ 8.48 (s, 1H), 8.09 (d, J=1.6 Hz, 1H), 8.06 (dd, J=8.8 Hz, 2 Hz, 1H), 7.8 (s, 1H), 7.7 (d, J=8.8 Hz, 1H), 4.55 (s, 4H), 2.71-2.69 (m, 2H), 2.44 (m, 2H), 1.58-1.68 (m, 3H), 0.93 (s, 6H); LCMS (ESI) m/e 323.2 [(M+H)⁺, calcd for C₁₉H₂₃N₄O, 323.2]; LC/MS retention time (method E): t_{R}=2.43 min.

### Example 84

### N-Cyclopropyl-2-isopentyl-6-(oxazol-5-yl)quinazolin-4-amine

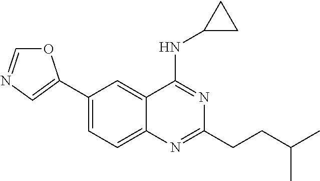 [SMILES: CC(C)CCc1nc(NC2CC2)c2cc(-c3cnco3)ccc2n1]

N-Cyclopropyl-2-isopentyl-6-(oxazol-5-yl)quinazolin-4-amine was prepared as described in Example 42 followed by the method described in Example 50, using cyclopropanamine. ¹H NMR (400 MHz, DMSO-d₆) δ 8.53-8.52 (m, 2H), 8.28 (m, 1H), 8.04 (dd, J=8.8 Hz, 2 Hz, 1H), 7.70-7.66 (m, 2H), 3.15-3.05 (m, 1H), 2.72-2.80 (m, 2H), 1.50-1.70 (m, 3H), 0.96-0.91 (m, 6H), 0.82 (m, 2H), 0.65 (m, 2H); LCMS (ESI) m/e 323.2 [(M+H)⁺, calcd for C₁₉H₂₃N₄O, 323.2]; LC/MS retention time (method E): t_{R}=2.44 min.

### Example 85

### 1-(4-Ethyl-7-methoxy-6-(oxazol-5-yl)quinazolin-2-yl)-3-methylbutan-1-amine

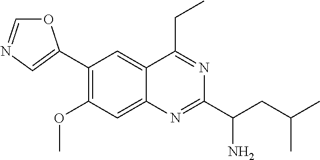 [SMILES: CCc1nc(C(N)CC(C)C)nc2cc(OC)c(-c3cnco3)cc12]

### Part A. 2-Amino-5-bromo-4-methoxybenzonitrile

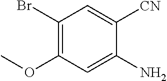

To a stirred solution of 2-amino-4-methoxy benzonitrile (19.4 g, 131 mmol) in acetonitrile (582 mL) was added NBS (18.66 g, 104 mmol) and the mixture was stirred for 1 h at 0° C. The reaction was quenched by addition of ice. The volatiles were concentrated under reduced pressure and the residue was extracted with ethyl acetate (2×200 mL). The combined organic layers were washed with brine solution (200 mL), dried over sodium sulfate and concentrated under reduced pressure to afford 2-amino-5-bromo-4-methoxy benzonitrile (26 g, 114 mmol, 87% yield) as a brown solid. LCMS (ESI) m/e 227.6 (bromo pattern) [(M+H)⁺, calcd for C₈H₈BrN₂O, 227.06]; LC/MS retention time (method C): t_{R}=1.46 min.

### Part B. 1-(2-Amino-5-bromo-4-methoxyphenyl)propan-1-one

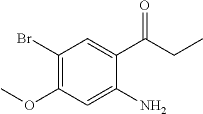

To a stirred solution of ethyl magnesium bromide (1M solution in THF, 493.3 mL, 493 mmol) in THF (400 mL) at 0° C. was added slowly 2-amino-5-bromo-4-methoxybenzonitrile (16 g, 70 mmol). After stirring for 2 h, the reaction mixture was then heated at 60° C. overnight. The reaction was quenched by addition of 6N HCl (100 mL) solution and stirred for 15 minutes. The volatiles were concentrated under reduced pressure and the residue was basified to pH 8 with saturated aq. sodium bicarbonate solution and extracted with ethyl acetate (2×250 mL). The combined organic layers were washed with brine solution (200 mL), dried over sodium sulfate and concentrated under reduced pressure. The product was purified by silica gel column chromatography using a gradient of ethyl acetate in hexanes as the mobile phase to afford 1-(2-amino-5-bromo-4-methoxyphenyl)propan-1-one (11.6 g, 44.9 mmol, 64% yield). ¹H NMR (400 MHz, DMSO-d₆) δ 7.87 (s, 1H), 7.43 (bs, 2H), 6.42 (s, 1H), 3.81 (s, 3H), 2.88 (q, J=7.2 Hz, 2H), 1.037 (t, J=7.2 Hz, 3H).

### Part C. Benzyl (1-chloro-4-methyl-1-oxopentan-2-yl)carbamate

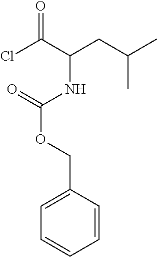 [SMILES: CC(C)CC(NC(=O)OCc1ccccc1)C(=O)Cl]

To a stirred solution of 2-(benzyloxycarbonylamino)-4-methylpentanoic acid (13.5 g, 50.88 mmol) in DCM (55 mL) at 0° C. was added 1-chloro-2-trimethyl-1-propenyl amine (8.1 mL, 61 mmol) dropwise and the reaction mixture was stirred at 0° C. for 30 min. The solution was taken to the next step without purification and analysis.

### Part D. Benzyl (1-((4-bromo-5-methoxy-2-propionylphenyl)amino)-4-methyl-1-oxopentan-2-yl)carbamate

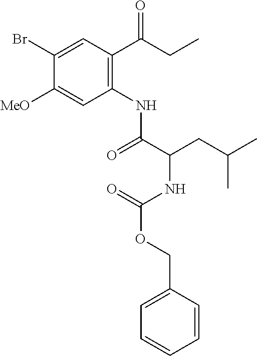 [SMILES: CCC(=O)c1cc(Br)c(OC)cc1NC(=O)C(CC(C)C)NC(=O)OCc1ccccc1]

To a solution of 1-(2-amino-5-bromo-4-methoxyphenyl)propan-1-one (11.0 g, 42.6 mmol) and DIPEA (14.8 mL, 80 mmol) in DCM (55 mL) at 0° C. was added a solution of benzyl (1-chloro-4-methyl-1-oxopentan-2-yl)carbamate (prepared as described in Part C) dropwise and the reaction was stirred at room temperature for 14 h. The reaction was quenched by the addition of water (50 mL) and extracted with DCM (3×125 mL). The combined organic extracts were dried over sodium sulfate and concentrated under reduced pressure. The residue so obtained was purified by silica gel column chromatography using a gradient of ethyl acetate in hexanes as the mobile phase to afford benzyl 1-(4-bromo-5-methoxy-2-propionylphenylamino)-4-methyl-1-oxopentan-2-ylcarbamate (27 g, 53.4 mmol) as an off-white solid. LCMS (ESI) m/e 507.1 (bromo pattern) [(M+H)⁺, calcd for C₂₄H₃₀BrN₂O₅, 505.4]; LC/MS retention time (method C): t_{R}=2.17 min.

### Part E. Benzyl 1-(6-bromo-4-ethyl-7-methoxyquinazolin-2-yl)-3-methylbutylcarbamate

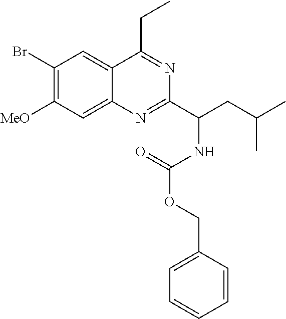 [SMILES: CCc1nc(C(CC(C)C)NC(=O)OCc2ccccc2)nc2cc(OC)c(Br)cc12]

To a solution of benzyl 1-(4-bromo-5-methoxy-2-propionylphenylamino)-4-methyl-1-oxopentan-2-ylcarbamate (27 g, 53 mmol) in acetic acid (200 mL) was added ammonium acetate (82.4 g, 1069 mmol). The reaction mixture was heated at 95° C. for 14 h. The mixture was cooled to room temperature and was concentrated under reduced pressure. The reaction mixture was transferred to a separatory funnel containing saturated aqueous NaHCO₃ solution (200 mL). The aqueous layer was extracted with ethyl acetate (3×400 mL). The combined organic layers were washed with brine (500 mL), dried over MgSO₄, filtered and concentrated. The residue was purified by column chromatography on silica gel (30%-60% ethyl acetate in hexanes) to afford benzyl 1-(6-bromo-4-ethyl-7-methoxyquinazolin-2-yl)-3-methylbutylcarbamate (12 g, 24.74 mmol) as an off-white solid. LCMS (ESI) m/e 486.3 [(M+H)⁺, calcd for C₂₄H₂₉BrN₃O₃, 486.13]; LC/MS retention time (method C): t_{R}=2.19 min.

### Part F. Benzyl 1-(4-ethyl-7-methoxy-6-vinylquinazolin-2-yl)-3-methylbutylcarbamate

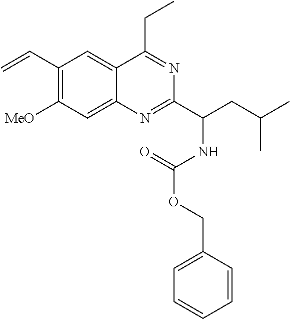 [SMILES: C=Cc1cc2c(CC)nc(C(CC(C)C)NC(=O)OCc3ccccc3)nc2cc1OC]

To a solution of benzyl 1-(6-bromo-4-ethyl-7-methoxyquinazolin-2-yl)-3-methylbutylcarbamate (7.35 g, 15.1 mmol) in 1,4-dioxane (73.5 mL) and water (7.3 mL) was added 2,4,6-trivinyl-1,3,5,2,4,6-trioxatriborinane pyridine complex (7.28 g, 30 mmol) and cesium carbonate (14.79 g, 45.0 mmol). The solution was purged with N₂ for 10 min. Pd(PPh₃)₄ (0.784 g, 0.75 mmol) was added to the reaction mixture and the mixture was heated at 100° C. overnight. The reaction mixture was cooled to room temperature and was transferred to a separatory funnel containing saturated aqueous NaHCO₃ solution (75 mL). The aqueous layer was extracted with ethyl acetate (3×100 mL). The combined organic layers were washed with brine (50 mL), dried over MgSO₄, filtered and concentrated. The residue was purified by column chromatography on silica gel (40%-60% ethyl acetate in hexanes) to afford benzyl 1-(4-ethyl-7-methoxy-6-vinylquinazolin-2-yl)-3-methylbutylcarbamate (4.7 g, 10.85 mmol, 72% yield). LCMS (ESI) m/e 434.0 [(M+H)⁺, calcd for C₂₆H₃₂N₃O₃, 434.2]; LC/MS retention time (method C): t_{R}=2.2 min.

### Part G. Benzyl 1-(4-ethyl-6-formyl-7-methoxyquinazolin-2-yl)-3-methylbutylcarbamate

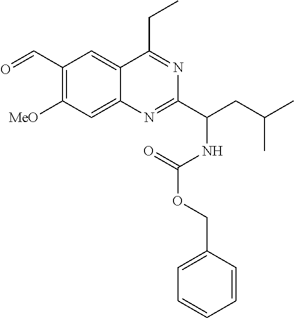 [SMILES: CCc1nc(C(CC(C)C)NC(=O)OCc2ccccc2)nc2cc(OC)c(C=O)cc12]

A stirred solution of benzyl 1-(4-ethyl-7-methoxy-6-vinylquinazolin-2-yl)-3-methylbutylcarbamate (4.7 g, 10 mmol) in dioxane (90 mL) and water (21 mL) was cooled to 0° C. To this solution, 2, 6-lutidine (2.5 mL, 21 mmol), osmium tetroxide (0.06 mL, 0.2 mmol) and sodium periodate (9.2 g, 43 mmol) were added. After 5 min, the reaction mixture was warmed to room temperature and was stirred for 3 h.

The reaction was quenched by addition of saturated aqueous sodium bicarbonate solution (100 mL). The product was extracted with ethyl acetate (2×100 mL). The combined ethyl acetate layers were washed with brine solution (100 mL), dried over sodium sulfate, filtered and concentrated. The crude product was purified by column chromatography using pet ether/ethyl acetate mobile phase to afford benzyl 1-(4-ethyl-6-formyl-7-methoxyquinazolin-2-yl)-3-methylbutylcarbamate (3.7 g, 8.50 mmol, 85%). LCMS (ESI) m/e 436.0 [(M+H)⁺, calcd for C₂₅H₃₀N₃O₄, 436.2]; LC/MS retention time (method C): t_{R}=2.04 min.

### Part H. Benzyl 1-(4-ethyl-7-methoxy-6-(oxazol-5-yl)quinazolin-2-yl)-3-methylbutylcarbamate

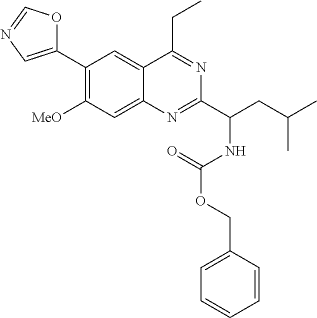 [SMILES: CCc1nc(C(CC(C)C)NC(=O)OCc2ccccc2)nc2cc(OC)c(-c3cnco3)cc12]

To a stirred solution of benzyl 1-(4-ethyl-6-formyl-7-methoxyquinazolin-2-yl)-3-methylbutylcarbamate (3.7 g, 8 mmol) in methanol (70 mL) was added TosMIC (1.6 g, 8.5 mmol) and potassium carbonate (1.29 g, 9.3 mmol). The reaction mixture was heated at 75° C. for 3 h. The reaction mixture was concentrated under reduced pressure. To the residue 10% aqueous sodium bicarbonate solution (50 mL) was added. The product was extracted with ethyl acetate (3×50 mL). The combined ethyl acetate layers were dried over sodium sulfate, filtered and concentrated under reduced pressure. The crude product was purified by preparative TLC using 40% ethyl acetate in pet ether as a mobile phase to give benzyl 1-(4-ethyl-7-methoxy-6-(oxazol-5-yl)quinazolin-2-yl)-3-methylbutylcarbamate (3.2 g, 6.75 mmol, 84% yield). LCMS (ESI) m/e 475.2 [(M+H)⁺, calcd for C₂₇H₃₁N₄O₄, 475.2]; LC/MS retention time (method F): t_{R}=1.82 min.

### Part I. 1-(4-Ethyl-7-methoxy-6-(oxazol-5-yl) quinazolin-2-yl)-3-methylbutan-1-amine hydrobromide salt

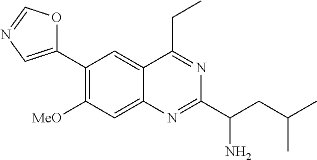 [SMILES: CCc1nc(C(N)CC(C)C)nc2cc(OC)c(-c3cnco3)cc12]

To a solution of benzyl 1-(4-ethyl-7-methoxy-6-(oxazol-5-yl) quinazolin-2-yl)-3-methylbutylcarbamate (200 mg, 0.4 mmol) in DCM (5 mL) was added HBr in acetic acid (0.4 ml) at 0° C. The reaction mixture was allowed to warm to room temperature and was stirred for 1 h. To the mixture was added ether (20 mL) and the solid obtained was washed with ether twice then dried under vacuum to afford 1-(4-ethyl-7-methoxy-6-(oxazol-5-yl) quinazolin-2-yl)-3-methylbutan-1-amine hydrobromide salt (120 mg, 0.29 mmol, 71% yield). LCMS (ESI) m/e 341.2 [(M+H)⁺, calcd for C₁₉H₂₅N₄O₂, 341.2]; LC/MS retention time (method B): t_{R}=1.71 min.

### Example 86

### 1-(4-Ethyl-7-methoxy-6-(oxazol-5-yl) quinazolin-2-yl)-3-methylbutan-1-amine (Enantiomer 1)

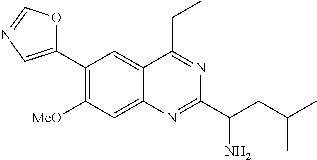 [SMILES: CCc1nc(C(N)CC(C)C)nc2cc(OC)c(-c3cnco3)cc12]

### Part A. tert-Butyl 1-(4-ethyl-7-methoxy-6-(oxazol-5-yl)quinazolin-2-yl)-3-methylbutylcarbamate

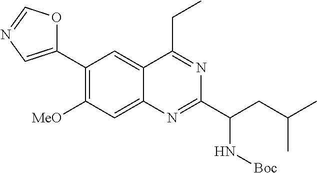 [SMILES: CCc1nc(C(CC(C)C)NC(=O)OC(C)(C)C)nc2cc(OC)c(-c3cnco3)cc12]

To a stirred solution of 1-(4-ethyl-7-methoxy-6-(oxazol-5-yl)quinazolin-2-yl)-3-methylbutan-1-amine HBr salt (0.128 g, 0.3 mmol) in THF (4 mL) was added DIPEA (0.3 mL, 1.8 mmol) at 0° C. and the mixture was stirred for 15 minutes. Boc anhydride (0.102 g, 0.4 mmol) was added and the reaction was stirred at room temperature for 15 h. The reaction mixture was concentrated under reduced pressure.

To the residue was added water and the mixture was extracted with ethyl acetate (2×20 mL). The combined organic extracts were dried over sodium sulfate and concentrated under reduced pressure. The product was purified by prep TLC using 5% methanol in DCM as a mobile phase to afford tert-butyl 1-(4-ethyl-7-methoxy-6-(oxazol-5-yl)quinazolin-2-yl)-3-methylbutylcarbamate (0.092 g, 0.21 mmol, 70% yield).

The isomers were separated by SFC purification.

Column: CHIRALPAK AD H (250×30) 5 micron; Co Solvent: 20% methanol; Total flow: 100 mL; Back pressure: 100 bar; Instrument: THAR SFC 350. tert-Butyl 1-(4-ethyl-7-methoxy-6-(oxazol-5-yl)quinazolin-2-yl)-3-methylbutylcarbamate, racemate (0.2 g) was subjected to SFC purification to afford tert-butyl 1-(4-ethyl-7-methoxy-6-(oxazol-5-yl)quinazolin-2-yl)-3-methylbutylcarbamate, Fraction 1 (Enantiomer 1) (0.07 g) and tert-butyl 1-(4-ethyl-7-methoxy-6-(oxazol-5-yl)quinazolin-2-yl)-3-methylbutylcarbamate, Fraction 2 (Enantiomer 2) (0.065 g). The separated enantiomers were subjected to Boc-deprotection (Example 86, Part B and Example 87).

### Part B. Enantiomer 1: 1-(4-Ethyl-7-methoxy-6-(oxazol-5-yl)quinazolin-2-yl)-3-methylbutan-1-amine

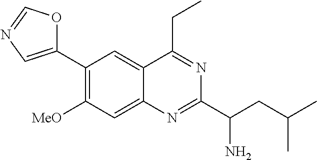 [SMILES: CCc1nc(C(N)CC(C)C)nc2cc(OC)c(-c3cnco3)cc12]

To a stirred solution of tert-butyl 1-(4-ethyl-7-methoxy-6-(oxazol-5-yl)quinazolin-2-yl)-3-methylbutylcarbamate (70 mg, 0.159 mmol) in methanol (5 mL) was added 2M HCl in diethyl ether (1.4 mL) at RT and reaction was stirred 6 h. The reaction mixture was concentrated under reduced pressure at lower temperature to obtain a solid, which was dissolved in distilled water and extracted with ethyl acetate (2×20 mL) to remove impurities. The aqueous layer was lyophilized to afford 1-(4-ethyl-7-methoxy-6-(oxazol-5-yl) quinazolin-2-yl)-3-methylbutan-1-amine hydrochloride salt (11 mg, 0.023 mmol) as a yellow solid. ¹H NMR (400 MHz, (CD₃OD) δ 8.69 (s, 1H), 8.5 (s, 1H,) 7.84 (s, 1H), 7.61 (s, 1H), 4.61 (t, J=7.2 Hz, 1H), 4.24 (s, 3H), 3.42-3.48 (m, 2H), 2.01-2.12 (m, 1H), 1.87-1.94 (m, 1H,) 1.75-1.81 (m, 1H), 1.45-1.55 (m, 3H,) 1.00-1.15 (m, 6H); LCMS (ESI) m/e 341.2 [(M+H)⁺, calcd for C₁₉H₂₅N₄O₂, 341.19]; LC/MS retention time (method A): t_{R}=1.38 min; HPLC retention time (method D): t_{R}=6.95 min; HPLC retention time (method C): t_{R}=6.15 min; Chiral SFC retention time (method A3): t_{R}=11.06 min.

### Example 87

### 1-(4-Ethyl-7-methoxy-6-(oxazol-5-yl) quinazolin-2-yl)-3-methylbutan-1-amine (Enantiomer 2)

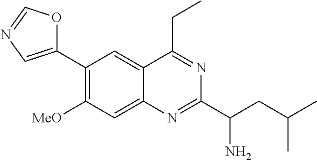 [SMILES: CCc1nc(C(N)CC(C)C)nc2cc(OC)c(-c3cnco3)cc12]

To a stirred solution of tert-butyl 1-(4-ethyl-7-methoxy-6-(oxazol-5-yl)quinazolin-2-yl)-3-methylbutylcarbamate (65 mg, 0.14 mmol) (Enantiomer 2 separated in Example 86, Part A) in methanol (5 mL) was added 2M HCl in diethyl ether (1.35 ml) at room temperature and h the reaction mixture was stirred for 6 h. The reaction mixture was concentrated under reduced pressure at lower temperature to obtain a solid, which was dissolved in distilled water and extracted with ethyl acetate (2×20 mL) to remove impurities. The aqueous layer was lyophilized to afford 1-(4-ethyl-7-methoxy-6-(oxazol-5-yl) quinazolin-2-yl)-3-methylbutan-1-amine hydrochloride salt (8 mg, 0.023 mmol) as a yellow solid. ¹H NMR (400 MHz, CD₃OD) δ 8.67 (s, 1H), 8.43 (s, 1H), 7.81 (s, 1H), 7.58 (s, 1H), 4.62 (t, J=7.6 Hz, 1H), 4.23 (s, 3H), 3.40-3.46 (m, 2H), 2.-2.10 (m, 1H), 1.76-1.93 (m, 2H), 1.45-1.55 (m, 3H), 1.00-1.11 (m, 6H); LCMS (ESI) m/e 341.2 [(M+H)⁺, calcd for C₁₉H₂₅N₄O₂, 341.19]; LC/MS retention time (method A): t_{R}=1.39 min; HPLC retention time (method C): t_{R}=6.14 min; HPLC retention time (method D): t_{R}=6.94 min; Chiral SFC retention time (method A4): t_{R}=6.83 min.

### Example 88

### 1-(4-Ethyl-7-methoxy-6-(pyridin-4-yl)quinazolin-2-yl)-3-methylbutan-1-amine

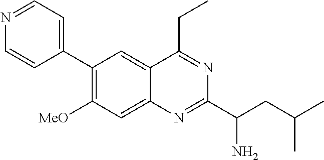 [SMILES: CCc1nc(C(N)CC(C)C)nc2cc(OC)c(-c3ccncc3)cc12]

To a stirred solution of benzyl 1-(4-ethyl-7-methoxy-6-(pyridin-4-yl)quinazolin-2-yl)-3-methylbutylcarbamate (150 mg, 0.30 mmol) (prepared in a similar fashion as described in Example 6, Parts A-D, using 2-amino-5-bromo-4-methoxybenzonitrile (Example 85, Part A) in Part A, benzyl 1-(4-bromo-2-propionylphenylamino)-4-methyl-1-oxopentan-2-ylcarbamate in Part B, and pyridin-4-yl boronic acid in Part D) in ethanol (15 mL) was added Pd/C (15 mg, 10% wt/wt). The reaction mixture was stirred at room temperature under H₂ (1 atm) for 48 h. The reaction mixture was filtered through Celite bed, 4M HCl in methanol (2 mL) was added to the filtrate and the filtrate was concentrated to dryness. The residue was dissolved in water (10 mL) and extracted with ethyl acetate (2×10 mL) to remove organic impurities. The aqueous layer was lyophilized to afford 1-(4-ethyl-7-methoxy-6-(pyridin-4-yl) quinazolin-2-yl)-3-methylbutan-1-amine (5 mg, 0.014 mmol) as a yellow solid HCl salt. ¹H NMR (400 MHz, DMSO-d₆) δ 8.96 (d, J=6.8 Hz, 2H), 8.55 (s, 1H), 8.45 (d, J=6.8 Hz, 2H), 7.65 (s, 1H), 4.65 (m, 1H), 4.12 (s, 3H), 3.47-3.36 (m, 2H), 2.03-2.10 (m, 1H), 1.94-1.88 (m, 1H), 1.81-1.78 (m, 1H), 1.47 (t, J=7.6 Hz, 3H), 0.9-1.0 (m, 6H); LCMS (ESI) m/e 351.2 [(M+H)⁺, calcd for C₂₁H₂₇N₄O, 351.21]; LC/MS retention time (method A): t_{R}=1.38 min; HPLC retention time (method A): t_{R}=8.88 min; HPLC retention time (method B): t_{R}=9.43 min.

### Methods

### AAK1 Kinase Assay

The assays were performed in U-bottom 384-well plates. The final assay volume was 30 μl prepared from 15 μl additions of enzyme and substrates (fluoresceinated peptide (5-FAM)-Aha-KEEQSQITSQVTGQIGWR-NH2 and ATP) and test compounds in assay buffer (10 mM Tris-HCL pH 7.4, 10 mM MgCl₂, 0.01% Tween-20 and 1.0 mM DTT). The reactions were initiated by the combination of bacterially expressed, GST-Xa-hAAK1 with substrates and test compounds. The reactions were incubated at room temperature for 3 hours and terminated by adding 60 μl of 35 mM EDTA buffer to each sample. The reactions were analyzed on the Caliper LabChip 3000 (Caliper, Hopkinton, Mass.) by electrophoretic separation of the fluorescent substrate and phosphorylated product. Inhibition data were calculated by comparison to EDTA quenched control reactions for 100% inhibition and vehicle-only reactions for 0% inhibition. The final concentration of reagents in the assays are ATP, 22 μM; (5-FAM)-Aha-KEEQSQITSQVTGQIGWR-NH2, 1.5 μM; GST-Xa-hAAK1, 3.5 nM; and DMSO, 1.6%. Dose response curves were generated to determine the concentration required inhibiting 50% of kinase activity (IC₅₀). Compounds were dissolved at 10 mM in dimethylsulfoxide (DMSO) and evaluated at eleven concentrations. IC₅₀ values were derived by non-linear regression analysis.

### HEK281 Cell-Based Assay

HEK293F cells were cultured in media containing DMEM (Gibco, cat. #11965), 10% FBS (SAFC Biosciences, cat. #12103C), 1×GPS (glutamine, penicillin and streptomycin). On day one, cells were plated on a 10 cm dish so that they are ˜80% confluent at time of transfection. Roughly 12 million cells were in a 10 cm dish at time of transfection. On day two, each dish was transfected with 48 ug DNA and 144 ul Lipofectamine 2000 (Invitrogen, cat.#11668-019). The DNA was comprised of a mixture (per 10 cm dish) containing 3 ug AAK1/HA/pIRES (full length human, NCBI accession no. NP_055726.2), 45 μg Flag/AP2MI/pcDNA (full length human), and 1.5 ml OPTI-MEM. The Lipofectamine 2000 is made up of a mixture (per 10 cm dish) containing 144 μl Lipofectamine 2000 and 1.5 ml OPTI-MEM. Each mixture was transferred to individual 15 ml tubes and incubated at RT for 5 minutes, and then the two mixes were combined and incubated at RT for 20 minutes. Growth media was then aspirated from each 10 cm plate and replaced with 10 ml of DMEM+10% FBS (no GPS). Finally, 3 ml DNA/Lipofectamine mix was added to each 10 cm dish and mix gently followed by incubate of plate overnight at 37° C. and 5% CO₂.

On day three, compounds were diluted in 100% DMSO at 1000× final concentration, followed by 3-fold serial dilutions for a total of 5 concentrations tested. Four compounds were tested per 10 cm dish. One ul of each compound dilution was then pipetted into a deep-well, 96-well plate, followed by addition of 500 μl DMEM+0.5% FBS into each well for a 2× final concentration of each compound. Cells were resuspended in a 10 cm dish by simple pipetting (HEK293 cells come off the plate that easy at this point) and then transferred to a 50 ml conical tube and pelleted by centrifugation at 1000 rpm for 5 min. Cell pellets were then resuspended in 2.75 ml DMEM+0.5% FBS per 10 cm dish and 100 μl of cell suspension transferred into each well of 96-well TC plate. Finally, 100 μl of 2× compound diluted in DMEM+0.5% FBS was then added into wells containing cell suspension for a 1× final concentration. Plates were then incubated at 37° C. and 5% CO₂ for 3 hours followed by transferring of cell suspensions from each well into 12-tube PCR strips. The PCR strips were spun in a tip rack at 1000 rpm for 5 minutes to pellet cells and media was then removed by pipetting without disturbing the cell pellet.

To prepare for Western Blot analysis, cell pellets were resuspend in 40 ul 1× LDS-PAGE sample buffer (Invitrogen, cat.# NP0008)+2× Halt phophatase and protease inhibitor cocktail (Thermo Scientific, cat.#1861284), followed by sonicating each with microtip sonicator set at 5 for 8-10 seconds. Five ul of 10× NuPage Sample Reducing Agent (with 50 mM DTT) was to each sample followed by heat denaturing at 70 C for 10 min on PCR machine. A total of 10 μl per sample was loaded into each lane of a 4-20% Tris-Glycine Criterion 26-well gel (Biorad, cat.#345-0034) for the phospho-mu2 blot and 10 μl per lane in a 4-12% Bis-Tris (+MES buffer) NuPAGE 26-well gel (Invitrogen, cat.# WG1403BX10) for the mu2 blot. For controls, 2 ng of phospho-mu2 or 20 ng mu2/Flag proteins were loaded in the last well of each gel. After SDS-PAGE, samples on each gel were transferred to PVDF membrane using an iBlot and membranes were blocked for one hour in TBST+5% milk, followed by wash 3× for 5-10 min with TBST. Criterion gels were probed with rabbit anti-phospho-mu2 (1:5000; a rabbit polyclonal antibody produced by New England Peptide and affinity purified at Lexicon) in TBST+5% BSA, whereas, NuPAGE gels were probed with mouse anti-Flag (1:500; Sigma, cat.# F1804) in TBST+5% milk, and these primary antibodies were incubated overnight at 4° C. on a rocker.

On day four, Western blots were washed 3× for 5-10 minutes with TBST, probe with anti-rabbit-HRP (1:2000; BioRad, cat.#170-6515) or anti-mouse-HRP (1:2000; Biorad, cat.#170-6516) in TBST+5% milk for 1 hour at RT, washed 3× for 10 minutes with TBST, and developed with ECL reagent (GE Healthcare, cat.#RPN2132) on a Versadoc. Finally, the camera was set up to take a picture every 30 seconds for 10 minutes and the best image saved for each blot with no saturated signal (when the signal is saturated, the bands will be highlighted red). A volume analysis on each band was performed to obtain density values. Percent inhibition was calculated for each sample by first normalizing to total Mu2 expression levels and then comparing to 0% and 100% controls. IC₅₀ values were then calculated using Excel fitting software. Functional potency for select compounds are listed in Table 2.

TABLE 2

| Example | IC₅₀ (nM) |
|---|---|
| 1 | 420 |
| 2 | 170 |
| 3 | 77 |
| 4 | 5,300 |
| 5 | 75 |
| 6 | 36 |
| 7 | 8 |
| 8 | 61 |
| 9 | 260 |
| 10 | 4,800 |
| 11 | 360 |
| 12 | 140 |
| 13 | 93 |
| 14 | 230 |
| 15 | 410 |
| 16 | 210 |
| 17 | 360 |
| 18 | 130 |
| 19 | 760 |
| 20 | 850 |
| 21 | 1,200 |
| 22 | 1,300 |
| 23 | 79 |
| 24 | 86 |
| 25 | 22 |
| 26 | 31 |
| 27 | 20 |
| 28 | 380 |
| 29 | 54 |
| 30 | 17 |
| 31 | 640 |
| 32 | 6 |
| 33 | 14 |
| 34 | 12 |
| 35 | 68 |
| 36 | 113 |
| 37 | 1007 |
| 38 | 1070 |
| 39 | 1136 |
| 40 | 412 |
| 41 | 3154 |
| 42 | 49 |
| 43 | 159 |
| 44 | 2009 |
| 45 | 400 |
| 46 | 1388 |
| 47 | 1335 |
| 48 | 1192 |
| 49 | 54 |
| 50 | 301 |
| 51 | 22 |
| 52 | 1272 |
| 53 | 354 |
| 54 | 2590 |
| 55 | 97 |
| 56 | 5 |
| 57 | 1862 |
| 58 | 20 |
| 59 | 2671 |
| 60 | 144 |
| 61 | 35 |
| 62 | 37 |
| 63 | 28 |
| 64 | 83 |
| 65 | 223 |
| 66 | 258 |
| 67 | 1502 |
| 68 | 144 |
| 69 | 32 |
| 70 | 126 |
| 71 | 32 |
| 72 | 13 |
| 73 | 44 |
| 74 | 263 |
| 75 | 590 |
| 76 | 1808 |
| 77 | 3458 |
| 78 | 1091 |
| 79 | 6600 |
| 80 | 3325 |
| 81 | 8608 |
| 82 | 4622 |
| 83 | 812 |
| 84 | 2578 |
| 85 | 24 |
| 86 | 87 |
| 87 | 13 |
| 88 | 932 |

1. A method for treating or managing a disease or a disorder mediated by AAK1 activity, the method comprising administering to a patient in need thereof a therapeutically effective amount of a compound of formula (I) 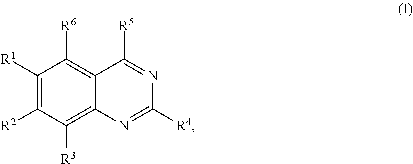
or a pharmaceutically acceptable salt thereof, wherein:
R¹ is selected from imidazopyridazine, isoquinolinyl, oxazolyl, pyridinyl, pyrimidinyl, pyrazolyl, pyrrolopyridinyl, and quinolinyl, wherein each ring is optionally substituted with C₁-C₃acylamino, C₁-C₃alkyl, amino, C₁-C₃alkoxy, C₁-C₃alkylamino, C₃-C₆cycloalkyl, C₃-C₆cycloalkylamino, C₁-C₃dialkylamino, —NHCO₂(C₁-C₃)alkyl, and phenylcarbonylamino optionally substituted with a halo or haloalkyl group;
R² is selected from hydrogen, C₁-C₃alkoxy, and C₁-C₃alkyl;
R³ is selected from hydrogen, C₁-C₃alkoxy, C₁-C₃alkyl, cyano, and halo;
R⁴ is selected from C₃-C₆alkyl optionally substituted with one or two groups independently selected from amino, haloalkyloxy, haloalkyl, hydroxy and oxo; and
C₃-C₆cycloalkylC₁-C₃alkyl optionally substituted with amino;
when 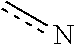
is a single bond, R⁵ is ═S or ═O.
when 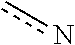
is a double bond, R⁵ is selected from hydrogen, C₁-C₆alkoxy, C₁-C₃alkoxyC₁-C₃alkyl, C₁-C₃alkoxyC₁-C₃alkylamino, C₁-C₃alkoxycarbonylC₁-C₃alkylamino, C₁-C₆alkyl, C₁-C₆alkylamino, C₁-C₆alkylsulfanyl, amido, aminoC₁-C₃alkylamino, cyano, C₃-C₆cycloalkyl, C₁-C₆dialkylamido, C₁-C₆dialkylamino, C₁-C₆dialkylaminoC₁-C₃alkylamino, halo, hydroxyC₁-C₃alkyl, hydroxyC₁-C₃alkylamino, pyrrolidinylC₁-C₃alkylamino, pyrazinylC₁-C₃alkylamino optionally substituted with methyl, and a ring selected from 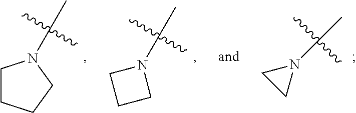
and
R⁶ is hydrogen or C₁-C₃alkoxy.

2. The method of claim 1, wherein R¹ is selected from oxazolyl, pyridinyl, and pyrazolyl, wherein each ring is optionally substituted with C₁-C₃acylamino, C₁-C₃alkoxy, and C₁-C₃alkylamino.

3. The method of claim 2 wherein R² and R³ are selected from hydrogen and C₁-C₃alkoxy.

4. The method of claim 3 wherein R⁴ is selected from C₃-C₆alkyl optionally substituted with one or two groups independently selected from amino, and haloalkyl; and C₃-C₆cycloalkylC₁-C₃alkyl optionally substituted with amino;

5. The method of claim 1, wherein the disease or disorder is selected from Alzheimer's disease, bipolar disorder, pain, Parkinson's disease, and schizophrenia.

6. The method of claim 5 wherein the pain is neuropathic pain.

7. The method of claim 6 wherein the neuropathic pain is fibromyalgia or peripheral neuropathy.

8. A method of inhibiting adaptor associated kinase 1 (AAK1) activity, comprising contacting AAK1 with a compound of formula (I) 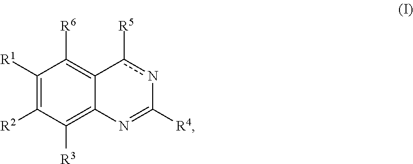
or a pharmaceutically acceptable salt thereof, wherein:
R¹ is selected from imidazopyridazine, isoquinolinyl, oxazolyl, pyridinyl, pyrimidinyl, pyrazolyl, pyrrolopyridinyl, and quinolinyl, wherein each ring is optionally substituted with C₁-C₃acylamino, C₁-C₃alkyl, amino, C₁-C₃alkoxy, C₁-C₃alkylamino, C₃-C₆cycloalkyl, C₃-C₆cycloalkylamino, C₁-C₃dialkylamino, —NHCO₂(C₁-C₃)alkyl, and phenylcarbonylamino optionally substituted with a halo or haloalkyl group;
R² is selected from hydrogen, C₁-C₃alkoxy, and C₁-C₃alkyl;
R³ is selected from hydrogen, C₁-C₃alkoxy, C₁-C₃alkyl, cyano, and halo;
R⁴ is selected from C₃-C₆alkyl optionally substituted with one or two groups independently selected from amino, haloalkyloxy, haloalkyl, hydroxy and oxo; and C₃-C₆cycloalkylC₁-C₃alkyl optionally substituted with amino;
when 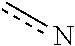
is a single bond, R⁵ is ═S or ═O.
when 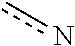
is a double bond, R⁵ is selected from hydrogen, C₁-C₆alkoxy, C₁-C₃alkoxyC₁-C₃alkyl, C₁-C₃alkoxyC₁-C₃alkylamino, C₁-C₃alkoxycarbonylC₁-C₃alkylamino, C₁-C₆alkyl, C₁-C₆alkylamino, C₁-C₆alkylsulfanyl, amido, aminoC₁-C₃alkylamino, cyano, C₃-C₆cycloalkyl, C₁-C₆dialkylamido, C₁-C₆dialkylamino, C₁-C₆dialkylaminoC₁-C₃alkylamino, halo, hydroxyC₁-C₃alkyl, hydroxyC₁-C₃alkylamino, pyrrolidinylC₁-C₃alkylamino, pyrazinylC₁-C₃alkylamino optionally substituted with methyl, and a ring selected from 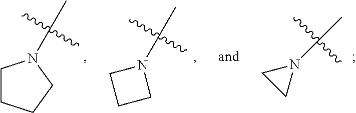
and
R⁶ is hydrogen or C₁-C₃alkoxy.
